SEQUENCE LISTING (583 sequences)

SEQ ID NO: 1 - DNA, 22 bp, synthetic construct
  misc_feature 1..22: note = Antisense oligomer
  source 1..22: mol_type = other DNA
cggtccacgtagactaacaact

SEQ ID NO: 2 - DNA, 23 bp, synthetic construct
  misc_feature 1..23: note = Antisense oligomer
  source 1..23: mol_type = other DNA
gaagttcacacagataaacttct

SEQ ID NO: 3 - DNA, 20 bp, synthetic construct
  misc_feature 1..20: note = Antisense oligomer
  source 1..20: mol_type = other DNA
cggttagaagactcatcttt

SEQ ID NO: 4 - DNA, 25 bp, synthetic construct
  misc_feature 1..25: note = Antisense oligomer
  source 1..25: mol_type = other DNA
tttcgacatcggttagaagactcat

SEQ ID NO: 5 - DNA, 22 bp, synthetic construct
  misc_feature 1..22: note = Antisense oligomer
  source 1..22: mol_type = other DNA
gagacgccatgatgtggatgtc

SEQ ID NO: 6 - DNA, 23 bp, synthetic construct
  misc_feature 1..23: note = Antisense oligomer
  source 1..23: mol_type = other DNA
gaaacacggacacccaaagtagt

SEQ ID NO: 7 - DNA, 22 bp, synthetic construct
  misc_feature 1..22: note = Antisense oligomer
  source 1..22: mol_type = other DNA
tcccagcgtcaatatgctgttt

SEQ ID NO: 8 - DNA, 20 bp, synthetic construct
  misc_feature 1..20: note = Antisense oligomer
  source 1..20: mol_type = other DNA
gcctaggatccacggtgcgc

SEQ ID NO: 9 - DNA, 40 bp, synthetic construct
  misc_feature 1..40: note = Antisense oligomer
  source 1..40: mol_type = other DNA
gggacaaaatggatcccattattaatggaaattctgctaa

SEQ ID NO: 10 - DNA, 21 bp, synthetic construct
  misc_feature 1..21: note = Antisense oligomer
  source 1..21: mol_type = other DNA
taatgggatccattttgtccc

SEQ ID NO: 11 - DNA, 23 bp, synthetic construct
  misc_feature 1..23: note = Antisense oligomer
  source 1..23: mol_type = other DNA
aataatgggatccattttgtccc

SEQ ID NO: 12 - DNA, 27 bp, synthetic construct
  misc_feature 1..27: note = Antisense oligomer
  source 1..27: mol_type = other DNA
cattaataatgggatccattttgtccc

SEQ ID NO: 13 - DNA, 30 bp, synthetic construct
  misc_feature 1..30: note = Antisense oligomer
  source 1..30: mol_type = other DNA
gaatttccattaataatgggatccattttg

SEQ ID NO: 14 - DNA, 29 bp, synthetic construct
  misc_feature 1..29: note = Antisense oligomer
  source 1..29: mol_type = other DNA
cagaatttccattaataatgggatccatt

SEQ ID NO: 15 - DNA, 25 bp, synthetic construct
  misc_feature 1..25: note = Antisense oligomer
  source 1..25: mol_type = other DNA
ggccaaacctcggcttacctgaaat

SEQ ID NO: 16 - DNA, 25 bp, synthetic construct
  misc_feature 1..25: note = Antisense oligomer
  source 1..25: mol_type = other DNA
ggccaaacctcggcttacctgaaat

SEQ ID NO: 17 - DNA, 18 bp, synthetic construct
  misc_feature 1..18: note = Antisense oligomer
  source 1..18: mol_type = other DNA
gctattaccttaacccag

SEQ ID NO: 18 - DNA, 52 bp, synthetic construct
  misc_feature 1..52: note = Antisense oligomer
  source 1..52: mol_type = other DNA
gaaaaaagattatattgattttaaaatcatgcaaaaactgcaactctgtgtt

SEQ ID NO: 19 - DNA, 25 bp, synthetic construct
  misc_feature 1..25: note = Antisense oligomer
  source 1..25: mol_type = other DNA
catacatttgcagtttttgcatcat

SEQ ID NO: 20 - DNA, 26 bp, synthetic construct
  misc_feature 1..26: note = Antisense oligomer
  source 1..26: mol_type = other DNA
tcatttttaaaaatcagcacaatctt

SEQ ID NO: 21 - DNA, 26 bp, synthetic construct
  misc_feature 1..26: note = Antisense oligomer
  source 1..26: mol_type = other DNA
cagtttttgcatcatttttaaaaatc

SEQ ID NO: 22 - DNA, 25 bp, synthetic construct
  misc_feature 1..25: note = Antisense oligomer
  source 1..25: mol_type = other DNA
gatctgtcaaatcgcctgcaggtaa

SEQ ID NO: 23 - DNA, 25 bp, synthetic construct
  misc_feature 1..25: note = Antisense oligomer
  source 1..25: mol_type = other DNA
aaactgttcagcttctgttagccac

SEQ ID NO: 24 - DNA, 25 bp, synthetic construct
  misc_feature 1..25: note = Antisense oligomer
  source 1..25: mol_type = other DNA
ttgtgtctttctgagaaactgttca

SEQ ID NO: 25 - DNA, 25 bp, synthetic construct
  misc_feature 1..25: note = Antisense oligomer
  source 1..25: mol_type = other DNA
ctgacaacagtttgccgctgcccaa

SEQ ID NO: 26 - DNA, 26 bp, synthetic construct
  misc_feature 1..26: note = Antisense oligomer
  source 1..26: mol_type = other DNA
ccaatgccatcctggagttcctgtaa

SEQ ID NO: 27 - DNA, 30 bp, synthetic construct
  misc_feature 1..30: note = Antisense oligomer
  source 1..30: mol_type = other DNA
cattcaatgttctgacaacagtttgccgct

SEQ ID NO: 28 - DNA, 25 bp, synthetic construct
  misc_feature 1..25: note = Antisense oligomer
  source 1..25: mol_type = other DNA
cttacaggctccaatagtggtcagt

SEQ ID NO: 29 - DNA, 29 bp, synthetic construct
  misc_feature 1..29: note = Antisense oligomer
  source 1..29: mol_type = other DNA
ccactcagagctcagatcttctaacttcc

SEQ ID NO: 30 - DNA, 25 bp, synthetic construct
  misc_feature 1..25: note = Antisense oligomer
  source 1..25: mol_type = other DNA
gggatccagtatacttacaggctcc

SEQ ID NO: 31 - DNA, 30 bp, synthetic construct
  misc_feature 1..30: note = Antisense oligomer
  source 1..30: mol_type = other DNA
acatcaaggaagatggcatttctagtttgg

SEQ ID NO: 32 - DNA, 30 bp, synthetic construct
  misc_feature 1..30: note = Antisense oligomer
  source 1..30: mol_type = other DNA
ctccaacatcaaggaagatggcatttctag

SEQ ID NO: 33 - DNA, 26 bp, synthetic construct
  misc_feature 1..26: note = Antisense oligomer
  source 1..26: mol_type = other DNA
gagcaggtacctccaacatcaaggaa

SEQ ID NO: 34 - DNA, 25 bp, synthetic construct
  misc_feature 1..25: note = Antisense oligomer
  source 1..25: mol_type = other DNA
ctgaaggtgttcttgtacttcatcc

SEQ ID NO: 35 - DNA, 30 bp, synthetic construct
  misc_feature 1..30: note = Antisense oligomer
  source 1..30: mol_type = other DNA
tgttcttgtacttcatcccactgattctga

SEQ ID NO: 36 - DNA, 18 bp, synthetic construct
  misc_feature 1..18: note = Antisense oligomer
  source 1..18: mol_type = other DNA
ctttcataatgctggcag

SEQ ID NO: 37 - DNA, 14 bp, synthetic construct
  misc_feature 1..14: note = Antisense oligomer
  source 1..14: mol_type = other DNA
cataatgctggcag

SEQ ID NO: 38-39 - skipped (2 entries)

SEQ ID NO: 40 - DNA, 12 bp, synthetic construct
  misc_feature 1..12: note = Antisense oligomer
  source 1..12: mol_type = other DNA
cagcagcagcag

SEQ ID NO: 41 - DNA, 15 bp, synthetic construct
  misc_feature 1..15: note = Antisense oligomer
  source 1..15: mol_type = other DNA
cagcagcagcagcag

SEQ ID NO: 42 - DNA, 18 bp, synthetic construct
  misc_feature 1..18: note = Antisense oligomer
  source 1..18: mol_type = other DNA
cagcagcagcagcagcag

SEQ ID NO: 43 - skipped

SEQ ID NO: 44 - DNA, 12 bp, synthetic construct
  misc_feature 1..12: note = Antisense oligomer
  source 1..12: mol_type = other DNA
agcagcagcagc

SEQ ID NO: 45 - DNA, 15 bp, synthetic construct
  misc_feature 1..15: note = Antisense oligomer
  source 1..15: mol_type = other DNA
agcagcagcagcagc

SEQ ID NO: 46 - DNA, 18 bp, synthetic construct
  misc_feature 1..18: note = Antisense oligomer
  source 1..18: mol_type = other DNA
agcagcagcagcagcagc

SEQ ID NO: 47 - skipped

SEQ ID NO: 48 - DNA, 12 bp, synthetic construct
  misc_feature 1..12: note = Antisense oligomer
  source 1..12: mol_type = other DNA
gcagcagcagca

SEQ ID NO: 49 - DNA, 15 bp, synthetic construct
  misc_feature 1..15: note = Antisense oligomer
  source 1..15: mol_type = other DNA
gcagcagcagcagca

SEQ ID NO: 50 - DNA, 18 bp, synthetic construct
  misc_feature 1..18: note = Antisense oligomer
  source 1..18: mol_type = other DNA
gcagcagcagcagcagca

SEQ ID NO: 51 - DNA, 25 bp, synthetic construct
  misc_feature 1..25: note = Antisense oligomer
  source 1..25: mol_type = other DNA
agcagcagcagcagcagcagcagca

SEQ ID NO: 52 - DNA, 25 bp, synthetic construct
  misc_feature 1..25: note = Antisense oligomer
  source 1..25: mol_type = other DNA
cagcagcagcagcagcagcagcagc

SEQ ID NO: 53 - skipped

SEQ ID NO: 54 - DNA, 12 bp, synthetic construct
  misc_feature 1..12: note = Antisense oligomer
  source 1..12: mol_type = other DNA
caggcaggcagg

SEQ ID NO: 55 - DNA, 24 bp, synthetic construct
  misc_feature 1..24: note = Antisense oligomer
  source 1..24: mol_type = other DNA
caggcaggcaggcaggcaggcagg

SEQ ID NO: 56 - protein, 9 aa, synthetic construct
  REGION 1..9: note = cell penetrating peptide
RRRQRRKKR

SEQ ID NO: 57 - protein, 9 aa, synthetic construct
  REGION 1..9: note = cell penetrating peptide
RKKRRQRRR

SEQ ID NO: 58 - protein, 11 aa, synthetic construct
  REGION 1..11: note = cell penetrating peptide
RRRRRRRRRFF

SEQ ID NO: 59 - protein, 11 aa, synthetic construct
  REGION 1..11: note = cell penetrating peptide
RRRRRFFRRRR

SEQ ID NO: 60 - protein, 4 aa, synthetic construct
  REGION 1..4: note = cell penetrating peptide
RRRR

SEQ ID NO: 61 - protein, 5 aa, synthetic construct
  REGION 1..5: note = cell penetrating peptide
RRRRR

SEQ ID NO: 62 - protein, 6 aa, synthetic construct
  REGION 1..6: note = cell penetrating peptide
RRRRRR

SEQ ID NO: 63 - protein, 7 aa, synthetic construct
  REGION 1..7: note = cell penetrating peptide
RRRRRRR

SEQ ID NO: 64 - protein, 8 aa, synthetic construct
  REGION 1..8: note = cell penetrating peptide
RRRRRRRR

SEQ ID NO: 65 - protein, 9 aa, synthetic construct
  REGION 1..9: note = cell penetrating peptide
RRRRRRRRR

SEQ ID NO: 66 - protein, 12 aa, synthetic construct
  REGION 1..12: note = cell penetrating peptide
  MOD_RES 2..11: note = Xaa = Acp
RXRRXRRXRRXR

SEQ ID NO: 67 - protein, 15 aa, synthetic construct
  REGION 1..15: note = cell penetrating peptide
  MOD_RES 2..14: note = Xaa = Acp
RXRRXRRXRRXRRXR

SEQ ID NO: 68 - protein, 12 aa, synthetic construct
  REGION 1..12: note = cell penetrating peptide
  MOD_RES 2..11: note = Xaa = bAla or Acp
RXRRXRRXRRXR

SEQ ID NO: 69 - protein, 15 aa, synthetic construct
  REGION 1..15: note = cell penetrating peptide
RARRARRARRARFFC

SEQ ID NO: 70 - protein, 15 aa, synthetic construct
  REGION 1..15: note = cell penetrating peptide
RGRRGRRGRRGRFFC

SEQ ID NO: 71 - protein, 10 aa, synthetic construct
  REGION 1..10: note = cell penetrating peptide
RRRQRRKKRC

SEQ ID NO: 72 - protein, 12 aa, synthetic construct
  REGION 1..12: note = cell penetrating peptide
RRRRRRRRRFFC

SEQ ID NO: 73 - protein, 13 aa, synthetic construct
  REGION 1..13: note = cell penetrating peptide
  MOD_RES 3..12: note = Xaa = Acp
  MOD_RES 13: note = Xaa = bAla
RRXRRXRRXRRXX

SEQ ID NO: 74 - protein, 14 aa, synthetic construct
  REGION 1..14: note = cell penetrating peptide
  MOD_RES 2..13: note = Xaa = Acp
  MOD_RES 14: note = Xaa = bAla
RXRRXRRXRRXRXX

SEQ ID NO: 75 - protein, 14 aa, synthetic construct
  REGION 1..14: note = cell penetrating peptide
  MOD_RES 1..13: note = Xaa = Acp
  MOD_RES 14: note = Xaa = bAla
XRRXRRXRRXRRXX

SEQ ID NO: 76 - protein, 13 aa, synthetic construct
  REGION 1..13: note = cell penetrating peptide
  MOD_RES 2..12: note = Xaa = Acp
  MOD_RES 13: note = Xaa = bAla
RXRXRXRXRXRXX

SEQ ID NO: 77 - protein, 15 aa, synthetic construct
  REGION 1..15: note = cell penetrating peptide
  MOD_RES 2..14: note = Xaa = Acp
  MOD_RES 15: note = Xaa = bAla
RXRXRXRXRXRXRXX

SEQ ID NO: 78 - protein, 17 aa, synthetic construct
  REGION 1..17: note = cell penetrating peptide
  MOD_RES 2..16: note = Xaa = Acp
  MOD_RES 17: note = Xaa = bAla
RXRRXRRXRRXRRXRXX

SEQ ID NO: 79 - protein, 14 aa, synthetic construct
  REGION 1..14: note = cell penetrating peptide
  MOD_RES 2..14: note = Xaa = bAla or Acp
RXRRXRRXRRXRXX

SEQ ID NO: 80 - protein, 7 aa, synthetic construct
  REGION 1..7: note = cell penetrating peptide
RRRRRRG

SEQ ID NO: 81 - protein, 8 aa, synthetic construct
  REGION 1..8: note = cell penetrating peptide
RRRRRRRG

SEQ ID NO: 82 - protein, 9 aa, synthetic construct
  REGION 1..9: note = cell penetrating peptide
RRRRRRRRG

SEQ ID NO: 83 - protein, 11 aa, synthetic construct
  REGION 1..11: note = cell penetrating peptide
RRRRRGRRRRG

SEQ ID NO: 84 - protein, 12 aa, synthetic construct
  REGION 1..12: note = cell penetrating peptide
RRRRRFFRRRRG

SEQ ID NO: 85 - protein, 10 aa, synthetic construct
  REGION 1..10: note = cell penetrating peptide
RKKRRQRRRG

SEQ ID NO: 86 - protein, 10 aa, synthetic construct
  REGION 1..10: note = cell penetrating peptide
RRRQRRKKRG

SEQ ID NO: 87 - protein, 12 aa, synthetic construct
  REGION 1..12: note = cell penetrating peptide
  VARIANT 2..8: note = Xaa = Ala, beta-alanine, Val, Leu, Ile, Ser, Gly, Thr, Phe, Trp, and 6-aminohexanoic acid
RXRRGGRXRRGG

SEQ ID NO: 88 - protein, 13 aa, synthetic construct
  REGION 1..13: note = cell penetrating peptide
  VARIANT 2..12: note = Xaa = Ala, beta-alanine, Val, Leu, Ile, Ser, Gly, Thr, Phe, Trp, and 6-aminohexanoic acid
RXRRRXRXRRRXG

SEQ ID NO: 89 - protein, 10 aa, synthetic construct
  REGION 1..10: note = cell penetrating peptide
  MOD_RES 10: note = Xaa = glycine or proline
RFFRFFRFFX

SEQ ID NO: 90 - protein, 11 aa, synthetic construct
  REGION 1..11: note = cell penetrating peptide
  MOD_RES 11: note = Xaa = glycine or proline
RTRTRFLRRTX

SEQ ID NO: 91 - protein, 11 aa, synthetic construct
  REGION 1..11: note = cell penetrating peptide
  MOD_RES 11: note = Xaa = glycine or proline
RFFRFFRFFRX

SEQ ID NO: 92 - protein, 11 aa, synthetic construct
  REGION 1..11: note = cell penetrating peptide
  MOD_RES 11: note = Xaa = glycine or proline
KTRTKFLKKTX

SEQ ID NO: 93 - protein, 10 aa, synthetic construct
  REGION 1..10: note = cell penetrating peptide
  MOD_RES 10: note = Xaa = glycine or proline
KFFKFFKFFX

SEQ ID NO: 94 - protein, 11 aa, synthetic construct
  REGION 1..11: note = cell penetrating peptide
  MOD_RES 11: note = Xaa = glycine or proline
KFFKFFKFFKX

SEQ ID NO: 95 - protein, 7 aa, synthetic construct
  REGION 1..7: note = cell penetrating peptide
  MOD_RES 7: note = Xaa = glycine or proline
RFFRFFX

SEQ ID NO: 96 - protein, 8 aa, synthetic construct
  REGION 1..8: note = cell penetrating peptide
  MOD_RES 8: note = Xaa = glycine or proline
RFFRFFRX

SEQ ID NO: 97 - skipped

SEQ ID NO: 98 - protein, 13 aa, synthetic construct
  REGION 1..13: note = cell penetrating peptide
  MOD_RES 2: note = Xaa = 6-aminohexanoic acid
  MOD_RES 5: note = Xaa = 6-aminohexanoic acid
  MOD_RES 8: note = Xaa = 6-aminohexanoic acid
  MOD_RES 11: note = Xaa = 6-aminohexanoic acid
  MOD_RES 13: note = Xaa = glycine or proline
RXRRXRRXRRXRX

SEQ ID NO: 99 - protein, 12 aa, synthetic construct
  REGION 1..12: note = cell penetrating peptide
  MOD_RES 12: note = Xaa = glycine or proline
YGRKKRRQRRRX

SEQ ID NO: 100 - protein, 11 aa, synthetic construct
  REGION 1..11: note = cell penetrating peptide
  MOD_RES 11: note = Xaa = glycine or proline
GRKKRRQRRRX

SEQ ID NO: 101 - protein, 10 aa, synthetic construct
  REGION 1..10: note = cell penetrating peptide
  MOD_RES 10: note = Xaa = glycine or proline
RKKRRQRRRX

SEQ ID NO: 102 - protein, 19 aa, synthetic construct
  REGION 1..19: note = cell penetrating peptide
  MOD_RES 19: note = Xaa = glycine or proline
RQIKIWFQNRRMKWKKGGX

SEQ ID NO: 103 - protein, 28 aa, synthetic construct
  REGION 1..28: note = cell penetrating peptide
  MOD_RES 28: note = Xaa = glycine or proline
GWTLNSAGYLLGKINLKALAALAKKILX

SEQ ID NO: 104 - protein, 23 aa, synthetic construct
  REGION 1..23: note = cell penetrating peptide
  MOD_RES 23: note = Xaa = glycine or proline
YARVRRRGPRGYARVRRRGPRRX

SEQ ID NO: 105 - protein, 12 aa, synthetic construct
  REGION 1..12: note = cell penetrating peptide
  MOD_RES 12: note = Xaa = glycine or proline
YARVRRRGPRRX

SEQ ID NO: 106 - protein, 10 aa, synthetic construct
  REGION 1..10: note = cell penetrating peptide
  MOD_RES 10: note = Xaa = glycine or proline
AKAARQAARX

SEQ ID NO: 107 - protein, 35 aa, synthetic construct
  REGION 1..35: note = cell penetrating peptide
  MOD_RES 35: note = Xaa = glycine or proline
DAATATRGRSAASRPTERPRAPARSASRPRRPVEX

SEQ ID NO: 108 - protein, 22 aa, synthetic construct
  REGION 1..22: note = cell penetrating peptide
  MOD_RES 22: note = Xaa = glycine or proline
KETWWETWWTEWSQPKKKRKVX

SEQ ID NO: 109 - protein, 22 aa, synthetic construct
  REGION 1..22: note = cell penetrating peptide
  MOD_RES 22: note = Xaa = glycine or proline
KETWFETWFTEWSQPKKKRKVX

SEQ ID NO: 110 - protein, 17 aa, synthetic construct
  REGION 1..17: note = cell penetrating peptide
  MOD_RES 17: note = Xaa = glycine or proline
RQIKIWFQNRRMKWKKX

SEQ ID NO: 111 - protein, 25 aa, synthetic construct
  REGION 1..25: note = cell penetrating peptide
  MOD_RES 25: note = Xaa = glycine or proline
RRRRRRRQIKIWFQNRRMKWKKGGX

SEQ ID NO: 112 - protein, 11 aa, synthetic construct
  REGION 1..11: note = cell penetrating peptide
  MOD_RES 11: note = Xaa = glycine or proline
RRRQRRKKRCX

SEQ ID NO: 113 - protein, 13 aa, synthetic construct
  REGION 1..13: note = cell penetrating peptide
  MOD_RES 13: note = Xaa = glycine or proline
CYGRKKRRQRRRX

SEQ ID NO: 114 - protein, 13 aa, synthetic construct
  REGION 1..13: note = cell penetrating peptide
  MOD_RES 13: note = Xaa = glycine or proline
RRRRRRRRRFFCX

SEQ ID NO: 115 - protein, 13 aa, synthetic construct
  REGION 1..13: note = cell penetrating peptide
  MOD_RES 13: note = Xaa = glycine or proline
RRRRRRRRRCFFX

SEQ ID NO: 116 - protein, 13 aa, synthetic construct
  REGION 1..13: note = cell penetrating peptide
  MOD_RES 13: note = Xaa = glycine or proline
RRRRRRRRCFFRX

SEQ ID NO: 117 - protein, 13 aa, synthetic construct
  REGION 1..13: note = cell penetrating peptide
  MOD_RES 13: note = Xaa = glycine or proline
RRRRRRCFFRRRX

SEQ ID NO: 118 - protein, 13 aa, synthetic construct
  REGION 1..13: note = cell penetrating peptide
  MOD_RES 13: note = Xaa = glycine or proline
RRRRRFCFRRRRX

SEQ ID NO: 119 - protein, 12 aa, synthetic construct
  REGION 1..12: note = cell penetrating peptide
  MOD_RES 12: note = Xaa = glycine or proline
RRRRRFFRRRRX

SEQ ID NO: 120 - protein, 13 aa, synthetic construct
  REGION 1..13: note = cell penetrating peptide
  MOD_RES 13: note = Xaa = glycine or proline
RRRRCFFRRRRRX

SEQ ID NO: 121 - protein, 13 aa, synthetic construct
  REGION 1..13: note = cell penetrating peptide
  MOD_RES 13: note = Xaa = glycine or proline
RRCFFRRRRRRRX

SEQ ID NO: 122 - protein, 13 aa, synthetic construct
  REGION 1..13: note = cell penetrating peptide
  MOD_RES 13: note = Xaa = glycine or proline
CFFRRRRRRRRRX

SEQ ID NO: 123 - protein, 13 aa, synthetic construct
  REGION 1..13: note = cell penetrating peptide
  MOD_RES 13: note = Xaa = glycine or proline
CRRRRRRRRRFFX

SEQ ID NO: 124 - protein, 12 aa, synthetic construct
  REGION 1..12: note = cell penetrating peptide
  MOD_RES 12: note = Xaa = glycine or proline
FFRRRRRRRRRX

SEQ ID NO: 125 - protein, 15 aa, synthetic construct
  REGION 1..15: note = cell penetrating peptide
  MOD_RES 15: note = Xaa = glycine or proline
RRRRRFFCFFRRRRX

SEQ ID NO: 126 - protein, 12 aa, synthetic construct
  REGION 1..12: note = cell penetrating peptide
  MOD_RES 12: note = Xaa = glycine or proline
RRRRRRRRRIIX

SEQ ID NO: 127 - protein, 12 aa, synthetic construct
  REGION 1..12: note = cell penetrating peptide
  MOD_RES 12: note = Xaa = glycine or proline
RRRRRRRRFFFX

SEQ ID NO: 128 - protein, 14 aa, synthetic construct
  REGION 1..14: note = cell penetrating peptide
  MOD_RES 14: note = Xaa = glycine or proline
RRRRRRRRRFFFFX

SEQ ID NO: 129 - protein, 11 aa, synthetic construct
  REGION 1..11: note = cell penetrating peptide
  MOD_RES 11: note = Xaa = glycine or proline
RRRRRRRRFFX

SEQ ID NO: 130 - protein, 9 aa, synthetic construct
  REGION 1..9: note = cell penetrating peptide
  MOD_RES 9: note = Xaa = glycine or proline
RRRRRRFFX

SEQ ID NO: 131 - protein, 8 aa, synthetic construct
  REGION 1..8: note = cell penetrating peptide
  MOD_RES 8: note = Xaa = glycine or proline
RRRRRFFX

SEQ ID NO: 132 - protein, 12 aa, synthetic construct
  REGION 1..12: note = cell penetrating peptide
  MOD_RES 3: note = Xaa = 6-aminohexanoic acid
  MOD_RES 6: note = Xaa = 6-aminohexanoic acid
  MOD_RES 9: note = Xaa = 6-aminohexanoic acid
  MOD_RES 12: note = Xaa = glycine or proline
RRXRRXRRXRRX

SEQ ID NO: 133 - protein, 13 aa, synthetic construct
  REGION 1..13: note = cell penetrating peptide
  MOD_RES 2: note = Xaa = 6-aminohexanoic acid
  MOD_RES 5: note = Xaa = 6-aminohexanoic acid
  MOD_RES 8: note = Xaa = 6-aminohexanoic acid
  MOD_RES 11: note = Xaa = 6-aminohexanoic acid
  MOD_RES 13: note = Xaa = glycine or proline
RXRRXRRXRRXRX

SEQ ID NO: 134 - protein, 13 aa, synthetic construct
  REGION 1..13: note = cell penetrating peptide
  MOD_RES 1: note = Xaa = 6-aminohexanoic acid
  MOD_RES 4: note = Xaa = 6-aminohexanoic acid
  MOD_RES 7: note = Xaa = 6-aminohexanoic acid
  MOD_RES 10: note = Xaa = 6-aminohexanoic acid
  MOD_RES 13: note = Xaa = glycine or proline
XRRXRRXRRXRRX

SEQ ID NO: 135 - protein, 12 aa, synthetic construct
  REGION 1..12: note = cell penetrating peptide
  MOD_RES 2: note = Xaa = 6-aminohexanoic acid
  MOD_RES 4: note = Xaa = 6-aminohexanoic acid
  MOD_RES 6: note = Xaa = 6-aminohexanoic acid
  MOD_RES 8: note = Xaa = 6-aminohexanoic acid
  MOD_RES 10: note = Xaa = 6-aminohexanoic acid
  MOD_RES 12: note = Xaa = glycine or proline
RXRXRXRXRXRX

SEQ ID NO: 136 - protein, 10 aa, synthetic construct
  REGION 1..10: note = cell penetrating peptide
  MOD_RES 2: note = Xaa = 6-aminohexanoic acid
  MOD_RES 5: note = Xaa = 6-aminohexanoic acid
  MOD_RES 8: note = Xaa = 6-aminohexanoic acid
  MOD_RES 10: note = Xaa = glycine or proline
RXRRXRRXRX

SEQ ID NO: 137 - protein, 8 aa, synthetic construct
  REGION 1..8: note = cell penetrating peptide
  MOD_RES 2: note = Xaa = 6-aminohexanoic acid
  MOD_RES 5: note = Xaa = 6-aminohexanoic acid
  MOD_RES 8: note = Xaa = glycine or proline
RXRRXRRX

SEQ ID NO: 138 - protein, 7 aa, synthetic construct
  REGION 1..7: note = cell penetrating peptide
  MOD_RES 2: note = Xaa = 6-aminohexanoic acid
  MOD_RES 5: note = Xaa = 6-aminohexanoic acid
  MOD_RES 7: note = Xaa = glycine or proline
RXRRXRX

SEQ ID NO: 139 - protein, 12 aa, synthetic construct
  REGION 1..12: note = cell penetrating peptide
  MOD_RES 3: note = Xaa = 6-aminohexanoic acid
  MOD_RES 6: note = Xaa = 6-aminohexanoic acid
  MOD_RES 9: note = Xaa = 6-aminohexanoic acid
  MOD_RES 12: note = Xaa = glycine or proline
RKXRKXRKXRKX

SEQ ID NO: 140 - protein, 12 aa, synthetic construct
  REGION 1..12: note = cell penetrating peptide
  MOD_RES 3: note = Xaa = 6-aminohexanoic acid
  MOD_RES 6: note = Xaa = 6-aminohexanoic acid
  MOD_RES 9: note = Xaa = 6-aminohexanoic acid
  MOD_RES 12: note = Xaa = glycine or proline
RHXRHXRHXRHX

SEQ ID NO: 141 - protein, 13 aa, synthetic construct
  REGION 1..13: note = cell penetrating peptide
  MOD_RES 13: note = Xaa = glycine or proline
RRRRRRRRCFFRX

SEQ ID NO: 142 - protein, 12 aa, synthetic construct
  REGION 1..12: note = cell penetrating peptide
  MOD_RES 3: note = Xaa = 6-aminohexanoic acid
  MOD_RES 6: note = Xaa = 6-aminohexanoic acid
  MOD_RES 9: note = Xaa = 6-aminohexanoic acid
  MOD_RES 12: note = Xaa = glycine or proline
RRXRRXRRXRRX

SEQ ID NO: 143 - protein, 13 aa, synthetic construct
  REGION 1..13: note = cell penetrating peptide
  MOD_RES 2..11: note = Xaa = 6-aminohexanoic acid
  MOD_RES 13: note = Xaa = glycine or proline
RXRRXRRXRRXRX

SEQ ID NO: 144 - protein, 13 aa, synthetic construct
  REGION 1..13: note = cell penetrating peptide
  MOD_RES 1..10: note = Xaa = 6-aminohexanoic acid
  MOD_RES 13: note = Xaa = glycine or proline
XRRXRRXRRXRRX

SEQ ID NO: 145 - protein, 12 aa, synthetic construct
  REGION 1..12: note = cell penetrating peptide
  MOD_RES 2..10: note = Xaa = 6-aminohexanoic acid
  MOD_RES 12: note = Xaa = glycine or proline
RXRXRXRXRXRX

SEQ ID NO: 146 - protein, 14 aa, synthetic construct
  REGION 1..14: note = cell penetrating peptide
  MOD_RES 2..12: note = Xaa = 6-aminohexanoic acid
  MOD_RES 14: note = Xaa = glycine or proline
RXRXRXRXRXRXRX

SEQ ID NO: 147 - protein, 16 aa, synthetic construct
  REGION 1..16: note = cell penetrating peptide
  MOD_RES 2..14: note = Xaa = 6-aminohexanoic acid
  MOD_RES 16: note = Xaa = glycine or proline
RXRRXRRXRRXRRXRX

SEQ ID NO: 148 - protein, 13 aa, synthetic construct
  REGION 1..13: note = cell penetrating peptide
  MOD_RES 2..11: note = Xaa = 6-aminohexanoic acid or beta-alanine
  MOD_RES 13: note = Xaa = glycine or proline
RXRRXRRXRRXRX

SEQ ID NO: 149 - protein, 13 aa, synthetic construct
  REGION 1..13: note = cell penetrating peptide
  MOD_RES 2..8: note = Xaa = 6-aminohexanoic acid
  MOD_RES 11: note = Xaa = beta-alanine
  MOD_RES 13: note = Xaa = glycine or proline
RXRRXRRXRRXRX

SEQ ID NO: 150 - protein, 16 aa, synthetic construct
  REGION 1..16: note = cell penetrating peptide
  MOD_RES 2..10: note = Xaa = beta-alanine
  MOD_RES 12: note = Xaa = 6-aminohexanoic acid
  MOD_RES 16: note = Xaa = glycine or proline
  MOD_RES 14: note = Xaa = beta-alanine
RXRXRXRXRXRXRXRX

SEQ ID NO: 151 - protein, 16 aa, synthetic construct
  REGION 1..16: note = cell penetrating peptide
  MOD_RES 2..6: note = Xaa =beta-alanine
  MOD_RES 8: note = Xaa = 6-aminohexanoic acid
  MOD_RES 16: note = Xaa = glycine or proline
  MOD_RES 10..14: note = Xaa =beta-alanine
RXRXRXRXRXRXRXRX

SEQ ID NO: 152 - protein, 17 aa, synthetic construct
  REGION 1..17: note = cell penetrating peptide
  MOD_RES 1..15: note = Xaa = 6-aminohexanoic acid or beta-alanine
  MOD_RES 17: note = Xaa = glycine or proline
XRXRXRXRXRXRXRXRX

SEQ ID NO: 153 - protein, 16 aa, synthetic construct
  REGION 1..16: note = cell penetrating peptide
  MOD_RES 2..14: note = Xaa = 6-aminohexanoic acid or beta-alanine
  MOD_RES 16: note = Xaa = glycine or proline
RXRXRXRXRXRXRXRX

SEQ ID NO: 154 - protein, 16 aa, synthetic construct
  REGION 1..16: note = cell penetrating peptide
  MOD_RES 10..14: note = Xaa = 6-aminohexanoic acid
  MOD_RES 2..8: note = Xaa = beta-alanine
  MOD_RES 16: note = Xaa = glycine or proline
RXRXRXRXRXRXRXRX

SEQ ID NO: 155 - protein, 16 aa, synthetic construct
  REGION 1..16: note = cell penetrating peptide
  MOD_RES 4..14: note = Xaa = 6-aminohexanoic acid or beta-alanine
  MOD_RES 16: note = Xaa = glycine or proline
RXRXRXRXRXRXRXRX

SEQ ID NO: 156 - protein, 16 aa, synthetic construct
  REGION 1..16: note = cell penetrating peptide
  MOD_RES 2..14: note = Xaa = beta-alanine
  MOD_RES 16: note = Xaa = glycine or proline
RXRXRXRXRXRXRXRX

SEQ ID NO: 157 - protein, 5 aa, synthetic construct
  REGION 1..5: note = cell penetrating peptide
  MOD_RES 5: note = Xaa = glycine or proline
RRRRX

SEQ ID NO: 158 - protein, 6 aa, synthetic construct
  REGION 1..6: note = cell penetrating peptide
  MOD_RES 6: note = Xaa = glycine or proline
RRRRRX

SEQ ID NO: 159 - protein, 7 aa, synthetic construct
  REGION 1..7: note = cell penetrating peptide
  MOD_RES 7: note = Xaa = glycine or proline
RRRRRRX

SEQ ID NO: 160 - protein, 8 aa, synthetic construct
  REGION 1..8: note = cell penetrating peptide
  MOD_RES 8: note = Xaa = glycine or proline
RRRRRRRX

SEQ ID NO: 161 - protein, 9 aa, synthetic construct
  REGION 1..9: note = cell penetrating peptide
  MOD_RES 9: note = Xaa = glycine or proline
RRRRRRRRX

SEQ ID NO: 162 - protein, 11 aa, synthetic construct
  REGION 1..11: note = cell penetrating peptide
  MOD_RES 11: note = Xaa = glycine or proline
RRRRRGRRRRX

SEQ ID NO: 163 - protein, 12 aa, synthetic construct
  REGION 1..12: note = cell penetrating peptide
  MOD_RES 12: note = Xaa = glycine or proline
RRRRRFFRRRRX

SEQ ID NO: 164 - protein, 10 aa, synthetic construct
  REGION 1..10: note = cell penetrating peptide
  MOD_RES 10: note = Xaa = glycine or proline
RKKRRQRRRX

SEQ ID NO: 165 - protein, 10 aa, synthetic construct
  REGION 1..10: note = cell penetrating peptide
  MOD_RES 10: note = Xaa = glycine or proline
RRRQRRKKRX

SEQ ID NO: 166 - protein, 7 aa, synthetic construct
  REGION 1..7: note = cell penetrating peptide
  MOD_RES 2..5: note = Xaa = 6-aminohexanoic acid
  MOD_RES 7: note = Xaa = glycine or proline
RXRRXRX

SEQ ID NO: 167 - protein, 7 aa, synthetic construct
  REGION 1..7: note = cell penetrating peptide
  MOD_RES 2..5: note = Xaa = beta-alanine
  MOD_RES 7: note = Xaa = glycine or proline
RXRRXRX

SEQ ID NO: 168 - protein, 7 aa, synthetic construct
  REGION 1..7: note = cell penetrating peptide
  MOD_RES 2: note = Xaa = 6-aminohexanoic acid
  MOD_RES 5: note = Xaa = beta-alanine
  MOD_RES 7: note = Xaa = glycine or proline
RXRRXRX

SEQ ID NO: 169 - protein, 7 aa, synthetic construct
  REGION 1..7: note = cell penetrating peptide
  MOD_RES 2: note = Xaa = beta-alanine
  MOD_RES 5: note = Xaa = 6-aminohexanoic acid
  MOD_RES 7: note = Xaa = glycine or proline
RXRRXRX

SEQ ID NO: 170 - protein, 8 aa, synthetic construct
  REGION 1..8: note = cell penetrating peptide
  MOD_RES 2: note = Xaa = 6-aminohexanoic acid
  MOD_RES 4: note = Xaa = NH-(CHRe)n-C(O)-wherein n is 2 to 7 and each Re is independently, at each occurrence, hydrogen or methyl
  MOD_RES 8: note = Xaa - glycine or proline
  MOD_RES 6: note = Xaa = 6-aminohexanoic acid
RXRXRXRX

SEQ ID NO: 171 - protein, 8 aa, synthetic construct
  REGION 1..8: note = cell penetrating peptide
  MOD_RES 2: note = Xaa = beta-alanine
  MOD_RES 4: note = Xaa = NH-(CHRe)n-C(O)-wherein n is 2 to 7 and each Re is independently, at each occurrence, hydrogen or methyl
  MOD_RES 8: note = Xaa = glycine or proline
  MOD_RES 6: note = Xaa = beta-alanine
RXRXRXRX

SEQ ID NO: 172 - protein, 8 aa, synthetic construct
  REGION 1..8: note = cell penetrating peptide
  MOD_RES 2: note = Xaa = 6-aminohexanoic acid
  MOD_RES 4: note = Xaa = NH-(CHRe)n-C(O)-wherein n is 2 to 7 and each Re is independently, at each occurrence, hydrogen or methyl
  MOD_RES 6: note = Xaa = beta-alanine
  MOD_RES 8: note = Xaa = glycine or proline
RXRXRXRX

SEQ ID NO: 173 - protein, 8 aa, synthetic construct
  REGION 1..8: note = cell penetrating peptide
  MOD_RES 2: note = Xaa = beta-alanine
  MOD_RES 4: note = Xaa = NH-(CHRe)n-C(O)-wherein n is 2 to 7 and each Re is independently, at each occurrence, hydrogen or methyl
  MOD_RES 6: note = Xaa = 6-aminohexanoic acid
  MOD_RES 8: note = Xaa = glycine or proline
RXRXRXRX

SEQ ID NO: 174 - protein, 12 aa, synthetic construct
  REGION 1..12: note = cell penetrating peptide
  MOD_RES 2..10: note = Xaa = 6-aminohexanoic acid
  MOD_RES 12: note = Xaa = glycine or proline
RXRILFQYRXRX

SEQ ID NO: 175 - protein, 12 aa, synthetic construct
  REGION 1..12: note = cell penetrating peptide
  MOD_RES 2..10: note = Xaa = beta-alanine
  MOD_RES 12: note = Xaa = glycine or proline
RXRILFQYRXRX

SEQ ID NO: 176 - protein, 12 aa, synthetic construct
  REGION 1..12: note = cell penetrating peptide
  MOD_RES 2: note = Xaa = 6-aminohexanoic acid
  MOD_RES 10: note = Xaa = beta-alanine
  MOD_RES 12: note = Xaa = glycine or proline
RXRILFQYRXRX

SEQ ID NO: 177 - protein, 12 aa, synthetic construct
  REGION 1..12: note = cell penetrating peptide
  MOD_RES 2: note = Xaa = beta-alanine
  MOD_RES 10: note = Xaa = 6-aminohexanoic acid
  MOD_RES 12: note = Xaa = glycine or proline
RXRILFQYRXRX

SEQ ID NO: 178 - protein, 10 aa, synthetic construct
  REGION 1..10: note = cell penetrating peptide
  MOD_RES 2..8: note = Xaa - 6-aminohexanoic acid
  MOD_RES 10: note = Xaa = glycine or proline
RXRRXRRXRX

SEQ ID NO: 179 - protein, 10 aa, synthetic construct
  REGION 1..10: note = cell penetrating peptide
  MOD_RES 2..8: note = Xaa = beta-alanine
  MOD_RES 10: note = Xaa = glycine or proline
RXRRXRRXRX

SEQ ID NO: 180 - protein, 10 aa, synthetic construct
  REGION 1..10: note = cell penetrating peptide
  MOD_RES 2: note = Xaa = 6-aminohexanoic acid
  MOD_RES 5: note = Xaa = beta-alanine
  MOD_RES 10: note = Xaa = glycine or proline
  MOD_RES 8: note = Xaa = 6-aminohexanoic acid
RXRRXRRXRX

SEQ ID NO: 181 - protein, 10 aa, synthetic construct
  REGION 1..10: note = cell penetrating peptide
  MOD_RES 2: note = Xaa = 6-aminohexanoic acid
  MOD_RES 5..8: note = Xaa = beta-alanine
  MOD_RES 10: note = Xaa = glycine or proline
RXRRXRRXRX

SEQ ID NO: 182 - protein, 10 aa, synthetic construct
  REGION 1..10: note = cell penetrating peptide
  MOD_RES 2..5: note = Xaa = 6-aminohexanoic acid
  MOD_RES 8: note = Xaa = beta-alanine
  MOD_RES 10: note = Xaa = glycine or proline
RXRRXRRXRX

SEQ ID NO: 183 - protein, 10 aa, synthetic construct
  REGION 1..10: note = cell penetrating peptide
  MOD_RES 2: note = Xaa = beta-alanine
  MOD_RES 5: note = Xaa = 6-aminohexanoic acid
  MOD_RES 10: note = Xaa = glycine or proline
  MOD_RES 8: note = Xaa = beta-alanine
RXRRXRRXRX

SEQ ID NO: 184 - protein, 10 aa, synthetic construct
  REGION 1..10: note = cell penetrating peptide
  MOD_RES 2: note = Xaa = beta-alanine
  MOD_RES 5..8: note = Xaa = 6-aminohexanoic acid
  MOD_RES 10: note = Xaa = glycine or proline
RXRRXRRXRX

SEQ ID NO: 185 - protein, 10 aa, synthetic construct
  REGION 1..10: note = cell penetrating peptide
  MOD_RES 2..5: note = Xaa = beta-alanine
  MOD_RES 8: note = Xaa = 6-aminohexanoic acid
  MOD_RES 10: note = Xaa = glycine or proline
RXRRXRRXRX

SEQ ID NO: 186 - protein, 11 aa, synthetic construct
  REGION 1..11: note = cell penetrating peptide
  MOD_RES 6..9: note = Xaa = 6-aminohexanoic acid
  MOD_RES 11: note = Xaa = glycine or proline
  MOD_RES 4: note = Xaa = -NH-(CHRe)n-C(O)-wherein n is 2 to 7 and each Re is independently, at each occurrence, hydrogen or methyl
  MOD_RES 2: note = Xaa = 6-aminohexanoic acid
RXRXRXRRXRX

SEQ ID NO: 187 - protein, 11 aa, synthetic construct
  REGION 1..11: note = cell penetrating peptide
  MOD_RES 2..5: note = Xaa = 6-aminohexanoic acid
  MOD_RES 7: note = Xaa = -NH-(CHRe)n-C(O)-wherein n is 2 to 7 and each Re is independently, at each occurrence, hydrogen or methyl
  MOD_RES 11: note = Xaa = glycine or proline
  MOD_RES 9: note = Xaa = 6-aminohexanoic acid
RXRRXRXRXRX

SEQ ID NO: 188 - protein, 15 aa, synthetic construct
  REGION 1..15: note = cell penetrating peptide
  MOD_RES 2..13: note = Xaa = 6-aminohexanoic acid
  MOD_RES 15: note = Xaa = glycine or proline
RXRILFQYRXRRXRX

SEQ ID NO: 189 - protein, 15 aa, synthetic construct
  REGION 1..15: note = cell penetrating peptide
  MOD_RES 2..13: note = Xaa = 6-aminohexanoic acid
  MOD_RES 15: note = Xaa = glycine or proline
RXRRXRILFQYRXRX

SEQ ID NO: 190 - protein, 12 aa, synthetic construct
  REGION 1..12: note = cell penetrating peptide
  MOD_RES 2: note = Xaa = 6-aminohexanoic acid
  MOD_RES 4: note = Xaa = -NH-(CHRe)n-C(O)-wherein n is 2 to 7 and each Re is independently, at each occurrence, hydrogen or methyl
  MOD_RES 12: note = Xaa = glycine or proline
  MOD_RES 6: note = Xaa = 6-aminohexanoic acid
  MOD_RES 10: note = Xaa = 6-aminohexanoic acid
  MOD_RES 8: note = Xaa = -NH-(CHRe)n-C(O)-wherein n is 2 to 7 and each Re is independently, at each occurrence, hydrogen or methyl
RXRXRXRXRXRX

SEQ ID NO: 191 - protein, 20 aa, synthetic construct
  REGION 1..20: note = cell penetrating peptide
  MOD_RES 2..18: note = Xaa = 6-aminohexanoic acid
  MOD_RES 20: note = Xaa = glycine or proline
RXRILFQYRXRILFQYRXRX

SEQ ID NO: 192 - protein, 16 aa, synthetic construct
  REGION 1..16: note = cell penetrating peptide
  MOD_RES 2..10: note = Xaa = 6-aminohexanoic acid
  MOD_RES 12: note = Xaa = -NH-(CHRe)n-C(O)-wherein n is 2 to 7 and each Re is independently, at each occurrence, hydrogen or methyl
  MOD_RES 16: note = Xaa = glycine or proline
  MOD_RES 14: note = Xaa = 6-aminohexanoic acid
RXRILFQYRXRXRXRX

SEQ ID NO: 193 - protein, 16 aa, synthetic construct
  REGION 1..16: note = cell penetrating peptide
  MOD_RES 6..14: note = Xaa = 6-aminohexanoic acid
  MOD_RES 4: note = Xaa = -NH-(CHRe)n-C(O)-wherein n is 2 to 7 and each Re is independently, at each occurrence, hydrogen or methyl
  MOD_RES 16: note = Xaa = glycine or proline
  MOD_RES 2: note = Xaa = 6-aminohexanoic acid
RXRXRXRILFQYRXRX

SEQ ID NO: 194 - protein, 11 aa, synthetic construct
  REGION 1..11: note = cell penetrating peptide
  MOD_RES 6..9: note = Xaa = beta-alanine
  MOD_RES 4: note = Xaa = -NH-(CHRe)n-C(O)-wherein n is 2 to 7 and each Re is independently, at each occurrence, hydrogen or methyl
  MOD_RES 11: note = Xaa = glycine or proline
  MOD_RES 2: note = Xaa = beta-alanine
RXRXRXRRXRX

SEQ ID NO: 195 - protein, 11 aa, synthetic construct
  REGION 1..11: note = cell penetrating peptide
  MOD_RES 2..5: note = Xaa = beta-alanine
  MOD_RES 7: note = Xaa = -NH-(CHRe)n-C(O)-wherein n is 2 to 7 and each Re is independently, at each occurrence, hydrogen or methyl
  MOD_RES 11: note = Xaa = glycine or proline
  MOD_RES 9: note = Xaa = beta-alanine
RXRRXRXRXRX

SEQ ID NO: 196 - protein, 15 aa, synthetic construct
  REGION 1..15: note = cell penetrating peptide
  MOD_RES 2..13: note = Xaa = beta-alanine
  MOD_RES 15: note = Xaa = glycine or proline
RXRILFQYRXRRXRX

SEQ ID NO: 197 - protein, 15 aa, synthetic construct
  REGION 1..15: note = cell penetrating peptide
  MOD_RES 2..13: note = Xaa = beta-alanine
  MOD_RES 15: note = Xaa = glycine or proline
RXRRBRILFQYRXRX

SEQ ID NO: 198 - protein, 12 aa, synthetic construct
  REGION 1..12: note = cell penetrating peptide
  MOD_RES 2..6: note = Xaa = beta-alanine
  MOD_RES 8: note = Xaa = -NH-(CHRe)n-C(O)-wherein n is 2 to 7 and each Re is independently, at each occurrence, hydrogen or methyl
  MOD_RES 12: note = Xaa = glycine or proline
  MOD_RES 10: note = Xaa = beta-alanine
RXRYRXRXRXRX

SEQ ID NO: 199 - protein, 20 aa, synthetic construct
  REGION 1..20: note = cell penetrating peptide
  MOD_RES 2..18: note = Xaa = beta-alanine
  MOD_RES 20: note = Xaa = glycine or proline
RXRILFQYRXRILFQYRXRX

SEQ ID NO: 200 - protein, 16 aa, synthetic construct
  REGION 1..16: note = cell penetrating peptide
  MOD_RES 6..14: note = Xaa = beta-alanine
  MOD_RES 4: note = Xaa = -NH-(CHRe)n-C(O)-wherein n is 2 to 7 and each Re is independently, at each occurrence, hydrogen or methyl
  MOD_RES 16: note = Xaa = glycine or alanine
  MOD_RES 2: note = Xaa = beta-alanine
RXRXRXRILFQYRXRX

SEQ ID NO: 201 - protein, 16 aa, synthetic construct
  REGION 1..16: note = cell penetrating peptide
  MOD_RES 2..10: note = Xaa = beta-alanine
  MOD_RES 12: note = Xaa = -NH-(CHRe)n-C(O)-wherein n is 2 to 7 and each Re is independently, at each occurrence, hydrogen or methyl
  MOD_RES 16: note = Xaa = glycine or proline
  MOD_RES 14: note = Xaa = beta-alanine
RXRILFQYRXRXRXRX

SEQ ID NO: 202 - protein, 11 aa, synthetic construct
  REGION 1..11: note = cell penetrating peptide
  MOD_RES 2: note = Xaa = 6-aminohexanoic acid
  MOD_RES 4: note = Xaa = -NH-(CHRe)n-C(O)-wherein n is 2 to 7 and each Re is independently, at each occurrence, hydrogen or methyl
  MOD_RES 6: note = Xaa = beta-alanine
  MOD_RES 11: note = Xaa = glycine or proline
  MOD_RES 9: note = Xaa = 6-aminohexanoic acid
RXRXRXRRXRX

SEQ ID NO: 203 - protein, 11 aa, synthetic construct
  REGION 1..11: note = cell penetrating peptide
  MOD_RES 2: note = Xaa = 6-aminohexanoic acid
  MOD_RES 5: note = Xaa = beta-alanine
  MOD_RES 7: note = Xaa = -NH-(CHRe)n-C(O)-wherein n is 2 to 7 and each Re is independently, at each occurrence, hydrogen or methyl
  MOD_RES 11: note = Xaa = glycine or proline
  MOD_RES 9: note = Xaa = 6-aminohexanoic acid
RXRRXRXRXRX

SEQ ID NO: 204 - protein, 15 aa, synthetic construct
  REGION 1..15: note = cell penetrating peptide
  MOD_RES 2: note = Xaa = 6-aminohexanoic acid
  MOD_RES 10: note = Xaa = beta-alanine
  MOD_RES 15: note = Xaa = glycine or proline
  MOD_RES 13: note = Xaa = 6-aminohexanoic acid
RXRILFQYRXRRXRX

SEQ ID NO: 205 - protein, 15 aa, synthetic construct
  REGION 1..15: note = cell penetrating peptide
  MOD_RES 5: note = Xaa = beta-alanine
  MOD_RES 2: note = Xaa = 6-aminohexanoic acid
  MOD_RES 15: note = Xaa = glycine or proline
  MOD_RES 13: note = Xaa = 6-aminohexanoic acid
RXRRXRILFQYRXRX

SEQ ID NO: 206 - protein, 12 aa, synthetic construct
  REGION 1..12: note = cell penetrating peptide
  MOD_RES 2: note = Xaa = 6-aminohexanoic acid
  MOD_RES 4: note = Xaa = -NH-(CHRe)n-C(O)-wherein n is 2 to 7 and each Re is independently, at each occurrence, hydrogen or methyl
  MOD_RES 6: note = Xaa = beta-alanine
  MOD_RES 12: note = Xaa = glycine or proline
  MOD_RES 8: note = Xaa = -NH-(CHRe)n-C(O)-wherein n is 2 to 7 and each Re is independently, at each occurrence, hydrogen or methyl
  MOD_RES 10: note = Xaa = 6-aminohexanoic acid
RXRXRXRXRXRX

SEQ ID NO: 207 - protein, 20 aa, synthetic construct
  REGION 1..20: note = cell penetrating peptide
  MOD_RES 2: note = Xaa = 6-aminohexanoic acid
  MOD_RES 10: note = Xaa = beta-alanine
  MOD_RES 20: note = Xaa = glycine or proline
  MOD_RES 18: note = Xaa = 6-aminohexanoic acid
RXRILFQYRXRILFQYRXRX

SEQ ID NO: 208 - protein, 16 aa, synthetic construct
  REGION 1..16: note = cell penetrating peptide
  MOD_RES 2: note = Xaa = 6-aminohexanoic acid
  MOD_RES 4: note = Xaa = -NH-(CHRe)n-C(O)-wherein n is 2 to 7 and each Re is independently, at each occurrence, hydrogen or methyl
  MOD_RES 6: note = Xaa = beta-alanine
  MOD_RES 16: note = Xaa = glycine or proline
  MOD_RES 14: note = Xaa = 6-aminohexanoic acid
RXRXRXRILFQYRXRX

SEQ ID NO: 209 - protein, 16 aa, synthetic construct
  REGION 1..16: note = cell penetrating peptide
  MOD_RES 2: note = Xaa = 6-aminohexanoic acid
  MOD_RES 10: note = Xaa = beta-alanine
  MOD_RES 12: note = Xaa = -NH-(CHRe)n-C(O)-wherein n is 2 to 7 and each Re is independently, at each occurrence, hydrogen or methyl
  MOD_RES 16: note = Xaa = glycine or proline
  MOD_RES 14: note = Xaa = 6-aminohexanoic acid
RXRILFQYRXRXRXRX

SEQ ID NO: 210 - protein, 11 aa, synthetic construct
  REGION 1..11: note = cell penetrating peptide
  MOD_RES 2: note = Xaa = 6-aminohexanoic acid
  MOD_RES 4: note = Xaa = -NH-(CHRe)n-C(O)-wherein n is 2 to 7 and each Re is independently, at each occurrence, hydrogen or methyl
  MOD_RES 6..9: note = Xaa = beta-alanine
  MOD_RES 11: note = Xaa = glycine or proline
RXRXRXRRXRX

SEQ ID NO: 211 - protein, 11 aa, synthetic construct
  REGION 1..11: note = cell penetrating peptide
  MOD_RES 2: note = Xaa = 6-aminohexanoic acid
  MOD_RES 5: note = Xaa = beta-alanine
  MOD_RES 7: note = Xaa = -NH-(CHRe)n-C(O)-wherein n is 2 to 7 and each Re is independently, at each occurrence, hydrogen or methyl
  MOD_RES 11: note = Xaa = glycine or proline
  MOD_RES 9: note = Xaa = beta-alanine
RXRRXRXRXRX

SEQ ID NO: 212 - protein, 15 aa, synthetic construct
  REGION 1..15: note = cell penetrating peptide
  MOD_RES 2: note = Xaa = 6-aminohexanoic acid
  MOD_RES 10..13: note = Xaa = beta-alanine
  MOD_RES 15: note = Xaa = glycine or proline
RXRILFQYRXRRXRX

SEQ ID NO: 213 - protein, 15 aa, synthetic construct
  REGION 1..15: note = cell penetrating peptide
  MOD_RES 2: note = Xaa = 6-aminohexanoic acid
  MOD_RES 5..13: note = Xaa = beta-alanine
  MOD_RES 15: note = Xaa = glycine or proline
RXRRXRILFQYRXRX

SEQ ID NO: 214 - protein, 12 aa, synthetic construct
  REGION 1..12: note = cell penetrating peptide
  MOD_RES 2: note = Xaa = 6-aminohexanoic acid
  MOD_RES 4: note = Xaa = -NH-(CHRe)n-C(O)-wherein n is 2 to 7 and each Re is independently, at each occurrence, hydrogen or methyl
  MOD_RES 6: note = Xaa = beta-alanine
  MOD_RES 12: note = Xaa = glycine or proline
  MOD_RES 10: note = Xaa = beta-alanine
  MOD_RES 8: note = Xaa = -NH-(CHRe)n-C(O)-wherein n is 2 to 7 and each Re is independently, at each occurrence, hydrogen or methyl
RXRXRXRXRXRX

SEQ ID NO: 215 - protein, 20 aa, synthetic construct
  REGION 1..20: note = cell penetrating peptide
  MOD_RES 2: note = Xaa = 6-aminohexanoic acid
  MOD_RES 10..18: note = Xaa = beta-alanine
  MOD_RES 20: note = Xaa = glycine or proline
RXRILFQYRXRILFQYRXRX

SEQ ID NO: 216 - protein, 16 aa, synthetic construct
  REGION 1..16: note = cell penetrating peptide
  MOD_RES 2: note = Xaa = 6-aminohexanoic acid
  MOD_RES 6..14: note = Xaa = beta-alanine
  MOD_RES 4: note = Xaa = -NH-(CHRe)n-C(O)-wherein n is 2 to 7 and each Re is independently, at each occurrence, hydrogen or methyl
  MOD_RES 16: note = Xaa = glycine or proline
RXRXRXRILFQYRXRX

SEQ ID NO: 217 - protein, 16 aa, synthetic construct
  REGION 1..16: note = cell penetrating peptide
  MOD_RES 2: note = Xaa = 6-aminohexanoic acid
  MOD_RES 10: note = Xaa = beta-alanine
  MOD_RES 12: note = Xaa = -NH-(CHRe)n-C(O)-wherein n is 2 to 7 and each Re is independently, at each occurrence, hydrogen or methyl
  MOD_RES 16: note = Xaa = glycine or proline
  MOD_RES 14: note = Xaa = beta-alanine
RXRILFQYRXRXRXRX

SEQ ID NO: 218 - protein, 11 aa, synthetic construct
  REGION 1..11: note = cell penetrating peptide
  MOD_RES 2: note = Xaa = 6-aminohexanoic acid
  MOD_RES 4: note = Xaa = -NH-(CHRe)n-C(O)-wherein n is 2 to 7 and each Re is independently, at each occurrence, hydrogen or methyl
  MOD_RES 9: note = Xaa = beta-alanine
  MOD_RES 11: note = Xaa = glycine or proline
  MOD_RES 6: note = Xaa = 6-aminohexanoic acid
RXRXRXRRXRX

SEQ ID NO: 219 - protein, 11 aa, synthetic construct
  REGION 1..11: note = cell penetrating peptide
  MOD_RES 2..5: note = Xaa = 6-aminohexanoic acid
  MOD_RES 7: note = Xaa = -NH-(CHRe)n-C(O)-wherein n is 2 to 7 and each Re is independently, at each occurrence, hydrogen or methyl
  MOD_RES 9: note = Xaa = beta-alanine
  MOD_RES 11: note = Xaa = glycine or proline
RXRRXRXRXRX

SEQ ID NO: 220 - protein, 15 aa, synthetic construct
  REGION 1..15: note = cell penetrating peptide
  MOD_RES 2..10: note = Xaa = 6-aminohexanoic acid
  MOD_RES 13: note = Xaa = beta-alanine
  MOD_RES 15: note = Xaa = glycine or alanine
RXRILFQYRXRRXRX

SEQ ID NO: 221 - protein, 15 aa, synthetic construct
  REGION 1..15: note = cell penetrating peptide
  MOD_RES 2..5: note = Xaa = 6-aminohexanoic acid
  MOD_RES 13: note = Xaa = beta-alanine
  MOD_RES 15: note = Xaa = glycine or proline
RXRRXRLLFQYRXRX

SEQ ID NO: 222 - protein, 12 aa, synthetic construct
  REGION 1..12: note = cell penetrating peptide
  MOD_RES 2: note = Xaa = 6-aminohexanoic acid
  MOD_RES 4: note = Xaa = -NH-(CHRe)n-C(O)-wherein n is 2 to 7 and each Re is independently, at each occurrence, hydrogen or methyl
  MOD_RES 10: note = Xaa = beta-alanine
  MOD_RES 12: note = Xaa = glycine or proline
  MOD_RES 8: note = Xaa = -NH-(CHRe)n-C(O)-wherein n is 2 to 7 and each Re is independently, at each occurrence, hydrogen or methyl
  MOD_RES 6: note = Xaa = 6-aminohexanoic acid
RXRXRXRXRXRX

SEQ ID NO: 223 - protein, 20 aa, synthetic construct
  REGION 1..20: note = cell penetrating peptide
  MOD_RES 2..10: note = Xaa = 6-aminohexanoic acid
  MOD_RES 18: note = Xaa = beta-alanine
  MOD_RES 20: note = Xaa = glycine or proline
RXRILFQYRXRILFQYRXRX

SEQ ID NO: 224 - protein, 16 aa, synthetic construct
  REGION 1..16: note = cell penetrating peptide
  MOD_RES 2: note = Xaa = 6-aminohexanoic acid
  MOD_RES 4: note = Xaa = -NH-(CHRe)n-C(O)-wherein n is 2 to 7 and each Re is independently, at each occurrence, hydrogen or methyl
  MOD_RES 14: note = Xaa = beta-alanine
  MOD_RES 16: note = Xaa = glycine or proline
  MOD_RES 6: note = Xaa = 6-aminohexanoic acid
RXRXRXRILFQYRXRX

SEQ ID NO: 225 - protein, 16 aa, synthetic construct
  REGION 1..16: note = cell penetrating peptide
  MOD_RES 2..10: note = Xaa = 6-aminohexanoic acid
  MOD_RES 12: note = Xaa = -NH-(CHRe)n-C(O)-wherein n is 2 to 7 and each Re is independently, at each occurrence, hydrogen or methyl
  MOD_RES 14: note = Xaa = beta-alanine
  MOD_RES 16: note = Xaa = glycine or proline
RXRILFQYRXRXRXRX

SEQ ID NO: 226 - protein, 11 aa, synthetic construct
  REGION 1..11: note = cell penetrating peptide
  MOD_RES 2: note = Xaa = beta-alanine
  MOD_RES 4: note = Xaa = -NH-(CHRe)n-C(O)-wherein n is 2 to 7 and each Re is independently, at each occurrence, hydrogen or methyl
  MOD_RES 6: note = Xaa = 6-aminohexanoic acid
  MOD_RES 11: note = Xaa = glycine or proline
  MOD_RES 9: note = Xaa = beta-alanine
RXRXRXRRXRX

SEQ ID NO: 227 - protein, 11 aa, synthetic construct
  REGION 1..11: note = cell penetrating peptide
  MOD_RES 2: note = Xaa = beta-alanine
  MOD_RES 5: note = Xaa = 6-aminohexanoic acid
  MOD_RES 7: note = Xaa = -NH-(CHRe)n-C(O)-wherein n is 2 to 7 and each Re is independently, at each occurrence, hydrogen or methyl
  MOD_RES 11: note = Xaa = glycine or proline
  MOD_RES 9: note = Xaa = beta-alanine
RXRRXRXRXRX

SEQ ID NO: 228 - protein, 15 aa, synthetic construct
  REGION 1..15: note = cell penetrating peptide
  MOD_RES 2: note = Xaa = beta-alanine
  MOD_RES 10: note = Xaa = 6-aminohexanoic acid
  MOD_RES 15: note = Xaa - glycine or proline
  MOD_RES 13: note = Xaa = beta-alanine
RXRILFQYRXRRXRX

SEQ ID NO: 229 - protein, 15 aa, synthetic construct
  REGION 1..15: note = cell penetrating peptide
  MOD_RES 2: note = Xaa = beta-alanine
  MOD_RES 5: note = Xaa = 6-aminohexanoic acid
  MOD_RES 15: note = Xaa = glycine or proline
  MOD_RES 13: note = Xaa = beta-alanine
RXRRXRILFQYRXRX

SEQ ID NO: 230 - protein, 12 aa, synthetic construct
  REGION 1..12: note = cell penetrating peptide
  MOD_RES 2: note = Xaa = beta-alanine
  MOD_RES 4: note = Xaa = -NH-(CHRe)n-C(O)-wherein n is 2 to 7 and each Re is independently, at each occurrence, hydrogen or methyl
  MOD_RES 6: note = Xaa = 6-aminohexanoic acid
  MOD_RES 12: note = Xaa = glycine or proline
  MOD_RES 10: note = Xaa = beta-alanine
  MOD_RES 8: note = Xaa = -NH-(CHRe)n-C(O)-wherein n is 2 to 7 and each Re is independently, at each occurrence, hydrogen or methyl
RXRXRXRXRXRX

SEQ ID NO: 231 - protein, 20 aa, synthetic construct
  REGION 1..20: note = cell penetrating peptide
  MOD_RES 2: note = Xaa = beta-alanine
  MOD_RES 10: note = Xaa = 6-aminohexanoic acid
  MOD_RES 20: note = Xaa = glycine or proline
  MOD_RES 18: note = Xaa = beta-alanine
RXRILFQYRXRILFQYRXRX

SEQ ID NO: 232 - protein, 16 aa, synthetic construct
  REGION 1..16: note = cell penetrating peptide
  MOD_RES 2: note = Xaa = beta-alanine
  MOD_RES 4: note = Xaa = -NH-(CHRe)n-C(O)-wherein n is 2 to 7 and each Re is independently, at each occurrence, hydrogen or methyl
  MOD_RES 6: note = Xaa = 6-aminihexanoic acid
  MOD_RES 16: note = Xaa = glycine or proline
  MOD_RES 14: note = Xaa = beta-alanine
RXRXRXRILFQYRXRX

SEQ ID NO: 233 - protein, 16 aa, synthetic construct
  REGION 1..16: note = cell penetrating peptide
  MOD_RES 2: note = Xaa = beta-alanine
  MOD_RES 10: note = Xaa = 6-aminohexanoic acid
  MOD_RES 12: note = Xaa = -NH-(CHRe)n-C(O)-wherein n is 2 to 7 and each Re is independently, at each occurrence, hydrogen or methyl
  MOD_RES 16: note = Xaa = glycine or proline
  MOD_RES 14: note = Xaa = beta-alanine
RXRILFQYRXRXRXRX

SEQ ID NO: 234 - protein, 11 aa, synthetic construct
  REGION 1..11: note = cell penetrating peptide
  MOD_RES 2: note = Xaa = beta-alanine
  MOD_RES 4: note = Xaa = -NH-(CHRe)n-C(O)-wherein n is 2 to 7 and each Re is independently, at each occurrence, hydrogen or methyl
  MOD_RES 6..9: note = Xaa = 6-aminohexanoic acid
  MOD_RES 11: note = Xaa = glycine or proline
RXRXRXRRXRX

SEQ ID NO: 235 - protein, 11 aa, synthetic construct
  REGION 1..11: note = cell penetrating peptide
  MOD_RES 2: note = Xaa = beta-alanine
  MOD_RES 5: note = Xaa = 6-aminohexanoic acid
  MOD_RES 7: note = Xaa = -NH-(CHRe)n-C(O)-wherein n is 2 to 7 and each Re is independently, at each occurrence, hydrogen or methyl
  MOD_RES 11: note = Xaa = glycine or proline
  MOD_RES 9: note = Xaa = 6-aminohexanoic acid
RXRRXRXRXRX

SEQ ID NO: 236 - protein, 15 aa, synthetic construct
  REGION 1..15: note = cell penetrating peptide
  MOD_RES 2: note = Xaa = beta-alanine
  MOD_RES 10..13: note = Xaa = 6-aminohexanoic acid
  MOD_RES 15: note = Xaa = glycine or proline
RXRILFQYRXRRXRX

SEQ ID NO: 237 - protein, 15 aa, synthetic construct
  REGION 1..15: note = cell penetrating peptide
  MOD_RES 2: note = Xaa = beta-alanine
  MOD_RES 5..13: note = Xaa = 6-aminohexanoic acid
  MOD_RES 15: note = Xaa = glycine or proline
RXRRXRILFQYRXRX

SEQ ID NO: 238 - protein, 12 aa, synthetic construct
  REGION 1..12: note = cell penetrating peptide
  MOD_RES 2: note = Xaa = beta-alanine
  MOD_RES 6: note = Xaa = 6-aminohexanoic acid
  MOD_RES 4: note = Xaa = -NH-(CHRe)n-C(O)-wherein n is 2 to 7 and each Re is independently, at each occurrence, hydrogen or methyl
  MOD_RES 12: note = Xaa = glycine or proline
  MOD_RES 8: note = Xaa = -NH-(CHRe)n-C(O)-wherein n is 2 to 7 and each Re is independently, at each occurrence, hydrogen or methyl
  MOD_RES 10: note = Xaa = 6-aminohexanoic acid
RXRXRXRXRXRX

SEQ ID NO: 239 - protein, 20 aa, synthetic construct
  REGION 1..20: note = cell penetrating peptide
  MOD_RES 2: note = Xaa = beta-alanine
  MOD_RES 10..18: note = Xaa = 6-aminohexanoic acid
  MOD_RES 20: note = Xaa = glycine or proline
RXRILFQYRXRILFQYRXRX

SEQ ID NO: 240 - protein, 16 aa, synthetic construct
  REGION 1..16: note = cell penetrating peptide
  MOD_RES 2: note = Xaa = beta-alanine
  MOD_RES 6: note = Xaa = 6-aminohexanoic acid
  MOD_RES 4: note = Xaa = -NH-(CHRe)n-C(O)-wherein n is 2 to 7 and each Re is independently, at each occurrence, hydrogen or methyl
  MOD_RES 16: note = Xaa = glycine or proline
  MOD_RES 14: note = Xaa = 6-aminohexanoic acid
RXRXRXRILFQYRXRX

SEQ ID NO: 241 - protein, 16 aa, synthetic construct
  REGION 1..16: note = cell penetrating peptide
  MOD_RES 2: note = Xaa = beta-alanine
  MOD_RES 10: note = Xaa = 6-aminohexanoic acid
  MOD_RES 12: note = Xaa = -NH-(CHRe)n-C(O)-wherein n is 2 to 7 and each Re is independently, at each occurrence, hydrogen or methyl
  MOD_RES 16: note = Xaa = glycine or proline
  MOD_RES 14: note = Xaa = 6-aminohexanoic acid
RXRILFQYRXRXRXRX

SEQ ID NO: 242 - protein, 11 aa, synthetic construct
  REGION 1..11: note = cell penetrating peptide
  MOD_RES 2: note = Xaa = beta-alanine
  MOD_RES 4: note = Xaa = -NH-(CHRe)n-C(O)-wherein n is 2 to 7 and each Re is independently, at each occurrence, hydrogen or methyl
  MOD_RES 9: note = Xaa = 6-aminohexanoic acid
  MOD_RES 11: note = Xaa = glycine or proline
  MOD_RES 6: note = Xaa = beta-alanine
RXRXRXRRXRX

SEQ ID NO: 243 - protein, 11 aa, synthetic construct
  REGION 1..11: note = cell penetrating peptide
  MOD_RES 2..5: note = Xaa = beta-alanine
  MOD_RES 7: note = Xaa = -NH-(CHRe)n-C(O)-wherein n is 2 to 7 and each Re is independently, at each occurrence, hydrogen or methyl
  MOD_RES 9: note = Xaa = 6-aminohexanoic acid
  MOD_RES 11: note = Xaa = glycine or proline
RXRRXRXRXRX

SEQ ID NO: 244 - protein, 15 aa, synthetic construct
  REGION 1..15: note = cell penetrating peptide
  MOD_RES 2..10: note = Xaa = beta-alanine
  MOD_RES 13: note = Xaa = 6-aminohexanoic acid
  MOD_RES 15: note = Xaa = glycine or proline
RXRILFQYRXRRXRX

SEQ ID NO: 245 - protein, 15 aa, synthetic construct
  REGION 1..15: note = cell penetrating peptide
  MOD_RES 2..5: note = Xaa = beta-alanine
  MOD_RES 13: note = Xaa = 6-aminohexanoic acid
  MOD_RES 15: note = Xaa = glycine or proline
RXRRXRILFQYRXRX

SEQ ID NO: 246 - protein, 12 aa, synthetic construct
  REGION 1..12: note = cell penetrating peptide
  MOD_RES 2: note = Xaa = beta-alanine
  MOD_RES 4: note = Xaa = -NH-(CHRe)n-C(O)-wherein n is 2 to 7 and each Re is independently, at each occurrence, hydrogen or methyl
  MOD_RES 10: note = Xaa = 6-aminohexanoic acid
  MOD_RES 12: note = Xaa = glycine or proline
  MOD_RES 8: note = Xaa = -NH-(CHRe)n-C(O)-wherein n is 2 to 7 and each Re is independently, at each occurrence, hydrogen or methyl
  MOD_RES 6: note = Xaa = beta-alanine
RXRXRXRXRXRX

SEQ ID NO: 247 - protein, 20 aa, synthetic construct
  REGION 1..20: note = cell penetrating peptide
  MOD_RES 2..10: note = Xaa = beta-alanine
  MOD_RES 18: note = Xaa = 6-aminohexanoic acid
  MOD_RES 20: note = Xaa = glycine or proline
RXRILFQYRXRILFQYRXRX

SEQ ID NO: 248 - protein, 16 aa, synthetic construct
  REGION 1..16: note = cell penetrating peptide
  MOD_RES 2: note = Xaa = beta-alanine
  MOD_RES 4: note = Xaa = -NH-(CHRe)n-C(O)-wherein n is 2 to 7 and each Re is independently, at each occurrence, hydrogen or methyl
  MOD_RES 14: note = Xaa = 6-aminohexanoic acid
  MOD_RES 16: note = Xaa = glycine or proline
  MOD_RES 6: note = Xaa = beta-alanine
RXRXRXRILFQYRXRX

SEQ ID NO: 249 - protein, 16 aa, synthetic construct
  REGION 1..16: note = cell penetrating peptide
  MOD_RES 2..10: note = Xaa = beta-alanine
  MOD_RES 12: note = Xaa = -NH-(CHRe)n-C(O)-wherein n is 2 to 7 and each Re is independently, at each occurrence, hydrogen or methyl
  MOD_RES 14: note = Xaa = 6-aminohexanoic acid
  MOD_RES 16: note = Xaa = glycine or proline
RXRILFQYRXRXRXRX

SEQ ID NO: 250 - protein, 13 aa, synthetic construct
  REGION 1..13: note = cell penetrating peptide
  MOD_RES 2..11: note = Xaa = 6-aminohexanoic acid
  MOD_RES 13: note = Xaa = glycine or proline
RXRRXRRXRRXRX

SEQ ID NO: 251 - protein, 22 aa, synthetic construct
  REGION 1..22: note = cell penetrating peptide
  MOD_RES 8..16: note = Xaa = 6-aminohexanoic acid
  MOD_RES 5: note = Xaa = beta-alanine
  MOD_RES 22: note = Xaa = glycine or proline
  MOD_RES 18: note = Xaa = beta-alanine
  MOD_RES 2: note = Xaa = 6-aminohexanoic acid
  MOD_RES 20: note = Xaa = 6-aminohexanoic acid
RXRRXRRXRILFQYRXRXRXRX

SEQ ID NO: 252 - protein, 13 aa, synthetic construct
  REGION 1..13: note = cell penetrating peptide
  MOD_RES 2: note = Xaa = 6-aminohexanoic acid
  MOD_RES 5: note = Xaa = beta-alanine
  MOD_RES 13: note = Xaa = glycine or proline
  MOD_RES 11: note = Xaa = beta-alanine
  MOD_RES 8: note = Xaa = 6-aminohexanoic acid
RXRRXRRXRRXRX

SEQ ID NO: 253 - protein, 13 aa, synthetic construct
  REGION 1..13: note = cell penetrating peptide
  MOD_RES 13: note = Xaa = glycine or proline
YGRKKRRQRRRPX

SEQ ID NO: 254 - protein, 24 aa, synthetic construct
  REGION 1..24: note = cell penetrating peptide
  MOD_RES 2..13: note = Xaa = 6-aminohexanoic acid
  MOD_RES 14: note = Xaa = beta-alanine
  MOD_RES 24: note = Xaa = glycine or proline
  MOD_RES 22: note = Xaa = 6-aminohexanoic acid
RXRRXRRXRRXRXXASSLNIAXCX

SEQ ID NO: 255 - protein, 32 aa, synthetic construct
  REGION 1..32: note = cell penetrating peptide
  MOD_RES 8..16: note = Xaa = 6-aminohexanoic acid
  MOD_RES 5: note = Xaa = beta-alanine
  MOD_RES 32: note = Xaa = glycine or proline
  MOD_RES 2: note = Xaa = 6-aminohexanoic acid
  MOD_RES 20: note = Xaa = 6-aminohexanoic acid
  MOD_RES 30: note = Xaa = 6-aminohexanoic acid
  MOD_RES 18: note = Xaa = beta-alanine
  MOD_RES 22: note = Xaa = beta-alanine
RXRRXRRXRILFQYRXRXRXRXASSLNIAXCX

SEQ ID NO: 256 - protein, 26 aa, synthetic construct
  REGION 1..26: note = cell penetrating peptide
  MOD_RES 5: note = Xaa = beta-alanine
  MOD_RES 8..18: note = Xaa = 6-aminohexanoic acid
  MOD_RES 26: note = Xaa = glycine or proline
  MOD_RES 2: note = Xaa = 6-aminohexanoic acid
  MOD_RES 22: note = Xaa = 6-aminohexanoic acid
  MOD_RES 20: note = Xaa = beta-alanine
  MOD_RES 24: note = Xaa = beta-alanine
RXRRXRRXRASSLNIARXRXRXRXCX

SEQ ID NO: 257 - protein, 22 aa, synthetic construct
  REGION 1..22: note = cell penetrating peptide
  MOD_RES 2: note = Xaa = 6-aminohexanoic acid
  MOD_RES 5: note = Xaa = beta-alanine
  MOD_RES 22: note = Xaa = glycine or proline
  MOD_RES 11: note = Xaa = beta-alanine
  MOD_RES 14: note = Xaa = beta-alanine
  MOD_RES 8: note = Xaa = 6-aminohexanoic acid
  MOD_RES 13: note = Xaa = 6-aminohexanoic acid
RXRRXRRXRRXRXXASSLNIAX

SEQ ID NO: 258 - protein, 13 aa, synthetic construct
  REGION 1..13: note = cell penetrating peptide
  MOD_RES 13: note = Xaa = glycine or proline
THRPPMWSPVWPX

SEQ ID NO: 259 - protein, 12 aa, synthetic construct
  REGION 1..12: note = cell penetrating peptide
  MOD_RES 12: note = Xaa = glycine or proline
HRPPMWSPVWPX

SEQ ID NO: 260 - protein, 11 aa, synthetic construct
  REGION 1..11: note = cell penetrating peptide
  MOD_RES 11: note = Xaa = glycine or proline
THRPPMWSPVX

SEQ ID NO: 261 - protein, 10 aa, synthetic construct
  REGION 1..10: note = cell penetrating peptide
  MOD_RES 10: note = Xaa = glycine or proline
THRPPMWSPX

SEQ ID NO: 262 - protein, 13 aa, synthetic construct
  REGION 1..13: note = cell penetrating peptide
  MOD_RES 13: note = Xaa = glycine or proline
THRPPMWSPVFPX

SEQ ID NO: 263 - protein, 13 aa, synthetic construct
  REGION 1..13: note = cell penetrating peptide
  MOD_RES 13: note = Xaa = glycine or proline
THRPPMWSPVYPX

SEQ ID NO: 264 - protein, 13 aa, synthetic construct
  REGION 1..13: note = cell penetrating peptide
  MOD_RES 13: note = Xaa = glycine or proline
THRPPMWSPAWPX

SEQ ID NO: 265 - protein, 13 aa, synthetic construct
  REGION 1..13: note = cell penetrating peptide
  MOD_RES 13: note = Xaa = glycine or proline
THRPPMWSPLWPX

SEQ ID NO: 266 - protein, 13 aa, synthetic construct
  REGION 1..13: note = cell penetrating peptide
  MOD_RES 13: note = Xaa = glycine or proline
THRPPMWSPIWPX

SEQ ID NO: 267 - protein, 14 aa, synthetic construct
  REGION 1..14: note = cell penetrating peptide
  MOD_RES 14: note = Xaa = glycine or proline
THRPPMWTPVVWPX

SEQ ID NO: 268 - protein, 13 aa, synthetic construct
  REGION 1..13: note = cell penetrating peptide
  MOD_RES 13: note = Xaa = glycine or proline
THRPPMFSPVWPX

SEQ ID NO: 269 - protein, 9 aa, synthetic construct
  REGION 1..9: note = cell penetrating peptide
  MOD_RES 9: note = Xaa = glycine or proline
THRPPMWSX

SEQ ID NO: 270 - protein, 11 aa, synthetic construct
  REGION 1..11: note = cell penetrating peptide
  MOD_RES 11: note = Xaa = glycine or proline
HRPPMWSPVWX

SEQ ID NO: 271 - protein, 13 aa, synthetic construct
  REGION 1..13: note = cell penetrating peptide
  MOD_RES 13: note = Xaa = glycine or proline
THRPPMYSPVWPX

SEQ ID NO: 272 - protein, 13 aa, synthetic construct
  REGION 1..13: note = cell penetrating peptide
  MOD_RES 13: note = Xaa = glycine or proline
  MOD_RES 6: note = Xaa = norleucine
THRPPXWSPVWPX

SEQ ID NO: 273 - protein, 13 aa, synthetic construct
  REGION 1..13: note = cell penetrating peptide
  MOD_RES 13: note = Xaa = glycine or proline
THKPPMWSPVWPX

SEQ ID NO: 274 - protein, 13 aa, synthetic construct
  REGION 1..13: note = cell penetrating peptide
  MOD_RES 13: note = Xaa = glycine or proline
SHRPPMWSPVWPX

SEQ ID NO: 275 - protein, 8 aa, synthetic construct
  REGION 1..8: note = cell penetrating peptide
  MOD_RES 8: note = Xaa = glycine or proline
STFTHPRX

SEQ ID NO: 276 - protein, 8 aa, synthetic construct
  REGION 1..8: note = cell penetrating peptide
  MOD_RES 8: note = Xaa = glycine or proline
YDIDNRRX

SEQ ID NO: 277 - protein, 8 aa, synthetic construct
  REGION 1..8: note = cell penetrating peptide
  MOD_RES 8: note = Xaa = glycine or proline
AYKPVGRX

SEQ ID NO: 278 - protein, 8 aa, synthetic construct
  REGION 1..8: note = cell penetrating peptide
  MOD_RES 8: note = Xaa = glycine or proline
HAIYPRHX

SEQ ID NO: 279 - protein, 8 aa, synthetic construct
  REGION 1..8: note = cell penetrating peptide
  MOD_RES 8: note = Xaa = glycine or proline
HTPNSTHX

SEQ ID NO: 280 - protein, 8 aa, synthetic construct
  REGION 1..8: note = cell penetrating peptide
  MOD_RES 8: note = Xaa = glycine or proline
ASSPVHRX

SEQ ID NO: 281 - protein, 8 aa, synthetic construct
  REGION 1..8: note = cell penetrating peptide
  MOD_RES 8: note = Xaa = glycine or proline
SSLPLRKX

SEQ ID NO: 282 - protein, 5 aa, synthetic construct
  REGION 1..5: note = cell penetrating peptide
  MOD_RES 5: note = Xaa = glycine or proline
KKRSX

SEQ ID NO: 283 - protein, 5 aa, synthetic construct
  REGION 1..5: note = cell penetrating peptide
  MOD_RES 5: note = Xaa = glycine or proline
KRSKX

SEQ ID NO: 284 - protein, 6 aa, synthetic construct
  REGION 1..6: note = cell penetrating peptide
  MOD_RES 6: note = Xaa = glycine or proline
KKRSKX

SEQ ID NO: 285 - protein, 5 aa, synthetic construct
  REGION 1..5: note = cell penetrating peptide
  MOD_RES 5: note = Xaa = glycine or proline
KSRKX

SEQ ID NO: 286 - protein, 5 aa, synthetic construct
  REGION 1..5: note = cell penetrating peptide
  MOD_RES 5: note = Xaa = glycine or proline
SRKRX

SEQ ID NO: 287 - protein, 5 aa, synthetic construct
  REGION 1..5: note = cell penetrating peptide
  MOD_RES 5: note = Xaa = glycine or proline
RKRKX

SEQ ID NO: 288 - protein, 6 aa, synthetic construct
  REGION 1..6: note = cell penetrating peptide
  MOD_RES 6: note = Xaa = glycine or proline
KSRKRX

SEQ ID NO: 289 - protein, 8 aa, synthetic construct
  REGION 1..8: note = cell penetrating peptide
  MOD_RES 8: note = Xaa = glycine or proline
QHPPWRVX

SEQ ID NO: 290 - protein, 8 aa, synthetic construct
  REGION 1..8: note = cell penetrating peptide
  MOD_RES 8: note = Xaa = glycine or proline
THPPTTHX

SEQ ID NO: 291 - protein, 8 aa, synthetic construct
  REGION 1..8: note = cell penetrating peptide
  MOD_RES 8: note = Xaa = glycine or proline
YKHTPTTX

SEQ ID NO: 292 - protein, 8 aa, synthetic construct
  REGION 1..8: note = cell penetrating peptide
  MOD_RES 8: note = Xaa = glycine or proline
QGMHRGTX

SEQ ID NO: 293 - protein, 6 aa, synthetic construct
  REGION 1..6: note = cell penetrating peptide
  MOD_RES 6: note = Xaa = glycine or proline
SRKRKX

SEQ ID NO: 294 - protein, 7 aa, synthetic construct
  REGION 1..7: note = cell penetrating peptide
  MOD_RES 7: note = Xaa = glycine or proline
KSRKRKX

SEQ ID NO: 295 - protein, 8 aa, synthetic construct
  REGION 1..8: note = cell penetrating peptide
  MOD_RES 8: note = Xaa = glycine or proline
PKKKRKVX

SEQ ID NO: 296 - protein, 8 aa, synthetic construct
  REGION 1..8: note = cell penetrating peptide
  MOD_RES 8: note = Xaa = glycine or proline
GKKRSKVX

SEQ ID NO: 297 - protein, 8 aa, synthetic construct
  REGION 1..8: note = cell penetrating peptide
  MOD_RES 8: note = Xaa = glycine or proline
KSRKRKLX

SEQ ID NO: 298 - protein, 8 aa, synthetic construct
  REGION 1..8: note = cell penetrating peptide
  MOD_RES 8: note = Xaa = glycine or proline
HSPSKIPX

SEQ ID NO: 299 - protein, 8 aa, synthetic construct
  REGION 1..8: note = cell penetrating peptide
  MOD_RES 8: note = Xaa = glycine or proline
HMATFHYX

SEQ ID NO: 300 - protein, 8 aa, synthetic construct
  REGION 1..8: note = cell penetrating peptide
  MOD_RES 8: note = Xaa = glycine or proline
AQPNKFKX

SEQ ID NO: 301 - protein, 8 aa, synthetic construct
  REGION 1..8: note = cell penetrating peptide
  MOD_RES 8: note = Xaa = glycine or proline
NLTRLHTX

SEQ ID NO: 302 - protein, 5 aa, synthetic construct
  REGION 1..5: note = cell penetrating peptide
  MOD_RES 5: note = Xaa = glycine or proline
KKKRX

SEQ ID NO: 303 - protein, 5 aa, synthetic construct
  REGION 1..5: note = cell penetrating peptide
  MOD_RES 5: note = Xaa = glycine or proline
KKRKX

SEQ ID NO: 304 - protein, 6 aa, synthetic construct
  REGION 1..6: note = cell penetrating peptide
  MOD_RES 6: note = Xaa = glycine or proline
KKKRKX

SEQ ID NO: 305 - protein, 25 aa, synthetic construct
  REGION 1..25: note = cell penetrating peptide
  MOD_RES 25: note = Xaa = glycine or proline
RRRRRRQIKIWFQNRRMKWKKGGCX

SEQ ID NO: 306 - protein, 26 aa, synthetic construct
  REGION 1..26: note = cell penetrating peptide
  MOD_RES 26: note = Xaa = glycine or proline
RRRRRRRQIKIWFQNRRMKWKKGGCX

SEQ ID NO: 307 - protein, 20 aa, synthetic construct
  REGION 1..20: note = cell penetrating peptide
  MOD_RES 20: note = Xaa = glycine or proline
RQIKIWFQNRRMKWKKGGCX

SEQ ID NO: 308 - protein, 24 aa, synthetic construct
  REGION 1..24: note = cell penetrating peptide
  MOD_RES 24: note = Xaa = glycine or proline
RRRRRRRQIKIWFQNRRMKWKKCX

SEQ ID NO: 309 - protein, 29 aa, synthetic construct
  REGION 1..29: note = cell penetrating peptide
  MOD_RES 2..8: note = Xaa = 6-aminohexanoic acid
  MOD_RES 29: note = Xaa = glycine or proline
RXRRXRRXRRQIKIWFQNRRMKWKKGGCX

SEQ ID NO: 310 - protein, 25 aa, synthetic construct
  REGION 1..25: note = cell penetrating peptide
  MOD_RES 17..23: note = Xaa = 6-aminohexanoic acid
  MOD_RES 25: note = Xaa = glycine or proline
RRRRRRRQIKILFQNRXRXRXRXCX

SEQ ID NO: 311 - protein, 14 aa, synthetic construct
  REGION 1..14: note = cell penetrating peptide
  MOD_RES 2..11: note = Xaa = 6-aminohexanoic acid
  MOD_RES 14: note = Xaa = glycine or proline
RXRRXRRXRRXRCX

SEQ ID NO: 312 - protein, 15 aa, synthetic construct
  REGION 1..15: note = cell penetrating peptide
  MOD_RES 2..13: note = Xaa = 6-aminohexanoic acid
  MOD_RES 15: note = Xaa = glycine or proline
RXRRXRRXRRXRXCX

SEQ ID NO: 313 - protein, 27 aa, synthetic construct
  REGION 1..27: note = cell penetrating peptide
  MOD_RES 2..8: note = Xaa = 6-aminohexanoic acid
  MOD_RES 27: note = Xaa = glycine or proline
RXRRXRRXRIKILFQNRRMKWKKGGCX

SEQ ID NO: 314 - protein, 25 aa, synthetic construct
  REGION 1..25: note = cell penetrating peptide
  MOD_RES 2..8: note = Xaa = 6-aminohexanoic acid
  MOD_RES 25: note = Xaa = glycine or proline
RXRRXRRXRIKILFQNRRMKWKKCX

SEQ ID NO: 315 - protein, 24 aa, synthetic construct
  REGION 1..24: note = cell penetrating peptide
  MOD_RES 2..8: note = Xaa = 6-aminohexanoic acid
  MOD_RES 24: note = Xaa = glycine or proline
RXRRXRRXRIKILFQNRMKWKKCX

SEQ ID NO: 316 - protein, 25 aa, synthetic construct
  REGION 1..25: note = cell penetrating peptide
  MOD_RES 2..17: note = Xaa = 6-aminohexanoic acid
  MOD_RES 25: note = Xaa = glycine or proline
RXRRXRRXRIKILFQNXRMKWKKCX

SEQ ID NO: 317 - protein, 25 aa, synthetic construct
  REGION 1..25: note = cell penetrating peptide
  MOD_RES 2..8: note = Xaa = 6-aminohexanoic acid
  MOD_RES 25: note = Xaa = glycine or proline
RXRRXRRXRIKILFQNHRMKWKKCX

SEQ ID NO: 318 - protein, 25 aa, synthetic construct
  REGION 1..25: note = cell penetrating peptide
  MOD_RES 2..17: note = Xaa = 6-aminohexanoic acid
  MOD_RES 25: note = Xaa = glycine or proline
RXRRXRRXRIKILFQNXRMKWKKCX

SEQ ID NO: 319 - protein, 25 aa, synthetic construct
  REGION 1..25: note = cell penetrating peptide
  MOD_RES 2..17: note = Xaa = 6-aminohexanoic acid
  MOD_RES 25: note = Xaa = glycine or proline
RXRRXRRXRIKILFQNXRMKWKKCX

SEQ ID NO: 320 - protein, 25 aa, synthetic construct
  REGION 1..25: note = cell penetrating peptide
  MOD_RES 2..17: note = Xaa = 6-aminohexanoic acid
  MOD_RES 25: note = Xaa = glycine or proline
RXRRXRRXRIKILFQNXRMKWKACX

SEQ ID NO: 321 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIKILFQNXRMKWHKACX

SEQ ID NO: 322 - protein, 25 aa, synthetic construct
  REGION 1..25: note = cell penetrating peptide
  MOD_RES 2..17: note = Xaa = 6-aminohexanoic acid
  MOD_RES 25: note = Xaa = glycine or proline
RXRRXRRXRIKILFQNXRMKWHRCX

SEQ ID NO: 323 - protein, 25 aa, synthetic construct
  REGION 1..25: note = cell penetrating peptide
  MOD_RES 2..8: note = Xaa = 6-aminohexanoic acid
  MOD_RES 25: note = Xaa = glycine or proline
RXRXRXRXRIKILFQNRRMKWKKCX

SEQ ID NO: 324 - protein, 25 aa, synthetic construct
  REGION 1..25: note = cell penetrating peptide
  MOD_RES 25: note = Xaa = glycine or proline
RARARARARIKILFQNRRMKWKKCX

SEQ ID NO: 325 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIXILFQNXRMKWHKACX

SEQ ID NO: 326 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIHILFQNXRMKWHKACX

SEQ ID NO: 327 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIRILFQNXRMKWHKACX

SEQ ID NO: 328 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIXILFQYXRMKWHKACX

SEQ ID NO: 329 - protein, 27 aa, synthetic construct
  REGION 1..27: note = cell penetrating peptide
  MOD_RES 2..18: note = Xaa = 6-aminohexanoic acid
  MOD_RES 27: note = Xaa = glycine or proline
RXRRXRRXRLYSPLSFQXRMKWHKACX

SEQ ID NO: 330 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRISILFQYXRMKWHKACX

SEQ ID NO: 331 - protein, 24 aa, synthetic construct
  REGION 1..24: note = cell penetrating peptide
  MOD_RES 2..15: note = Xaa = 6-aminohexanoic acid
  MOD_RES 24: note = Xaa = glycine or proline
RXRRXRRXRILFQYXRMKWHKACX

SEQ ID NO: 332 - protein, 23 aa, synthetic construct
  REGION 1..23: note = cell penetrating peptide
  MOD_RES 2..14: note = Xaa = 6-aminohexanoic acid
  MOD_RES 23: note = Xaa = glycine or proline
RXRRXRIXILFQYXRMKWHKACX

SEQ ID NO: 333 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRARRXRIHILFQYXRMKWHKACX

SEQ ID NO: 334 - protein, 26 aa, synthetic construct
  REGION 1..26: note = cell penetrating peptide
  MOD_RES 5..17: note = Xaa = 6-aminohexanoic acid
  MOD_RES 26: note = Xaa = glycine or proline
RARRXRRARIHILFQYXRMKWHKACX

SEQ ID NO: 335 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIHILFQYXRMKWHKACX

SEQ ID NO: 336 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIXILFQNXRMKWHKACX

SEQ ID NO: 337 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIHILFQNXRMKWHKACX

SEQ ID NO: 338 - protein, 24 aa, synthetic construct
  REGION 1..24: note = cell penetrating peptide
  MOD_RES 2..8: note = Xaa = 6-aminohexanoic acid
  MOD_RES 24: note = Xaa = glycine or proline
RXRRXRRXRIKILFQNRRMKWHKX

SEQ ID NO: 339 - protein, 24 aa, synthetic construct
  REGION 1..24: note = cell penetrating peptide
  MOD_RES 2..17: note = Xaa = 6-aminohexanoic acid
  MOD_RES 24: note = Xaa = glycine or proline
RXRRXRRXRIKILFQNXRMKWHKX

SEQ ID NO: 340 - protein, 24 aa, synthetic construct
  REGION 1..24: note = cell penetrating peptide
  MOD_RES 2..11: note = Xaa = 6-aminohexanoic acid
  MOD_RES 24: note = Xaa = glycine or proline
RXRRXRRXRIXILFQNRRMKWHKX

SEQ ID NO: 341 - protein, 24 aa, synthetic construct
  REGION 1..24: note = cell penetrating peptide
  MOD_RES 2..17: note = Xaa = 6-aminohexanoic acid
  MOD_RES 24: note = Xaa = glycine or proline
RXRRXRRXRIXILFQNXRMKWHKX

SEQ ID NO: 342 - protein, 24 aa, synthetic construct
  REGION 1..24: note = cell penetrating peptide
  MOD_RES 2..8: note = Xaa = 6-aminohexanoic acid
  MOD_RES 24: note = Xaa = glycine or proline
RXRRXRRXRIHILFQNRRMKWHKX

SEQ ID NO: 343 - protein, 24 aa, synthetic construct
  REGION 1..24: note = cell penetrating peptide
  MOD_RES 2..17: note = Xaa = 6-aminohexanoic acid
  MOD_RES 24: note = Xaa = glycine or proline
RXRRXRRXRIHILFQNXRMKWHKX

SEQ ID NO: 344 - protein, 24 aa, synthetic construct
  REGION 1..24: note = cell penetrating peptide
  MOD_RES 2..8: note = Xaa = 6-aminohexanoic acid
  MOD_RES 24: note = Xaa = glycine or proline
RXRRXRRXRIRILFQNRRMKWHKX

SEQ ID NO: 345 - protein, 24 aa, synthetic construct
  REGION 1..24: note = cell penetrating peptide
  MOD_RES 2..17: note = Xaa = 6-aminohexanoic acid
  MOD_RES 24: note = Xaa = glycine or proline
RXRRXRRXRIRILFQNXRMKWHKX

SEQ ID NO: 346 - protein, 23 aa, synthetic construct
  REGION 1..23: note = cell penetrating peptide
  MOD_RES 2..8: note = Xaa = 6-aminohexanoic acid
  MOD_RES 23: note = Xaa = glycine or proline
RXRRXRRXRIILFQNRRMKWHKX

SEQ ID NO: 347 - protein, 23 aa, synthetic construct
  REGION 1..23: note = cell penetrating peptide
  MOD_RES 2..16: note = Xaa = 6-aminohexanoic acid
  MOD_RES 23: note = Xaa = glycine or proline
RXRRXRRXRIILFQNXRMKWHKX

SEQ ID NO: 348 - protein, 23 aa, synthetic construct
  REGION 1..23: note = cell penetrating peptide
  MOD_RES 2..8: note = Xaa = 6-aminohexanoic acid
  MOD_RES 23: note = Xaa = glycine or proline
RXRRXRRXRKILFQNRRMKWHKX

SEQ ID NO: 349 - protein, 23 aa, synthetic construct
  REGION 1..23: note = cell penetrating peptide
  MOD_RES 2..16: note = Xaa = 6-aminohexanoic acid
  MOD_RES 23: note = Xaa = glycine or proline
RXRRXRRXRKILFQNXRMKWHKX

SEQ ID NO: 350 - protein, 23 aa, synthetic construct
  REGION 1..23: note = cell penetrating peptide
  MOD_RES 2..10: note = Xaa = 6-aminohexanoic acid
  MOD_RES 23: note = Xaa = glycine or proline
RXRRXRRXRXILFQNRRMKWHKX

SEQ ID NO: 351 - protein, 23 aa, synthetic construct
  REGION 1..23: note = cell penetrating peptide
  MOD_RES 2..16: note = Xaa = 6-aminohexanoic acid
  MOD_RES 23: note = Xaa = glycine or proline
RXRRXRRXRXILFQNXRMKWHKX

SEQ ID NO: 352 - protein, 23 aa, synthetic construct
  REGION 1..23: note = cell penetrating peptide
  MOD_RES 2..8: note = Xaa = 6-aminohexanoic acid
  MOD_RES 23: note = Xaa = glycine or proline
RXRRXRRXRHILFQNRRMKWHKX

SEQ ID NO: 353 - protein, 23 aa, synthetic construct
  REGION 1..23: note = cell penetrating peptide
  MOD_RES 2..16: note = Xaa = 6-aminohexanoic acid
  MOD_RES 23: note = Xaa = glycine or proline
RXRRXRRXRHILFQNXRMKWHKX

SEQ ID NO: 354 - protein, 23 aa, synthetic construct
  REGION 1..23: note = cell penetrating peptide
  MOD_RES 2..8: note = Xaa = 6-aminohexanoic acid
  MOD_RES 23: note = Xaa = glycine or proline
RXRRXRRXRRILFQNRRMKWHKX

SEQ ID NO: 355 - protein, 23 aa, synthetic construct
  REGION 1..23: note = cell penetrating peptide
  MOD_RES 2..16: note = Xaa = 6-aminohexanoic acid
  MOD_RES 23: note = Xaa = glycine or proline
RXRRXRRXRRILFQNXRMKWHKX

SEQ ID NO: 356 - protein, 22 aa, synthetic construct
  REGION 1..22: note = cell penetrating peptide
  MOD_RES 2..8: note = Xaa = 6-aminohexanoic acid
  MOD_RES 22: note = Xaa = glycine or proline
RXRRXRRXRILFQNRRMKWHKX

SEQ ID NO: 357 - protein, 22 aa, synthetic construct
  REGION 1..22: note = cell penetrating peptide
  MOD_RES 2..15: note = Xaa = 6-aminohexanoic acid
  MOD_RES 22: note = Xaa = glycine or proline
RXRRXRRXRILFQNXRMKWHKX

SEQ ID NO: 358 - protein, 24 aa, synthetic construct
  REGION 1..24: note = cell penetrating peptide
  MOD_RES 2..8: note = Xaa = 6-aminohexanoic acid
  MOD_RES 24: note = Xaa = glycine or proline
RXRRXRRXRIKILFQYRRMKWHKX

SEQ ID NO: 359 - protein, 24 aa, synthetic construct
  REGION 1..24: note = cell penetrating peptide
  MOD_RES 2..17: note = Xaa = 6-aminohexanoic acid
  MOD_RES 24: note = Xaa = glycine or proline
RXRRXRRXRIKILFQYXRMKWHKX

SEQ ID NO: 360 - protein, 24 aa, synthetic construct
  REGION 1..24: note = cell penetrating peptide
  MOD_RES 2..11: note = Xaa = 6-aminohexanoic acid
  MOD_RES 24: note = Xaa = glycine or proline
RXRRXRRXRIXILFQYRRMKWHKX

SEQ ID NO: 361 - protein, 24 aa, synthetic construct
  REGION 1..24: note = cell penetrating peptide
  MOD_RES 2..17: note = Xaa = 6-aminohexanoic acid
  MOD_RES 24: note = Xaa = glycine or proline
RXRRXRRXRIXILFQYXRMKWHKX

SEQ ID NO: 362 - protein, 24 aa, synthetic construct
  REGION 1..24: note = cell penetrating peptide
  MOD_RES 2..8: note = Xaa = 6-aminohexanoic acid
  MOD_RES 24: note = Xaa = glycine or proline
RXRRXRRXRIHILFQYRRMKWHKX

SEQ ID NO: 363 - protein, 24 aa, synthetic construct
  REGION 1..24: note = cell penetrating peptide
  MOD_RES 2..17: note = Xaa = 6-aminohexanoic acid
  MOD_RES 24: note = Xaa = glycine or proline
RXRRXRRXRIHILFQYXRMKWHKX

SEQ ID NO: 364 - protein, 24 aa, synthetic construct
  REGION 1..24: note = cell penetrating peptide
  MOD_RES 2..8: note = Xaa = 6-aminohexanoic acid
  MOD_RES 24: note = Xaa = glycine or proline
RXRRXRRXRIRILFQYRRMKWHKX

SEQ ID NO: 365 - protein, 24 aa, synthetic construct
  REGION 1..24: note = cell penetrating peptide
  MOD_RES 2..17: note = Xaa = 6-aminohexanoic acid
  MOD_RES 24: note = Xaa = glycine or proline
RXRRXRRXRIRILFQYXRMKWHKX

SEQ ID NO: 366 - protein, 23 aa, synthetic construct
  REGION 1..23: note = cell penetrating peptide
  MOD_RES 2..8: note = Xaa = 6-aminohexanoic acid
  MOD_RES 23: note = Xaa - glycine or proline
RXRRXRRXRIILFQYRRMKWHKX

SEQ ID NO: 367 - protein, 23 aa, synthetic construct
  REGION 1..23: note = cell penetrating peptide
  MOD_RES 2..16: note = Xaa = 6-aminohexanoic acid
  MOD_RES 23: note = Xaa = glycine or proline
RXRRXRRXRIILFQYXRMKWHKX

SEQ ID NO: 368 - protein, 23 aa, synthetic construct
  REGION 1..23: note = cell penetrating peptide
  MOD_RES 2..8: note = Xaa = 6-aminohexanoic acid
  MOD_RES 23: note = Xaa = glycine or proline
RXRRXRRXRKILFQYRRMKWHKX

SEQ ID NO: 369 - protein, 23 aa, synthetic construct
  REGION 1..23: note = cell penetrating peptide
  MOD_RES 2..16: note = Xaa = 6-aminohexanoic acid
  MOD_RES 23: note = Xaa = glycine or proline
RXRRXRRXRKILFQYXRMKWHKX

SEQ ID NO: 370 - protein, 23 aa, synthetic construct
  REGION 1..23: note = cell penetrating peptide
  MOD_RES 2..10: note = Xaa = 6-aminohexanoic acid
  MOD_RES 23: note = Xaa = glycine or proline
RXRRXRRXRXILFQYRRMKWHKX

SEQ ID NO: 371 - protein, 23 aa, synthetic construct
  REGION 1..23: note = cell penetrating peptide
  MOD_RES 2..16: note = Xaa = 6-aminohexanoic acid
  MOD_RES 23: note = Xaa = glycine or proline
RXRRXRRXRXILFQYXRMKWHKX

SEQ ID NO: 372 - protein, 23 aa, synthetic construct
  REGION 1..23: note = cell penetrating peptide
  MOD_RES 2..8: note = Xaa = 6-aminohexanoic acid
  MOD_RES 23: note = Xaa = glycine or proline
RXRRXRRXRHILFQYRRMKWHKX

SEQ ID NO: 373 - protein, 23 aa, synthetic construct
  REGION 1..23: note = cell penetrating peptide
  MOD_RES 2..16: note = Xaa = 6-aminohexanoic acid
  MOD_RES 23: note = Xaa = glycine or proline
RXRRXRRXRHILFQYXRMKWHKX

SEQ ID NO: 374 - protein, 23 aa, synthetic construct
  REGION 1..23: note = cell penetrating peptide
  MOD_RES 2..8: note = Xaa = 6-aminohexanoic acid
  MOD_RES 23: note = Xaa = glycine or proline
RXRRXRRXRRILFQYRRMKWHKX

SEQ ID NO: 375 - protein, 23 aa, synthetic construct
  REGION 1..23: note = cell penetrating peptide
  MOD_RES 2..16: note = Xaa = 6-aminohexanoic acid
  MOD_RES 23: note = Xaa = glycine or proline
RXRRXRRXRRILFQYXRMKWHKX

SEQ ID NO: 376 - protein, 22 aa, synthetic construct
  REGION 1..22: note = cell penetrating peptide
  MOD_RES 2..8: note = Xaa = 6-aminohexanoic acid
  MOD_RES 22: note = Xaa = glycine or proline
RXRRXRRXRILFQYRRMKWHKX

SEQ ID NO: 377 - protein, 22 aa, synthetic construct
  REGION 1..22: note = cell penetrating peptide
  MOD_RES 2..15: note = Xaa = 6-aminohexanoic acid
  MOD_RES 22: note = Xaa = glycine or proline
RXRRXRRXRILFQYXRMKWHKX

SEQ ID NO: 378 - protein, 10 aa, synthetic construct
  REGION 1..10: note = cell penetrating peptide
  MOD_RES 2..8: note = Xaa = 6-aminohexanoic acid
  MOD_RES 10: note = Xaa = glycine or proline
RXRRXRRXRX

SEQ ID NO: 379 - protein, 13 aa, synthetic construct
  REGION 1..13: note = cell penetrating peptide
  MOD_RES 2..11: note = Xaa = 6-aminohexanoic acid
  MOD_RES 13: note = Xaa = glycine or proline
RXRRXRRXRRXRX

SEQ ID NO: 380 - protein, 7 aa, synthetic construct
  REGION 1..7: note = cell penetrating peptide
  MOD_RES 7: note = Xaa = glycine or proline
RARRARX

SEQ ID NO: 381 - protein, 10 aa, synthetic construct
  REGION 1..10: note = cell penetrating peptide
  MOD_RES 10: note = Xaa = glycine or proline
RARRARRARX

SEQ ID NO: 382 - protein, 13 aa, synthetic construct
  REGION 1..13: note = cell penetrating peptide
  MOD_RES 13: note = Xaa = glycine or proline
RARRARRARRARX

SEQ ID NO: 383 - protein, 9 aa, synthetic construct
  REGION 1..9: note = cell penetrating peptide
  MOD_RES 2..8: note = Xaa = 6-aminohexanoic acid
  MOD_RES 9: note = Xaa = glycine or proline
RXRRXRIXX

SEQ ID NO: 384 - protein, 10 aa, synthetic construct
  REGION 1..10: note = cell penetrating peptide
  MOD_RES 2..8: note = Xaa = 6-aminohexanoic acid
  MOD_RES 10: note = Xaa = glycine or proline
RXRRARRXRX

SEQ ID NO: 385 - protein, 10 aa, synthetic construct
  REGION 1..10: note = cell penetrating peptide
  MOD_RES 5: note = Xaa = 6-aminohexanoic acid
  MOD_RES 10: note = Xaa = glycine or proline
RARRXRRARX

SEQ ID NO: 386 - protein, 6 aa, synthetic construct
  REGION 1..6: note = cell penetrating peptide
  MOD_RES 6: note = Xaa = glycine or proline
RRRRRX

SEQ ID NO: 387 - protein, 7 aa, synthetic construct
  REGION 1..7: note = cell penetrating peptide
  MOD_RES 7: note = Xaa = glycine or proline
RRRRRRX

SEQ ID NO: 388 - protein, 8 aa, synthetic construct
  REGION 1..8: note = cell penetrating peptide
  MOD_RES 8: note = Xaa = glycine or proline
RRRRRRRX

SEQ ID NO: 389 - protein, 14 aa, synthetic construct
  REGION 1..14: note = cell penetrating peptide
  MOD_RES 2..11: note = Xaa = 6-aminohexanoic acid
  MOD_RES 14: note = Xaa = glycine or proline
RXRRXRRXRRXRCX

SEQ ID NO: 390 - protein, 15 aa, synthetic construct
  REGION 1..15: note = cell penetrating peptide
  MOD_RES 2..13: note = Xaa = 6-aminohexanoic acid
  MOD_RES 15: note = Xaa = glycine or proline
RXRRXRRXRRXRXCX

SEQ ID NO: 391 - protein, 27 aa, synthetic construct
  REGION 1..27: note = cell penetrating peptide
  MOD_RES 2..8: note = Xaa = 6-aminohexanoic acid
  MOD_RES 27: note = Xaa = glycine or proline
RXRRXRRXRIKILFQNRRMKWKKGGCX

SEQ ID NO: 392 - protein, 25 aa, synthetic construct
  REGION 1..25: note = cell penetrating peptide
  MOD_RES 2..8: note = Xaa = 6-aminohexanoic acid
  MOD_RES 25: note = Xaa = glycine or proline
RXRRXRRXRIKILFQNRRMKWKKCX

SEQ ID NO: 393 - protein, 24 aa, synthetic construct
  REGION 1..24: note = cell penetrating peptide
  MOD_RES 2..8: note = Xaa = 6-aminohexanoic acid
  MOD_RES 24: note = Xaa = glycine or proline
RXRRXRRXRIKILFQNRMKWKKCX

SEQ ID NO: 394 - protein, 25 aa, synthetic construct
  REGION 1..25: note = cell penetrating peptide
  MOD_RES 2..17: note = Xaa = 6-aminohexanoic acid
  MOD_RES 25: note = Xaa = glycine or proline
RXRRXRRXRIKILFQNXRMKWKKCX

SEQ ID NO: 395 - protein, 25 aa, synthetic construct
  REGION 1..25: note = cell penetrating peptide
  MOD_RES 2..8: note = Xaa = 6-aminohexanoic acid
  MOD_RES 25: note = Xaa = glycine or proline
RXRRXRRXRIKILFQNHRMKWKKCX

SEQ ID NO: 396 - protein, 25 aa, synthetic construct
  REGION 1..25: note = cell penetrating peptide
  MOD_RES 2..17: note = Xaa = 6-aminohexanoic acid
  MOD_RES 25: note = Xaa = glycine or proline
RXRRXRRXRIKILFQNXRMKWKKCX

SEQ ID NO: 397 - protein, 25 aa, synthetic construct
  REGION 1..25: note = cell penetrating peptide
  MOD_RES 2..17: note = Xaa = 6-aminohexanoic acid
  MOD_RES 25: note = Xaa = glycine or proline
RXRRXRRXRIKILFQNXRMKWKKCX

SEQ ID NO: 398 - protein, 25 aa, synthetic construct
  REGION 1..25: note = cell penetrating peptide
  MOD_RES 2..17: note = Xaa = 6-aminohexanoic acid
  MOD_RES 25: note = Xaa = glycine or proline
RXRRXRRXRIKILFQNXRMKWKACX

SEQ ID NO: 399 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIKILFQNXRMKWHKACX

SEQ ID NO: 400 - protein, 25 aa, synthetic construct
  REGION 1..25: note = cell penetrating peptide
  MOD_RES 2..17: note = Xaa = 6-aminohexanoic acid
  MOD_RES 25: note = Xaa = glycine or proline
RXRRXRRXRIKILFQNXRMKWHRCX

SEQ ID NO: 401 - protein, 25 aa, synthetic construct
  REGION 1..25: note = cell penetrating peptide
  MOD_RES 2..8: note = Xaa = 6-aminohexanoic acid
  MOD_RES 25: note = Xaa = glycine or proline
RXRXRXRXRIKILFQNRRMKWKKCX

SEQ ID NO: 402 - protein, 25 aa, synthetic construct
  REGION 1..25: note = cell penetrating peptide
  MOD_RES 25: note = Xaa = glycine or proline
RARARARARIKILFQNRRMKWKKCX

SEQ ID NO: 403 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIXILFQNXRMKWHKACX

SEQ ID NO: 404 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIHILFQNXRMKWHKACX

SEQ ID NO: 405 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIRILFQNXRMKWHKACX

SEQ ID NO: 406 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIXILFQYXRMKWHKACX

SEQ ID NO: 407 - protein, 27 aa, synthetic construct
  REGION 1..27: note = cell penetrating peptide
  MOD_RES 2..18: note = Xaa = 6-aminohexanoic acid
  MOD_RES 27: note = Xaa = glycine or proline
RXRRXRRXRLYSPLSFQXRMKWHKACX

SEQ ID NO: 408 - protein, 8 aa, synthetic construct
  REGION 1..8: note = cell penetrating peptide
  MOD_RES 8: note = Xaa = glycine or proline
RRMKWHKX

SEQ ID NO: 409 - protein, 8 aa, synthetic construct
  REGION 1..8: note = cell penetrating peptide
  MOD_RES 1: note = Xaa = 6-aminohexanoic acid
  MOD_RES 8: note = Xaa = glycine or proline
XRMKWHKX

SEQ ID NO: 410 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILFQXXRMKWHKX

SEQ ID NO: 411 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILFQXXRMKWHKX

SEQ ID NO: 412 - protein, 26 aa, synthetic construct
  REGION 1..26: note = cell penetrating peptide
  MOD_RES 26: note = Xaa = glycine or proline
RRRRRRRQIKILFQNPKKKRKVGGCX

SEQ ID NO: 413 - protein, 17 aa, synthetic construct
  REGION 1..17: note = cell penetrating peptide
  MOD_RES 17: note = Xaa = glycine or proline
HHFFRRRRRRRRRFFCX

SEQ ID NO: 414 - protein, 19 aa, synthetic construct
  REGION 1..19: note = cell penetrating peptide
  MOD_RES 19: note = Xaa = glycine or proline
HHHHHHRRRRRRRRRFFCX

SEQ ID NO: 415 - protein, 21 aa, synthetic construct
  REGION 1..21: note = cell penetrating peptide
  MOD_RES 21: note = Xaa = glycine or proline
HHHHHHFFRRRRRRRRRFFCX

SEQ ID NO: 416 - protein, 20 aa, synthetic construct
  REGION 1..20: note = cell penetrating peptide
  MOD_RES 20: note = Xaa = glycine or proline
  MOD_RES 6..7: note = Xaa = 6-aminohexanoic acid
HHHHHXXRRRRRRRRRFFCX

SEQ ID NO: 417 - protein, 23 aa, synthetic construct
  REGION 1..23: note = cell penetrating peptide
  MOD_RES 23: note = Xaa = glycine or proline
  MOD_RES 7..8: note = Xaa = 6-aminohexanoic acid
HHHHHHXXFFRRRRRRRRRFFCX

SEQ ID NO: 418 - protein, 21 aa, synthetic construct
  REGION 1..21: note = cell penetrating peptide
  MOD_RES 4..16: note = Xaa = 6-aminohexanoic acid
  MOD_RES 21: note = Xaa = glycine or proline
HHHXRRRRRRRRRFFXHHHCX

SEQ ID NO: 419 - protein, 8 aa, synthetic construct
  REGION 1..8: note = cell penetrating peptide
  MOD_RES 1: note = Xaa = 6-aminohexanoic acid
  MOD_RES 8: note = Xaa = glycine or proline
XRMKWHKX

SEQ ID NO: 420 - protein, 8 aa, synthetic construct
  REGION 1..8: note = cell penetrating peptide
  MOD_RES 1: note = Xaa = 6-aminohexanoic acid
  MOD_RES 8: note = Xaa = glycine or proline
XRWKWHKX

SEQ ID NO: 421 - protein, 8 aa, synthetic construct
  REGION 1..8: note = cell penetrating peptide
  MOD_RES 2..6: note = Xaa = 6-aminohexanoic acid
  MOD_RES 8: note = Xaa = glycine or proline
RXRARXRX

SEQ ID NO: 422 - protein, 8 aa, synthetic construct
  REGION 1..8: note = cell penetrating peptide
  MOD_RES 2..6: note = Xaa = 6-aminohexanoic acid
  MOD_RES 8: note = Xaa = glycine or proline
RXRXRXRX

SEQ ID NO: 423 - protein, 8 aa, synthetic construct
  REGION 1..8: note = cell penetrating peptide
  MOD_RES 4: note = Xaa = 6-aminohexanoic acid
  MOD_RES 8: note = Xaa = glycine or proline
RARXRARX

SEQ ID NO: 424 - protein, 6 aa, synthetic construct
  REGION 1..6: note = cell penetrating peptide
  MOD_RES 2: note = Xaa = 6-aminohexanoic acid
  MOD_RES 6: note = Xaa = glycine or proline
RXRARX

SEQ ID NO: 425 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILFQXXHMKWHKX

SEQ ID NO: 426 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILFQXXRWKWHKX

SEQ ID NO: 427 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILFQXXHWKWHKX

SEQ ID NO: 428 - protein, 27 aa, synthetic construct
  REGION 1..27: note = cell penetrating peptide
  MOD_RES 1..25: note = Xaa = 6-aminohexanoic acid
  MOD_RES 27: note = Xaa = glycine or proline
XXXXXXXXXXXXXXILFQXRXRARXRX

SEQ ID NO: 429 - protein, 27 aa, synthetic construct
  REGION 1..27: note = cell penetrating peptide
  MOD_RES 1..25: note = Xaa = 6-aminohexanoic acid
  MOD_RES 27: note = Xaa = glycine or proline
XXXXXXXXXXXXXXILFQXRXRXRXRX

SEQ ID NO: 430 - protein, 26 aa, synthetic construct
  REGION 1..26: note = cell penetrating peptide
  MOD_RES 1..24: note = Xaa = 6-aminohexanoic acid
  MOD_RES 26: note = Xaa = glycine or proline
XXXXXXXXXXXXXXILFQXRXRRXRX

SEQ ID NO: 431 - protein, 27 aa, synthetic construct
  REGION 1..27: note = cell penetrating peptide
  MOD_RES 1..23: note = Xaa = 6-aminohexanoic acid
  MOD_RES 27: note = Xaa = glycine or proline
XXXXXXXXXXXXXXILFQXRARXRARX

SEQ ID NO: 432 - protein, 27 aa, synthetic construct
  REGION 1..27: note = cell penetrating peptide
  MOD_RES 1..25: note = Xaa = 6-aminohexanoic acid
  MOD_RES 27: note = Xaa = glycine or proline
XXXXXXXXXXXXXXILFQXRXRARXRX

SEQ ID NO: 433 - protein, 25 aa, synthetic construct
  REGION 1..25: note = cell penetrating peptide
  MOD_RES 1..21: note = Xaa = 6-aminohexanoic acid
  MOD_RES 25: note = Xaa = glycine or proline
XXXXXXXXXXXXXXILFQXRXRARX

SEQ ID NO: 434 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILIQXXRMKWHKX

SEQ ID NO: 435 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILIQXXHMKWHKX

SEQ ID NO: 436 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILIQXXRWKWHKX

SEQ ID NO: 437 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILIQXXHWKWHKX

SEQ ID NO: 438 - protein, 27 aa, synthetic construct
  REGION 1..27: note = cell penetrating peptide
  MOD_RES 1..25: note = Xaa = 6-aminohexanoic acid
  MOD_RES 27: note = Xaa = glycine or proline
XXXXXXXXXXXXXXILIQXRXRARXRX

SEQ ID NO: 439 - protein, 27 aa, synthetic construct
  REGION 1..27: note = cell penetrating peptide
  MOD_RES 1..25: note = Xaa = 6-aminohexanoic acid
  MOD_RES 27: note = Xaa = glycine or proline
XXXXXXXXXXXXXXILIQXRXRXRXRX

SEQ ID NO: 440 - protein, 26 aa, synthetic construct
  REGION 1..26: note = cell penetrating peptide
  MOD_RES 1..24: note = Xaa = 6-aminohexanoic acid
  MOD_RES 26: note = Xaa = glycine or proline
XXXXXXXXXXXXXXILIQXRXRRXRX

SEQ ID NO: 441 - protein, 27 aa, synthetic construct
  REGION 1..27: note = cell penetrating peptide
  MOD_RES 1..23: note = Xaa = 6-aminohexanoic acid
  MOD_RES 27: note = Xaa = glycine or proline
XXXXXXXXXXXXXXILIQXRARXRARX

SEQ ID NO: 442 - protein, 27 aa, synthetic construct
  REGION 1..27: note = cell penetrating peptide
  MOD_RES 1..25: note = Xaa = 6-aminohexanoic acid
  MOD_RES 27: note = Xaa = glycine or proline
XXXXXXXXXXXXXXILIQXRXRARXRX

SEQ ID NO: 443 - protein, 25 aa, synthetic construct
  REGION 1..25: note = cell penetrating peptide
  MOD_RES 1..21: note = Xaa = 6-aminohexanoic acid
  MOD_RES 25: note = Xaa = glycine or proline
XXXXXXXXXXXXXXILIQXRXRARX

SEQ ID NO: 444 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILFQXXHMKWHKX

SEQ ID NO: 445 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILFQXXRWKWHKX

SEQ ID NO: 446 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILFQXXHWKWHKX

SEQ ID NO: 447 - protein, 27 aa, synthetic construct
  REGION 1..27: note = cell penetrating peptide
  MOD_RES 1..25: note = Xaa = 6-aminohexanoic acid
  MOD_RES 27: note = Xaa = glycine or proline
XXXXXXXXXXXXXXILFQXRXRARXRX

SEQ ID NO: 448 - protein, 27 aa, synthetic construct
  REGION 1..27: note = cell penetrating peptide
  MOD_RES 1..25: note = Xaa = 6-aminohexanoic acid
  MOD_RES 27: note = Xaa = glycine or proline
XXXXXXXXXXXXXXILFQXRXRXRXRX

SEQ ID NO: 449 - protein, 26 aa, synthetic construct
  REGION 1..26: note = cell penetrating peptide
  MOD_RES 1..24: note = Xaa = 6-aminohexanoic acid
  MOD_RES 26: note = Xaa = glycine or proline
XXXXXXXXXXXXXXILFQXRXRRXRX

SEQ ID NO: 450 - protein, 27 aa, synthetic construct
  REGION 1..27: note = cell penetrating peptide
  MOD_RES 1..23: note = Xaa = 6-aminohexanoic acid
  MOD_RES 27: note = Xaa = glycine or proline
XXXXXXXXXXXXXXILFQXRARXRARX

SEQ ID NO: 451 - protein, 27 aa, synthetic construct
  REGION 1..27: note = cell penetrating peptide
  MOD_RES 1..25: note = Xaa = 6-aminohexanoic acid
  MOD_RES 27: note = Xaa = glycine or proline
XXXXXXXXXXXXXXILFQXRXRARXRX

SEQ ID NO: 452 - protein, 25 aa, synthetic construct
  REGION 1..25: note = cell penetrating peptide
  MOD_RES 1..21: note = Xaa = 6-aminohexanoic acid
  MOD_RES 25: note = Xaa = glycine or proline
XXXXXXXXXXXXXXILFQXRXRARX

SEQ ID NO: 453 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILIQXXRMKWHKX

SEQ ID NO: 454 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILIQXXHMKWHKX

SEQ ID NO: 455 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILIQXXRWKWHKX

SEQ ID NO: 456 - protein, 27 aa, synthetic construct
  REGION 1..27: note = cell penetrating peptide
  MOD_RES 1..20: note = Xaa = 6-aminohexanoic acid
  MOD_RES 27: note = Xaa = glycine or proline
XXXXXXXXXXXXXXILIQXXHWKWHKX

SEQ ID NO: 457 - protein, 27 aa, synthetic construct
  REGION 1..27: note = cell penetrating peptide
  MOD_RES 1..25: note = Xaa = 6-aminohexanoic acid
  MOD_RES 27: note = Xaa = glycine or proline
XXXXXXXXXXXXXXILIQXRXRARXRX

SEQ ID NO: 458 - protein, 27 aa, synthetic construct
  REGION 1..27: note = cell penetrating peptide
  MOD_RES 1..25: note = Xaa = 6-aminohexanoic acid
  MOD_RES 27: note = Xaa = glycine or proline
XXXXXXXXXXXXXXILIQXRXRXRXRX

SEQ ID NO: 459 - protein, 26 aa, synthetic construct
  REGION 1..26: note = cell penetrating peptide
  MOD_RES 1..24: note = Xaa = 6-aminohexanoic acid
  MOD_RES 26: note = Xaa = glycine or proline
XXXXXXXXXXXXXXILIQXRXRRXRX

SEQ ID NO: 460 - protein, 27 aa, synthetic construct
  REGION 1..27: note = cell penetrating peptide
  MOD_RES 1..23: note = Xaa = 6-aminohexanoic acid
  MOD_RES 27: note = Xaa = glycine or proline
XXXXXXXXXXXXXXILIQXRARXRARX

SEQ ID NO: 461 - protein, 27 aa, synthetic construct
  REGION 1..27: note = cell penetrating peptide
  MOD_RES 1..25: note = Xaa = 6-aminohexanoic acid
  MOD_RES 27: note = Xaa = glycine or proline
XXXXXXXXXXXXXXILIQXRXRARXRX

SEQ ID NO: 462 - protein, 25 aa, synthetic construct
  REGION 1..25: note = cell penetrating peptide
  MOD_RES 1..21: note = Xaa = 6-aminohexanoic acid
  MOD_RES 25: note = Xaa = glycine or proline
XXXXXXXXXXXXXXILIQXRXRARX

SEQ ID NO: 463 - protein, 15 aa, synthetic construct
  REGION 1..15: note = cell penetrating peptide
  MOD_RES 2..13: note = Xaa = 6-aminohexanoic acid
  MOD_RES 15: note = Xaa = glycine or proline
RXRRARRXRRARXAX

SEQ ID NO: 464 - protein, 24 aa, synthetic construct
  REGION 1..24: note = cell penetrating peptide
  MOD_RES 2..15: note = Xaa = 6-aminohexanoic acid
  MOD_RES 24: note = Xaa = glycine or proline
RXRRARRXRILFQYXHMKWHKACX

SEQ ID NO: 465 - protein, 24 aa, synthetic construct
  REGION 1..24: note = cell penetrating peptide
  MOD_RES 2..15: note = Xaa = 6-aminohexanoic acid
  MOD_RES 24: note = Xaa = glycine or proline
RXRRARRXRILFQYXRMKWHKACX

SEQ ID NO: 466 - protein, 24 aa, synthetic construct
  REGION 1..24: note = cell penetrating peptide
  MOD_RES 2..15: note = Xaa = 6-aminohexanoic acid
  MOD_RES 24: note = Xaa = glycine or proline
RXRRARRXRILFQYXRWKWHKACX

SEQ ID NO: 467 - protein, 14 aa, synthetic construct
  REGION 1..14: note = cell penetrating peptide
  MOD_RES 2..11: note = Xaa = 6-aminohexanoic acid
  MOD_RES 14: note = Xaa = glycine or proline
RXRRXRRXRRXRCX

SEQ ID NO: 468 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIXILFQNXRMKWHKACX

SEQ ID NO: 469 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIHILFQNXRMKWHKACX

SEQ ID NO: 470 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRXRRXRIXILFQYXRMKWHKACX

SEQ ID NO: 471 - protein, 27 aa, synthetic construct
  REGION 1..27: note = cell penetrating peptide
  MOD_RES 2..18: note = Xaa = 6-aminohexanoic acid
  MOD_RES 27: note = Xaa = glycine or proline
RXRRXRRXRLYSPLSFQXRMKWHKACX

SEQ ID NO: 472 - protein, 24 aa, synthetic construct
  REGION 1..24: note = cell penetrating peptide
  MOD_RES 2..15: note = Xaa = 6-aminohexanoic acid
  MOD_RES 24: note = Xaa = glycine or proline
RXRRXRRXRILFQYXRMKWHKACX

SEQ ID NO: 473 - protein, 23 aa, synthetic construct
  REGION 1..23: note = cell penetrating peptide
  MOD_RES 2..14: note = Xaa = 6-aminohexanoic acid
  MOD_RES 23: note = Xaa = glycine or proline
RXRRXRIXILFQYXRMKWHKACX

SEQ ID NO: 474 - protein, 24 aa, synthetic construct
  REGION 1..24: note = cell penetrating peptide
  MOD_RES 5..15: note = Xaa = 6-aminohexanoic acid
  MOD_RES 24: note = Xaa = glycine or proline
RARRXRRARILFQYXRMKWHKACX

SEQ ID NO: 475 - protein, 24 aa, synthetic construct
  REGION 1..24: note = cell penetrating peptide
  MOD_RES 2..15: note = Xaa = 6-aminohexanoic acid
  MOD_RES 24: note = Xaa = glycine or proline
RXRRARRXRILFQYXRMKWHKACX

SEQ ID NO: 476 - protein, 24 aa, synthetic construct
  REGION 1..24: note = cell penetrating peptide
  MOD_RES 5..15: note = Xaa = 6-aminohexanoic acid
  MOD_RES 24: note = Xaa = glycine or proline
RARRXRRARILFQYXRMKWHKACX

SEQ ID NO: 477 - protein, 24 aa, synthetic construct
  REGION 1..24: note = cell penetrating peptide
  MOD_RES 2..15: note = Xaa = 6-aminohexanoic acid
  MOD_RES 24: note = Xaa = glycine or proline
RXRRARRXRILFQYXRMKWHKACX

SEQ ID NO: 478 - protein, 24 aa, synthetic construct
  REGION 1..24: note = cell penetrating peptide
  MOD_RES 2..15: note = Xaa = 6-aminohexanoic acid
  MOD_RES 24: note = Xaa = glycine or proline
RXRRARRXRILFQYXHMKWHKACX

SEQ ID NO: 479 - protein, 24 aa, synthetic construct
  REGION 1..24: note = cell penetrating peptide
  MOD_RES 2..15: note = Xaa = 6-aminohexanoic acid
  MOD_RES 24: note = Xaa = glycine or proline
RXRRARRXRILFQYXRMKWHKACX

SEQ ID NO: 480 - protein, 24 aa, synthetic construct
  REGION 1..24: note = cell penetrating peptide
  MOD_RES 2..15: note = Xaa = 6-aminohexanoic acid
  MOD_RES 24: note = Xaa = glycine or proline
RXRRARRXRILFQYXRWKWHKACX

SEQ ID NO: 481 - protein, 24 aa, synthetic construct
  REGION 1..24: note = cell penetrating peptide
  MOD_RES 2..15: note = Xaa = 6-aminohexanoic acid
  MOD_RES 24: note = Xaa = glycine or proline
RXRRARRXRILFQYXHWKWHKACX

SEQ ID NO: 482 - protein, 24 aa, synthetic construct
  REGION 1..24: note = cell penetrating peptide
  MOD_RES 2..20: note = Xaa = 6-aminohexanoic acid
  MOD_RES 24: note = Xaa = glycine or proline
RXRRARRXRILFQYRXRARXRACX

SEQ ID NO: 483 - protein, 24 aa, synthetic construct
  REGION 1..24: note = cell penetrating peptide
  MOD_RES 2..20: note = Xaa = 6-aminohexanoic acid
  MOD_RES 24: note = Xaa = glycine or proline
RXRRARRXRILFQYRXRXRXRACX

SEQ ID NO: 484 - protein, 24 aa, synthetic construct
  REGION 1..24: note = cell penetrating peptide
  MOD_RES 2..15: note = Xaa = 6-aminohexanoic acid
  MOD_RES 24: note = Xaa = glycine or proline
RXRRARRXRILIQYXRMKWHKACX

SEQ ID NO: 485 - protein, 19 aa, synthetic construct
  REGION 1..19: note = cell penetrating peptide
  MOD_RES 2..16: note = Xaa = 6-aminohexanoic acid
  MOD_RES 19: note = Xaa = glycine or proline
RXRRXRILFQYRXRRXRCX

SEQ ID NO: 486 - protein, 24 aa, synthetic construct
  REGION 1..24: note = cell penetrating peptide
  MOD_RES 2..20: note = Xaa = 6-aminohexanoic acid
  MOD_RES 24: note = Xaa = glycine or proline
RXRRARRXRILFQYRXRARXRACX

SEQ ID NO: 487 - protein, 24 aa, synthetic construct
  REGION 1..24: note = cell penetrating peptide
  MOD_RES 2..20: note = Xaa = 6-aminohexanoic acid
  MOD_RES 24: note = Xaa = glycine or proline
RXRRARRXRILFQYRXRXRXRACX

SEQ ID NO: 488 - protein, 24 aa, synthetic construct
  REGION 1..24: note = cell penetrating peptide
  MOD_RES 2..15: note = Xaa = 6-aminohexanoic acid
  MOD_RES 24: note = Xaa = glycine or proline
RXRRARRXRILIQYXRMKWHKACX

SEQ ID NO: 489 - protein, 21 aa, synthetic construct
  REGION 1..21: note = cell penetrating peptide
  MOD_RES 2..16: note = Xaa = 6-aminohexanoic acid
  MOD_RES 21: note = Xaa = glycine or proline
RXRRXRILFQYRXRRXRCYSX

SEQ ID NO: 490 - protein, 24 aa, synthetic construct
  REGION 1..24: note = cell penetrating peptide
  MOD_RES 5..18: note = Xaa = 6-aminohexanoic acid
  MOD_RES 24: note = Xaa = glycine or proline
RARRXRRARILFQYRARXRARACX

SEQ ID NO: 491 - protein, 24 aa, synthetic construct
  REGION 1..24: note = cell penetrating peptide
  MOD_RES 5..20: note = Xaa = 6-aminohexanoic acid
  MOD_RES 24: note = Xaa = glycine or proline
RARRXRRARILFQYRXRARXRACX

SEQ ID NO: 492 - protein, 23 aa, synthetic construct
  REGION 1..23: note = cell penetrating peptide
  MOD_RES 5..19: note = Xaa = 6-aminohexanoic acid
  MOD_RES 23: note = Xaa = glycine or proline
RARRXRRARILFQYRXRRXRACX

SEQ ID NO: 493 - protein, 23 aa, synthetic construct
  REGION 1..23: note = cell penetrating peptide
  MOD_RES 5..20: note = Xaa = 6-aminohexanoic acid
  MOD_RES 23: note = Xaa = glycine or proline
RARRXRRARILFQYRXRARXACX

SEQ ID NO: 494 - protein, 23 aa, synthetic construct
  REGION 1..23: note = cell penetrating peptide
  MOD_RES 2..19: note = Xaa = 6-aminohexanoic acid
  MOD_RES 23: note = Xaa = glycine or proline
RXRRARRXRILFQYRXRRXRACX

SEQ ID NO: 495 - protein, 23 aa, synthetic construct
  REGION 1..23: note = cell penetrating peptide
  MOD_RES 2..20: note = Xaa = 6-aminohexanoic acid
  MOD_RES 23: note = Xaa = glycine or proline
RXRRARRXRILFQYRXRARXACX

SEQ ID NO: 496 - protein, 26 aa, synthetic construct
  REGION 1..26: note = cell penetrating peptide
  MOD_RES 2..17: note = Xaa = 6-aminohexanoic acid
  MOD_RES 26: note = Xaa = glycine or proline
RXRRARRXRIHILFQNXRMKWHKACX

SEQ ID NO: 497 - protein, 16 aa, synthetic construct
  REGION 1..16: note = cell penetrating peptide
  MOD_RES 2..13: note = Xaa = 6-aminohexanoic acid
  MOD_RES 16: note = Xaa = glycine or proline
RXRRARRXRRARXACX

SEQ ID NO: 498 - protein, 22 aa, synthetic construct
  REGION 1..22: note = cell penetrating peptide
  MOD_RES 2..15: note = Xaa = 6-aminohexanoic acid
  MOD_RES 22: note = Xaa = glycine or proline
RXRRARRXRILFQYXHMKWHKX

SEQ ID NO: 499 - protein, 22 aa, synthetic construct
  REGION 1..22: note = cell penetrating peptide
  MOD_RES 2..15: note = Xaa = 6-aminohexanoic acid
  MOD_RES 22: note = Xaa = glycine or proline
RXRRARRXRILFQYXRMKWHKX

SEQ ID NO: 500 - protein, 22 aa, synthetic construct
  REGION 1..22: note = cell penetrating peptide
  MOD_RES 2..15: note = Xaa = 6-aminohexanoic acid
  MOD_RES 22: note = Xaa = glycine or proline
RXRRARRXRILFQYXRWKWHKX

SEQ ID NO: 501 - protein, 22 aa, synthetic construct
  REGION 1..22: note = cell penetrating peptide
  MOD_RES 2..15: note = Xaa = 6-aminohexanoic acid
  MOD_RES 22: note = Xaa = glycine or proline
RXRRARRXRILFQYXRMKWHKX

SEQ ID NO: 502 - protein, 22 aa, synthetic construct
  REGION 1..22: note = cell penetrating peptide
  MOD_RES 2..20: note = Xaa = 6-aminohexanoic acid
  MOD_RES 22: note = Xaa = glycine or proline
RXRRARRXRILFQYRXRARXRX

SEQ ID NO: 503 - protein, 22 aa, synthetic construct
  REGION 1..22: note = cell penetrating peptide
  MOD_RES 2..20: note = Xaa = 6-aminohexanoic acid
  MOD_RES 22: note = Xaa = glycine or proline
RXRRARRXRILFQYRXRXRXRX

SEQ ID NO: 504 - protein, 21 aa, synthetic construct
  REGION 1..21: note = cell penetrating peptide
  MOD_RES 2..19: note = Xaa = 6-aminohexanoic acid
  MOD_RES 21: note = Xaa = glycine or proline
RXRRARRXRILFQYRXRRXRX

SEQ ID NO: 505 - protein, 22 aa, synthetic construct
  REGION 1..22: note = cell penetrating peptide
  MOD_RES 2..18: note = Xaa = 6-aminohexanoic acid
  MOD_RES 22: note = Xaa = glycine or proline
RXRRARRXRILFQYRARXRARX

SEQ ID NO: 506 - protein, 20 aa, synthetic construct
  REGION 1..20: note = cell penetrating peptide
  MOD_RES 2..16: note = Xaa = 6-aminohexanoic acid
  MOD_RES 20: note = Xaa = glycine or proline
RXRRARRXRILFQYRXRARX

SEQ ID NO: 507 - protein, 22 aa, synthetic construct
  REGION 1..22: note = cell penetrating peptide
  MOD_RES 2..15: note = Xaa = 6-aminohexanoic acid
  MOD_RES 22: note = Xaa = glycine or proline
RXRRARRXRILIQYXHMKWHKX

SEQ ID NO: 508 - protein, 22 aa, synthetic construct
  REGION 1..22: note = cell penetrating peptide
  MOD_RES 2..15: note = Xaa = 6-aminohexanoic acid
  MOD_RES 22: note = Xaa = glycine or proline
RXRRARRXRILIQYXRMKWHKX

SEQ ID NO: 509 - protein, 22 aa, synthetic construct
  REGION 1..22: note = cell penetrating peptide
  MOD_RES 2..15: note = Xaa = 6-aminohexanoic acid
  MOD_RES 22: note = Xaa = glycine or proline
RXRRARRXRILIQYXRWKWHKX

SEQ ID NO: 510 - protein, 22 aa, synthetic construct
  REGION 1..22: note = cell penetrating peptide
  MOD_RES 2..15: note = Xaa = 6-aminohexanoic acid
  MOD_RES 22: note = Xaa = glycine or proline
RXRRARRXRILIQYXRMKWHKX

SEQ ID NO: 511 - protein, 22 aa, synthetic construct
  REGION 1..22: note = cell penetrating peptide
  MOD_RES 2..20: note = Xaa = 6-aminohexanoic acid
  MOD_RES 22: note = Xaa = glycine or proline
RXRRARRXRILIQYRXRARXRX

SEQ ID NO: 512 - protein, 22 aa, synthetic construct
  REGION 1..22: note = cell penetrating peptide
  MOD_RES 2..20: note = Xaa = 6-aminohexanoic acid
  MOD_RES 22: note = Xaa = glycine or proline
RXRRARRXRILIQYRXRXRXRX

SEQ ID NO: 513 - protein, 21 aa, synthetic construct
  REGION 1..21: note = cell penetrating peptide
  MOD_RES 2..19: note = Xaa = 6-aminohexanoic acid
  MOD_RES 21: note = Xaa = glycine or proline
RXRRARRXRILIQYRXRRXRX

SEQ ID NO: 514 - protein, 22 aa, synthetic construct
  REGION 1..22: note = cell penetrating peptide
  MOD_RES 2..18: note = Xaa = 6-aminohexanoic acid
  MOD_RES 22: note = Xaa = glycine or proline
RXRRARRXRILIQYRARXRARX

SEQ ID NO: 515 - protein, 20 aa, synthetic construct
  REGION 1..20: note = cell penetrating peptide
  MOD_RES 2..16: note = Xaa = 6-aminohexanoic acid
  MOD_RES 20: note = Xaa = glycine or proline
RXRRARRXRILIQYRXRARX

SEQ ID NO: 516 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILFQYXHMKWHKX

SEQ ID NO: 517 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILFQYXRMKWHKX

SEQ ID NO: 518 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILFQYXRWKWHKX

SEQ ID NO: 519 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILFQYXRMKWHKX

SEQ ID NO: 520 - protein, 22 aa, synthetic construct
  REGION 1..22: note = cell penetrating peptide
  MOD_RES 5..20: note = Xaa = 6-aminohexanoic acid
  MOD_RES 22: note = Xaa = glycine or proline
RARRXRRARILFQYRXRARXRX

SEQ ID NO: 521 - protein, 22 aa, synthetic construct
  REGION 1..22: note = cell penetrating peptide
  MOD_RES 5..20: note = Xaa = 6-aminohexanoic acid
  MOD_RES 22: note = Xaa = glycine or proline
RARRXRRARILFQYRXRXRXRX

SEQ ID NO: 522 - protein, 21 aa, synthetic construct
  REGION 1..21: note = cell penetrating peptide
  MOD_RES 5..19: note = Xaa = 6-aminohexanoic acid
  MOD_RES 21: note = Xaa = glycine or proline
RARRXRRARILFQYRXRRXRX

SEQ ID NO: 523 - protein, 22 aa, synthetic construct
  REGION 1..22: note = cell penetrating peptide
  MOD_RES 5..18: note = Xaa = 6-aminohexanoic acid
  MOD_RES 22: note = Xaa = glycine or proline
RARRXRRARILFQYRARXRARX

SEQ ID NO: 524 - protein, 20 aa, synthetic construct
  REGION 1..20: note = cell penetrating peptide
  MOD_RES 5..16: note = Xaa = 6-aminohexanoic acid
  MOD_RES 20: note = Xaa = glycine or proline
RARRXRRARILFQYRXRARX

SEQ ID NO: 525 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILIQYXHMKWHKX

SEQ ID NO: 526 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILIQYXRMKWHKX

SEQ ID NO: 527 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILIQYXRWKWHKX

SEQ ID NO: 528 - protein, 22 aa, synthetic construct
  REGION 1..22: note = cell penetrating peptide
  MOD_RES 5..15: note = Xaa = 6-aminohexanoic acid
  MOD_RES 22: note = Xaa = glycine or proline
RARRXRRARILIQYXRMKWHKX

SEQ ID NO: 529 - protein, 22 aa, synthetic construct
  REGION 1..22: note = cell penetrating peptide
  MOD_RES 5..20: note = Xaa = 6-aminohexanoic acid
  MOD_RES 22: note = Xaa = glycine or proline
RARRXRRARILIQYRXRARXRX

SEQ ID NO: 530 - protein, 22 aa, synthetic construct
  REGION 1..22: note = cell penetrating peptide
  MOD_RES 5..20: note = Xaa = 6-aminohexanoic acid
  MOD_RES 22: note = Xaa = glycine or proline
RARRXRRARILIQYRXRXRXRX

SEQ ID NO: 531 - protein, 21 aa, synthetic construct
  REGION 1..21: note = cell penetrating peptide
  MOD_RES 5..19: note = Xaa = 6-aminohexanoic acid
  MOD_RES 21: note = Xaa = glycine or proline
RARRXRRARILIQYRXRRXRX

SEQ ID NO: 532 - protein, 22 aa, synthetic construct
  REGION 1..22: note = cell penetrating peptide
  MOD_RES 5..18: note = Xaa = 6-aminohexanoic acid
  MOD_RES 22: note = Xaa = glycine or proline
RARRXRRARILIQYRARXRARX

SEQ ID NO: 533 - protein, 20 aa, synthetic construct
  REGION 1..20: note = cell penetrating peptide
  MOD_RES 5..16: note = Xaa = 6-aminohexanoic acid
  MOD_RES 20: note = Xaa = glycine or proline
RARRXRRARILIQYRXRARX

SEQ ID NO: 534 - protein, 19 aa, synthetic construct
  REGION 1..19: note = cell penetrating peptide
  MOD_RES 2..12: note = Xaa = glycine or proline
RXRRXRILFQYXHMKWHKX

SEQ ID NO: 535 - protein, 19 aa, synthetic construct
  REGION 1..19: note = cell penetrating peptide
  MOD_RES 2..12: note = Xaa = 6-aminohexanoic acid
  MOD_RES 19: note = Xaa = glycine or proline
RXRRXRILFQYXRMKWHKX

SEQ ID NO: 536 - protein, 19 aa, synthetic construct
  REGION 1..19: note = cell penetrating peptide
  MOD_RES 2..12: note = Xaa = 6-aminohexanoic acid
  MOD_RES 19: note = Xaa = glycine or proline
RXRRXRILFQYXRWKWHKX

SEQ ID NO: 537 - protein, 19 aa, synthetic construct
  REGION 1..19: note = cell penetrating peptide
  MOD_RES 2..12: note = Xaa = 6-aminohexanoic acid
  MOD_RES 19: note = Xaa = glycine or proline
RXRRXRILFQYXRMKWHKX

SEQ ID NO: 538 - protein, 19 aa, synthetic construct
  REGION 1..19: note = cell penetrating peptide
  MOD_RES 2..17: note = Xaa = 6-aminohexanoic acid
  MOD_RES 19: note = Xaa = glycine or proline
RXRRXRILFQYRXRARXRX

SEQ ID NO: 539 - protein, 19 aa, synthetic construct
  REGION 1..19: note = cell penetrating peptide
  MOD_RES 2..17: note = Xaa = 6-aminohexanoic acid
  MOD_RES 19: note = Xaa = glycine or proline
RXRRXRILFQYRXRXRXRX

SEQ ID NO: 540 - protein, 18 aa, synthetic construct
  REGION 1..18: note = cell penetrating peptide
  MOD_RES 2..16: note = Xaa = 6-aminohexanoic acid
  MOD_RES 18: note = Xaa = glycine or proline
RXRRXRILFQYRXRRXRX

SEQ ID NO: 541 - protein, 19 aa, synthetic construct
  REGION 1..19: note = cell penetrating peptide
  MOD_RES 2..15: note = Xaa = 6-aminohexanoic acid
  MOD_RES 19: note = Xaa = glycine or proline
RXRRXRILFQYRARXRARX

SEQ ID NO: 542 - protein, 17 aa, synthetic construct
  REGION 1..17: note = cell penetrating peptide
  MOD_RES 2..13: note = Xaa = 6-aminohexanoic acid
  MOD_RES 17: note = Xaa = glycine or proline
RXRRXRILFQYRXRARX

SEQ ID NO: 543 - protein, 19 aa, synthetic construct
  REGION 1..19: note = cell penetrating peptide
  MOD_RES 2..12: note = Xaa = 6-aminohexanoic acid
  MOD_RES 19: note = Xaa = glycine or proline
RXRRXRILIQYXHMKWHKX

SEQ ID NO: 544 - protein, 19 aa, synthetic construct
  REGION 1..19: note = cell penetrating peptide
  MOD_RES 2..12: note = Xaa = 6-aminohexanoic acid
  MOD_RES 19: note = Xaa = glycine or proline
RXRRXRILIQYXRMKWHKX

SEQ ID NO: 545 - protein, 19 aa, synthetic construct
  REGION 1..19: note = cell penetrating peptide
  MOD_RES 2..12: note = Xaa = 6-aminohexanoic acid
  MOD_RES 19: note = Xaa = glycine or proline
RXRRXRILIQYXRWKWHKX

SEQ ID NO: 546 - protein, 19 aa, synthetic construct
  REGION 1..19: note = cell penetrating peptide
  MOD_RES 2..12: note = Xaa = 6-aminohexanoic acid
  MOD_RES 19: note = Xaa = glycine or proline
RXRRXRILIQYXRMKWHKX

SEQ ID NO: 547 - protein, 19 aa, synthetic construct
  REGION 1..19: note = cell penetrating peptide
  MOD_RES 2..17: note = Xaa = 6-aminohexanoic acid
  MOD_RES 19: note = Xaa = glycine or proline
RXRRXRILIQYRXRARXRX

SEQ ID NO: 548 - protein, 19 aa, synthetic construct
  REGION 1..19: note = cell penetrating peptide
  MOD_RES 2..17: note = Xaa = 6-aminohexanoic acid
  MOD_RES 19: note = Xaa = glycine or proline
RXRRXRILIQYRXRXRXRX

SEQ ID NO: 549 - protein, 18 aa, synthetic construct
  REGION 1..18: note = cell penetrating peptide
  MOD_RES 2..16: note = Xaa = 6-aminohexanoic acid
  MOD_RES 18: note = Xaa = glycine or proline
RXRRXRILIQYRXRRXRX

SEQ ID NO: 550 - protein, 19 aa, synthetic construct
  REGION 1..19: note = cell penetrating peptide
  MOD_RES 2..15: note = Xaa = 6-aminohexanoic acid
  MOD_RES 19: note = Xaa = glycine or proline
RXRRXRILIQYRARXRARX

SEQ ID NO: 551 - protein, 17 aa, synthetic construct
  REGION 1..17: note = cell penetrating peptide
  MOD_RES 2..13: note = Xaa = 6-aminohexanoic acid
  MOD_RES 17: note = Xaa = glycine or proline
RXRRXRILIQYRXRARX

SEQ ID NO: 552 - protein, 18 aa, synthetic construct
  REGION 1..18: note = cell penetrating peptide
  MOD_RES 4..14: note = Xaa = 6-aminohexanoic acid
  MOD_RES 18: note = Xaa - glycine or proline
PRPXXXXXXXXXXXPRGX

SEQ ID NO: 553 - protein, 9 aa, synthetic construct
  REGION 1..9: note = cell penetrating peptide
  MOD_RES 9: note = Xaa = glycine or proline
RRRRRRRRX

SEQ ID NO: 554 - protein, 8 aa, synthetic construct
  REGION 1..8: note = cell penetrating peptide
  MOD_RES 8: note = Xaa = glycine or proline
RRMKWKKX

SEQ ID NO: 555 - protein, 8 aa, synthetic construct
  REGION 1..8: note = cell penetrating peptide
  MOD_RES 8: note = Xaa = glycine or proline
PKKKRKVX

SEQ ID NO: 556 - protein, 33 aa, synthetic construct
  REGION 1..33: note = cell penetrating peptide
  MOD_RES 33: note = Xaa = glycine or proline
CKDEPQRRSARLSAKPAPPKPEPKPKKAPAKKX

SEQ ID NO: 557 - protein, 10 aa, synthetic construct
  REGION 1..10: note = cell penetrating peptide
  MOD_RES 10: note = Xaa = glycine or proline
RKKRRQRRRX

SEQ ID NO: 558 - protein, 9 aa, synthetic construct
  REGION 1..9: note = cell penetrating peptide
  MOD_RES 9: note = Xaa = glycine or proline
RKKRRQRRX

SEQ ID NO: 559 - protein, 8 aa, synthetic construct
  REGION 1..8: note = cell penetrating peptide
  MOD_RES 8: note = Xaa = glycine or proline
RKKRRQRX

SEQ ID NO: 560 - protein, 9 aa, synthetic construct
  REGION 1..9: note = cell penetrating peptide
  MOD_RES 9: note = Xaa = glycine or proline
KKRRQRRRX

SEQ ID NO: 561 - protein, 9 aa, synthetic construct
  REGION 1..9: note = cell penetrating peptide
  MOD_RES 9: note = Xaa = glycine or proline
KKRRQRRRX

SEQ ID NO: 562 - protein, 10 aa, synthetic construct
  REGION 1..10: note = cell penetrating peptide
  MOD_RES 10: note = Xa = glycine or proline
AKKRRQRRRX

SEQ ID NO: 563 - protein, 10 aa, synthetic construct
  REGION 1..10: note = cell penetrating peptide
  MOD_RES 10: note = Xaa = glycine or proline
RAKRRQRRRX

SEQ ID NO: 564 - protein, 10 aa, synthetic construct
  REGION 1..10: note = cell penetrating peptide
  MOD_RES 10: note = Xaa = glycine or proline
RKARRQRRRX

SEQ ID NO: 565 - protein, 10 aa, synthetic construct
  REGION 1..10: note = cell penetrating peptide
  MOD_RES 10: note = Xaa = glycine or proline
RKKARQRRRX

SEQ ID NO: 566 - protein, 11 aa, synthetic construct
  REGION 1..11: note = cell penetrating peptide
  MOD_RES 11: note = Xaa = glycine or proline
CRWRWKCCKKX

SEQ ID NO: 567 - DNA, 22 bp, synthetic construct
  misc_feature 1..22: note = synthetic oligonucleotide
  source 1..22: mol_type = other DNA
cggtccacgtagactaacaact

SEQ ID NO: 568 - DNA, 23 bp, synthetic construct
  misc_feature 1..23: note = synthetic oligonucleotide
  source 1..23: mol_type = other DNA
gaagttcacacagataaacttct

SEQ ID NO: 569 - DNA, 25 bp, synthetic construct
  misc_feature 1..25: note = synthetic oligonucleotide
  source 1..25: mol_type = other DNA
tttcgacatcggttagaagactcat

SEQ ID NO: 570 - DNA, 25 bp, synthetic construct
  misc_feature 1..25: note = synthetic oligonucleotide
  source 1..25: mol_type = other DNA
tttcgacatcggttagaagactcat

SEQ ID NO: 571 - DNA, 22 bp, synthetic construct
  misc_feature 1..22: note = synthetic oligonucleotide
  source 1..22: mol_type = other DNA
gagacgccatgatgtggatgtc

SEQ ID NO: 572 - DNA, 23 bp, synthetic construct
  misc_feature 1..23: note = synthetic oligonucleotide
  source 1..23: mol_type = other DNA
gaaacacggacacccaaagtagt

SEQ ID NO: 573 - DNA, 22 bp, synthetic construct
  misc_feature 1..22: note = synthetic oligonucleotide
  source 1..22: mol_type = other DNA
tcccagcgtcaatatgctgttt

SEQ ID NO: 574 - DNA, 20 bp, synthetic construct
  misc_feature 1..20: note = synthetic oligonucleotide
  source 1..20: mol_type = other DNA
gcctaggatccacggtgcgc

SEQ ID NO: 575 - DNA, 40 bp, synthetic construct; identical to SEQ ID NO: 9
  misc_feature 1..40: note = synthetic oligonucleotide
  source 1..40: mol_type = other DNA

SEQ ID NO: 576 - protein, 5 aa, synthetic construct
  REGION 1..5: note = carrier peptide
ILFQY

SEQ ID NO: 577 - protein, 4 aa, synthetic construct
  REGION 1..4: note = carrier peptide
ILFQ

SEQ ID NO: 578 - protein, 4 aa, synthetic construct
  REGION 1..4: note = carrier peptide
IWFQ

SEQ ID NO: 579 - protein, 4 aa, synthetic construct
  REGION 1..4: note = carrier peptide
ILIQ

SEQ ID NO: 580 - protein, 5 aa, synthetic construct
  REGION 1..5: note = carrier peptide
PPMWS

SEQ ID NO: 581 - protein, 5 aa, synthetic construct
  REGION 1..5: note = carrier peptide
PPMWT

SEQ ID NO: 582 - protein, 5 aa, synthetic construct
  REGION 1..5: note = carrier peptide
PPMFS

SEQ ID NO: 583 - protein, 5 aa, synthetic construct
  REGION 1..5: note = carrier peptide
PPMYS

(19) United States
(12) Patent Application Publication
(10) Pub. No.: US 2026/0366022 A1
(43) Pub. Date: Nov. 19, 2026

(54) PEPTIDE OLIGONUCLEOTIDE CONJUGATES

(71) Applicant: Sarepta Therapeutics, Inc., Cambridge, MA (US)

(72) Inventor: Gunnar J. HANSON, Cambridge, MA (US)

(73) Assignee: Sarepta Therapeutics, Inc., Cambridge, MA (US)

(21) Appl. No.: 19/306,774

(22) Filed: Aug. 21, 2025

Related U.S. Application Data
(63) Continuation of application No. 19/008,432, filed on Jan. 2, 2025, now abandoned.
Application No. 19/008,432 is a continuation of application No. 18/670,345, filed on May 21, 2024, now abandoned.
Application No. 18/670,345 is a continuation of application No. 18/537,064, filed on Dec. 12, 2023, now abandoned.
Application No. 18/537,064 is a continuation of application No. 18/343,920, filed on Jun. 29, 2023, now abandoned.
Application No. 18/343,920 is a continuation of application No. 16/869,213, filed on May 7, 2020, now Pat. No. 11,732,259.
Application No. 16/869,213 is a continuation of application No. 16/593,615, filed on Oct. 4, 2019, now Pat. No. 11,225,662.
Application No. 16/593,615 is a continuation of application No. 15/827,431, filed on Nov. 30, 2017, now Pat. No. 10,487,326.
Application No. 15/827,431 is a continuation of application No. 14/851,434, filed on Sep. 11, 2015, now Pat. No. 9,862,946.
Application No. 14/851,434 is a division of application No. 13/299,310, filed on Nov. 17, 2011, now Pat. No. 9,161,948.
Application No. 13/299,310 is a continuation-in-part of application No. 13/107,528, filed on May 13, 2011, now Pat. No. 9,238,042.
Application No. 13/107,528 is a continuation-in-part of application No. 13/101,942, filed on May 5, 2011, now abandoned.

(51) Int. Cl. C12N 15/113; A61K 31/712; A61K 31/713; A61K 47/64; C12N 15/87

(52) U.S. Cl. CPC C12N 15/113; A61K 31/712; A61K 31/713; A61K 47/64; A61K 47/645; C12N 15/1136; C12N 15/1138; C12N 15/87; C12N 2310/11; C12N 2310/3233; C12N 2310/33; C12N 2310/3513; C12N 2320/33

(57) ABSTRACT

Oligonucleotide analogues conjugated to carrier peptides are provided. The disclosed compounds are useful for the treatment of various diseases, for example diseases where inhibition of protein expression or correction of aberrant mRNA splice products produces beneficial therapeutic effects.

### CROSS-REFERENCE TO RELATED APPLICATIONS

This application is a Continuation of U.S. application Ser. No. 19/008,432, filed Jan. 2, 2025, pending, which is a Continuation of U.S. application Ser. No. 18/670,345, filed May 21, 2024, now abandoned, which is a Continuation of U.S. application Ser. No. 18/537,064, filed Dec. 12, 2023, now abandoned, which is a Continuation of U.S. application Ser. No. 18/343,920, filed Jun. 29, 2023, now abandoned, which is a Continuation of U.S. application Ser. No. 16/869,213, filed May 7, 2020, now issued as U.S. Pat. No. 11,732,259, which is a Continuation of U.S. application Ser. No. 16/593,615, filed Oct. 4, 2019, now issued as U.S. Pat. No. 11,225,662, which is a Continuation of U.S. application Ser. No. 15/827,431, filed Nov. 30, 2017, now issued as U.S. Pat. No. 10,487,326, which is a Continuation of U.S. application Ser. No. 14/851,434, filed Sep. 11, 2015, now issued as U.S. Pat. No. 9,862,946, which is a Divisional of U.S. application Ser. No. 13/299,310, filed Nov. 17, 2011, now issued as U.S. Pat. No. 9,161,948, which is a Continuation-in-part of U.S. patent application Ser. No. 13/101,942, filed on May 5, 2011, now abandoned, and a Continuation-in-part of U.S. patent application Ser. No. 13/107,528, filed on May 13, 2011, now issued as U.S. Pat. No. 9,238,042. The above referenced applications are incorporated herein by reference in their entireties.

### SEQUENCE LISTING

The content of the electronically submitted sequence listing (Name: 4140_082000L_SequenceListing_ST26.xml; Size 1,137,921 bytes; and Date of Creation: Aug. 12, 2025) is herein incorporated by reference in its entirety.

### BACKGROUND

### Technical Field

The present invention is generally related to oligonucleotide compounds (oligomers) useful as antisense compounds, and more particularly to oligomer compounds conjugated to cell-penetrating peptides, and the use of such oligomer compounds in antisense applications.

### Description of the Related Art

The practical utility of many drugs having potentially useful biological activity is often stymied by difficulty in delivering such drugs to their targets. Compounds to be delivered into cells must generally be delivered from a largely aqueous extracellular environment and then penetrate a lipophilic cell membrane to gain entry to the cell. Unless the substance is actively transported by a specific transport mechanism, many molecules, particularly large molecules, are either too lipophilic for practical solubilization or are too hydrophilic to penetrate the membrane.

A segment of the HIV Tat protein consisting of amino acid residues 49-57 (Tat 49 57, having the sequence RKKRRQRRR(SEQ ID NO: 57)) has been used to deliver biologically active peptides and proteins to cells (e.g. Barsoum et al., 1994, PCT Pubn. No. WO 94/04686). Tat (49 60) has been used to enhance delivery of phosphorothioate oligonucleotides (Astriab-Fisher, Sergueev et al. 2000; Astriab-Fisher, Sergueev et al. 2002). Reverse Tat, or rTat(57-49) (RRRQRRKKR) (SEQ ID NO: 56), has been reported to deliver fluorescein into cells with enhanced efficacy compared to Tat (49 57) (Wender, Mitchell et al. 2000; Rothbard, Kreider et al. 2002). Rothbard and Wender have also disclosed other arginine-rich transport polymers (PCT Pubn. No. WO 01/62297; U.S. Pat. No. 6,306,993; US Patent Appn. Pubn. No. 2003/0032593).

Oligonucleotides are one class of potentially useful drug compounds whose delivery has often been an impediment to therapeutic use. Phosphorodiamidate-linked morpholino oligomers (PMOs; see e.g. Summerton and Weller, 1997) have been found more promising in this regard than charged oligonucleotide analogs such as phosphorothioates. The PMOs are water-soluble, uncharged or substantially uncharged antisense molecules that inhibit gene expression by preventing binding or progression of splicing or translational machinery components. PMOs have also been to shown to inhibit or block viral replication (Stein, Skilling et al. 2001; McCaffrey, Meuse et al. 2003). They are highly resistant to enzymatic digestion (Hudziak, Barofsky et al. 1996). PMOs have demonstrated high antisense specificity and efficacy in vitro in cell-free and cell culture models (Stein, Foster et al. 1997; Summerton and Weller 1997), and in vivo in zebrafish, frog and sea urchin embryos (Heasman, Kofron et al. 2000; Nasevicius and Ekker 2000), as well as in adult animal models, such as rats, mice, rabbits, dogs, and pigs (see e.g. Arora and Iversen 2000; Qin, Taylor et al. 2000; Iversen 2001; Kipshidze, Keane et al. 2001; Devi 2002; Devi, Oldenkamp et al. 2002; Kipshidze, Kim et al. 2002; Ricker, Mata et al. 2002).

Antisense PMO oligomers have been shown to be taken up into cells and to be more consistently effective in vivo, with fewer nonspecific effects, than other widely used antisense oligonucleotides (see e.g. P. Iversen, “Phosphoramidite Morpholino Oligomers”, in Antisense Drug Technology, S. T. Crooke, ed., Marcel Dekker, Inc., New York, 2001). Conjugation of PMOs to arginine rich peptides has been shown to increase their cellular uptake (see e.g., U.S. Pat. No. 7,468,418); however, the toxicity of the conjugates has slowed their development as viable drug candidates.

Although significant progress has been made, there remains a need in the art for oligonucleotide conjugates with improved antisense or antigene performance. Such improved antisense or antigene performance includes; lower toxicity, stronger affinity for DNA and RNA without compromising sequence selectivity; improved pharmacokinetics and tissue distribution; improved cellular delivery and reliable and controllable in vivo distribution.

### BRIEF SUMMARY

Compounds of the present invention address these issues and provide improvements over existing antisense molecules in the art. By linking a cell-penetrating peptide to a substantially uncharged nucleic acid analogue via a glycine or proline amino acid, the present inventors have addressed the toxicity issues associated with other peptide oligomer conjugates. Furthermore, modification of the intersubunit linkages and/or conjugation of terminal moieties to the 5′ and/or 3′ terminus of an oligonucleotide analogue, for example a morpholino oligonucleotide, may also improve the properties of the conjugates. For example, in certain embodiments the disclosed conjugates have decreased toxicity and/or enhanced cell delivery, potency, and/or tissue distribution compared to other oligonucleotide analogues and/or can be more effectively delivered to the target organs. These superior properties give rise to favorable therapeutic indices, reduced clinical dosing, and lower cost of goods.

Accordingly, in one embodiment the present disclosure provides a conjugate comprising:
(a) a carrier peptide comprising amino acid subunits; and
(b) a nucleic acid analogue comprising a substantially uncharged backbone and a targeting base sequence for sequence-specific binding to a target nucleic acid;
   wherein:
two or more of the amino acid subunits are positively charged amino acids, the carrier peptide comprises a glycine (G) or proline (P) amino acid at a carboxy terminus of the carrier peptide, and the carrier peptide is covalently attached to the nucleic acid analogue. A composition comprising the above conjugate and a pharmaceutically acceptable vehicle are also provided.

In another embodiment, the present disclosure provides a method of inhibiting production of a protein, the method comprising exposing a nucleic acid encoding the protein to a conjugate of the present disclosure.

Another aspect of the present disclosure includes a method for enhancing the transport of a nucleic acid analogue into a cell, the method comprising conjugating the carrier peptide of claim 1 to a nucleic acid analogue, and wherein the transport of the nucleic acid analogue into the cell is enhanced relative to the nucleic acid analogue in unconjugated form.

In another embodiment, the disclosure is directed to a method of treating a disease in a subject, the method comprising administering a therapeutically effective amount of a disclosed conjugate to the subject. Methods of making the conjugates, methods for their use and carrier peptides useful for conjugating to nucleic acid analogues are also provided.

These and other aspects of the invention will be apparent upon reference to the following detailed description. To this end, various references are set forth herein which describe in more detail certain background information, procedures, compounds and/or compositions, and are each hereby incorporated by reference in their entirety.

### BRIEF DESCRIPTION OF THE DRAWINGS

FIG. 1A shows an exemplary morpholino oligomer structure comprising a phosphorodiamidate linkage.

FIG. 1B shows a morpholino oligomer conjugated to a carrier peptide at the 5′ end.

FIG. 1C shows a morpholino oligomer conjugated to a carrier peptide at the 3′ end.

FIGS. 1D-G show the repeating subunit segment of exemplary morpholino oligonucleotides, designated 1D through 1G.

FIG. 2 depicts exemplary intersubunit linkages linked to a morpholino −T moiety.

FIG. 3 is a reaction scheme showing preparation of a linker for solid-phase synthesis.

FIG. 4 demonstrates preparation of a solid support for oligomer synthesis.

FIGS. 5A, 5B and 5C show exon skipping data for exemplary conjugates compared to a known conjugate in mouse quadriceps, diaphragm and heart, respectively.

FIGS. 6A, 6B and 6C are alternate representations of exon skipping data for exemplary conjugates compared to a known conjugate in mouse quadriceps, diaphragm and heart, respectively.

FIGS. 7A and 7B are graphs depicting blood urea nitrogen (BUN) levels and survival rate of mice treated with various peptide-oligomer conjugates, respectively.

FIGS. 8A and 8B show kidney injury marker (KIM) data and Clusterin (Clu) data for mice treated with various peptide-oligomer conjugates, respectively.

FIGS. 9A, 9B, 9C and 9D are graphs comparing the exon skipping, BUN levels, precent survival and KIM levels, respectively, in mice treated with an exemplary conjugate compared to a known conjugate.

FIG. 10 presents KIM data for mice treated with various conjugates.

FIG. 11 shows results of BUN analysis of mice treated with various conjugates.

FIG. 12 is a graph showing the concentration of various oligomers in mouse kidney tissue.

### DETAILED DESCRIPTION

### I. Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“Amino” refers to the —NH₂ radical.

“Cyano” or “nitrile” refers to the —CN radical.

“Hydroxy” or “hydroxyl” refers to the —OH radical.

“Imino” refers to the ═NH substituent.

“Guanidinyl” refers to the —NHC(═NH)NH₂ substituent.

“Amidinyl” refers to the —C(═NH)NH₂ substituent.

“Nitro” refers to the —NO₂ radical.

“Oxo” refers to the ═O substituent.

“Thioxo” refers to the ═S substituent.

“Cholate” refers to the following structure:

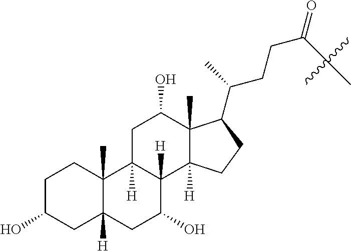

“Deoxycholate” refers to the following structure:

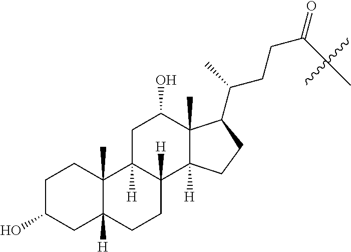

“Alkyl” refers to a straight or branched hydrocarbon chain radical which is saturated or unsaturated (i.e., contains one or more double and/or triple bonds), having from one to thirty carbon atoms, and which is attached to the rest of the molecule by a single bond. Alkyls comprising any number of carbon atoms from 1 to 30 are included. An alkyl comprising up to 30 carbon atoms is referred to as a C₁-C₃₀ alkyl, likewise, for example, an alkyl comprising up to 12 carbon atoms is a C₁-C₁₂ alkyl. Alkyls (and other moieties defined herein) comprising other numbers of carbon atoms are represented similarly. Alkyl groups include, but are not limited to, C₁-C₃₀ alkyl, C₁-C₂₀ alkyl, C₁-C₁₅ alkyl, C₁-C₁₀ alkyl, C₁-C₈ alkyl, C₁-C₆ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (iso-propyl), n-butyl, i-butyl, s-butyl, n-pentyl, 1,1-dimethylethyl (t-butyl), 3-methylhexyl, 2-methylhexyl, ethenyl, prop-1-enyl, but-1-enyl, pent-1-enyl, penta-1,4-dienyl, ethynyl, propynyl, but-2-ynyl, but-3-ynyl, pentynyl, hexynyl, and the like. Unless stated otherwise specifically in the specification, an alkyl group may be optionally substituted as described below.

“Alkylene” or “alkylene chain” refers to a straight or branched divalent hydrocarbon chain linking the rest of the molecule to a radical group. Alkylenes may be saturated or unsaturated (i.e., contains one or more double and/or triple bonds). Representative alkylenes include, but are not limited to, C₁-C₁₂ alkylene, C₁-C₈ alkylene, C₁-C₆ alkylene, C₁-C₄ alkylene, C₁-C₃ alkylene, C₁-C₂ alkylene, C₁ alkylene. Representative alkylene groups include, but are not limited to, methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted as described below.

“Alkoxy” refers to a radical of the formula —ORₐ where Rₐ is an alkyl radical as defined. Unless stated otherwise specifically in the specification, an alkoxy group may be optionally substituted as described below.

“Alkoxyalkyl” refers to a radical of the formula —R_{b}ORₐ where Rₐ is an alkyl radical as defined and where R^{b} is an alkylene radical as defined. Unless stated otherwise specifically in the specification, an alkoxyalkyl group may be optionally substituted as described below.

“Alkylcarbonyl” refers to a radical of the formula —C(═O)Rₐ where Rₐ is an alkyl radical as defined above. Unless stated otherwise specifically in the specification, an alkylcarbonyl group may be optionally substituted as described below.

“Alkyloxycarbonyl” refers to a radical of the formula —C(═O)ORₐ where Rₐ is an alkyl radical as defined. Unless stated otherwise specifically in the specification, an alkyloxycarbonyl group may be optionally substituted as described below.

“Alkylamino” refers to a radical of the formula —NHRₐ or —NRₐRₐ where each Rₐ is, independently, an alkyl radical as defined above. Unless stated otherwise specifically in the specification, an alkylamino group may be optionally substituted as described below.

“Amidyl” refers to a radical of the formula —N(H)C(═O) Rₐ where Rₐ is an alkyl or aryl radical as defined herein. Unless stated otherwise specifically in the specification, an amidyl group may be optionally substituted as described below.

“Amidinylalkyl” refers a radical of the formula —R_{b}— C(═NH)NH₂ where R_{b} is an alkylene radical as defined above. Unless stated otherwise specifically in the specification, an amidinylalkyl group may be optionally substituted as described below.

“Amidinylalkylcarbonyl” refers a radical of the formula —C(═O)R_{b}— C(═NH)NH₂ where R_{b} is an alkylene radical as defined above. Unless stated otherwise specifically in the specification, an amidinylalkylcarbonyl group may be optionally substituted as described below.

“Aminoalkyl” refers to a radical of the formula —R_{b}—NRₐRₐ where R_{b} is an alkylene radical as defined above, and each Rₐ is independently a hydrogen or an alkyl radical.

“Thioalkyl” refers to a radical of the formula —SRₐ where Rₐ is an alkyl radical as defined above. Unless stated otherwise specifically in the specification, a thioalkyl group may be optionally substituted.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused or bridged ring systems. Aryl radicals include, but are not limited to, aryl radicals derived from the hydrocarbon ring systems of aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Aralkyl” refers to a radical of the formula —R_{b}—R_{c} where R_{b} is an alkylene chain as defined above and R_{c} is one or more aryl radicals as defined above, for example, benzyl, diphenylmethyl, trityl and the like. Unless stated otherwise specifically in the specification, an aralkyl group may be optionally substituted.

“Arylcarbonyl” refers to a radical of the formula —C(═O)R_{c} where R_{c} is one or more aryl radicals as defined above, for example, phenyl. Unless stated otherwise specifically in the specification, an arylcarbonyl group may be optionally substituted.

“Aryloxycarbonyl” refers to a radical of the formula —C(═O)OR_{c} where R_{c} is one or more aryl radicals as defined above, for example, phenyl. Unless stated otherwise specifically in the specification, an aryloxycarbonyl group may be optionally substituted.

“Aralkylcarbonyl” refers to a radical of the formula —C(═O)R_{b}—R_{c} where R_{b} is an alkylene chain as defined above and R_{c} is one or more aryl radicals as defined above, for example, phenyl. Unless stated otherwise specifically in the specification, an aralkylcarbonyl group may be optionally substituted.

“Aralkyloxycarbonyl” refers to a radical of the formula —C(═O)OR_{b}—R_{c} where R_{b} is an alkylene chain as defined above and R_{c} is one or more aryl radicals as defined above, for example, phenyl. Unless stated otherwise specifically in the specification, an aralkyloxycarbonyl group may be optionally substituted.

“Aryloxy” refers to a radical of the formula —OR_{c} where R_{c} is one or more aryl radicals as defined above, for example, phenyl. Unless stated otherwise specifically in the specification, an arylcarbonyl group may be optionally substituted.

“Cycloalkyl” refers to a stable, non-aromatic, monocyclic or polycyclic carbocyclic ring, which may include fused or bridged ring systems, which is saturated or unsaturated, and attached to the rest of the molecule by a single bond. Representative cycloalkyls include, but are not limited to, cycloaklyls having from three to fifteen carbon atoms and from three to eight carbon atoms, Monocyclic cycloalkyl radicals include, for example, cyclopropyl, cyclobutyl, cyclopentyl, cyclohexyl, cycloheptyl, and cyclooctyl. Polycyclic radicals include, for example, adamantyl, norbornyl, decalinyl, and 7,7-dimethyl-bicyclo[2.2.1]heptanyl. Unless otherwise stated specifically in the specification, a cycloalkyl group may be optionally substituted.

“Cycloalkylalkyl” refers to a radical of the formula —R_{b}R_{d} where R_{b} is an alkylene chain as defined above and R_{d} is a cycloalkyl radical as defined above. Unless stated otherwise specifically in the specification, a cycloalkylalkyl group may be optionally substituted.

“Cycloalkylcarbonyl” refers to a radical of the formula —C(═O)R_{d} where R_{d} is a cycloalkyl radical as defined above. Unless stated otherwise specifically in the specification, a cycloalkylcarbonyl group may be optionally substituted.

Cycloalkyloxycarbonyl” refers to a radical of the formula —C(═O)OR_{d} where R_{d} is a cycloalkyl radical as defined above. Unless stated otherwise specifically in the specification, a cycloalkyloxycarbonyl group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Guanidinylalkyl” refers a radical of the formula —R_{b}—NHC(═NH)NH₂ where R_{b} is an alkylene radical as defined above. Unless stated otherwise specifically in the specification, a guanidinylalkyl group may be optionally substituted as described below.

“Guanidinylalkylcarbonyl” refers a radical of the formula —C(═O)R_{b}—NHC(═NH)NH₂ where R_{b} is an alkylene radical as defined above. Unless stated otherwise specifically in the specification, a guanidinylalkylcarbonyl group may be optionally substituted as described below.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Perhalo” or “perfluoro” refers to a moiety in which each hydrogen atom has been replaced by a halo atom or fluorine atom, respectively.

“Heterocyclyl”, “heterocycle” or “heterocyclic ring” refers to a stable 3- to 24-membered non-aromatic ring radical comprising 2 to 23 carbon atoms and from one to 8 heteroatoms selected from the group consisting of nitrogen, oxygen, phosphorous and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 12-crown-4, 15-crown-5, 18-crown-6, 21-crown-7, aza-18-crown-6, diaza-18-crown-6, aza-21-crown-7, and diaza-21-crown-7. Unless stated otherwise specifically in the specification, a heterocyclyl group may be optionally substituted.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group may be optionally substituted.

All the above groups may be either substituted or unsubstituted. The term “substituted” as used herein means any of the above groups (i.e., alkyl, alkylene, alkoxy, alkoxyalkyl, alkylcarbonyl, alkyloxycarbonyl, alkylamino, amidyl, amidinylalkyl, amidinylalkylcarbonyl, aminoalkyl, aryl, aralkyl, arylcarbonyl, aryloxycarbonyl, aralkylcarbonyl, aralkyloxycarbonyl, aryloxy, cycloalkyl, cycloalkylalkyl, cycloalkylcarbonyl, cycloalkylalkylcarbonyl, cycloalkyloxycarbonyl, guanidinylalkyl, guanidinylalkylcarbonyl, haloalkyl, heterocyclyl and/or heteroaryl), may be further functionalized wherein at least one hydrogen atom is replaced by a bond to a non-hydrogen atom substituent. Unless stated specifically in the specification, a substituted group may include one or more substituents selected from: oxo, —CO₂H, nitrile, nitro, —CONH₂, hydroxyl, thiooxy, alkyl, alkylene, alkoxy, alkoxyalkyl, alkylcarbonyl, alkyloxycarbonyl, aryl, aralkyl, arylcarbonyl, aryloxycarbonyl, aralkylcarbonyl, aralkyloxycarbonyl, aryloxy, cycloalkyl, cycloalkylalkyl, cycloalkylcarbonyl, cycloalkylalkylcarbonyl, cycloalkyloxycarbonyl, heterocyclyl, heteroaryl, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, triarylsilyl groups, perfluoroalkyl or perfluoroalkoxy, for example, trifluoromethyl or trifluoromethoxy. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —S OR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —CH₂SO₂R_{g}, —CH₂SO₂NR_{g}Rₕ, —SH, —SR_{g} or —SSR_{g}. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. In addition, each of the foregoing substituents may also be optionally substituted with one or more of the above substituents. Furthermore, any of the above groups may be substituted to include one or more internal oxygen or sulfur atoms. For example, an alkyl group may be substituted with one or more internal oxygen atoms to form an ether or polyether group. Similarly, an alkyl group may be substituted with one or more internal sulfur atoms to form a thioether, disulfide, etc. Amidyl moieties may be substituted with up to 2 halo atoms, while other groups above may be substituted with one or more halo atoms. Any of the above groups may also be substituted with amino, monoalklyamino, guanidinyl or amidynyl. Optional substitutents for any of the above groups also include arylphosphoryl, for example —RₐP(Ar)₃ wherein Rₐ is an alkylene and Ar is aryl moiety, for example phenyl.

The terms “antisense oligomer” or “antisense compound” are used interchangeably and refer to a sequence of subunits, each having a base carried on a backbone subunit composed of ribose or other pentose sugar or morpholino group, and where the backbone groups are linked by intersubunit linkages that allow the bases in the compound to hybridize to a target sequence in a nucleic acid (typically an RNA) by Watson-Crick base pairing, to form a nucleic acid:oligomer heteroduplex within the target sequence. The oligomer may have exact sequence complementarity to the target sequence or near complementarity. Such antisense oligomers are designed to block or inhibit translation of the mRNA containing the target sequence, and may be said to be “directed to” a sequence with which it hybridizes.

A “morpholino oligomer” or “PMO” refers to a polymeric molecule having a backbone which supports bases capable of hydrogen bonding to typical polynucleotides, wherein the polymer lacks a pentose sugar backbone moiety, and more specifically a ribose backbone linked by phosphodiester bonds which is typical of nucleotides and nucleosides, but instead contains a ring nitrogen with coupling through the ring nitrogen. An exemplary“morpholino” oligomer comprises morpholino subunit structures linked together by (thio)phosphoramidate or (thio)phosphorodiamidate linkages, joining the morpholino nitrogen of one subunit to the 5′ exocyclic carbon of an adjacent subunit, each subunit comprising a purine or pyrimidine base-pairing moiety effective to bind, by base-specific hydrogen bonding, to a base in a polynucleotide. Morpholino oligomers (including antisense oligomers) are detailed, for example, in U.S. Pat. Nos. 5,698,685; 5,217,866; 5,142,047; 5,034,506; 5,166,315; 5,185,444; 5,521,063; 5,506,337 and pending U.S. patent application Ser. Nos. 12/271,036; 12/271,040; and PCT publication number WO/2009/064471 all of which are incorporated herein by reference in their entirety. Representative PMOs include PMOs wherein the intersubunit linkages are linkage (A1).

“PMO+” refers to phosphorodiamidate morpholino oligomers comprising any number of (1-piperazino)phosphinylideneoxy, (1-(4-(ω-guanidino-alkanoyl))-piperazino)phosphinylideneoxy linkages (A2 and A3) that have been described previously (see e.g., PCT publication WO/2008/036127 which is incorporated herein by reference in its entirety.

“PMO-X” refers to phosphorodiamidate morpholino oligomers disclosed herein comprising at least one (B) linkage or at least one of the disclosed terminal modifications.

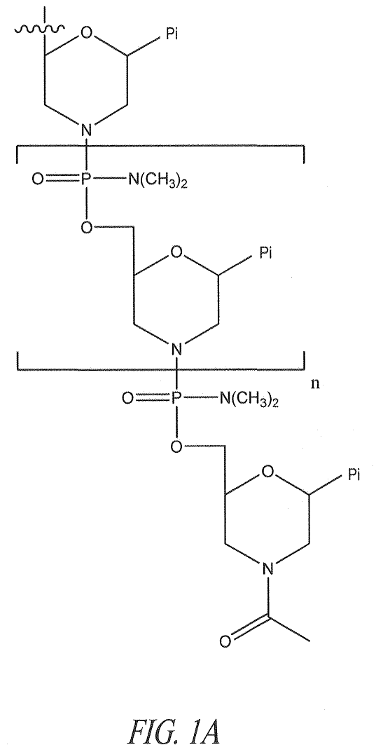

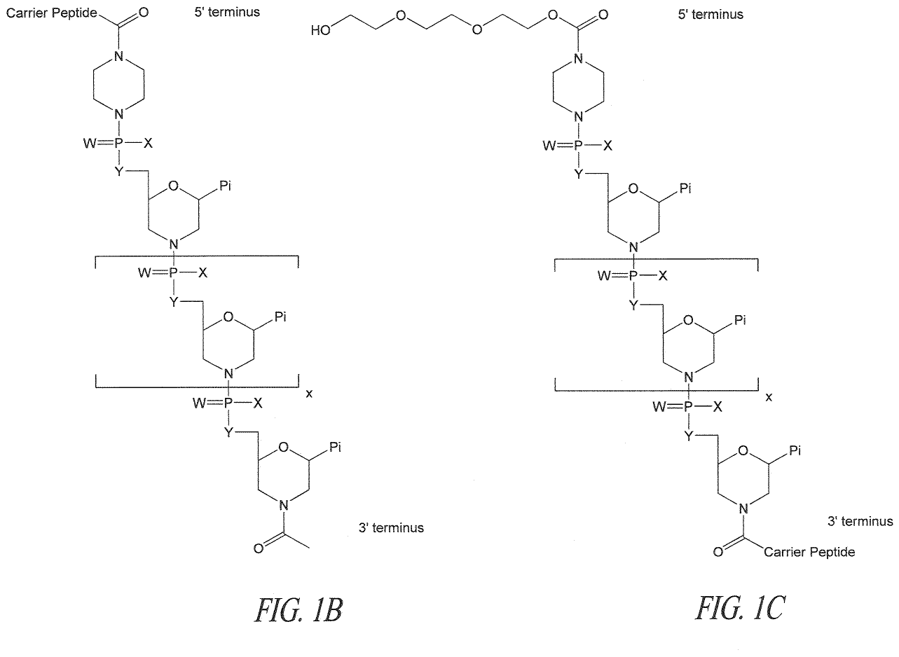

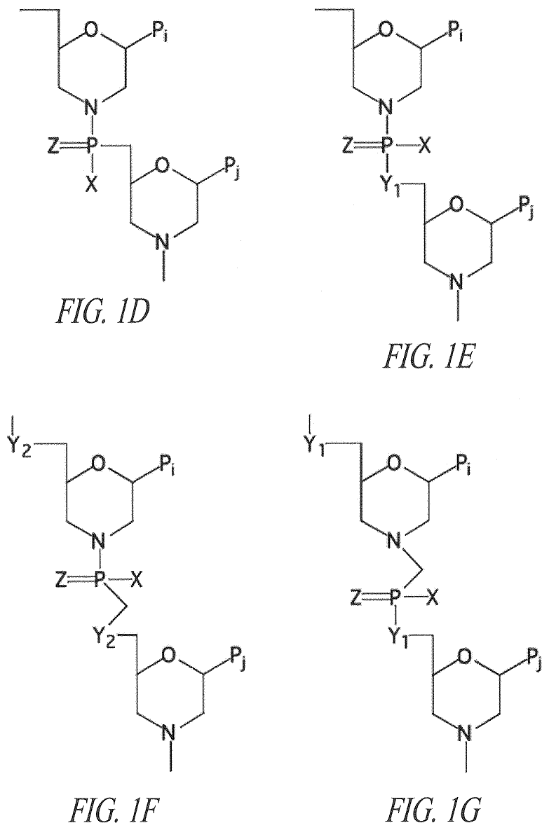

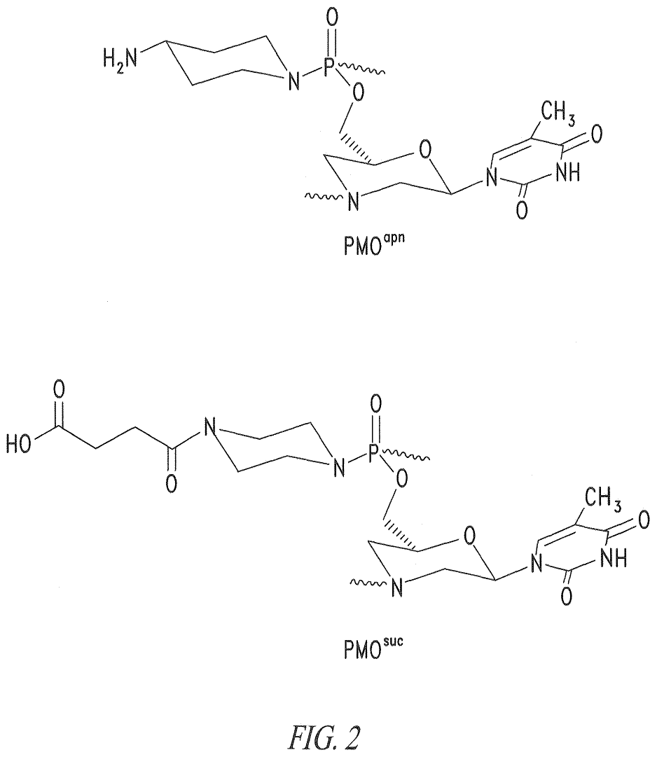

A “phosphoramidate” group comprises phosphorus having three attached oxygen atoms and one attached nitrogen atom, while a “phosphorodiamidate” group (see e.g., FIGS. 1D-E) comprises phosphorus having two attached oxygen atoms and two attached nitrogen atoms. In the uncharged or the modified intersubunit linkages of the oligomers described herein and co-pending US Patent Application No. 61/349,783 and Ser. No. 11/801,885, one nitrogen is always pendant to the backbone chain. The second nitrogen, in a phosphorodiamidate linkage, is typically the ring nitrogen in a morpholino ring structure.

“Thiophosphoramidate” or “thiophosphorodiamidate” linkages are phosphoramidate or phosphorodiamidate linkages, respectively, wherein one oxygen atom, typically the oxygen pendant to the backbone, is replaced with sulfur.

“Intersubunit linkage” refers to the linkage connecting two morpholino subunits, for example structure (I).

“Charged”, “uncharged”, “cationic” and “anionic” as used herein refer to the predominant state of a chemical moiety at near-neutral pH, e.g., about 6 to 8. For example, the term may refer to the predominant state of the chemical moiety at physiological pH, that is, about 7.4.

“Lower alkyl” refers to an alkyl radical of one to six carbon atoms, as exemplified by methyl, ethyl, n-butyl, i-butyl, t-butyl, isoamyl, n-pentyl, and isopentyl. In certain embodiments, a “lower alkyl” group has one to four carbon atoms. In other embodiments a “lower alkyl” group has one to two carbon atoms; i.e. methyl or ethyl. Analogously, “lower alkenyl” refers to an alkenyl radical of two to six, preferably three or four, carbon atoms, as exemplified by allyl and butenyl.

A “non-interfering” substituent is one that does not adversely affect the ability of an antisense oligomer as described herein to bind to its intended target. Such substituents include small and/or relatively non-polar groups such as methyl, ethyl, methoxy, ethoxy, or fluoro.

An oligonucleotide or antisense oligomer “specifically hybridizes” to a target polynucleotide if the oligomer hybridizes to the target under physiological conditions, with a Tm greater than 37 °C, greater than 45 °C, preferably at least 50 °C, and typically 60 °C-80 °C or higher. The “Tm” of an oligomer is the temperature at which 50% hybridizes to a complementary polynucleotide. Tm is determined under standard conditions in physiological saline, as described, for example, in Miyada et al., Methods Enzymol. 154:94-107 (1987). Such hybridization may occur with “near” or “substantial” complementary of the antisense oligomer to the target sequence, as well as with exact complementarity.

Polynucleotides are described as “complementary” to one another when hybridization occurs in an antiparallel configuration between two single-stranded polynucleotides. Complementarity (the degree that one polynucleotide is complementary with another) is quantifiable in terms of the proportion of bases in opposing strands that are expected to form hydrogen bonds with each other, according to generally accepted base-pairing rules.

A first sequence is an “antisense sequence” with respect to a second sequence if a polynucleotide whose sequence is the first sequence specifically binds to, or specifically hybridizes with, the second polynucleotide sequence under physiological conditions.

The term “targeting sequence” is the sequence in the oligonucleotide analog that is complementary (meaning, in addition, substantially complementary) to the target sequence in the RNA genome. The entire sequence, or only a portion, of the analog compound may be complementary to the target sequence. For example, in an analog having 20 bases, only 12-14 may be targeting sequences. Typically, the targeting sequence is formed of contiguous bases in the analog, but may alternatively be formed of non-contiguous sequences that when placed together, e.g., from opposite ends of the analog, constitute sequence that spans the target sequence.

The “backbone” of an oligonucleotide analog (e.g., an uncharged oligonucleotide analogue) refers to the structure supporting the base-pairing moieties; e.g., for a morpholino oligomer, as described herein, the “backbone” includes morpholino ring structures connected by intersubunit linkages (e.g., phosphorus-containing linkages). A “substantially uncharged backbone” refers to the backbone of an oligonuceltoide analogue wherein less than 50% of the intersubunit linkages are charged at near-neutral pH. For example, a substantially uncharged backbone may comprise less than 50%, less than 40%, less than 30%, less than 20%, less than 10%, less than 5% or even 0% intersubunit linkages which are charged at near neutral pH. In some embodiments, the substantially uncharged backbone comprises at most one charged (at physiological pH) intersubunit linkage for every four uncharged (at physiological pH) linkages, at most one for every eight or at most one for every sixteen uncharged linkages. In some embodiments, the nucleic acid analogs described herein are fully uncharged.

Target and targeting sequences are described as “complementary” to one another when hybridization occurs in an antiparallel configuration. A targeting sequence may have “near” or “substantial” complementarity to the target sequence and still function for the purpose of the presently described methods, that is, still be “complementary.” Preferably, the oligonucleotide analog compounds employed in the presently described methods have at most one mismatch with the target sequence per every 10 nucleotides, and preferably at most one mismatch out of 20. Alternatively, the antisense oligomers employed have at least 80%, at least 90% sequence homology or at least 95% sequence homology, with the exemplary targeting sequences as designated herein. For purposes of complementary binding to an RNA target, and as discussed below, a guanine base may be complementary to either a cytosineor uracil RNA base.

A “heteroduplex” refers to a duplex between an oligonculeotide analog and the complementary portion of a target RNA. A “nuclease-resistant heteroduplex” refers to a heteroduplex formed by the binding of an antisense oligomer to its complementary target, such that the heteroduplex is substantially resistant to in vivo degradation by intracellular and extracellular nucleases, such as RNAse H, which are capable of cutting double-stranded RNA/RNA or RNA/DNA complexes.

An agent is “actively taken up by mammalian cells” when the agent can enter the cell by a mechanism other than passive diffusion across the cell membrane. The agent may be transported, for example, by “active transport”, referring to transport of agents across a mammalian cell membrane by e.g. an ATP-dependent transport mechanism, or by “facilitated transport”, referring to transport of antisense agents across the cell membrane by a transport mechanism that requires binding of the agent to a transport protein, which then facilitates passage of the bound agent across the membrane.

The terms “modulating expression” and/or “antisense activity” refer to the ability of an antisense oligomer to either enhance or, more typically, reduce the expression of a given protein, by interfering with the expression or translation of RNA. In the case of reduced protein expression, the antisense oligomer may directly block expression of a given gene, or contribute to the accelerated breakdown of the RNA transcribed from that gene. Morpholino oligomers as described herein are believed to act via the former (steric blocking) mechanism. Preferred antisense targets for steric blocking oligomers include the ATG start codon region, splice sites, regions closely adjacent to splice sites, and 5′-untranslated region of mRNA, although other regions have been successfully targeted using morpholino oligomers.

An “amino acid subunit” is generally an α-amino acid residue (—CO—CHR—NH—); but may also be a β- or other amino acid residue (e.g. —CO—CH₂CHR—NH—), where R is an amino acid side chain.

The term “naturally occurring amino acid” refers to an amino acid present in proteins found in nature. The term “non-natural amino acids” refers to those amino acids not present in proteins found in nature; examples include beta-alanine (β-Ala) and 6-aminohexanoic acid (Ahx).

An “effective amount” or “therapeutically effective amount” refers to an amount of antisense oligomer administered to a mammalian subject, either as a single dose or as part of a series of doses, which is effective to produce a desired therapeutic effect, typically by inhibiting translation of a selected target nucleic acid sequence.

“Treatment” of an individual (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent.

### II. Carrier Peptides

### A. Properties of the Carrier Peptide

As noted above, the present disclosure is directed to conjugates of carrier peptides and nucleic acid analogues. The carrier peptides are generally effective to enhance cell penetration of the nucleic acid analogues. Furthermore, Applicants have surprisingly discovered that including a glycine (G) or proline (P) amino acid subunit between the nucleic acid analogue and the remainder of the carrier peptide (e.g., at the carboxy or amino terminus of the carrier peptide) reduces the toxicity of the conjugate, while the efficacy remains the same or is improved relative to conjugates with different linkages between the carrierpeptide and nucleic acid analogue. Thus the presently disclosed conjugates have a better therapeutic window and are more promising drug candidates than other peptide-oligomer conjugates.

In addition to reduced toxicity, the presence of a glycine or proline amino acid subunit between the nucleic acid analogue and the carrier peptide is believed to provide additional advantages. For example, glycine is inexpensive and is easily coupled to the nucleic acid analogue (or optional linker) without any possibility of racemization. Similarly, proline is easily coupled without racemization and also provides carrier peptides which are not helix formers. The hydrophobicity of proline may also confer certain advantages with respect to interaction of the carrier peptide with the lipid bilayer of cells, and carrier peptides comprising multiple prolines (for example in certain embodiments) may resist G-tetraplex formation. Finally, in certain embodiments, when the proline moiety is adjacent to an arginine amino acid subunit, the proline moiety confers metabolic to the conjugates since the argine-proline amide bond is not cleavable by common endopeptidases.

As noted above, conjugates comprising carrier peptides linked to nucleic acid analogues via a glycine or proline amino acid subunit have lower toxicity and similar efficacy compared to other known conjugates. Experiments performed in support of the present application show that kidney toxicity markers are much lower with the presently disclosed conjugates compared to other conjugates (see e.g., kidney injury marker (KIM) and blood urea nitrogen (BUN) data described in Example 30). While not wishing to be bound by theory, the present inventors believe the reduced toxicity of the disclosed conjugates may be related to the absence of unnatural amino acids such as aminohexanoic acid or β-alanine in the portion of the peptide which is attached to the nucleic acid analogue (e.g., the carboxy terminus). Since these unnatural amino acids are not cleaved in vivo, it is believed that toxic concentrations of the uncleaved peptides may accumulate and cause toxic effects.

The glycine or proline moiety may be at either the amino or carboxy terminus of the carrier peptide, and in some instances, the carrier peptide may be linked to the nucleic acid analogue directly via the glycine or proline subunit or the carrier peptide may be linked to the nucleic acid analogue via an optional linker.

In one embodiment, the present disclosure is directed to a conjugate comprising:
(a) a carrier peptide comprising amino acid subunits; and
(b) a nucleic acid analogue comprising a substantially uncharged backbone and a targeting base sequence for sequence-specific binding to a target nucleic acid;
   wherein:
two or more of the amino acid subunits are positively charged amino acids, the carrier peptide comprises a glycine (G) or proline (P) amino acid subunit at a carboxy terminus of the carrier peptide and the carrier peptide is covalently attached to the nucleic acid analogue. In some embodiments, no more than seven contiguous amino acid subunits are arginine, for example 6 or fewer contiguous amino acid subunits are arginine. In some embodiments, the carrier peptide comprises a glycine amino acid subunit at the carboxy terminus. In other embodiments, the carrier peptide comprises a proline amino acid subunit at the carboxy terminus. In still other embodiments, the carrier peptide comprises a single glycine or proline at the carboxy terminus (i.e., does not comprise a glycine or proline dimmer or trimer, etc. at the carboxy terminus).

In certain embodiments, the carrier peptide, when conjugated to an antisense oligomer having a substantially uncharged backbone, is effective to enhance the binding of the antisense oligomer to its target sequence, relative to the antisense oligomer in unconjugated form, as evidenced by:
(i) a decrease in expression of an encoded protein, relative to that provided by the unconjugated oligomer, when binding of the antisense oligomer to its target sequence is effective to block a translation start codon for the encoded protein, or
(ii) an increase in expression of an encoded protein, relative to that provided by the unconjugated oligomer, when binding of the antisense oligomer to its target sequence is effective to block an aberrant splice site in a pre-mRNA which encodes said protein when correctly spliced. Assays suitable for measurement of these effects are described further below. In one embodiment, conjugation of the peptide provides this activity in a cell-free translation assay, as described herein. In some embodiments, activity is enhanced by a factor of at least two, a factor of at least five or a factor of at least ten.

Alternatively or in addition, the carrier peptide is effective to enhance the transport of the nucleic acid analog into a cell, relative to the analog in unconjugated form. In certain embodiments, transport is enhanced by a factor of at least two, a factor of at least two, a factor of at least five or a factor of at least ten.

In other embodiments, the carrier peptide is effective to decrease the toxicity (i.e., increase maximum tolerated dose) of the conjugate, relative to a conjugate comprising a carrier peptide lacking the terminal glycine or proline amino subunits. In certain embodiments, toxicity is decreased by a factor of at least two, a factor of at least two, a factor of at least five or a factor of at least ten.

A further benefit of the peptide transport moiety is its expected ability to stabilize a duplex between an antisense oligomer and its target nucleic acid sequence. While not wishing to be bound by theory, this ability to stabilize a duplex may result from the electrostatic interaction between the positively charged transport moiety and the negatively charged nucleic acid.

The length of the carrier peptide is not particularly limited and varies in different embodiments. In some embodiments, the carrier peptide comprises from 4 to 40 amino acid subunits. In other embodiments, the carrier peptide comprises from 6 to 30, from 6 to 20, from 8 to 25 or from 10 to 20 amino acid subunits. In some embodiments, the carrier peptide is straight, while in other embodiments it is branched.

In some embodiments, the carrier peptides are rich in positively charged amino acid subunits, for example arginine amino acid subunits. A carrier peptide is “rich” in positively charged amino acids if at least 10% of the amino acid subunits are positively charged. For example, in some embodiments at least 20%, at least 30%, at least 40%, at least 50%, at least 60%, at least 70%, at least 80% or at least 90% of the amino acid subunits are positively charged. In even other embodiments, all the amino acid subunits, except the glycine or proline amino acid subunit, are positively charged. In still other embodiment, all of the positively charged amino acid subunits are arginine.

In other embodiments, the number of positively charged amino acid subunits in the carrier peptide ranges from 1 to 20, for example from 1 to 10 or from 1 to 6. In certain embodiments, the number of positively charged amino acids in the carrier peptide is 1, 2, 3, 4, 5, 6, 7, 8, 9, 10, 11, 12, 13, 14, 15, 16, 17, 18, 19 or 20.

The positively charged amino acids can be naturally occurring, non-naturally occurring, synthetic, modified or analogues of naturally occurring amino acids. For instance, modified amino acids with a net positive charge may be specifically designed for use in the invention as described in more detail below. A number of different types of modification to amino acids are well known in the art. In certain embodiments, the positively charged amino acids are histidine (H), lysine (K) or arginine (R). In other embodiments, the carrier peptide comprises only natural amino acid subunits (i.e., does not contain unnatural amino acids). In other embodiments, the terminal amino acids may be capped, for example with an acetyl, benzoyl or stearyl moiety, for example on the N-terminal end.

Any number, combination and/or sequence of H, K and/or R may be present in the carrier peptide. In some embodiments, all of the amino acid subunits, except the carboxy terminal glycine or proline, are positively charged amino acids. In other embodiments, at least one of the positively charged amino acids is arginine. For example, in some embodiments, all of the positively charged amino acids are arginine, and in even other embodiments the carrier peptide consists of arginine and the carboxy terminal glycine or proline. In yet other embodiments, the carrier peptide comprises no more than seven contiguous arginines, for example no more than six contiguous arginines.

Other types of positively charged amino acids are also envisioned. For example, in certain embodiments, at least one of the positively charged amino acids is an arginine analog. For example, the arginine analog may be a cationic α-amino acid comprising a side chain of the structure R^{a}N═C(NH₂)R^{b}, where Rₐ is H or R^{c}; R^{b} is R^{c}, NH₂, NHR, or N(R^{c})₂, where R^{c} is lower alkyl or lower alkenyl and optionally comprises oxygen or nitrogen or Rₐ and R^{b} may together form a ring; and wherein the side chain is linked to the amino acid via Rₐ or R^{b}. The carrier peptides may comprise any number of these arginine analogues.

The positively charged amino acids may occur in any sequence within the carrier peptide. For example, in some embodiments the positively charged amino acids may alternate or may be sequential. For example, the carrier peptide may comprise the sequence (R^{d})ₘ, wherein R^{d} is independently, at each occurrence, a positively charged amino acid and m is an integer ranging from 2 to 12, from 2 to 10, from 2 to 8 or from 2 to 6. For example, in certain embodiments, R^{d} is arginine, and the carrier peptide comprises a sequence selected from (R)₄, (R)₅, (R)₆, (R)₇ and (R)₅, or selected from (R)₄, (R)₅, (R)₆ and (R)₇ for example in specific embodiments the carrier peptide comprises the sequence (R)₆, for example (R)₆G or (R)₆P.

In other embodiments, the carrier peptide consists of the sequence (R^{d}). and the carboxy terminal glycine or proline, wherein R^{d} is independently, at each occurrence, a positively charged amino acid and m is an integer ranging from 2 to 12, from 2 to 10, from 2 to 8 or from 2 to 6. In certain embodiments R_{d} is independently, at each occurrence, arginine, histidine or lysine. For example, in certain embodiments, R^{d} is arginine, and the carrier peptide consists of a sequence selected from (R)₄, (R)₅, (R)₆, (R)₇ and (R)₅ and the carboxy terminal glycine or proline. For example in specific embodiments the carrier peptide consists of the sequence (R)₆G or (R)₆P.

In some other embodiments, the carrier peptide may comprise one or more hydrophobic amino acid subunits, the hydrophobic amino acid subunits comprising a substituted or unsubstituted alkyl, alkenyl, alkynyl, aryl or aralkyl side chain wherein the alkyl, alkenyl and alkynyl side chain includes at most one heteroatom for every six carbon atoms acid. In some embodiments, the hydrophobic amino acid is phenylalanine (F). For example, the carrier peptide may comprise two or more contingous hydrophobic amino acids such as phenylalanine (F), for example two contiguous phenylalanine moieties. The hydrophobic amino acid(s) may be at any point in the carrier peptide sequence.

In other embodiments, the carrier peptide comprises the sequence [(R^{d}Y^{b}R^{d})ₓ(R^{d}R^{d}Y^{b})_{y}]_{z}, or [(R^{d}R^{d}Y^{b})_{y}(R^{d}Y^{b}R^{d})ₓ]_{z} wherein R^{d} is independently, at each occurrence, a positively charged amino acid, x and y are independently, at each occurrence, 0 or 1, provided that x+y is 1 or 2, z is 1, 2, 3, 4, 5 or 6 and Y^{b} is

—C(O)—(CHR^{e})ₙ—NH— (Y^{b})

wherein n is 2 to 7 and each R^{e} is independently, at each occurrence, hydrogen or methyl. In some of these embodiments, R^{d} is independently, at each occurrence arginine, histidine or lysine. In other embodiments, each R^{d} is arginine. In other embodiments, n is 5 and Y^{b} is an aminohexanoic acid moiety. In other embodiments, n is 2 and Y^{b} is a β-alanine moiety. In yet other embodiments, R^{e} is hydrogen.

In certain embodiments of the foregoing, x is 1 and y is 0, and the carrier peptide comprises the sequence (R^{d}Y^{b}R^{d})_{z}. In other embodiments, n is 5 and Y^{b} is an aminohexanoic acid moiety. In other embodiments, n is 2 and Y^{b} is a β-alanine moiety. In yet other embodiments, R^{e} is hydrogen.

In still other embodiments of the foregoing, x is 0 and y is 1, and the carrier peptide comprises the sequence (R^{d}R^{d}Y^{b})_{z}. In other embodiments, n is 5 and Y^{b} is an aminohexanoic acid moiety. In other embodiments, n is 2 and Y^{b} is a β-alanine moiety. In yet other embodiments, R^{e} is hydrogen.

In other embodiments, the carrier peptide comprises the sequence (R^{d}Y^{b})ₚ, wherein R^{d} and Y^{b} are as defined above and p is an integer ranging from 2 to 8. In other embodiments, each R^{d} is arginine. In other embodiments, n is 5 and Y^{b} is an aminohexanoic acid moiety. In other embodiments, n is 2 and Y^{b} is a β-alanine moiety. In yet other embodiments, R^{e} is hydrogen.

In other embodiments, the carrier peptide comprises the sequence ILFQY (SEQ ID NO: 576). The peptides may comprise the ILFQY (SEQ ID NO: 576) sequence in addition to any of the other sequences disclosed herein. For example the carrier peptide may comprise ILFQY (SEQ ID NO: 576) and [(R^{d}Y^{b}R^{d})ₓ(R^{d}R^{d}Y^{b})_{y}]_{z}, [(R^{d}R^{d}Y^{b})_{y}(R^{d}Y^{b}R^{d})ₓ]_{z}, (R^{d}Y^{b})ₚ or combinations thereof wherein R^{d}, x, y and Y^{b} are as defined above. The [(R^{d}Y^{b}R^{d})ₓ(R^{d}R^{d}Y^{b})_{y}]_{z}, [(R^{d}R^{d}Y^{b})_{y}(R^{d}Y^{b}R^{d})ₓ]_{z} or (R^{d}Y^{b})ₚ sequence may be on the amino terminus, carboxy terminus or both of the ILFQY (SEQ ID NO: 576) sequence. In certain embodiments, x is 1 and y is 0 and the carrier peptide comprises (R^{d}Y^{b}R^{d})_{z} linked to the ILFQY (SEQ ID NO: 576) sequence via an optional Z linker.

In other related embodiments, the carrier peptide comprises the sequence ILFQ (SEQ ID NO: 577), IWFQ (SEQ ID NO: 578) or ILIQ (SEQ ID NO: 579). Other embodiments include carrier peptides which comprise the sequence PPMWS (SEQ ID NO: 580), PPMWT (SEQ ID NO: 581), PPMFS (SEQ ID NO: 582) or PPMYS (SEQ ID NO: 583). The carrier peptide may comprise these sequences in addition to any of the other sequences described herein, for example in addition to the sequences [(R^{d}Y^{b}R^{d})ₓ(R^{d}R^{d}Y^{b})_{y}]_{z}, [(R^{d}R^{d}Y^{b})_{y}(R^{d}Y^{b}R^{d})ₓ]_{z} or (R^{d}Y^{b})ₚ wherein R_{d}, x, y and Y^{b} are as defined above.

Some embodiments of the carrier peptide include modifications to naturally occurring amino acid subunits, for example the amino terminal or carboxy terminal amino acid subunit may be modified. Such modifications include capping the free amino or free carboxy with a hydrophobic group. For example, the amino terminus may be capped with an acetyl, benzoyl or stearoyl moiety. For example, any of the peptide sequences in Table 1 may have such modifications even if not specifically depicted in the table. In these embodiments, the amino terminus of the carrier peptide can be depicted as follows:

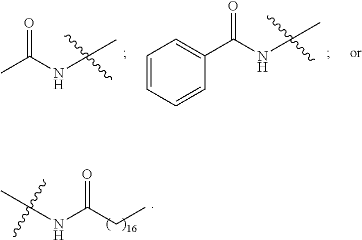

In yet other embodiments, the carrier peptide comprises at least one of alanine, asparagine, cysteine, glutamine, glycine, histidine, lysine, methionine, serine or threonine.

In some of the embodiments disclosed herein, the carrier peptide consists of the noted sequences and the carboxy terminal glycine or proline amino acid subunit.

In some embodiments the carrier peptide does not consist of the following sequences (amino terminal to carboxy terminal): R₆G, R₇G, R₈G, R₅GR₄G, R₅F₂R₄G, Tat-G, rTat-G, (RXR₂G₂)₂ or (RXR₃X)₂G. In yet other embodiments, the carrier peptide does not consist of R₈G, R₉G or R₉F₂G. In still other embodiments, the carrier peptide does not consist of the following sequences: Tat-G, rTat-G, R₉F₂G, R₅F₂R₄, R₄G, R₅G, R₆G, R₇G, R₈G, R₉G, (RXR)₄G, (RXR)₅G, (RXRRBR)₂G, (RAR)₄F₂ or (RGR)₄F₂. In other embodiments, the carrier peptide does not consist of “Penetratin” or “R₆Pen”.

In another aspect, the present disclosure provides a peptide-nucleic acid analog conjugate, comprising
a nucleic acid analog having a substantially uncharged backbone and a targeting base sequence, and
covalently linked to the nucleic acid analog, a peptide comprising a carboxy terminal glycine or proiline amino acid subunit and consisting of 8 to 16 additional other subunits selected from R^{d} subunits, Y subunits, and optional Z subunits, including at least eight R^{d} subunits, at least two Y subunits, and at most three Z subunits, where >50% of said subunits are R^{d} subunits, and where
(a) each R^{d} subunit independently represents arginine or an arginine analog, the arginine analog being a cationic α-amino acid comprising a side chain of the structure R^{a}N═C(NH₂)R^{b}, where Rₐ is H or R^{c}; R^{b} is R^{c}, NH₂, NHR, or N(R^{c})₂, where R^{c} is lower alkyl or lower alkenyl and optionally comprises oxygen or nitrogen or Rₐ and R^{b} may together form a ring; and wherein the side chain is linked to the amino acid via Rₐ or R^{b};
(b) the at least two Y subunits are Y^{a} or Y^{b}, wherein:
(i) each Y^{a} is independently a neutral α-amino acid subunits having side chains independently selected from substituted or unsubstituted alkyl, alkenyl, alkynyl, aryl, and aralkyl, wherein said side chain, when selected from substituted alkyl, alkenyl, and alkynyl, includes at most one heteroatom for every two, preferably every four, and more preferably every six carbon atoms, and wherein said subunits are contiguous or are flanking a linker moiety, and
(ii) Y^{b} is:

—C(O)—(CHR^{e})ₙ—NH— (Y^{b})
wherein n is 2 to 7 and each R^{e} is independently, at each occurrence, hydrogen or methyl; and
(c) Z represents an amino acid subunit selected from alanine, asparagine, cysteine, glutamine, glycine, histidine, lysine, methionine, serine, threonine and amino acids having side chains which are one- or two-carbon homologs of naturally occurring side chains, excluding side chains which are negatively charged at physiological pH (e.g. carboxylate side chains). In some embodiments, the side chains are neutral. In other embodiments, the Z side chains are side chains of naturally occurring amino acids. The optional Z subunits in some embodiments are selected from alanine, glycine, methionine, serine, and threonine. The carrier peptide may include zero, one, two, or three Z subunits, and in some embodiments includes at most two Z subunits.

In selected embodiments, the carrier peptide has exactly two Y subunits of type Y^{a}, which are contiguous or are flanking a cysteine subunit. In some embodiments, the two Y^{a} subunits are contiguous. In other embodiments, side chains for Y^{a} subunits include side chains of naturally occurring amino acids and one- or two-carbon homologs thereof, excluding side chains which are charged at physiological pH. Other possible side chains are side chains of naturally occurring amino acids. In further embodiments, the side chain is an aryl or aralkyl side chain; for example, each Y^{a} may be independently selected from phenylalanine, tyrosine, tryptophan, leucine, isoleucine, and valine.

In selected embodiments, each Y^{a} is independently selected from phenylalanine and tyrosine; in further embodiments, each Y^{a} is phenylalanine. This includes, for example, conjugates which consist of arginine subunits, phenylalanine subunits, the glycine or proline amino acid subunit, an optional linker moiety, and the nucleic acid analog. One such conjugate includes a peptide having the formula Arg₉Phe₂aa, where aa is glycine or proline.

The foregoing carrier peptides may also comprise ILFQY (SEQ ID NO: 576), TLFQ (SEQ ID NO: 577), IWFQ (SEQ ID NO: 578) or ILIQ (SEQ ID NO: 579). Other embodiments include the foregoing carrier peptides which comprise the sequence PPMWS (SEQ ID NO: 580), PPMWT (SEQ ID NO: 581), PPMFS (SEQ ID NO: 582) or PPMYS (SEQ ID NO: 583).

The peptide-oligomer conjugates of the invention are more effective than the unconjugated oligomer in various functions, including: inhibiting expression of targeted mRNA in a protein expression system, including cell free translation systems; inhibiting splicing of targeted pre-mRNA; and inhibiting replication of a virus, by targeting cis-acting elements which control nucleic acid replication or mRNA transcription of the virus.

Also included within the scope of the present invention are conjugates of other pharmacological agents (i.e., not a nucleic acid analog) and the carrier peptide. Specifically, some embodiments provide a conjugate comprising:
(a) a carrier peptide comprising amino acid subunits; and
(b) a pharmacological agent;
   wherein:
two or more of the amino acid subunits are positively charged amino acids, the carrier peptide comprises a glycine (G) or proline (P) amino acid subunit at a carboxy terminus of the carrier peptide and the carrier peptide is covalently attached to the pharmacological agent. The carrier peptide in these embodiments may be any of the carrier peptides described herein. Methods for delivering the pharmacological agent by conjugating it to the carrier peptide are also provided.

The pharmacological agent to be delivered is may be a biologically active agent, e.g. a therapeutic or diagnostic agent, although it may be a compound employed for detection, such as a fluorescent compound. Biologically active agents include drug substances selected from biomolecules, e.g. peptides, proteins, saccharides, or nucleic acids, particularly antisense oligonucleotides, or “small molecule” organic or inorganic compounds. A “small molecule” compound may be defined broadly as an organic, inorganic, or organometallic compound which is not a biomolecule as described above. Typically, such compounds have molecular weights of less than 1000, or, in one embodiment, less than 500.

In one embodiment, the pharmacological agent to be delivered does not include single amino acids, dipeptides, or tripeptides. In another embodiment, it does not include short oligopeptides; that is, oligopeptides having fewer than six amino acid subunits. In a further embodiment, it does not include longer oligopeptides; that is, oligopeptides having between seven and 20 amino acid subunits. In a still further embodiment, it does not include polypeptides, having greater than 20 amino acid subunits, or proteins.

The carrier peptide is effective to enhance the transport of the pharmacological agent into a cell relative to the pharmacological agent in unconjugated form and/or with less toxicity, relative to the pharmacological agent conjugated to a corresponding peptide lacking the glycing or proline subunits. In some embodiments, transport is enhanced by a factor of at least two, at least five or at least ten. In other embodiments, toxicity is decreased (i.e., maximum tolerated dose increased) by a factor of at least two, at least five or at least ten.

### B. Peptide Linkers

The carrier peptide can be linked to the agent to be delivered (e.g., nuceleic acid analogue, pharmacological agent, etc.) by a variety of methods available to one of skill in the art. In some embodiments, the carrier peptide is linked to the nucleic acid analogue directly without an intervening linker. In this regard, formation of an amide bond between the terminal amino acid and a free amine of free carboxyl on the nucleic acid analogue may be useful for forming the conjugate. In certain embodiments, the carboxy terminal glycine or proline subunit is linked directly to the 3′ end of the nucleic acid analogue, for example the carrier peptide may be linked by forming an amide bond between the carboxy terminal glycine or proline moiety and the 3′ morpholino ring nitrogen (see e.g., FIG. 1C).

In some embodiments, the nucleic acid analog is conjugated to the carrier peptide via a linker moiety selected from a Y^{a} or Y^{b} subunit, a cysteine subunit, and an uncharged, non-amino acid linker moiety. In other embodiments, the nucleic acid analogue is linked to the carrier peptide directly via the glycine or proline moiety at either the 5′ or 3′ end of the nucleic acid analogue. In some embodiments, the carrier peptide is linked directly via the glycine or proline amino acid subunit to the 3′ of the nucleic acid analogue, for example directly linked to the 3′ morpholino nitrogen via an amide bond.

In other embodiments, the conjugates comprise a linking moiety between the terminal glycine or proline amino acid subunit. In some of the embodiments, the linker is up to 18 atoms in length comprising bonds selected from alkyl, hydroxyl, alkoxy, alkylamino, amide, ester, carbonyl, carbamate, phosphorodiamidate, phosphoroamidate, phosphorothioate and phosphodiester. In certain embodiments, the linker comprises phosphorodiamidate and piperazine bonds. For example, in some embodiments the linker has the following structure (XXIX):

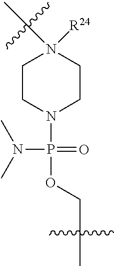

wherein R²⁴ is absent, H or C₁-C₆ alkyl. In certain embodiments, R²⁴ is absent and in other embodiments structure (XXIX) links the 5′ end of a nucleic acid analogue (e.g., a morpholino oligomer) to the carrier peptide (see e.g., FIG. 1).

In some embodiments, the side chain moieties of the R^{d} subunits are independently selected from guanidyl (HN═C(NH₂)NH—), amidinyl (HN═C(NH₂)C<), 2-aminodihydropyrimidyl, 2-aminotetrahydropyrimidyl, 2-aminopyridinyl and 2-amino pyrimidinyl.

Multiple carrier peptides can be attached to a single compound if desired; alternatively, multiple compounds can be conjugated to a single transporter. The linker between the carrier peptide and the nucleic acid analogue may also consist of natural or non-natural amino acids (e.g., 6-aminohexanoic acid or β-alanine). The linker may also comprise a direct bond between the carboxy terminus of a transporter peptide and an amine or hydroxy group of the nucleic acid analogue (e.g., at the 3′ morpholino nitrogen or 5′ OH), formed by condensation promoted by e.g. carbodiimide.

In general, the linker may comprise any nonreactive moiety which does not interfere with transport or function of the conjugate. Linkers can be selected from those which are non-cleavable under normal conditions of use, e.g., containing an ether, thioether, amide, or carbamate bond. In other embodiments, it may be desirable to include a linkage between the carrier peptide and compound (e.g., oligonucleotide analogue, pharmacological agent, etc.) which is cleavable in vivo. Bonds which are cleavable in vivo are known in the art and include, for example, carboxylic acid esters, which are hydrolyzed enzymatically, and disulfides, which are cleaved in the presence of glutathione. It may also be feasible to cleave a photolytically cleavable linkage, such as an ortho-nitrophenyl ether, in vivo by application of radiation of the appropriate wavelength. Exemplary heterobifunctional linking agents which further contain a cleavable disulfide group include N-hydroxysuccinimidyl 3-[(4-azidophenyl)dithio]propionate and others described in Vanin, E. F. and Ji, T. H., Biochemistry 20:6754-6760 (1981).’

### C. Exemplary Carrier Peptides

A Table of sequences of exemplary carrier peptides and oligonucleotide sequences is provided below in Table 1. In some embodiments, the present disclosure provides a peptide oligomer conjugate, wherein the peptide comprises or consists of any one of the peptide sequences in Table 1. In another embodiment, the the nucleic acid analogue comprises or consists of any of the oligonucleotide sequences in Table 1. In still other embodiments, the present disclosure provides a peptide oligomer conjugate, wherein the peptide comprises or consists of any one of the peptide sequences in Table 1, and the nucleic acid analogue comprises or consists of any of the oligonucleotide sequences in Table 1. In other embodiments, the disclosure provides a peptide comprising or consisting of any one of the sequences in Table 1.

TABLE 1
Exemplary Carrier Peptides and
Oligonucleotide Sequences

| | | SEQ |
|---|---|---|
| | Sequence (Amino to | ID |
| Name | Carboxy Terminus or 5′ to 3′) | NO. |
| (RFF)₃; | RFFRFFRFF-aa | 89 |
| CP0407 | | |
| RTR | RTRTRFLRRT-aa | 90 |
| RFFR | RFFRFFRFFR-aa | 91 |
| KTR | KTRTKFLKKT-aa | 92 |
| KFF | KFFKFFKFF-aa | 93 |
| KFFK | KFFKFFKFFK-aa | 94 |
| (RFF)₂ | RFFRFF-aa | 95 |
| (RFF)₂R | RFFRFFR-aa | 96 |
| RX | RXXRXXR-aa | 97 |
| (RXR)₄; | RXRRXRRXRRXR-aa | 98 |
| P007 | | |
| Tat₄₇₋₅₈ | YGRKKRRQRRR-aa | 99 |
| Tat₄₈₋₅₈ | GRKKRRQRRR-aa | 100 |
| Tat₄₉₋₅₈ | RKKRRQRRR-aa | 101 |
| Penetratin | RQIKIWFQNRRMKWKKGG-aa | 102 |
| Transportan | GWTLNSAGYLLGKINLKALAALAKKIL-aa | 103 |
| 2XHph-1 | YARVRRRGPRGYARVRRRGPRR-aa | 104 |
| Hph-1 | YARVRRRGPRR-aa | 105 |
| Sim-2 | AKAARQAAR-aa | 106 |
| HSV1 VP22 | DAATATRGRSAASRPTERPRAPARSASRPRRPV | 107 |
| | E-aa | |
| Pep-1 | KETWWETWWTEWSQPKKKRKV-aa | 108 |
| Pep-2 | KETWFETWFTEWSQPKKKRKV-aa | 109 |
| ANTP | RQIKIWFQNRRMKWKK-aa | 110 |
| R₆Pen | RRRRRR-RQIKIWFQNRRMKWKKGG-aa | 111 |
| rTat | RRRQRRKKRC-aa | 112 |
| pTat | CYGRKKRRQRRR-aa | 113 |
| R₉F₂ | RRRRRRRRRFFC-aa | 114 |
| R₉CF₂ | RRRRRRRRRCFF-aa | 115 |
| RRRRRRRR | | |
| RCFF | | |
| R₈CF₂R | RRRRRRRRCFFR-aa | 116 |
| R₆CF₂R₃ | RRRRRRCFFRRR-aa | 117 |
| R₅FCFR₄ | RRRRRFCFRRRR-aa | 118 |
| R₅F₂R₄ | RRRRRFFRRRR-aa | 119 |
| R₄CF₂R₅ | RRRRCFFRRRRR-aa | 120 |
| R₂CF₂R₇ | RRCFFRRRRRRR-aa | 121 |
| CF₂R₉ | CFFRRRRRRRRR-aa | 122 |
| CR₉F₂ | CRRRRRRRRRFF-aa | 123 |
| F₂R₉ | FFRRRRRRRRR-aa | 124 |
| R₅F₂CF₂R₄ | RRRRRFFCFFRRRR-aa | 125 |
| R₉I₂ | RRRRRRRRRII-aa | 126 |
| R₈F₃ | RRRRRRRRFFF-aa | 127 |
| R₉F₄ | RRRRRRRRRFFFF-aa | 128 |
| R₈F₂ | RRRRRRRRFF-aa | 129 |
| R₆F₂ | RRRRRRFF-aa | 130 |
| R₅F₂ | RRRRRFF-aa | 131 |
| (RRX)₃RR | RRXRRXRRXRR-aa | 132 |
| (RXR)₄ | RXRRXRRXRRXR-aa | 133 |
| (XRR)₄ | XRRXRRXRRXRR-aa | 134 |
| (RX)₅RR | RXRXRXRXRXR-aa | 135 |
| (RXR)₃ | RXRRXRRXR-aa | 136 |
| (RXR)₂R | RXRRXRR-aa | 137 |
| (RXR)₂ | RXRRXR-aa | 138 |
| (RKX)₃RK | RKXRKXRKXRK-aa | 139 |
| (RHX)₃RH | RHXRHXRHXRH-aa | 140 |
| R₈CF₂R | RRRRRRRRCFFR-aa | 141 |
| (RRX)₃RR | RRXRRXRRXRR-aa | 142 |
| (RXR)₄; | RXRRXRRXRRXR-aa | 143 |
| P007 | | |
| (XRR)₄ | XRRXRRXRRXRR-aa | 144 |
| (RX)₅R | RXRXRXRXRXR-aa | 145 |
| (RX)₇R | RXRXRXRXRXRXR-aa | 146 |
| (RXR)₅ | RXRRXRRXRRXRRXR-aa | 147 |
| (RXRRBR)₂; | RXRRBRRXRRBR-aa | 148 |
| B | | |
| (RXR)₃RBR | RXRRXRRXRRBR-aa | 149 |
| (RB)₅RXRB | RBRBRBRBRBRXRBR-aa | 150 |
| R | | |
| RBRBRBRX | RBRBRBRXRBRBRBR-aa | 151 |
| RBRBRBR | | |
| X(RB)₃RX | XRBRBRBRXRBRBRBR-aa | 152 |
| (RB)₃R-X | | |
| (RBRX)₄ | RBRXRBRXRBRXRBR-aa | 153 |
| (RB)₄(RX)₃R | RBRBRBRBRXRXRXR-aa | 154 |
| RX(RB)₂RX | RXRBRBRXRBRBRBR-aa | 155 |
| (RB)₃R | | |
| (RB)₇R | RBRBRBRBRBRBRBR-aa | 156 |
| R₄ | tg-RRRR-aa | 157 |
| R₅ | tg-RRRRR-aa | 158 |
| R₆ | tg-RRRRRR-aa | 159 |
| R₇ | tg-RRRRRRR-aa | 160 |
| R₈ | tg-RRRRRRRR-aa | 161 |
| R₅GR₄ | tg-RRRRRGRRRR-aa | 162 |
| R₅F₂R₄ | tg-RRRRRFFRRRR-aa | 163 |
| Tat | tg-RKKRRQRRR-aa | 164 |
| rTat | tg-RRRQRRKKR-aa | 165 |
| | RXRRXR-aa | 166 |
| | RBRRBR-aa | 167 |
| | RXRRBR-aa | 168 |
| | RBRRXR-aa | 169 |
| | RXRY^{b}RXR-aa | 170 |
| | RBRY^{b}RBR-aa | 171 |
| | RXRY^{b}RBR-aa | 172 |
| | RBRY^{b}RXR-aa | 173 |
| | RXRILFQYRXR-aa | 174 |
| | RBRILFQYRBR-aa | 175 |
| | RXRILFQYRBR-aa | 176 |
| | RBRILFQYRXR-aa | 177 |
| | RXRRXRRXR-aa | 178 |
| | RBRRBRRBR-aa | 179 |
| | RXRRBRRXR-aa | 180 |
| | RXRRBRRBR-aa | 181 |
| | RXRRXRRBR-aa | 182 |
| | RBRRXRRBR-aa | 183 |
| | RBRRXRRXR-aa | 184 |
| | RBRRBRRXR-aa | 185 |
| | RXRY^{b}RXRRXR-aa | 186 |
| | RXRRXRY^{b}RXR-aa | 187 |
| | RXRILFQYRXRRXR-aa | 188 |
| | RXRRXRILFQYRXR-aa | 189 |
| | RXRY^{b}RXRY^{b}RXR-aa | 190 |
| | RXRILFQYRXRILFQYRXR-aa | 191 |
| | RXRILFQYRXRY^{b}RXR-aa | 192 |
| | RXRY^{b}RXRILFQYRXR-aa | 193 |
| | RBRY^{b}RBRRBR-aa | 194 |
| | RBRRBRY^{b}RBR-aa | 195 |
| | RBRILFQYRBRRBR-aa | 196 |
| | RBRRBRILFQYRBR-aa | 197 |
| | RBRYRBRY^{b}RBR-aa | 198 |
| | RBRILFQYRBRILFQYRBR-aa | 199 |
| | RBRY^{b}RBRILFQYRBR-aa | 200 |
| | RBRILFQYRBRY^{b}RBR-aa | 201 |
| | RXRY^{b}RBRRXR-aa | 202 |
| | RXRRBRY^{b}RXR-aa | 203 |
| | RXRILFQYRBRRXR-aa | 204 |
| | RXRRBRILFQYRXR-aa | 205 |
| | RXRY^{b}RBRY^{b}RXR-aa | 206 |
| | RXRILFQYRBRILFQYRXR-aa | 207 |
| | RXRY^{b}RBRILFQYRXR-aa | 208 |
| | RXRILFQYRBRY^{b}RXR-aa | 209 |
| | RXRY^{b}RBRRBR-aa | 210 |
| | RXRRBRY^{b}RBR-aa | 211 |
| | RXRILFQYRBRRBR-aa | 212 |
| | RXRRBRILFQYRBR-aa | 213 |
| | RXRY^{b}RBRY^{b}RBR-aa | 214 |
| | RXRILFQYRBRILFQYRBR-aa | 215 |
| | RXRY^{b}RBRILFQYRBR-aa | 216 |
| | RXRILFQYRBRY^{b}RBR-aa | 217 |
| | RXRY^{b}RXRRBR-aa | 218 |
| | RXRRXRY^{b}RBR-aa | 219 |
| | RXRILFQYRXRRBR-aa | 220 |
| | RXRRXRILFQYRBR-aa | 221 |
| | RXRY^{b}RXRY^{b}RBR-aa | 222 |
| | RXRILFQYRXRILFQYRBR-aa | 223 |
| | RXRY^{b}RXRILFQYRBR-aa | 224 |
| | RXRILFQYRXRY^{b}RBR-aa | 225 |
| | RBRY^{b}RXRRBR-aa | 226 |
| | RBRRXRY^{b}RBR-aa | 227 |
| | RBRILFQYRXRRBR-aa | 228 |
| | RBRRXRILFQYRBR-aa | 229 |
| | RBRY^{b}RXRY^{b}RBR-aa | 230 |
| | RBRILFQYRXRILFQYRBR-aa | 231 |
| | RBRY^{b}RXRILFQYRBR-aa | 232 |
| | RBRILFQYRXRY^{b}RBR-aa | 233 |
| | RBRY^{b}RXRRXR-aa | 234 |
| | RBRRXRY^{b}RXR-aa | 235 |
| | RBRILFQYRXRRXR-aa | 236 |
| | RBRRXRILFQYRXR-aa | 237 |
| | RBRY^{b}RXRY^{b}RXR-aa | 238 |
| | RBRILFQYRXRILFQYRXR-aa | 239 |
| | RBRY^{b}RXRILFQYRXR-aa | 240 |
| | RBRILFQYRXRY^{b}RXR-aa | 241 |
| | RBRY^{b}RBRRXR-aa | 242 |
| | RBRRBRY^{b}RXR-aa | 243 |
| | RBRILFQYRBRRXR-aa | 244 |
| | RBRRBRILFQYRXR-aa | 245 |
| | RBRY^{b}RBRY^{b}RXR-aa | 246 |
| | RBRILFQYRBRILFQYRXR-aa | 247 |
| | RBRY^{b}RBRILFQYRXR-aa | 248 |
| | RBRILFQYRBRY^{b}RXR-aa | 249 |
| | RXRRXRRXRRXR-aa | 250 |
| | RXRRBRRXRILFQYRXRBRXR-aa | 251 |
| | RXRRBRRXRRBR-aa | 252 |
| | YGRKKRRQRRRP-aa | 253 |
| | RXRRXRRXRRXRXBASSLNIAXC-aa | 254 |
| | RXRRBRRXRILFQYRXRBRXRBASSLNIAXC- | 255 |
| | aa | |
| | RXRRBRRXRASSLNIARXRBRXRBC-aa | 256 |
| | RXRRBRRXRRBRXBAS SLNIA-aa | 257 |
| | THRPPMWSPVWP-aa | 258 |
| | HRPPMWSPVWP-aa | 259 |
| | THRPPMWSPV-aa | 260 |
| | THRPPMWSP-aa | 261 |
| | THRPPMWSPVFP-aa | 262 |
| | THRPPMWSPVYP-aa | 263 |
| | THRPPMWSPAWP-aa | 264 |
| | THRPPMWSPLWP-aa | 265 |
| | THRPPMWSPIWP-aa | 266 |
| | THRPPMWTPVVWP-aa | 267 |
| | THRPPMFSPVWP-aa | 268 |
| | THRPPMWS-aa | 269 |
| | HRPPMWSPVW-aa | 270 |
| | THRPPMYSPVWP-aa | 271 |
| | THRPPnleWSPVWP-aa | 272 |
| | (nle = norleucine) | |
| | THKPPMWSPVWP-aa | 273 |
| | SHRPPMWSPVWP-aa | 274 |
| | STFTHPR-aa | 275 |
| | YDIDNRR-aa | 276 |
| | AYKPVGR-aa | 277 |
| | HAIYPRH-aa | 278 |
| | HTPNSTH-aa | 279 |
| | ASSPVHR-aa | 280 |
| | SSLPLRK-aa | 281 |
| | KKRS-aa | 282 |
| | KRSK-aa | 283 |
| | KKRSK-aa | 284 |
| | KSRK-aa | 285 |
| | SRKR-aa | 286 |
| | RKRK-aa | 287 |
| | KSRKR-aa | 288 |
| | QHPPWRV-aa | 289 |
| | THPPTTH-aa | 290 |
| | YKHTPTT-aa | 291 |
| | QGMHRGT-aa | 292 |
| | SRKRK-aa | 293 |
| | KSRKRK-aa | 294 |
| | PKKKRKV-aa | 295 |
| | GKKRSKV-aa | 296 |
| | KSRKRKL-aa | 297 |
| | HSPSKIP-aa | 298 |
| | HMATFHY-aa | 299 |
| | AQPNKFK-aa | 300 |
| | NLTRLHT-aa | 301 |
| | KKKR-aa | 302 |
| | KKRK-aa | 303 |
| | KKKRK-aa | 304 |
| | RRRRRRQIKIWFQNRRMKWKKGGC-aa | 305 |
| | RRRRRRRQIKIWFQNRRMKWKKGGC-aa | 306 |
| | RQIKIWFQNRRMKWKKGGC-aa | 307 |
| | RRRRRRRQIKIWFQNRRMKWKKC-aa | 308 |
| | RXRRXRRXRRQIKIWFQNRRMKWKKGGC-aa | 309 |
| | RRRRRRRQIKILFQNRXRXRXRXC-aa | 310 |
| | RXRRXRRXRRXRC-aa | 311 |
| | RXRRXRRXRRXRXC-aa | 312 |
| | RXRRXRRXRIKILFQNRRMKWKKGGC-aa | 313 |
| | RXRRXRRXRIKILFQNRRMKWKKC-aa | 314 |
| | RXRRXRRXRIKILFQNRMKWKKC-aa | 315 |
| | RXRRXRRXRIKILFQNXRMKWKKC-aa | 316 |
| | RXRRXRRXRIKILFQNHRMKWKKC-aa | 317 |
| | RXRRXRRXRIKILFQNXRMKWKKC-aa | 318 |
| | RXRRXRRXRIKILFQNXRMKWKKC-aa | 319 |
| | RXRRXRRXRIKILFQNXRMKWKAC-aa | 320 |
| | RXRRXRRXRIKILFQNXRMKWHKAC-aa | 321 |
| | RXRRXRRXRIKILFQNXRMKWHRC-aa | 322 |
| | RXRXRXRXRIKILFQNRRMKWKKC-aa | 323 |
| | RARARARARIKILFQNRRMKWKKC-aa | 324 |
| | RXRRXRRXRIXILFQNXRMKWHKAC-aa | 325 |
| | RXRRXRRXRIHILFQNXRMKWHKAC-aa | 326 |
| | RXRRXRRXRIRILFQNXRMKWHKAC-aa | 327 |
| | RXRRXRRXRIXILFQYXRMKWHKAC-aa | 328 |
| | RXRRXRRXRLYSPLSFQXRMKWHKAC-aa | 329 |
| | RXRRXRRXRISILFQYXRMKWHKAC-aa | 330 |
| | RXRRXRRXRILFQYXRMKWHKAC-aa | 331 |
| | RXRRXRIXILFQYXRMKWHKAC-aa | 332 |
| | RXRRARRXRIHILFQYXRMKWHKAC-aa | 333 |
| | RARRXRRARIHILFQYXRMKWHKAC-aa | 334 |
| | RXRRXRRXRIHILFQYXRMKWHKAC-aa | 335 |
| | RXRRXRRXRIXILFQNXRMKWHKAC-aa | 336 |
| | RXRRXRRXRIHILFQNXRMKWHKAC-aa | 337 |
| | RXRRXRRXRIKILFQNRRMKWHK-aa | 338 |
| | RXRRXRRXRIKILFQNXRMKWHK-aa | 339 |
| | RXRRXRRXRIXILFQNRRMKWHK-aa | 340 |
| | RXRRXRRXRIXILFQNXRMKWHK-aa | 341 |
| | RXRRXRRXRIHILFQNRRMKWHK-aa | 342 |
| | RXRRXRRXRIHILFQNXRMKWHK-aa | 343 |
| | RXRRXRRXRIRILFQNRRMKWHK-aa | 344 |
| | RXRRXRRXRIRILFQNXRMKWHK-aa | 345 |
| | RXRRXRRXRIILFQNRRMKWHK-aa | 346 |
| | RXRRXRRXRIILFQNXRMKWHK-aa | 347 |
| | RXRRXRRXRKILFQNRRMKWHK-aa | 348 |
| | RXRRXRRXRKILFQNXRMKWHK-aa | 349 |
| | RXRRXRRXRXILFQNRRMKWHK-aa | 350 |
| | RXRRXRRXRXILFQNXRMKWHK-aa | 351 |
| | RXRRXRRXRHILFQNRRMKWHK-aa | 352 |
| | RXRRXRRXRHILFQNXRMKWHK-aa | 353 |
| | RXRRXRRXRRILFQNRRMKWHK-aa | 354 |
| | RXRRXRRXRRILFQNXRMKWHK-aa | 355 |
| | RXRRXRRXRILFQNRRMKWHK-aa | 356 |
| | RXRRXRRXRILFQNXRMKWHK-aa | 357 |
| | RXRRXRRXRIKILFQYRRMKWHK-aa | 358 |
| | RXRRXRRXRIKILFQYXRMKWHK-aa | 359 |
| | RXRRXRRXRIXILFQYRRMKWHK-aa | 360 |
| | RXRRXRRXRIXILFQYXRMKWHK-aa | 361 |
| | RXRRXRRXRIHILFQYRRMKWHK-aa | 362 |
| | RXRRXRRXRIHILFQYXRMKWHK-aa | 363 |
| | RXRRXRRXRIRILFQYRRMKWHK-aa | 364 |
| | RXRRXRRXRIRILFQYXRMKWHK-aa | 365 |
| | RXRRXRRXRIILFQYRRMKWHK-aa | 366 |
| | RXRRXRRXRIILFQYXRMKWHK-aa | 367 |
| | RXRRXRRXRKILFQYRRMKWHK-aa | 368 |
| | RXRRXRRXRKILFQYXRMKWHK-aa | 369 |
| | RXRRXRRXRXILFQYRRMKWHK-aa | 370 |
| | RXRRXRRXRXILFQYXRMKWHK-aa | 371 |
| | RXRRXRRXRHILFQYRRMKWHK-aa | 372 |
| | RXRRXRRXRHILFQYXRMKWHK-aa | 373 |
| | RXRRXRRXRRILFQYRRMKWHK-aa | 374 |
| | RXRRXRRXRRILFQYXRMKWHK-aa | 375 |
| | RXRRXRRXRILFQYRRMKWHK-aa | 376 |
| | RXRRXRRXRILFQYXRMKWHK-aa | 377 |
| | RXRRXRRXR-aa | 378 |
| | RXRRXRRXRRXR-aa | 379 |
| | RARRAR-aa | 380 |
| | RARRARRAR-aa | 381 |
| | RARRARRARRAR-aa | 382 |
| | RXRRXRI-aa | 383 |
| | RXRRARRXR-aa | 384 |
| | RARRXRRAR-aa | 385 |
| | RRRRR-aa | 386 |
| | RRRRRR-aa | 387 |
| | RRRRRRR-aa | 388 |
| | RXRRXRRXRRXRC-aa | 389 |
| | RXRRXRRXRRXRXC-aa | 390 |
| | RXRRXRRXRIKILFQNRRMKWKKGGC-aa | 391 |
| | RXRRXRRXRIKILFQNRRMKWKKC-aa | 392 |
| | RXRRXRRXRIKILFQNRMKWKKC-aa | 393 |
| | RXRRXRRXRIKILFQNXRMKWKKC-aa | 394 |
| | RXRRXRRXRIKILFQNHRMKWKKC-aa | 395 |
| | RXRRXRRXRIKILFQNXRMKWKKC-aa | 396 |
| | RXRRXRRXRIKILFQNXRMKWKKC-aa | 397 |
| | RXRRXRRXRIKILFQNXRMKWKAC-aa | 398 |
| | RXRRXRRXRIKILFQNXRMKWHKAC-aa | 399 |
| | RXRRXRRXRIKILFQNXRMKWHRC-aa | 400 |
| | RXRXRXRXRIKILFQNRRMKWKKC-aa | 401 |
| | RARARARARIKILFQNRRMKWKKC-aa | 402 |
| | RXRRXRRXRIXILFQNXRMKWHKAC-aa | 403 |
| | RXRRXRRXRIHILFQNXRMKWHKAC-aa | 404 |
| | RXRRXRRXRIRILFQNXRMKWHKAC-aa | 405 |
| | RXRRXRRXRIXILFQYXRMKWHKAC-aa | 406 |
| | RXRRXRRXRLYSPLSFQXRMKWHKAC-aa | 407 |
| | RRMKWHK-aa | 408 |
| | XRMKWHK-aa | 409 |
| | XXXXXXXXXXXXXXILFQXXRMKWHK-aa | 410 |
| | XXXXXXXXXXXXXXILFQXXRMKWHK-aa | 411 |
| | RRRRRRRQIKILFQNPKKKRKVGGC-aa | 412 |
| | HHFFRRRRRRRRRFFC-aa | 413 |
| | HHHHHHRRRRRRRRRFFC-aa | 414 |
| | HHHHHEIFFRRRRRRRRRFFC-aa | 415 |
| | HHHHHXXRRRRRRRRRFFC-aa | 416 |
| | HHHHHFIXXFFRRRRRRRRRFFC-aa | 417 |
| | HHHXRRRRRRRRRFFXHHHC-aa | 418 |
| | XRMKWHK-aa | 419 |
| | XRWKWHK-aa | 420 |
| | RXRARXR-aa | 421 |
| | RXRXRXR-aa | 422 |
| | RARXRAR-aa | 423 |
| | RXRAR-aa | 424 |
| | XXXXXXXXXXXXXXILFQXXHMKWHK-aa | 425 |
| | XXXXXXXXXXXXXXILFQXXRWKWHK-aa | 426 |
| | XXXXXXXXXXXXXXILFQXXHWKWHK-aa | 427 |
| | XXXXXXXXXXXXXXILFQXRXRARXR-aa | 428 |
| | XXXXXXXXXXXXXXILFQXRXRXRXR-aa | 429 |
| | XXXXXXXXXXXXXXILFQXRXRRXR-aa | 430 |
| | XXXXXXXXXXXXXXILFQXRARXRAR-aa | 431 |
| | XXXXXXXXXXXXXXILFQXRXRARXR-aa | 432 |
| | XXXXXXXXXXXXXXILFQXRXRAR-aa | 433 |
| | XXXXXXXXXXXXXXILIQXXRMKWHK-aa | 434 |
| | XXXXXXXXXXXXXXILIQXXHMKWHK-aa | 435 |
| | XXXXXXXXXXXXXXILIQXXRKWHK-aa | 436 |
| | XXXXXXXXXXXXXXILIQXXHWKWHK-aa | 437 |
| | XXXXXXXXXXXXXXILIQXRXRARXR-aa | 438 |
| | XXXXXXXXXXXXXXILIQXRXRXRXR-aa | 439 |
| | XXXXXXXXXXXXXXILIQXRXRRXR-aa | 440 |
| | XXXXXXXXXXXXXXILIQXRARXRAR-aa | 441 |
| | XXXXXXXXXXXXXXILIQXRXRARXR-aa | 442 |
| | XXXXXXXXXXXXXXILIQXRXRAR-aa | 443 |
| | XXXXXXXXXXXXXXILFQXXHMKWHK-aa | 444 |
| | XXXXXXXXXXXXXXILFQXXRWKWHK-aa | 445 |
| | XXXXXXXXXXXXXXILFQXXHWKWHK-aa | 446 |
| | XXXXXXXXXXXXXXILFQXRXRARXR-aa | 447 |
| | XXXXXXXXXXXXXXILFQXRXRXRXR-aa | 448 |
| | XXXXXXXXXXXXXXILFQXRXRRXR-aa | 449 |
| | XXXXXXXXXXXXXXILFQXRARXRAR-aa | 450 |
| | XXXXXXXXXXXXXXILFQXRXRARXR-aa | 451 |
| | XXXXXXXXXXXXXXILFQXRXRAR-aa | 452 |
| | XXXXXXXXXXXXXXILIQXXRMKWHK-aa | 453 |
| | XXXXXXXXXXXXXXILIQXXHMKWHK-aa | 454 |
| | XXXXXXXXXXXXXXILIQXXRWKWHK-aa | 455 |
| | XXXXXXXXXXXXXXILIQXXHWKWHK-aa | 456 |
| | XXXXXXXXXXXXXXILIQXRXRARXR-aa | 457 |
| | XXXXXXXXXXXXXXILIQXRXRXRXR-aa | 458 |
| | XXXXXXXXXXXXXXILIQXRXRRXR-aa | 459 |
| | XXXXXXXXXXXXXXILIQXRARXRAR-aa | 460 |
| | XXXXXXXXXXXXXXILIQXRXRARXR-aa | 461 |
| | XXXXXXXXXXXXXXILIQXRXRAR-aa | 462 |
| | RXRRARRXRRARXA-aa | 463 |
| | RXRRARRXRILFQYXHMKWHKAC-aa | 464 |
| | RXRRARRXRILFQYXRMKWHKAC-aa | 465 |
| | RXRRARRXRILFQYXRWKWHKAC-aa | 466 |
| | RXRRXRRXRRXRC-aa | 467 |
| | RXRRXRRXRIXILFQNXRMKWHKAC-aa | 468 |
| | RXRRXRRXRIHILFQNXRMKWHKAC-aa | 469 |
| | RXRRXRRXRIXILFQYXRMKWHKAC-aa | 470 |
| | RXRRXRRXRLYSPLSFQXRMKWHKAC-aa | 471 |
| | RXRRXRRXRILFQYXRMKWHKAC-aa | 472 |
| | RXRRXRIXILFQYXRMKWHKAC-aa | 473 |
| | RARRXRRARILFQYXRMKWHKAC-aa | 474 |
| | RXRRARRXRILFQYXRMKWHKAC-aa | 475 |
| | RARRXRRARILFQYXRMKWHKAC-aa | 476 |
| | RXRRARRXRILFQYXRMKWHKAC-aa | 477 |
| | RXRRARRXRILFQYXHMKWHKAC-aa | 478 |
| | RXRRARRXRILFQYXRMKWHKAC-aa | 479 |
| | RXRRARRXRILFQYXRWKWHKAC-aa | 480 |
| | RXRRARRXRILFQYXHWKWHKAC-aa | 481 |
| | RXRRARRXRILFQYRXRARXRAC-aa | 482 |
| | RXRRARRXRILFQYRXRXRXRAC-aa | 483 |
| | RXRRARRXRILIQYXRMKWHKAC-aa | 484 |
| | RXRRXRILFQYRXRRXRC-aa | 485 |
| | RXRRARRXRILFQYRXRARXRAC-aa | 486 |
| | RXRRARRXRILFQYRXRXRXRAC-aa | 487 |
| | RXRRARRXRILIQYXRMKWHKAC-aa | 488 |
| | RXRRXRILFQYRXRRXRCYS-aa | 489 |
| | RARRXRRARILFQYRARXRARAC-aa | 490 |
| | RARRXRRARILFQYRXRARXRAC-aa | 491 |
| | RARRXRRARILFQYRXRRXRAC-aa | 492 |
| | RARRXRRARILFQYRXRARXAC-aa | 493 |
| | RXRRARRXRILFQYRXRRXRAC-aa | 494 |
| | RXRRARRXRILFQYRXRARXAC-aa | 495 |
| | RXRRARRXRIHILFQNXRMKWHKAC-aa | 496 |
| | RXRRARRXRRARXAC-aa | 497 |
| | RXRRARRXRILFQYXHMKWHK-aa | 498 |
| | RXRRARRXRILFQYXRMKWHK-aa | 499 |
| | RXRRARRXRILFQYXRWKWHK-aa | 500 |
| | RXRRARRXRILFQYXRMKWHK-aa | 501 |
| | RXRRARRXRILFQYRXRARXR-aa | 502 |
| | RXRRARRXRILFQYRXRXRXR-aa | 503 |
| | RXRRARRXRILFQYRXRRXR-aa | 504 |
| | RXRRARRXRILFQYRARXRAR-aa | 505 |
| | RXRRARRXRILFQYRXRAR-aa | 506 |
| | RXRRARRXRILIQYXHMKWHK-aa | 507 |
| | RXRRARRXRILIQYXRMKWHK-aa | 508 |
| | RXRRARRXRILIQYXRWKWHK-aa | 509 |
| | RXRRARRXRILIQYXRMKWHK-aa | 510 |
| | RXRRARRXRILIQYRXRARXR-aa | 511 |
| | RXRRARRXRILIQYRXRXRXR-aa | 512 |
| | RXRRARRXRILIQYRXRRXR-aa | 513 |
| | RXRRARRXRILIQYRARXRAR-aa | 514 |
| | RXRRARRXRILIQYRXRAR-aa | 515 |
| | RARRXRRARILFQYXHMKWHK-aa | 516 |
| | RARRXRRARILFQYXRMKWHK-aa | 517 |
| | RARRXRRARILFQYXRWKWHK-aa | 518 |
| | RARRXRRARILFQYXRMKWHK-aa | 519 |
| | RARRXRRARILFQYRXRARXR-aa | 520 |
| | RARRXRRARILFQYRXRXRXR-aa | 521 |
| | RARRXRRARILFQYRXRRXR-aa | 522 |
| | RARRXRRARILFQYRARXRAR-aa | 523 |
| | RARRXRRARILFQYRXRAR-aa | 524 |
| | RARRXRRARILIQYXHMKWHK-aa | 525 |
| | RARRXRRARILIQYXRMKWHK-aa | 526 |
| | RARRXRRARILIQYXRWKWHK-aa | 527 |
| | RARRXRRARILIQYXRMKWHK-aa | 528 |
| | RARRXRRARILIQYRXRARXR-aa | 529 |
| | RARRXRRARILIQYRXRXRXR-aa | 530 |
| | RARRXRRARILIQYRXRRXR-aa | 531 |
| | RARRXRRARILIQYRARXRAR-aa | 532 |
| | RARRXRRARILIQYRXRAR-aa | 533 |
| | RXRRXRILFQYXHMKWHK-aa | 534 |
| | RXRRXRILFQYXRMKWHK-aa | 535 |
| | RXRRXRILFQYXRWKWHK-aa | 536 |
| | RXRRXRILFQYXRMKWHK-aa | 537 |
| | RXRRXRILFQYRXRARXR-aa | 538 |
| | RXRRXRILFQYRXRXRXR-aa | 539 |
| | RXRRXRILFQYRXRRXR-aa | 540 |
| | RXRRXRILFQYRARXRAR-aa | 541 |
| | RXRRXRILFQYRXRAR-aa | 542 |
| | RXRRXRILIQYXHMKWHK-aa | 543 |
| | RXRRXRILIQYXRMKWHK-aa | 544 |
| | RXRRXRILIQYXRWKWHK-aa | 545 |
| | RXRRXRILIQYXRMKWHK-aa | 546 |
| | RXRRXRILIQYRXRARXR-aa | 547 |
| | RXRRXRILIQYRXRXRXR-aa | 548 |
| | RXRRXRILIQYRXRRXR-aa | 549 |
| | RXRRXRILIQYRARXRAR-aa | 550 |
| | RXRRXRILIQYRXRAR-aa | 551 |
| | PRPXXXXXXXXXXXPRG-aa | 552 |
| | RRRRRRRR-aa | 553 |
| | RRMKWKK-aa | 554 |
| | PKKKRKV-aa | 555 |
| | CKDEPQRRSARLSAKPAPPKPEPKPKKAPAKK- | 556 |
| | aa | |
| | RKKRRQRRR-aa | 557 |
| | RKKRRQRR-aa | 558 |
| | RKKRRQR-aa | 559 |
| | KKRRQRRR-aa | 560 |
| | KKRRQRRR-aa | 561 |
| | AKKRRQRRR-aa | 562 |
| | RAKRRQRRR-aa | 563 |
| | RKARRQRRR-aa | 564 |
| | RKKARQRRR-aa | 565 |
| | CRWRWKCCKK-aa | 566 |
| Dengue | CGGTCCACGTAGACTAACAACT | 1 |
| JEV | GAAGTTCACACAGATAAACTTCT | 2 |
| M1/M2AUG. | CGGTTAGAAGACTCATCTTT | 3 |
| 20.22 | | |
| M1/M2AUG. | TTTCGACATCGGTTAGAAGACTCAT | 4 |
| 25.26 | | |
| NP-AUG | GAGACGCCATGATGTGGATGTC | 5 |
| Picornavirus | GAAACACGGACACCCAAAGTAGT | 6 |
| Dengue 3′-CS | TCCCAGCGTCAATATGCTGTTT | 7 |
| Arenaviruses | GCCTAGGATCCACGGTGCGC | 8 |
| RSV-L target | GGGACAAAATGGATCCCATTATTAATGGAAATT | 9 |
| | CTGCTAA | |
| RSV-AUG-2 | TAATGGGATCCATTTTGTCCC | 10 |
| RSV-AUG3 | AATAATGGGATCCATTTTGTCCC | 11 |
| RSV-AUG4 | CATTAATAATGGGATCCATTTTGTCCC | 12 |
| RSV-AUG5 | GAATTTCCATTAATAATGGGATCCATTTTG | 13 |
| RSV-AUG6 | CAGAATTTCCATTAATAATGGGATCCATT | 14 |
| M23D | GGCCAAACCTCGGCTTACCTGAAAT | 15 |
| AVI-5225 | GGCCAAACCTCGGCTTACCTGAAAT- | 16 |
| | RXRRBRRXRRBRXB | |
| eGFP654 | GCTATTACCTTAACCCAG | 17 |
| huMSTN | GAAAAAAGATTATATTGATTTTAAAATCATG | 18 |
| target | CAAAAACTGCAACTCTGTGTT | |
| muMSTN25-104 | CATACATTTGCAGTTTTTGCATCAT | 19 |
| muMSTN25-183 | TCATTTTTAAAAATCAGCACAATCTT | 20 |
| muMSTN25-194 | CAGTTTTTGCATCATTTTTAAAAATC | 21 |
| Exon44-A | GATCTGTCAAATCGCCTGCAGGTAA | 22 |
| Exon44-B | AAACTGTTCAGCTTCTGTTAGCCAC | 23 |
| Exon44-C | TTGTGTCTTTCTGAGAAACTGTTCA | 24 |
| Exon45-A | CTGACAACAGTTTGCCGCTGCCCAA | 25 |
| Exon45-B | CCAATGCCATCCTGGAGTTCCTGTAA | 26 |
| Exon45-C | CATTCAATGTTCTGACAACAGTTTGCCGCT | 27 |
| Exon50-A | CTTACAGGCTCCAATAGTGGTCAGT | 28 |
| Exon50-B | CCACTCAGAGCTCAGATCTTCTAACTTCC | 29 |
| Exon50-C | GGGATCCAGTATACTTACAGGCTCC | 30 |
| Exon51-A | ACATCAAGGAAGATGGCATTTCTAGTTTGG | 31 |
| Exon51-B | CTCCAACATCAAGGAAGATGGCATTTCTAG | 32 |
| Exon51-C | GAGCAGGTACCTCCAACATCAAGGAA | 33 |
| Exon53-A | CTGAAGGTGTTCTTGTACTTCATCC | 34 |
| Exon53-B | TGTTCTTGTACTTCATCCCACTGATTCTGA | 35 |
| SMN2-A | CTTTCATAATGCTGGCAG | 36 |
| SMN2-B | CATAATGCTGGCAG | 37 |
| SMN2-C | GCTGGCAG | 38 |
| CAG 9mer | CAG CAG CAG | 39 |
| CAG 12mer | CAG CAG CAG CAG | 40 |
| CAG 15mer | CAG CAG CAG CAG CAG | 41 |
| CAG 18mer | CAG CAG CAG CAG CAG CAG | 42 |
| AGC 9mer | AGC AGC AGC | 43 |
| AGC 12mer | AGC AGC AGC AGC | 44 |
| AGC 15mer | AGC AGC AGC AGC AGC | 45 |
| AGC 18mer | AGC AGC AGC AGC AGC AGC | 46 |
| GCA 9mer | GCA GCA GCA | 47 |
| GCA 12mer | GCA GCA GCA GCA | 48 |
| GCA 15mer | GCA GCA GCA GCA GCA | 49 |
| GCA 18mer | GCA GCA GCA GCA GCA GCA | 50 |
| AGC 25mer | AGC AGC AGC AGC AGC AGC AGC AGC A | 51 |
| CAG 25mer | CAG CAG CAG CAG CAG CAG CAG CAG C | 52 |
| CAGG 9mer | CAG GCA GGC | 53 |
| CAGG 12mer | CAG GCA GGC AGG | 54 |
| CAGG 24mer | CAG GCA GGC AGG CAG GCA GGC AGG | 55 |

aa = glycine or proline; B = β-alanine; X = 6-aminohexanoic acid; tg = unmodified amino terminus, or the amino terminal capped with an acetyl benzoyl or steroyl group (i.e., an acetyl amide, benzoyl amide or stearoyl amide) and Y^{b} is: —C(O)—(CHR^{e})ₙ—NH— wherein n is 2 to 7 and each R^{e} is independently, at each occurrence, hydrogen or methyl. For simplicity, not all sequences are noted with a terminal tg group; however, each of the above sequences may comprise an unmodified amino terminus or an amino terminus capped with an acetyl, benzoyl or stearoyl group

### III. Antisense Oligomers

Nucleic acid analogs included in the conjugates of the invention are substantially uncharged synthetic oligomers capable of base-specific binding to a target sequence of a polynucleotide, e.g. antisense oligonucleotide analogs. Such analogs include, for example, methylphosphonates, peptide nucleic acids, substantially uncharged N3′→P5′ phosphoramidates, and morpholino oligomers.

The base sequence of the nucleic acid analog, provided by base pairing groups supported by the analog backbone, can be any sequence, where the supported base pairing groups include standard or modified A, T, C, G and U bases or the non-standard inosine (I) and 7-deaza-G bases.

In some embodiments, the nucleic acid analog is a morpholino oligomer, i.e. an oligonucleotide analog composed of morpholino subunit structures of the form shown in FIG. 1, where (i) the structures are linked together by phosphorus-containing linkages, one to three atoms long, preferably two atoms long, joining the morpholino nitrogen of one subunit to the 5′ exocyclic carbon of an adjacent subunit, and (ii) Pi and Pj are purine or pyrimidine base-pairing moieties effective to bind, by base-specific hydrogen bonding, to a base in a polynucleotide. The purine or pyrimidine base-pairing moiety is typically adenine, cytosine, guanine, uracil or thymine. The synthesis, structures, and binding characteristics of morpholino oligomers are described further below and detailed in U.S. Pat. Nos. 5,698,685, 5,217,866, 5,142,047, 5,034,506, 5,166,315, 5,521,063, and 5,506,337, all of which are incorporated herein by reference.

Desirable chemical properties of the morpholino-based oligomers include the ability to selectively hybridize with a complementary-base target nucleic acid, including target RNA, with high Tm, even with oligomers as short as 8-14 bases, the ability to be actively transported into mammalian cells, and the ability of an oligomer:RNA heteroduplex to resist RNAse degradation.

In a preferred embodiment, the morpholino oligomer is about 8-40 subunits in length. More typically, the oligomer is about 8-20, about 8-16, about 10-30, or about 12-25 subunits in length. For some applications, such as antibacterial, short oligomers, e.g. from about 8-12 subunits in length, can be especially advantageous, particularly when attached to a peptide transporter as disclosed herein.

A. Oligomers with Modified Intersubunit Linkages

One embodiment of the present disclosure is directed to peptide-oligomer conjugates comprising nucleic acid analogues (e.g., morpholino oligomers) comprising modified intersubunit linkages. In some embodiments, the conjugates have higher affinity for DNA and RNA than do the corresponding unmodified oligomers and demonstrate improved cell delivery, potency, and/or tissue distribution properties compared to oligomers having other intersubunit linkages. In one embodiment, the conjugates comprise one or more intersubunit linkages of type (A) as defined below. In other embodiments, the conjugates comprise at least one intersubunit linkage of type (B) as defined below. In still other embodiments, the conjugates comprise intersubunit linkages of type (A) and type (B). In yet other embodiments, the conjugates comprise a morpholino oligomer as described in more detail below. The structural features and properties of the various linkage types and oligomers are described in more detail in the following discussion.

### 1. Linkage (A)

Applicants have found that enhancement of antisense activity, biodistribution and/or other desirable properties can be optimized by preparing oligomers having various intersubunit linkages. For example, the oligomers may optionally comprise one or more intersubunit linkages of type (A), and in certain embodiments the oligomers comprise at least one linkage of type (A), for example each linkage may be of type (A). In some other embodiments each linkage of type (A) has the same structure. Linkages of type (A) may include linkages disclosed in co-owned U.S. Pat. No. 7,943,762 which is hereby incorporated by reference in its entirety. Linkage (A) has the following structure (I), wherein 3′ and 5′ indicate the point of attachment to the 3′ and 5′ ends, respectively, of the morpholino ring (i.e., structure (i) discussed below):

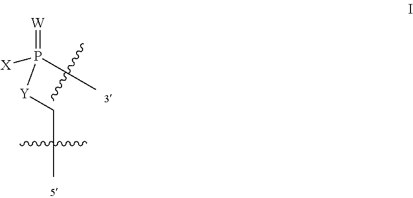

or a salt or isomer thereof, wherein:
W is, at each occurrence, independently S or O;
X is, at each occurrence, independently —N(CH₃)₂, —NR¹R², —OR³ or;

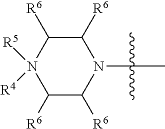

Y is, at each occurrence, independently O or —NR²,
R¹ is, at each occurrence, independently hydrogen or methyl;
R² is, at each occurrence, independently hydrogen or -LNR⁴R⁵R⁷;
R³ is, at each occurrence, independently hydrogen or C₁-C₆ alkyl;
R⁴ is, at each occurrence, independently hydrogen, methyl, —C(═NH)NH₂, —Z-L-NHC(═NH)NH₂ or —[C(═O)CHR′NH]ₘH, where Z is —C(═O)— or a direct bond, R′ is a side chain of a naturally occurring amino acid or a one- or two-carbon homolog thereof, and m is 1 to 6;
R⁵ is, at each occurrence, independently hydrogen, methyl or an electron pair;
R⁶ is, at each occurrence, independently hydrogen or methyl;
R⁷ is, at each occurrence, independently hydrogen C₁-C₆ alkyl or C₁-C₆ alkoxyalkyl; and
L is an optional linker up to 18 atoms in length comprising alkyl, alkoxy or alkylamino groups, or combinations thereof.

In some examples, the oligomer comprises at least one linkage of type (A). In some other embodiments, the oligomer includes at least two consecutive linkages of type (A). In further embodiments, at least 5% of the linkages in the oligomer are type (A); for example in some embodiments, 5%-95%, 10% to 90%, 10% to 50%, or 10% to 35% of the linkages may be linkage type (A). In some specific embodiments, at least one type (A) linkage is —N(CH₃)₂. In other embodiments, each linkage of type (A) is —N(CH₃)₂, and in even other embodiments each linkage in the oligomer is —N(CH₃)₂. In other embodiments, at least one type (A) linkage is piperizin-1-yl, for example unsubstituted piperazin-1-yl (e.g., A2 or A3). In other embodiments, each linkage of type (A) is piperizin-1-yl, for example unsubstituted piperazin-1-yl.

In some embodiments, W is, at each occurrence, independently S or O, and in certain embodiments W is O.

In some embodiments, X is, at each occurrence, independently —N(CH₃)₂, —NR¹R², —OR³. In some embodiments X is —N(CH₃)₂. In other aspects X is —NR¹R², and in other examples X is —OR³.

In some embodiments, R¹ is, at each occurrence, independently hydrogen or methyl. In some embodiments, R¹ is hydrogen. In other embodiments X is methyl.

In some embodiments, R² is, at each occurrence, hydrogen. In other embodiments R² is, at each occurrence, -LNR⁴R⁵R⁷. In some embodiments, R³ is, at each occurrence, independently hydrogen or C₁-C₆ alkyl. In other embodiments, R³ is methyl. In yet other embodiments, R³ is ethyl. In some other embodiments, R³ is n-propyl or isopropyl. In some other embodiments, R³ is C₄ alkyl. In other embodiments, R³ is C₈ alkyl. In some embodiments, R³ is C₆ alkyl.

In certain embodiments, R⁴ is, at each occurrence, independently hydrogen. In other embodiments, R⁴ is methyl. In yet other embodiments, R⁴ is —C(═NH)NH₂, and in other embodiments, R⁴ is —Z-L-NHC(═NH)NH₂. In still other embodiments, R⁴ is —[C(═O)CHR′NH]ₘH. Z is —C(═O)— in one embodiment and Z is a direct bond in another embodiment. R′ is a side chain of a naturally occurring amino acid. In some embodiments R′ is a one- or two-carbon homolog of a side chain of a naturally occurring amino acid.
m is and integer from 1 to 6. m may be 1. m may be 2 m may be 3 m may be 4 m may be 5 m may be 6

In some embodiments, R′ is, at each occurrence, independently hydrogen, methyl or an electron pair. In some embodiments, R′ is hydrogen. In other embodiments, R′ is methyl. In yet other embodiments, R′ is an electron pair.

In some embodiments, R⁶ is, at each occurrence, independently hydrogen or methyl. In some embodiments, R⁶ is hydrogen. In other embodiments, R⁶ is methyl.

In other embodiments, R⁷ is, at each occurrence, independently hydrogen C₁-C₆ alkyl or C₂-C₆ alkoxyalkyl. In some embodiments R7 is hydrogen. In other embodiments, R⁷ is C₁-C₆ alkyl. In yet other embodiments, R⁷ is C₂-C₆ alkoxyalkyl. In some embodiments, R⁷ is methyl. In other embodiments, R⁷ is ethyl. In yet other embodiments, R⁷ is n-propyl or isopropyl. In some other embodiments, R⁷ is C₄ alkyl. In some embodiments, R⁷ is C₅ alkyl. In some embodiments, R⁷ is C₆ alkyl. In yet other embodiments, R⁷ is C₂ alkoxyalkyl. In some other embodiments, R⁷ is C₃ alkoxyalkyl. In yet other embodiments, R⁷ is C₄ alkoxyalkyl. In some embodiments, R⁷ is C₅ alkoxyalkyl. In other embodiments, R⁷ is C₆ alkoxyalkyl.

The linker group L, as noted above, contains bonds in its backbone selected from alkyl (e.g. —CH₂—CH₂—), alkoxy (e.g., —C—O—C—), and alkylamino (e.g. —CH₂—NH—), with the proviso that the terminal atoms in L (e.g., those adjacent to carbonyl or nitrogen) are carbon atoms. Although branched linkages (e.g. —CH₂—CHCH₃—) are possible, the linker is generally unbranched. In one embodiment, the linker is a hydrocarbon linker. Such a linker may have the structure (CH₂)ₙ—, where n is 1-12, preferably 2-8, and more preferably 2-6.

Oligomers having any number of linkage type (A) are provided. In some embodiments, the oligomer contains no linkages of type (A). In certain embodiments, 5, 10, 20, 30, 40, 50, 60, 70, 80 or 90 percent of the linkages are linkage (A). In selected embodiments, 10 to 80, 20 to 80, 20 to 60, 20 to 50, 20 to 40, or 20 to 35 percent of the linkages are linkage (A). In still other embodiments, each linkage is type (A).

### 2. Linkage (B)

In some embodiments, the oligomers comprise at least one linkage of type (B). For example the oligomers may comprise 1, 2, 3, 4, 5, 6 or more linkages of type (B). The type (B) linkages may be adjacent or may be interspersed throughout the oligomer. Linkage type (B) has the following structure (I):

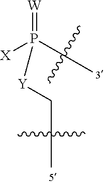

or a salt or isomer thereof, wherein:
   W is, at each occurrence, independently S or O;
   X is, at each occurrence, independently —NR⁸R⁹ or —OR³; and
   Y is, at each occurrence, independently O or —NR¹⁰,
   R³ is, at each occurrence, independently hydrogen or C₁-C₆ alkyl;
   R⁸ is, at each occurrence, independently hydrogen or C₂-C₁₂ alkyl;
   R⁹ is, at each occurrence, independently hydrogen, C₁-C₁₂ alkyl, C₁-C₁₂ aralkyl or aryl;
   R¹⁰ is, at each occurrence, independently hydrogen, C₁-C₁₂ alkyl or -LNR⁴R⁵R⁷;
   wherein R⁸ and R⁹ may join to form a 5-18 membered mono or bicyclic heterocycle or R⁸, R⁹ or R³ may join with R¹⁰ to form a 5-7 membered heterocycle, and wherein when X is 4-piperazino, X has the following structure (III):

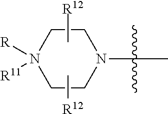

wherein:
   R¹¹ is, at each occurrence, independently C₂-C₁₂ alkyl, C₁-C₁₂ aminoalkyl, C₁-C₁₂ alkylcarbonyl, aryl, heteroaryl or heterocyclyl;
   R is, at each occurrence, independently an electron pair, hydrogen or C₁-C₁₂ alkyl; and
   R¹² is, at each occurrence, independently, hydrogen, C₁-C₁₂ alkyl, C₁-C₁₂ aminoalkyl, —NH₂, —CONH₂, —NR¹³R¹⁴, —NR¹³R¹⁴R¹⁵, C₁-C₁₂ alkylcarbonyl, oxo, —CN, trifluoromethyl, amidyl, amidinyl, amidinylalkyl, amidinylalkylcarbonyl guanidinyl, guanidinylalkyl, guanidinylalkylcarbonyl, cholate, deoxycholate, aryl, heteroaryl, heterocycle, —SR¹³ or C₁-C₁₂ alkoxy, wherein R¹³, R¹⁴ and R¹⁵ are, at each occurrence, independently C₁-C₁₂ alkyl.

In some examples, the oligomer comprises one linkage of type (B). In some other embodiments, the oligomer comprises two linkages of type (B). In some other embodiments, the oligomer comprises three linkages of type (B). In some other embodiments, the oligomer comprises four linkages of type (B). In still other embodiments, the linkages of type (B) are consecutive (i.e., the type (B) linkages are adjacent to each other). In further embodiments, at least 5% of the linkages in the oligomer are type (B); for example in some embodiments, 5%-95%, 10% to 90%, 10% to 50%, or 10% to 35% of the linkages may be linkage type (B).

In other embodiments, R³ is, at each occurrence, independently hydrogen or C₁-C₆ alkyl. In yet other embodiments, R³ may be methyl. In some embodiments, R³ may be ethyl. In some other embodiments, R³ may be n-propyl or isopropyl. In yet other embodiments, R³ may be C₄ alkyl. In some embodiments, R³ may be C₅ alkyl. In some embodiments, R³ may be C₆ alkyl.

In some embodiments, R⁸ is, at each occurrence, independently hydrogen or C₂-C₁₂ alkyl. In some embodiments, R⁸ is hydrogen. In yet other embodiments, R⁸ is ethyl. In some other embodiments, R⁸ is n-propyl or isopropyl. In some embodiments, R⁸ is C₄ alkyl. In yet other embodiments, R⁸ is C₈ alkyl. In other embodiments, R⁸ is C₆ alkyl. In some embodiments, R⁸ is C₇ alkyl. In yet other embodiments, R⁸ is C₈ alkyl. In other embodiments, R⁸ is C₉ alkyl. In yet other embodiments, R⁸ is C₁₀ alkyl. In some other embodiments, R⁸ is C₁₁ alkyl. In yet other embodiments, R⁸ is C₁₂ alkyl. In some other embodiments, R⁸ is C₂-C₁₂ alkyl and the C₂-C₁₂ alkyl includes one or more double bonds (e.g., alkene), triple bonds (e.g., alkyne) or both. In some embodiments, R⁸ is unsubstituted C₂-C₁₂ alkyl.

In some embodiments, R⁹ is, at each occurrence, independently hydrogen, C₁-C₁₂ alkyl, C₁-C₁₂ aralkyl or aryl. In some embodiments, R⁹ is hydrogen.

In yet other embodiments, R⁹ is C₁-C₁₂ alkyl. In other embodiments, R⁹ is methyl. In yet other embodiments, R⁹ is ethyl. In some other embodiments, R⁹ is n-propyl or isopropyl. In some embodiments, R⁹ is C₄ alkyl. In some embodiments, R⁹ is C₅ alkyl. In yet other embodiments, R⁹ is C₆ alkyl. In some other embodiments, R⁹ is C₇ alkyl. In some embodiments, R⁹ is C₈ alkyl. In some embodiments, R⁹ is C₉ alkyl. In some other embodiments, R⁹ is C₁₀ alkyl. In some other embodiments, R⁹ is C₁₁ alkyl. In yet other embodiments, R⁹ is C₁₂ alkyl.

In some other embodiments, R⁹ is C₁-C₁₂ aralkyl. For example, n some embodiments R⁹ is benzyl and the benzyl may be optionally substituted on either the phenyl ring or the benzylic carbon. Substituents in this regards include alkyl and alkoxy groups, for example methyl or methoxy. In some embodiments, the benzyl group is substituted with methyl at the benzylic carbon. For example, in some embodiments, R⁹ has the following structure (XIV):

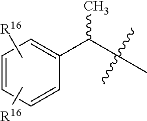

In other embodiments, R⁹ is aryl. For example, in some embodiments R⁹ is phenyl, and the phenyl may be optionally substituted. Substituents in this regard substitutents include alkyl and alkoxy groups, for example methyl or methoxy. In other embodiments, R⁹ is phenyl and the phenyl comprises a crown ether moiety, for example a 12-18 membered crown ether. In one embodiment the crown ether is 18 membered and may further comprise and additional phenyl moiety. For example, in one embodiment R⁹ has one of the following structures (XV) or XVI):

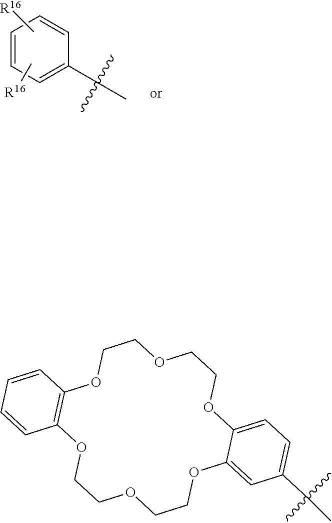

In some embodiments, R¹⁰ is, at each occurrence, independently hydrogen, C₁-C₁₂ alkyl or -LNR⁴R⁵R⁷, wherein R⁴, R⁵ and R⁷ are as defined above with respect to linkage (A). In other embodiments, R¹⁰ is hydrogen. In other embodiments, R¹⁰ is C₁-C₁₂ alkyl, and in other embodiments R¹⁰ is -LNR⁴R⁵R⁷. In some embodiments, R¹⁰ is methyl. In yet other embodiments, R¹⁰ is ethyl. In some embodiments, R¹⁰ is C₃ alkyl. In some embodiments, R¹⁰ is C₄ alkyl. In yet other embodiments, R¹⁰ is C₅ alkyl. In some other embodiments, R¹⁰ is C₆ alkyl. In other embodiments, R¹⁰ is C₇ alkyl. In yet other embodiments, R¹⁰ is C₅ alkyl. In some embodiments, R¹⁰ is C₉ alkyl. In other embodiments, R¹⁰ is C₁₀ alkyl. In yet other embodiments, R¹⁰ is C₁₁ alkyl. In some other embodiments, R¹⁰ is C₁₂ alkyl.

In some embodiments, R⁸ and R⁹ join to form a 5-18 membered mono or bicyclic heterocycle. In some embodiments the heterocycle is a 5 or 6 membered monocyclic heterocycle. For example, in some embodiments linkage (B) has the following structure (IV):

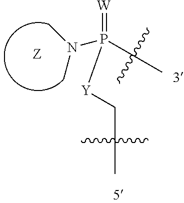

wherein Z represents a 5 or 6 membered monocyclic heterocycle.

In other embodiments, heterocycle is bicyclic, for example a 12-membered bicyclic heterocycle. The heterocycle may be piperizinyl. The heterocycle may be morpholino. The heterocycle may be piperidinyl. The heterocycle may be decahydroisoquinoline. Representative heterocycles include the following:

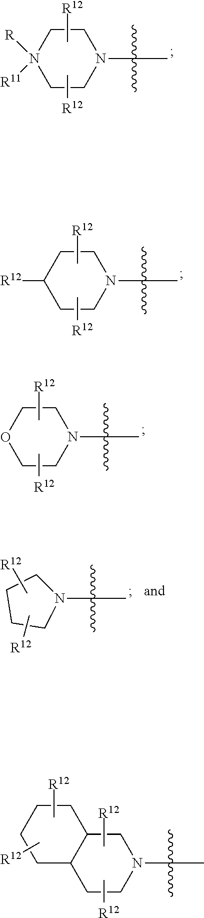

In some embodiments, R¹¹ is, at each occurrence, independently C₂-C₁₂ alkyl, C₁-C₁₂ aminoalkyl, aryl, heteroaryl or heterocyclyl.

In some embodiments, R¹¹ is C₂-C₁₂ alkyl. In some embodiments, R¹¹ is ethyl. In other embodiments, R¹¹ is C₃ alkyl. In yet other embodiments, R¹¹ is isopropyl. In some other embodiments, R¹¹ is C₄ alkyl. In other embodiments, R¹¹ is C₅ alkyl. In some embodiments, R¹¹ is C₆ alkyl. In other embodiments, R¹¹ is C₇ alkyl. In some embodiments, R¹¹ is C₈ alkyl. In other embodiments, R¹¹ is C₉ alkyl. In yet other embodiments, R¹¹ is C₁₀ alkyl. In some other embodiments, R¹¹ is C₁₁ alkyl. In some embodiments, R¹¹ is C₁₂ alkyl.

In other embodiments, R¹¹ is C₁-C₁₂ aminoalkyl. In some embodiments, R¹¹ is methylamino. In some embodiments, R¹¹ is ethylamino. In other embodiments, R¹¹ is C₃ aminoalkyl. In yet other embodiments, R¹¹ is C₄ aminoalkyl. In some other embodiments, R¹¹ is C₅ aminoalkyl. In other embodiments, R¹¹ is C₆ aminoalkyl. In yet other embodiments, R¹¹ is C₇ aminoalkyl. In some embodiments, R¹¹ is C₈ aminoalkyl. In other embodiments, R¹¹ is C₉ aminoalkyl. In yet other embodiments, R¹¹ is C₁₀ aminoalkyl. In some other embodiments, R¹¹ is C₁₁ aminoalkyl. In other embodiments, R¹¹ is C₁₂ aminoalkyl.

In other embodiments, R¹¹ is C₁-C₁₂ alkylcarbonyl. In yet other embodiments, R¹¹ is C₁ alkylcarbonyl. In other embodiments, R¹¹ is C₂ alkylcarbonyl. In some embodiments, R¹¹ is C₃ alkylcarbonyl. In yet other embodiments, R¹¹ is C₄ alkylcarbonyl. In some embodiments, R¹¹ is C₅ alkylcarbonyl. In some other embodiments, R¹¹ is C₆ alkylcarbonyl. In other embodiments, R¹¹ is C₇ alkylcarbonyl. In yet other embodiments, R¹¹ is C₈ alkylcarbonyl. In some embodiments, R¹¹ is C₉ alkylcarbonyl. In yet other embodiments, R¹¹ is C₁₀ alkylcarbonyl. In some other embodiments, R¹¹ is C₁₁ alkylcarbonyl. In some embodiments, R¹¹ is C₁₂ alkylcarbonyl. In yet other embodiments, R¹¹ is —C(═O)(CH₂)ₙCO₂H, where n is 1 to 6.

For example, in some embodiments, n is 1. In other embodiments, n is 2. In yet other embodiments, n is 3. In some other embodiments, n is 4. In yet other embodiments, n is 5. In other embodiments, n is 6.

In other embodiments, R¹¹ is aryl. For example, in some embodiments, R¹¹ is phenyl. In some embodiments, the phenyl is substituted, for example with a nitro group.

In other embodiments, R¹¹ is heteroaryl. For example, in some embodiments, R¹¹ is pyridinyl. In other embodiments, R¹¹ is pyrimidinyl.

In other embodiments, R¹¹ is heterocyclyl. For example, in some embodiments, R¹¹ is piperidinyl, for example piperidin-4-yl.

In some embodiments, R¹¹ is ethyl, isopropyl, piperidinyl, pyrimidinyl, cholate, deoxycholate, or —C(═O)(CH₂)ₙCO₂H, where n is 1 to 6.

In some embodiments, R is an electron pair. In other embodiments, R is hydrogen, and in other embodiments R is C₁-C₁₂ alkyl. In some embodiments, R is methyl. In some embodiments, R is ethyl. In other embodiments, R is C₃ alkyl. In yet other embodiments, R is isopropyl. In some other embodiments, R is C₄ alkyl. In yet other embodiments, R is C₅ alkyl. In some embodiments, R is C₆ alkyl. In other embodiments, R is C₇ alkyl. In yet other embodiments, R is C₈ alkyl. In other embodiments, R is C₉ alkyl. In some embodiments, R is C₁₀ alkyl. In yet other embodiments, R is C₁₁ alkyl. In some embodiments, R is C₁₂ alkyl.

In some embodiments, R₁₂ is, at each occurrence, independently, hydrogen, C₁-C₁₂ alkyl, C₁-C₁₂ aminoalkyl, —NH₂, —CONH₂, —NR¹³R¹⁴, —NR¹³R¹⁴R¹⁵, oxo, —CN, trifluoromethyl, amidyl, amidinyl, amidinylalkyl, amidinylalkylcarbonyl guanidinyl, guanidinylalkyl, guanidinylalkylcarbonyl, cholate, deoxycholate, aryl, heteroaryl, heterocycle, —SR¹³ or C₁-C₁₂ alkoxy, wherein R¹³, R¹⁴ and R¹⁵ are, at each occurrence, independently C₁-C₁₂ alkyl

In some embodiments, R¹² is hydrogen. In some embodiments, R¹² is C₁-C₁₂ alkyl. In some embodiments, R¹² is C₁-C₁₂ aminoalkyl. In some embodiments, R¹² is —NH₂. In some embodiments, R¹² is —CONH₂. In some embodiments, R¹² is —NR¹³R¹⁴. In some embodiments, R¹² is —NR¹³R¹⁴R¹⁵. In some embodiments, R¹² is C₁-C₁₂ alkylcarbonyl. In some embodiments, R¹² is oxo. In some embodiments, R¹² is —CN. In some embodiments, R¹² is trifluoromethyl. In some embodiments, R¹² is amidyl. In some embodiments, R¹² is amidinyl. In some embodiments, R¹² is amidinylalkyl. In some embodiments, R¹² is amidinylalkylcarbonyl. In some embodiments, R¹² is guanidinyl, for example mono methylguanidynyl or dimethylguanidinyl. In some embodiments, R¹² is guanidinylalkyl. In some embodiments, R¹² is amidinylalkylcarbonyl. In some embodiments, R¹² is cholate. In some embodiments, R¹² is deoxycholate. In some embodiments, R¹² is aryl. In some embodiments, R¹² is heteroaryl. In some embodiments, R¹² is heterocycle. In some embodiments, R¹² is —SR¹³. In some embodiments, R¹² is C₁-C₁₂ alkoxy. In some embodiments, R¹² is dimethyl amine.

In other embodiments, R¹² is methyl. In yet other embodiments, R¹² is ethyl. In some embodiments, R¹² is C₃ alkyl. In some embodiments, R¹² is isopropyl. In some embodiments, R¹² is C₄ alkyl. In other embodiments, R¹² is C₅ alkyl. In yet other embodiments, R¹² is C₆ alkyl. In some other embodiments, R¹² is C₇ alkyl. In some embodiments, R¹² is C₈ alkyl. In yet other embodiments, R¹² is C₉ alkyl. In some embodiments, R¹² is C₁₀ alkyl. In yet other embodiments, R¹² is C₁₁ alkyl. In other embodiments, R¹² is C₁₂ alkyl. In yet other embodiments, the alkyl moiety is substituted with one or more oxygen atom to form an ether moiety, for example a methoxymethyl moiety.

In some embodiments, R¹² is methylamino. In other embodiments, R¹² is ethylamino. In yet other embodiments, R¹² is C₃ aminoalkyl. In some embodiments, R¹² is C₄ aminoalkyl. In yet other embodiments, R¹² is C₅ aminoalkyl. In some other embodiments, R¹¹ is C₆ aminoalkyl. In some embodiments, R¹¹ is C₇ aminoalkyl. In some embodiments, R¹² is C₈ aminoalkyl. In yet other embodiments, R¹² is C₉ aminoalkyl. In some other embodiments, R¹² is C₁₀ aminoalkyl. In yet other embodiments, R¹² is C₁₁ aminoalkyl. In other embodiments, R¹² is C₁₂ aminoalkyl. In some embodiments, the amino alkyl is a dimethylamino alkyl.

In yet other embodiments, R¹² is acetyl. In some other embodiments, R¹² is C₂ alkylcarbonyl. In some embodiments, R¹² is C₃ alkylcarbonyl. In yet other embodiments, R¹² is C₄ alkylcarbonyl. In some embodiments, R¹² is C₅ alkylcarbonyl. In yet other embodiments, R¹² is C₆ alkylcarbonyl. In some other embodiments, R¹² is C₇ alkylcarbonyl. In some embodiments, R¹² is C₈ alkylcarbonyl. In yet other embodiments, R¹² is C₉ alkylcarbonyl. In some other embodiments, R¹² is C₁₀ alkylcarbonyl. In some embodiments, R¹² is C₁₁ alkylcarbonyl. In other embodiments, R¹² is C₁₂ alkylcarbonyl. The alkylcarbonyl is substituted with a carboxy moiety, for example the alkylcarbonyl is substituted to form a succinic acid moiety (i.e., a 3-carboxyalkylcarbonyl). In other embodiments, the alkylcarbonyl is substituted with a terminal —SH group.

In some embodiments, R¹² is amidyl. In some embodiments, the amidyl comprises an alkyl moiety which is further substituted, for example with —SH, carbamate, or combinations thereof. In other embodiments, the amidyl is substituted with an aryl moiety, for example phenyl. In certain embodiments, R¹² may have the following structure (IX):

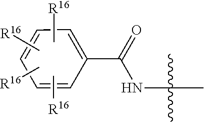

wherein R¹⁶ is, at each occurrence, independently hydrogen, C₁-C₁₂ alkyl, C₁-C₁₂ alkoxy, —CN, aryl or heteroaryl.

In some embodiments, R¹² is methoxy. In other embodiments, R¹² is ethoxy. In yet other embodiments, R¹² is C₃ alkoxy. In some embodiments, R¹² is C₄ alkoxy. In some embodiments, R¹² is C₅ alkoxy. In some other embodiments, R¹² is C₆ alkoxy. In other embodiments, R¹² is C₇ alkoxy. In some other embodiments, R¹¹ is C₈ alkoxy. In some embodiments, R¹² is C₉ alkoxy. In other embodiments, R¹² is C₁₀ alkoxy. In some embodiments, R¹² is C₁₁ alkoxy. In yet other embodiments, R¹² is C₁₂ alkoxy.

In certain embodiments, R¹² is pyrrolidinyl, for example pyrrolidin-1-yl. In other embodiments, R¹² is piperidinyl, for example piperidin-1-yl or piperidin-4-yl. In other embodiment, R¹² is morpholino, for example morpholin-4-yl. In other embodiments, R¹² is phenyl, and in even further embodiments, the phenyl is substituted, for example with a nitro group. In still other embodiments, R¹² is pyrimidinyl, for example pyrimidin-2-yl.

In other embodiments, R¹³, R¹⁴ and R¹⁵ are, at each occurrence, independently C₁-C₁₂ alkyl. In some embodiments, R³, R¹⁴ or R¹⁵ is methyl. In yet other embodiments, R³, R¹⁴ or R¹⁵ is ethyl. In other embodiments, R³, R¹⁴ or R¹⁵ is C₃ alkyl. In yet other embodiments, R¹³, R¹⁴ or R¹⁵ is isopropyl. In other embodiments, R¹³, R¹⁴ or R¹⁵ is C₄ alkyl. In some embodiments, R¹³, R¹⁴ or R¹⁵ is C₅ alkyl. In some other embodiments, R¹³, R¹⁴ or R¹⁵ is C₆ alkyl. In other embodiments, R¹³, R¹⁴ or R¹⁵ is C₇ alkyl. In yet other embodiments, R¹³, R¹⁴ or R¹⁵ is C₈ alkyl. In other embodiments, R¹³, R¹⁴ or R¹⁵ is C₉ alkyl. In some embodiments, R¹³, R¹⁴ or R¹⁵ is C₁₀ alkyl. In some embodiments, R¹³, R¹⁴ or R¹⁵ is C₁₁ alkyl. In yet other embodiments, R¹³, R¹⁴ or R¹⁵ is C₁₂ alkyl.

As noted above, in some embodiments, R¹² is amidyl substituted with an aryl moiety. In this regard, each occurrence of R¹⁶ may be the same or different. In certain of these embodiments, R¹⁶ is hydrogen. In other embodiments, R¹⁶ is —CN. In other embodiments, R¹⁶ is heteroaryl, for example tetrazolyl. In certain other embodiments, R¹⁶ is methoxy. In other embodiments, R¹⁶ is aryl, and the aryl is optionally substituted. Optional substitutents in this regard include: C₁-C₁₂ alkyl, C₁-C₁₂ alkoxy, for example methoxy; trifluoromethoxy; halo, for example chloro; and trifluoromethyl.

In other embodiments, R¹⁶ is methyl. In yet other embodiments, R¹⁶ is ethyl. In some embodiments, R¹⁶ is C₃ alkyl. In some other embodiments, R¹⁶ is isopropyl. In yet other embodiments, R¹⁶ is C₄ alkyl. In other embodiments, R¹⁶ is C₆ alkyl. In yet other embodiments, R¹⁶ is C₆ alkyl. In some other embodiments, R¹⁶ is C₇ alkyl. In some embodiments, R¹⁶ is C₈ alkyl. In yet other embodiments, R¹⁶ is C₉ alkyl.

In some other embodiments, R¹⁶ is C₁₀ alkyl. In other embodiments, R¹⁶ is C₁₁ alkyl. In some other embodiments, R¹⁶ is C₁₂ alkyl.

In some embodiments, R¹⁶ is methoxy. In some embodiments, R¹⁶ is ethoxy. In yet other embodiments, R¹⁶ is C₃ alkoxy. In some other embodiments, R¹⁶ is C₄ alkoxy. In other embodiments, R¹⁶ is C₅ alkoxy. In some other embodiments, R¹⁶ is C₆ alkoxy. In yet other embodiments, R¹⁶ is C₇ alkoxy. In some other embodiments, R¹⁶ is C₈ alkoxy. In yet other embodiments, R¹⁶ is C₉ alkoxy. In some other embodiments, R¹⁶ is C₁₀ alkoxy. In some embodiments, R¹⁶ is C₁₁ alkoxy. In some other embodiments, R¹⁶ is C₁₂ alkoxy.

In some other embodiments, R⁸ and R⁹ join to form a 12-18 membered crown ether. For example, in some embodiments, the crown ether s 18 membered, and in other embodiments the crown ether is 15 membered. In certain embodiments, R⁸ and R⁹ join to form a heterocycle having one of the following structures (X) or (XI):

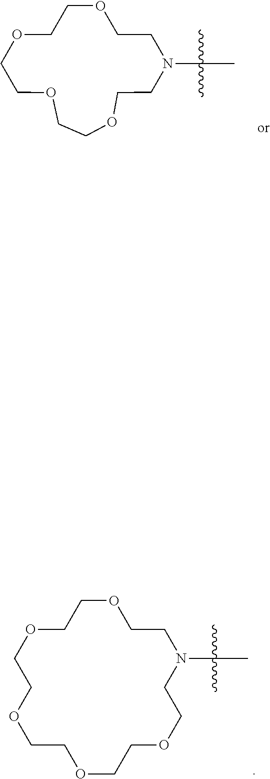 [SMILES: *N1CCOCCOCCOCCOCC1 ; *N1CCOCCOCCOCCOCCOCC1]

In some embodiments, R⁸, R⁹ or R³ join with R¹⁰ to form a 5-7 membered heterocycle. For example, in some embodiments, R³ joins with R¹⁰ to form a 5-7 membered heterocycle. In some embodiments, the heterocycle is 5-membered. In other embodiments, the heterocycle is 6-membered. In other embodiments, the heterocycle is 7-membered. In some embodiments, the heterocycle is represented by the following structure (XII):

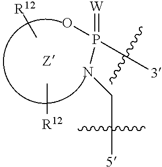

wherein Z′ represents a 5-7 membered heterocycle. In certain embodiments of structure (XI), R¹² is hydrogen at each occurrence. For example, linkage (B) may have one of the following structures (B1), (B2) or (B3):

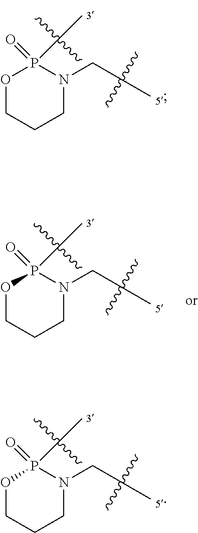 [SMILES: *CN1CCCOP1(*)=O ; *CN1CCCO[P@]1(*)=O ; *CN1CCCO[P@@]1(*)=O]

In certain other embodiments, R¹² is C₁-C₁₂ alkylcarbonyl or amidyl which is further substituted with an arylphosphoryl moiety, for example a triphenyl phosphoryl moiety. Examples of linkages having this structure include B56 and B55.

In certain embodiment, linkage (B) does not have any of the the structures A1-A5. Table 2 shows representative linkages of type (A) and (B).

TABLE 2
Representative Intersubunit Linkages

| No. | Name | Structure |
|---|---|---|
| A1 | PMO | 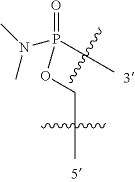 [SMILES: *COP(*)(=O)N(C)C] |
| A2 | PMO⁺ (unprotonated form depicted) | 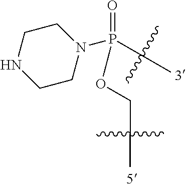 [SMILES: *COP(*)(=O)N1CCNCC1] |
| A3 | PMO⁺ (+) | 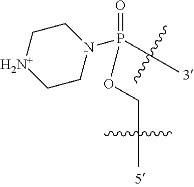 [SMILES: *COP(*)(=O)N1CC[NH2+]CC1] |
| A4 | PMO^{mepip} (m+) | 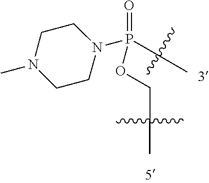 [SMILES: *COP(*)(=O)N1CCN(C)CC1] |
| A5 | PMO^{GUX} | 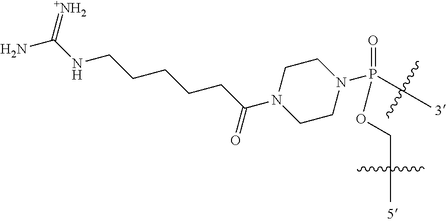 [SMILES: *COP(*)(=O)N1CCN(C(=O)CCCCCNC(N)=[NH2+])CC1] |
| B1 | PMO^{cp} | 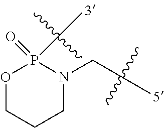 [SMILES: *CN1CCCOP1(*)=O] |
| B2 | PMO^{cps} | 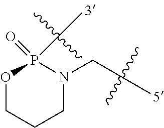 [SMILES: *CN1CCCO[P@]1(*)=O] |
| B3 | PMO^{cpr} | 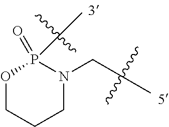 [SMILES: *CN1CCCO[P@@]1(*)=O] |
| B4 | PMO^{Shc} | 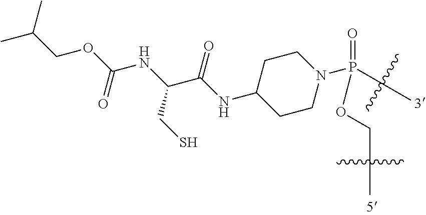 [SMILES: *COP(*)(=O)N1CCC(NC(=O)[C@H](CS)NC(=O)OCC(C)C)CC1] |
| B5 | PMO^{morpholino} (m) | 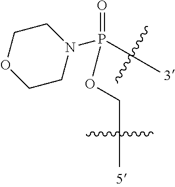 [SMILES: *COP(*)(=O)N1CCOCC1] |
| B6 | PMO^{tri} (t) | 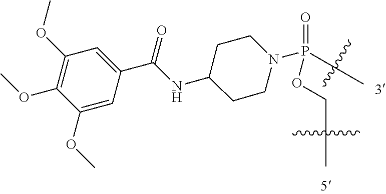 [SMILES: *COP(*)(=O)N1CCC(NC(=O)c2cc(OC)c(OC)c(OC)c2)CC1] |
| B7 | PMO^{hex} (h) | 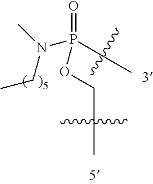 |
| B8 | PMO^{dodec} | 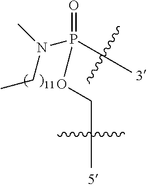 |
| B9 | PMO^{dihex} | 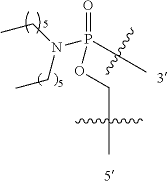 |
| B10 | PMO^{apn} (a) | 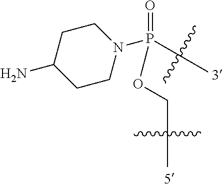 [SMILES: *COP(*)(=O)N1CCC(N)CC1] |
| B11 | PMO^{pyr} (p) | 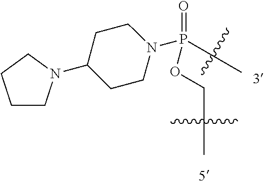 [SMILES: *COP(*)(=O)N1CCC(N2CCCC2)CC1] |
| B12 | PMO^{pyr} (HCl Salt) | 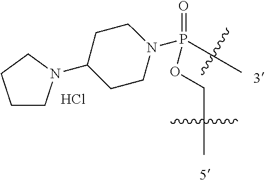 [SMILES: *COP(*)(=O)N1CCC(N2CCCC2)CC1 ; Cl] |
| B13 | PMO^{rba} | 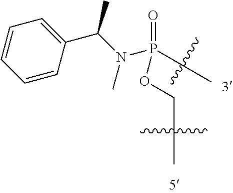 [SMILES: *COP(*)(=O)N(C)[C@H](C)c1ccccc1] |
| B14 | PMO^{sba} | 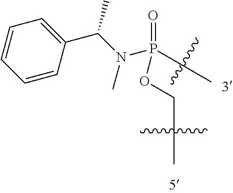 [SMILES: *COP(*)(=O)N(C)[C@@H](C)c1ccccc1] |
| B15 | PMO^{dimethylapn} | 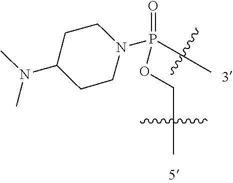 [SMILES: *COP(*)(=O)N1CCC(N(C)C)CC1] |
| B16 | PMO^{etpip} | 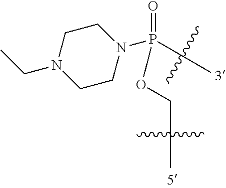 [SMILES: *COP(*)(=O)N1CCN(CC)CC1] |
| B17 | PMO^{iprpip} | 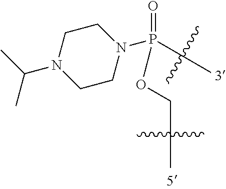 [SMILES: *COP(*)(=O)N1CCN(C(C)C)CC1] |
| B18 | PMO^{pyrQMe} | 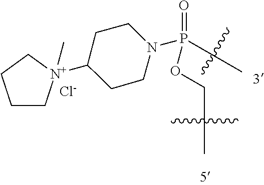 [SMILES: *COP(*)(=O)N1CCC([N+]2(C)CCCC2)CC1 ; [Cl-]] |
| B19 | PMO^{cb} | 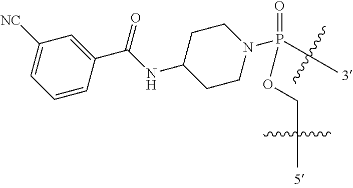 [SMILES: *COP(*)(=O)N1CCC(NC(=O)c2cccc(C#N)c2)CC1] |
| B20 | PMO^{ma} | 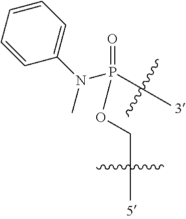 [SMILES: *COP(*)(=O)N(C)c1ccccc1] |
| B21 | PMO^{bu} | 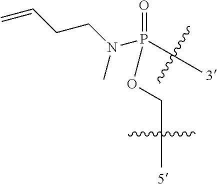 [SMILES: *COP(*)(=O)N(C)CCC=C] |
| B22 | PMO^{bi} | 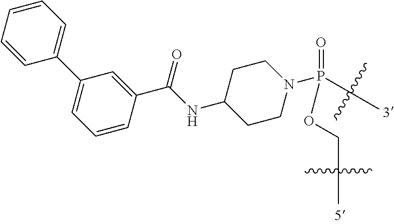 [SMILES: *COP(*)(=O)N1CCC(NC(=O)c2cccc(-c3ccccc3)c2)CC1] |
| B23 | PMO^{pip} | 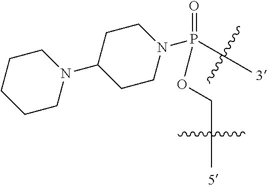 [SMILES: *COP(*)(=O)N1CCC(N2CCCCC2)CC1] |
| B24 | PMO^{obmb} | 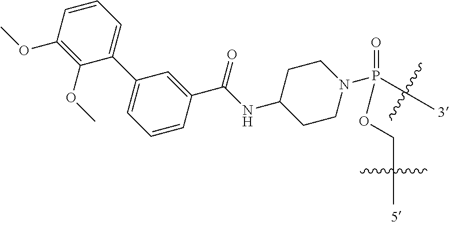 [SMILES: *COP(*)(=O)N1CCC(NC(=O)c2cccc(-c3cccc(OC)c3OC)c2)CC1] |
| B25 | PMO^{tfb} | 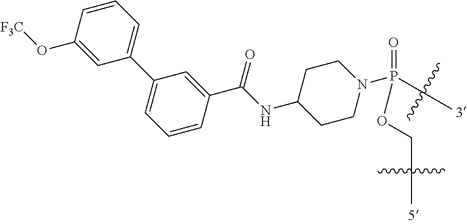 [SMILES: *COP(*)(=O)N1CCC(NC(=O)c2cccc(-c3cccc(OC(F)(F)F)c3)c2)CC1] |
| B26 | PMO^{ctfb} | 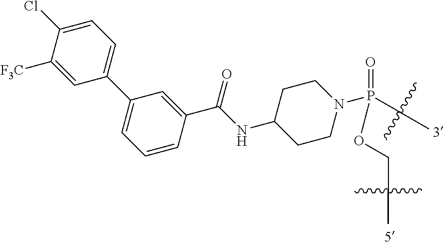 [SMILES: *COP(*)(=O)N1CCC(NC(=O)c2cccc(-c3ccc(Cl)c(C(F)(F)F)c3)c2)CC1] |
| B27 | PMO^{ptfb} | 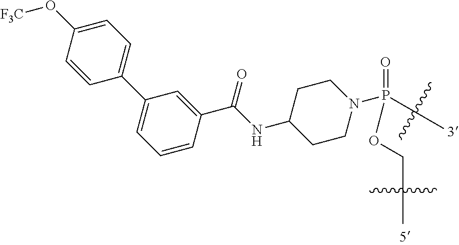 [SMILES: *COP(*)(=O)N1CCC(NC(=O)c2cccc(-c3ccc(OC(F)(F)F)cc3)c2)CC1] |
| B28 | PMO^{dcb} | 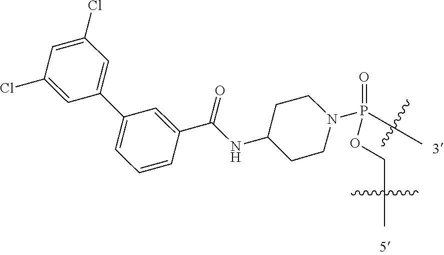 [SMILES: *COP(*)(=O)N1CCC(NC(=O)c2cccc(-c3cc(Cl)cc(Cl)c3)c2)CC1] |
| B29 | PMO^{dmb} | 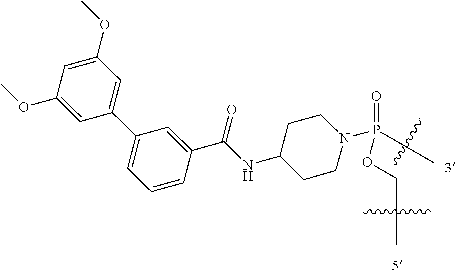 [SMILES: *COP(*)(=O)N1CCC(NC(=O)c2cccc(-c3cc(OC)cc(OC)c3)c2)CC1] |
| B30 | PMO^{hy} | 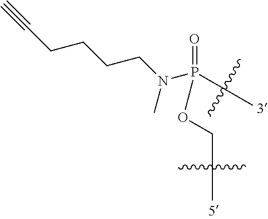 [SMILES: *COP(*)(=O)N(C)CCCCC#C] |
| B31 | PMO^{6ce} | 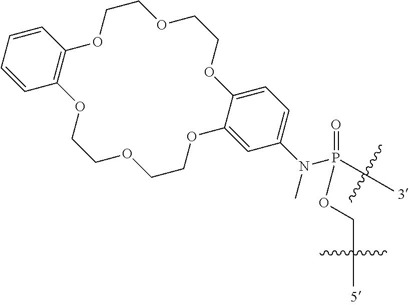 [SMILES: *COP(*)(=O)N(C)c1ccc2c(c1)OCCOCCOc1ccccc1OCCOCCO2] |
| B32 | PMO^{b} | 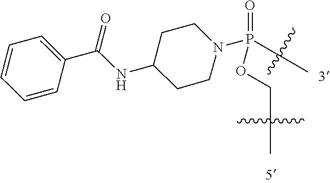 [SMILES: *COP(*)(=O)N1CCC(NC(=O)c2ccccc2)CC1] |
| B33 | PMO^{q} | 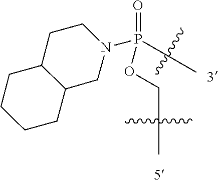 [SMILES: *COP(*)(=O)N1CCC2CCCCC2C1] |
| B34 | PMO^{npp} | 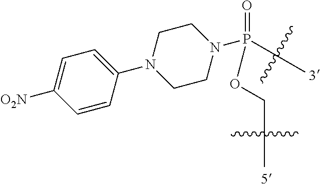 [SMILES: *COP(*)(=O)N1CCN(c2ccc([N+](=O)[O-])cc2)CC1] |
| B35 | PMO^{o} | 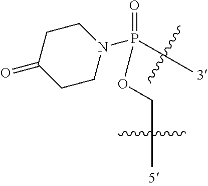 [SMILES: *COP(*)(=O)N1CCC(=O)CC1] |
| B36 | PMO^{4ce} | 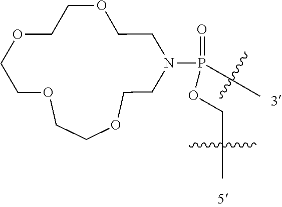 [SMILES: *COP(*)(=O)N1CCOCCOCCOCCOCC1] |
| B37 | PMO^{5ce} | 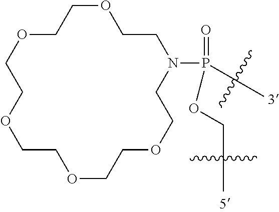 [SMILES: *COP(*)(=O)N1CCOCCOCCOCCOCCOCC1] |
| B38 | PMO^{f3p} | 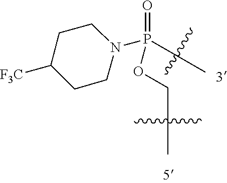 [SMILES: *COP(*)(=O)N1CCC(C(F)(F)F)CC1] |
| B39 | PMO^{cyp} | 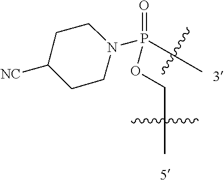 [SMILES: *COP(*)(=O)N1CCC(C#N)CC1] |
| B40 | PMO^{mop} | 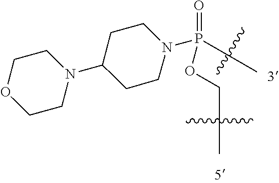 [SMILES: *COP(*)(=O)N1CCC(N2CCOCC2)CC1] |
| B41 | PMO^{pp} | 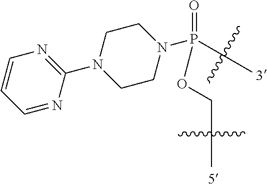 [SMILES: *COP(*)(=O)N1CCN(c2ncccn2)CC1] |
| B42 | PMO^{dmepip} | 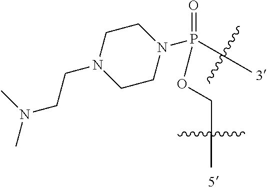 [SMILES: *COP(*)(=O)N1CCN(CCN(C)C)CC1] |
| B43 | PMO^{NPpip} | 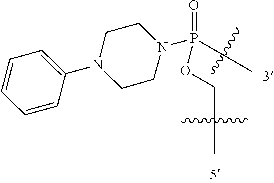 [SMILES: *COP(*)(=O)N1CCN(c2ccccc2)CC1] |
| B44 | PMO^{bipip} | 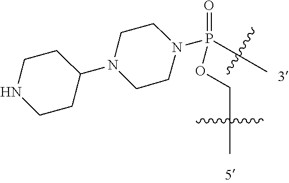 [SMILES: *COP(*)(=O)N1CCN(C2CCNCC2)CC1] |
| B45 | PMO^{suc} | 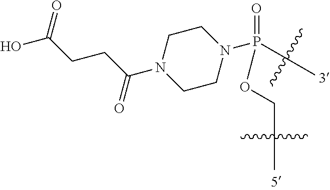 [SMILES: *COP(*)(=O)N1CCN(C(=O)CCC(=O)O)CC1] |
| 46 | PMO^{glutaric} | 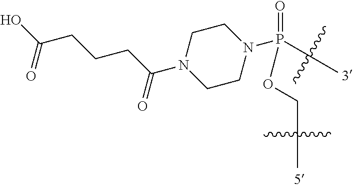 [SMILES: *COP(*)(=O)N1CCN(C(=O)CCCC(=O)O)CC1] |
| B47 | PMO^{tet} | 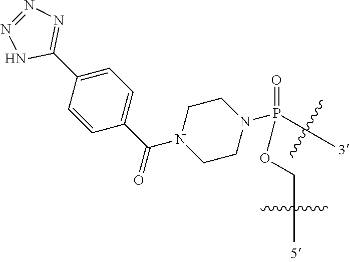 [SMILES: *COP(*)(=O)N1CCN(C(=O)c2ccc(-c3nnn[nH]3)cc2)CC1] |
| B48 | PMO^{thiol} (SH) | 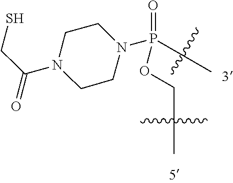 [SMILES: *COP(*)(=O)N1CCN(C(=O)CS)CC1] |
| B49 | PMO^{pros} | 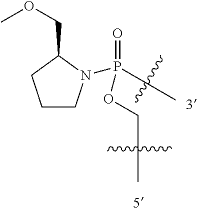 [SMILES: *COP(*)(=O)N1CCC[C@H]1COC] |
| B50 | PMO^{pror} | 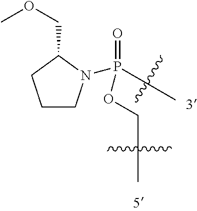 [SMILES: *COP(*)(=O)N1CCC[C@@H]1COC] |
| B51 | PMO^{tme} | 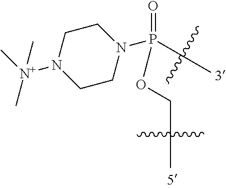 [SMILES: *COP(*)(=O)N1CCN([N+](C)(C)C)CC1] |
| B52 | PMO^{ca} | 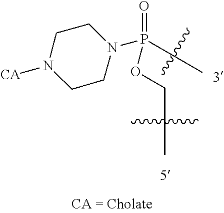 |
| B53 | PMO^{dca} | 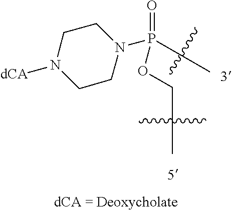 |
| B54 | PMO^{guan} (g) | 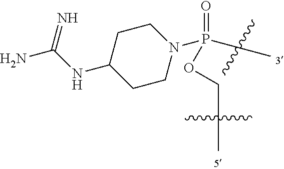 [SMILES: *COP(*)(=O)N1CCC(NC(=N)N)CC1] |
| B55 | PMO^{+phos} | 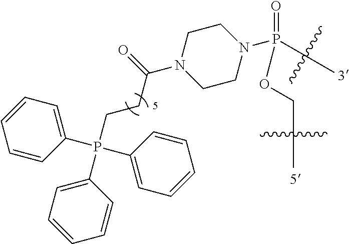 |
| B56 | PMO^{apnphos} | 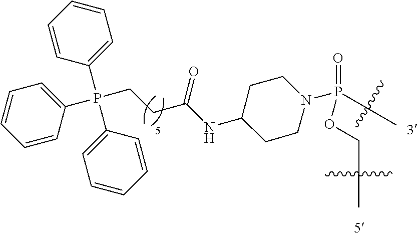 |

In the sequences and discussion that follows, the above names for the linkages are often used. For example, a base comprising a PMO^{apn} linkage is illustrated as ^{apn}B, where B is a base. Other linkages are designated similarly. In addition, abbreviated designations may be used, for example, the abbreviated designations in parentheses above may be used (e.g., ^{a}B, refers to ^{apn}B). Other readily identifiable abbreviations may also be used.

B. Oligomers with Modified Terminal Groups

In addition to the carrier peptide, the conjugate may also comprise an oligomer comprising modified terminal groups. Applicants have found that modification of the 3′ and/or 5′ end of the oligomer with various chemical moieties provides beneficial therapeutic properties (e.g., enhanced cell delivery, potency, and/or tissue distribution, etc.) to the conjugates. In various embodiments, the modified terminal groups comprise a hydrophobic moiety, while in other embodiments the modified terminal groups comprise a hydrophilic moiety. The modified terminal groups may be present with or without the linkages described above. For example, in some embodiments, the oligomers to which the carrier peptide is conjugated comprise one or more modified terminal groups and linkages of type (A), for example linkages wherein X is —N(CH₃)₂. In other embodiments, the oligomers comprise one or more modified terminal group and linkages of type (B), for example linkages wherein X is 4-aminopiperidin-1-yl (i.e., APN). In yet other embodiments, the oligomers comprise one or more modified terminal group and a mixture of linkages (A) and (B). For example, the oligomers may comprise one or more modified terminal group (e.g., trityl or triphenyl acetyl) and linkages wherein X is —N(CH₃)₂ and linkages wherein X is 4-aminopiperidin-1-yl. Other combinations of modified terminal groups and modified linkages also provide favorable therapeutic properties to the oligomers.

In one embodiment, the oligomers comprising terminal modifications have the following structure (XVII):

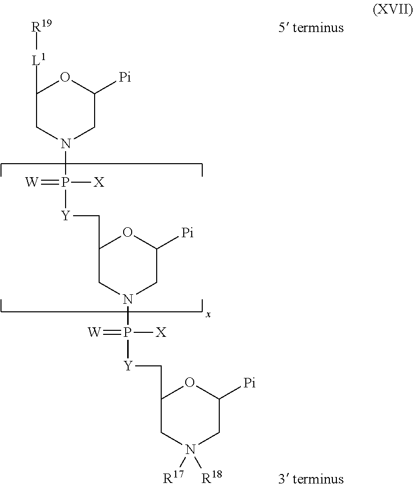

or a salt or isomer thereof, wherein X, W and Y are as defined above for any of linkages (A) and (B) and:
R¹⁷ is, at each occurrence, independently absent, hydrogen or C₁-C₆ alkyl;
R¹⁸ and R¹⁹ are, at each occurrence, independently absent, hydrogen, the carrier peptide, a natural or non-natural amino acid, C₂-C₃₀ alkylcarbonyl, —C(═O)OR²¹ or R²⁰;
R²⁰ is, at each occurrence, independently guanidinyl, heterocyclyl, C₁-C₃₀ alkyl, C₃-C₈ cycloalkyl; C₆-C₃₀ aryl, C₇-C₃₀ aralkyl, C₃-C₃₀ alkylcarbonyl, C₃-C₈ cycloalkylcarbonyl, C₃-C₈ cycloalkylalkylcarbonyl, C₇-C₃₀ arylcarbonyl, C₇-C₃₀ aralkylcarbonyl, C₂-C₃₀ alkyloxycarbonyl, C₃-C₈ cycloalkyloxycarbonyl, C₇-C₃₀ aryloxycarbonyl, C₈-C₃₀ aralkyloxycarbonyl, or —P(═O)(R₂₂)₂;
Pi is independently, at each occurrence, a base-pairing moiety;
L¹ is an optional linker up to 18 atoms in length comprising bonds selected from alkyl, hydroxyl, alkoxy, alkylamino, amide, ester, disulfide, carbonyl, carbamate, phosphorodiamidate, phosphoroamidate, phosphorothioate, piperazine and phosphodiester; and
x is an integer of 0 or greater; and wherein at least one of R¹⁸ or R¹⁹ is R²⁰; and
wherein at least one of R¹⁸ or R¹⁹ is R²⁰ and provided that both of R¹⁷ and R¹⁸ are not absent.

The oligomers with modified terminal groups may comprise any number of linkages of types (A) and (B). For example, the oligomers may comprise only linkage type (A). For example, X in each linkage may be —N(CH₃)₂. Alternatively, the oligomers may only comprise linkage (B). In certain embodiments, the oligomers comprise a mixture of linkages (A) and (B), for example from 1 to 4 linkages of type (B) and the remainder of the linkages being of type (A). Linkages in this regard include, but are not limited to, linkages wherein X is aminopiperidinyl for type (B) and dimethyl amino for type (A).

In some embodiments, R¹⁷ is absent. In some embodiments, R¹⁷ is hydrogen. In some embodiments, R¹⁷ is C₁-C₆ alkyl. In some embodiments, R¹⁷ is methyl. In yet other embodiments, R¹⁷ is ethyl. In some embodiments, R¹⁷ is C₃ alkyl. In some other embodiments, R¹⁷ is isopropyl. In other embodiments, R¹⁷ is C₄ alkyl. In yet other embodiments, R¹⁷ is C₅ alkyl. In some other embodiments, R¹⁷ is C₆ alkyl.

In other embodiments, R¹⁸ is absent. In some embodiments, R¹⁸ is hydrogen. In some embodiments, R¹⁸ is the carrier peptide. In some embodiments, R¹⁸ is a natural or non-natural amino acid, for example trimethylglycine. In some embodiments, R¹⁸ is R²⁰.

In other embodiments, R¹⁹ is absent. In some embodiments, R¹⁹ is hydrogen. In some embodiments, R¹⁹ is the carrier peptide. In some embodiments, R¹⁹ is a natural or non-natural amino acid, for example trimethylglycine. In some embodiments, R¹⁹ is —C(═O)OR¹⁷, for example R¹⁹ may have the following structure:

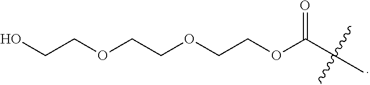 [SMILES: *C(=O)OCCOCCOCCO]

In other embodiments R¹⁸ or R¹⁹ is C₂-C₃₀ alkylcarbonyl, for example —C(═O)(CH₂)ₙCO₂H, where n is 1 to 6, for example 2. In other examples, R¹⁸ or R¹⁹ is acetyl.

In some embodiments, R²⁰ is, at each occurrence, independently guanidinyl, heterocyclyl, C₁-C₃₀ alkyl, C₃-C₈ cycloalkyl; C₆-C₃₀ aryl, C₇-C₃₀ aralkyl, C₃-C₃₀ alkylcarbonyl, C₃-C₈ cycloalkylcarbonyl, C₃-C₈ cycloalkylalkylcarbonyl, C₆-C₃₀ arylcarbonyl, C₇-C₃₀ aralkylcarbonyl, C₂-C₃₀ alkyloxycarbonyl, C₃-C₈ cycloalkyloxycarbonyl, C₇-C₃₀ aryloxycarbonyl, C₈-C₃₀ aralkyloxycarbonyl, —C(═O)OR²¹, or —P(═O)(R²²)₂, wherein R²¹ is C₁-C₃₀ alkyl comprising one or more oxygen or hydroxyl moieties or combinations thereof and each R²² is C⁶-C¹² aryloxy.

In certain other embodiments, R¹⁹ is —C(═O)OR²¹ and R¹⁸ is hydrogen, guanidinyl, heterocyclyl, C₁-C₃₀ alkyl, C₃-C₈ cycloalkyl; C₆-C₃₀ aryl, C₃-C₃₀ alkylcarbonyl, C₃-C₈ cycloalkylcarbonyl, C₃-C₈ cycloalkylalkylcarbonyl, C₇-C₃₀ arylcarbonyl, C₇-C₃₀ aralkylcarbonyl, C₂-C₃₀ alkyloxycarbonyl, C₃-C₈ cycloalkyloxycarbonyl, C₇-C₃₀ aryloxycarbonyl, C₈-C₃₀ aralkyloxycarbonyl, or —P(═O)(R²²)₂, wherein each R²² is C⁶-C¹² aryloxy.

In other embodiments, R²⁰ is, at each occurrence, independently guanidinyl, heterocyclyl, C₁-C₃₀ alkyl, C₃-C₈ cycloalkyl; C₆-C₃₀ aryl, C₃-C₃₀ alkylcarbonyl, C₃-C₈ cycloalkylcarbonyl, C₃-C₈ cycloalkylalkylcarbonyl, C₇-C₃₀ arylcarbonyl, C₇-C₃₀ aralkylcarbonyl, C₂-C₃₀ alkyloxycarbonyl, C₃-C₈ cycloalkyloxycarbonyl, C₇-C₃₀ aryloxycarbonyl, C₈-C₃₀ aralkyloxycarbonyl, or —P(═O)(R²²)₂. While in other examples, R²⁰ is, at each occurrence, independently guanidinyl, heterocyclyl, C₁-C₃₀ alkyl, C₃-C₈ cycloalkyl; C₆-C₃₀ aryl, C₇-C₃₀ aralkyl, C₃-C₈cycloalkylcarbonyl, C₃-C₈ cycloalkylalkylcarbonyl, C₇-C₃₀ arylcarbonyl, C₇-C₃₀ aralkylcarbonyl, C₂-C₃₀ alkyloxycarbonyl, C₃-C₈ cycloalkyloxycarbonyl, C₇-C₃₀ aryloxycarbonyl, C₈-C₃₀ aralkyloxycarbonyl, or —P(═O)(R²²)₂.

In some embodiments R²⁰ is guanidinyl, for example mono methylguanidynyl or dimethylguanidinyl. In other embodiments, R²⁰ is heterocyclyl. For example, in some embodiments, R²⁰ is piperidin-4-yl. In some embodiments, the piperidin-4-yl is substituted with trityl or Boc groups. In other embodiments, R²⁰ is C₃-C₈ cycloalkyl. In other embodiments, R²⁰ is C₆-C₃₀ aryl.

In some embodiments, R²⁰ is C₇-C₃₀ arylcarbonyl. For example, In some embodiments, R²⁰ has the following structure (XVIII):

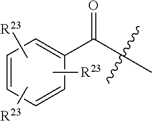

wherein R²³ is, at each occurrence, independently hydrogen, halo, C₁-C₃₀ alkyl, C₁-C₃₀ alkoxy, C₁-C₃₀ alkyloxycarbonyl, C₇-C₃₀ aralkyl, aryl, heteroaryl, heterocyclyl or heterocyclalkyl, and wherein one R²³ may join with another R²³ to form a heterocyclyl ring. In some embodiments, at least one R²³ is hydrogen, for example, in some embodiments, each R²³ is hydrogen. In other embodiments, at least one R²³ is C₁-C₃₀ alkoxy, for example in some embodiments, each R²³ is methoxy. In other embodiments, at least one R²³ is heteroaryl, for example in some embodiments, at least one R²³ has one of the following structures (XVIIIa) of (XVIIIb):

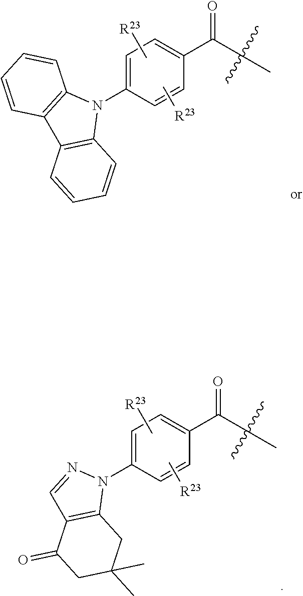

In still other embodiments, one R²³ joins with another R²³ to form a heterocyclyl ring. For example, in one embodiment, R²⁰ is 5-carboxyfluorescein.

In other embodiments, R²⁰ is C₇-C₃₀ aralkylcarbonyl. For example, in various embodiments, R²⁰ has one of the following structures (XIX), (XX) or (XXI):

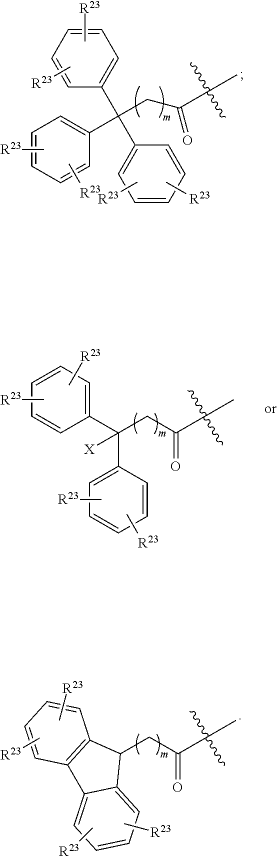

wherein R²³ is, at each occurrence, independently hydrogen, halo, C₁-C₃₀ alkyl, C₁-C₃₀ alkoxy, C₁-C₃₀ alkyloxycarbonyl, C₇-C₃₀ aralkyl, aryl, heteroaryl, heterocyclyl or heterocyclalkyl, wherein one R²³ may join with another R²³ to form a heterocyclyl ring, X is —OH or halo and m is an integer from 0 to 6. In some specific embodiments, m is 0. In other embodiments, m is 1, while in other embodiments, m is 2. In other embodiments, at least one R²³ is hydrogen, for example in some embodiments each R²³ is hydrogen. In some embodiments, X is hydrogen. In other embodiments, X is —OH. In other embodiments, X is C₁. In other embodiments, at least one R²³ is C₁-C₃₀ alkoxy, for example methoxy.

In still other embodiments, R²⁰ is C₇-C₃₀ aralkyl, for example trityl. In other embodiments, R²⁰ is methoxy trityl. In some embodiments, R²⁰ has the following structure (XXII):

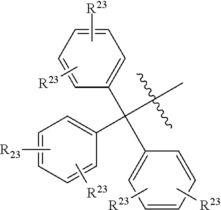

wherein R²³ is, at each occurrence, independently hydrogen, halo, C₁-C₃₀ alkyl, C₁-C₃₀ alkoxy, C₁-C₃₀ alkyloxycarbonyl, C₇-C₃₀ aralkyl, aryl, heteroaryl, heterocyclyl or heterocyclalkyl, and wherein one R²³ may join with another R²³ to form a heterocyclyl ring. For example, in some embodiments each R²³ is hydrogen. In other embodiments, at least one R²³ is C₁-C₃₀ alkoxy, for example methoxy.

In yet other embodiments, R²⁰ is C₇-C₃₀ aralkyl and R²⁰ has the following structure (XXIII):

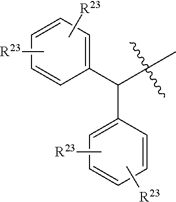

In some embodiments, at least one R²³ is halo, for example chloro. In some other embodiments, one R²³ is chloro in the para position.

In other embodiments, R²⁰ is C₁-C₃₀ alkyl. For example, In some embodiments, R²⁰ is a C₄-C₂₀ alkyl and optionally comprises one or more double bonds.

For example, In some embodiments, R²⁰ is a C₄₋₁₀ alkyl comprising a triple bond, for example a terminal triple bond. In some embodiments, R²⁰ is hexyn-6-yl. In some embodiments, R²⁰ has one of the following structures (XXIV), (XXV), (XXVI) or (XXVII):

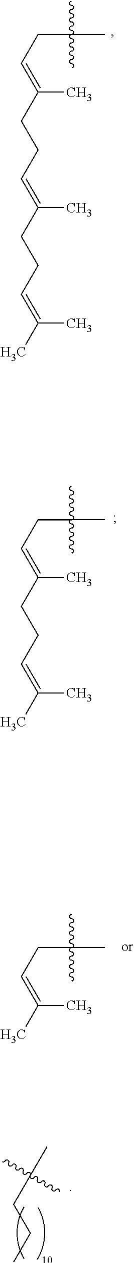

In still other embodiments, R²⁰ is a C₃-C₃₀ alkylcarbonyl, for example a C₃-C₁₀ alkyl carbonyl. In some embodiments, R²⁰ is —C(═O)(CH₂)ₚSH or —C(═O)(CH₂)ₚSSHet, wherein p is an integer from 1 to 6 and Het is a heteroaryl. For example, p may be 1 or p may be 2. In other example Het is pyridinyl, for example pyridin-2-yl. In other embodiments, the C₃-C₃₀ alkylcarbonyl is substituted with a further oligomer, for example in some embodiments the oligomer comprises a C₃-C₃₀ alkyl carbonyl at the 3′ position which links the oligomer to the 3′ position of another oligomer. Such terminal modifications are included within the scope of the present disclosure.

In other embodiments, R²⁰ is a C₃-C₃₀ alkyl carbonyl which is further substituted with an arylphosphoryl moiety, for example triphenyl phosphoryl. Examples of such R²⁰ groups include structure 33 in Table 3.

In other examples, R²⁰ is C₃-C₈ cycloalkylcarbonyl, for example C₅-C₇ alkyl carbonyl. In these embodiments, R²⁰ has the following structure (XXVIII):

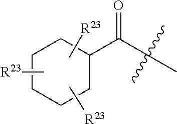

wherein R²³ is, at each occurrence, independently hydrogen, halo, C₁-C₃₀ alkyl, C₁-C₃₀ alkoxy, C₁-C₃₀ alkyloxycarbonyl, C₇-C₃₀ aralkyl, aryl, heteroaryl, heterocyclyl or heterocyclalkyl, and wherein one R²³ may join with another R²³ to form a heterocyclyl ring. In some embodiments, R²³ is heterocyclylalkyl, for example in some embodiments R²³ has the following structure:

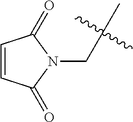 [SMILES: *CN1C(=O)C=CC1=O]

In some other embodiments, R²⁰ is C₃-C₈ cycloalkylalkylcarbonyl. In other embodiments, R²⁰ is C₂-C₃₀ alkyloxycarbonyl. In other embodiments, R²⁰ is C₃-C₈ cycloalkyloxycarbonyl. In other embodiments, R²⁰ is C₇-C₃₀ aryloxycarbonyl. In other embodiments, R²⁰ is C₈-C₃₀ aralkyloxycarbonyl. In other embodiments, R²⁰ is —P(═O)(R²²)₂, wherein each R²² is C⁶-C¹² aryloxy, for example in some embodiments R²⁰ has the following structure (C₂₄):

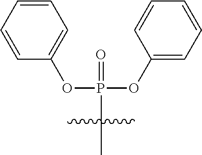 [SMILES: *P(=O)(Oc1ccccc1)Oc1ccccc1]

In other embodiments, R²⁰ comprises one or more halo atoms. For example, in some embodiments R²⁰ comprises a perfluoro analogue of any of the above R²⁰ moieties. In other embodiments, R²⁰ is p-trifluoromethylphenyl, trifluoromethyltrityl, perfluoropentyl or pentafluorophenyl.

In some embodiments the 3′ terminus comprises a modification and in other embodiments the 5′ terminus comprises a modification. In other embodiments both the 3′ and 5′ termini comprise modifications. Accordingly, in some embodiments, R¹⁸ is absent and R¹⁹ is R²⁰. In other embodiments, R¹⁹ is absent and R¹⁸ is R²⁰. In yet other embodiments, R¹⁸ and R¹⁹ are each R²⁰.

In some embodiments, the oligomer comprises a cell-penetrating peptide in addition to a 3′ or 5′ modification. Accordingly, in some embodiments R¹⁹ is a cell-penetrating peptide and R¹⁸ is R²⁰. In other embodiments, R¹⁸ is a cell-penetrating peptide and R¹⁹ is R²⁰. In further embodiments of the foregoing, the cell-penetrating peptide is an arginine-rich peptide.

In some embodiments, the linker Lⁱ which links the 5′terminal group (i.e., R¹⁹) to the oligomer may be present or absent. The linker comprises any number of functional groups and lengths provided the linker retains its ability to link the 5′ terminal group to the oligomer and provided that the linker does not interfere with the oligomer's ability to bind to a target sequence in a sequence specific manner. In one embodiment, L comprises phosphorodiamidate and piperazine bonds. For example, in some embodiments L has the following structure (XXIX):

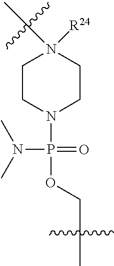

wherein R²⁴ is absent, hydrogen or C₁-C₆ alkyl. In some embodiments, R²⁴ is absent. In some embodiments, R²⁴ is hydrogen. In some embodiments, R²⁴ is C₁-C₆ alkyl. In some embodiments, R²⁴ is methyl. In other embodiments, R²⁴ is ethyl. In yet other embodiments, R²⁴ is C₃ alkyl. In some other embodiments, R²⁴ is isopropyl. In yet other embodiments, R²⁴ is C₄ alkyl. In some embodiments, R²⁴ is C₅ alkyl. In yet other embodiments, R²⁴ is C₆ alkyl.

In yet other embodiments, R²⁰ is C₃-C₃₀ alkylcarbonyl, and R²⁰ has the following structure (XXX):

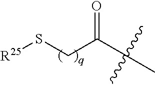

wherein R²⁵ is hydrogen or —SR²⁶, wherein R²⁶ is hydrogen, C₁-C₃₀ alkyl, heterocyclyl, aryl or heteroaryl, and q is an integer from 0 to 6.

In further embodiments of any of the above, R²³ is, at each occurrence, independently hydrogen, halo, C₁-C₃₀ alkyl, C₁-C₃₀ alkoxy, aryl, heteroaryl, heterocyclyl or heterocyclalkyl.

In some other embodiments, only the 3′ terminus of the oligomer is conjugated to one of the groups noted above. In some other embodiments, only the 5′ terminus of the oligomer is conjugated to one of the groups noted above. In other embodiments, both the 3′ and 5′ termini comprise one of the groups noted above. The terminal group may be selected from any one of the groups noted above or any of the specific groups illustrated in Table 3.

TABLE 3
Representative Terminal Groups

| No. | Name | Structure |
|---|---|---|
| C1 | Trimethoxybenzoyl | 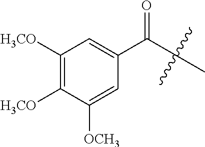 [SMILES: *C(=O)c1cc(OC)c(OC)c(OC)c1] |
| C2 | 9-fluorene-carboxyl | 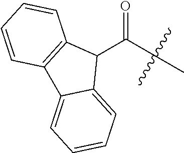 [SMILES: *C(=O)C1c2ccccc2-c2ccccc21] |
| C3 | 4-carbazolylbenzoyl | 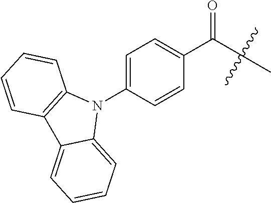 [SMILES: *C(=O)c1ccc(-n2c3ccccc3c3ccccc32)cc1] |
| C4 | 4-indazolylonebenzoyl | 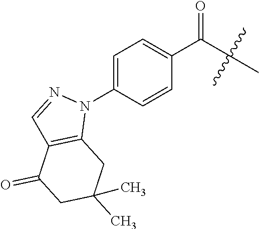 [SMILES: *C(=O)c1ccc(-n2ncc3c2CC(C)(C)CC3=O)cc1] |
| C5 | Farnesyl | 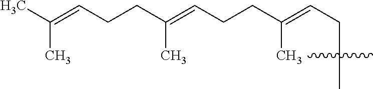 [SMILES: *C/C=C(\C)CC/C=C(\C)CCC=C(C)C] |
| C6 | Geranyl | 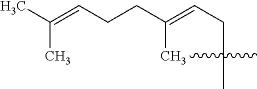 [SMILES: *C/C=C(\C)CCC=C(C)C] |
| C7 | Prenyl | 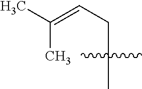 [SMILES: *CC=C(C)C] |
| C8 | Diphenylacetyl | 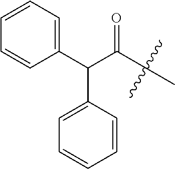 [SMILES: *C(=O)C(c1ccccc1)c1ccccc1] |
| C9 | Chlorodiphenylacetyl | 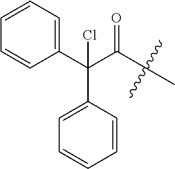 [SMILES: *C(=O)C(Cl)(c1ccccc1)c1ccccc1] |
| C10 | Hydroxydiphenylacetyl | 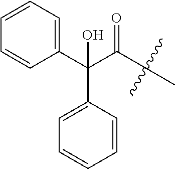 [SMILES: *C(=O)C(O)(c1ccccc1)c1ccccc1] |
| C11 | Triphenylpropionyl | 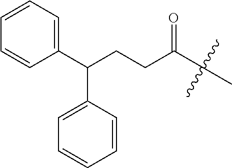 [SMILES: *C(=O)CCC(c1ccccc1)c1ccccc1] |
| C12 | Triphenylpropyl | 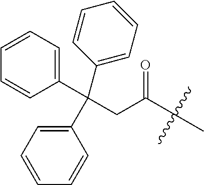 [SMILES: *C(=O)CC(c1ccccc1)(c1ccccc1)c1ccccc1] |
| C13 | Triphenylacetyl | 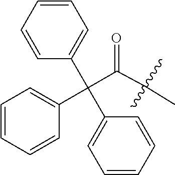 [SMILES: *C(=O)C(c1ccccc1)(c1ccccc1)c1ccccc1] |
| C14 | Trityl (Tr) | 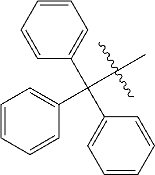 [SMILES: *C(c1ccccc1)(c1ccccc1)c1ccccc1] |
| C15 | Methoxytrityl (MeOTr) | 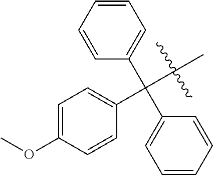 [SMILES: *C(c1ccccc1)(c1ccccc1)c1ccc(OC)cc1] |
| C16 | Methylsuccinimidyl- cyclohexoyl | 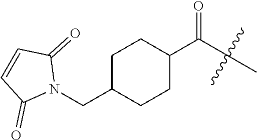 [SMILES: *C(=O)C1CCC(CN2C(=O)C=CC2=O)CC1] |
| C17 | Thioacetyl | 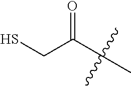 [SMILES: *C(=O)CS] |
| C18 | COCH₂CH₂SSPy | 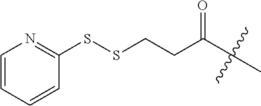 [SMILES: *C(=O)CCSSc1ccccn1] |
| C19 | Guanidinyl | 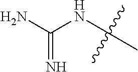 [SMILES: *NC(=N)N] |
| C20 | Trimethylglycine | 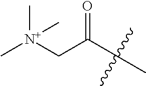 [SMILES: *C(=O)C[N+](C)(C)C] |
| C21 | Lauroyl | 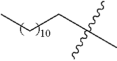 |
| C22 | Triethyleneglycoloyl (EG3) | 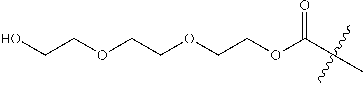 [SMILES: *C(=O)OCCOCCOCCO] |
| C23 | Succinicacetyl | 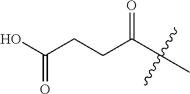 [SMILES: *C(=O)CCC(=O)O] |
| C24 | Diphenylphosphoryl | 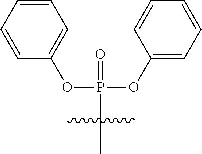 [SMILES: *P(=O)(Oc1ccccc1)Oc1ccccc1] |
| C25 | Piperidin-4-yl | 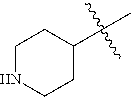 [SMILES: *C1CCNCC1] |
| C26 | Tritylpiperidin-4-yl | 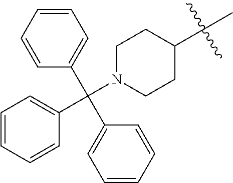 [SMILES: *C1CCN(C(c2ccccc2)(c2ccccc2)c2ccccc2)CC1] |
| C27 | Boc-Piperidin-4-yl | 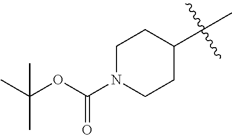 [SMILES: *C1CCN(C(=O)OC(C)(C)C)CC1] |
| C28 | Hexyl-6-yl | 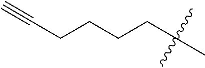 [SMILES: *CCCCC#C] |
| C29 | 5-carboxyfluorescein | 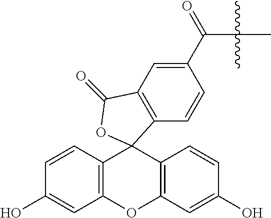 [SMILES: *C(=O)c1ccc2c(c1)C(=O)OC21c2ccc(O)cc2Oc2cc(O)ccc21] |
| C30 | Benzhydryl | 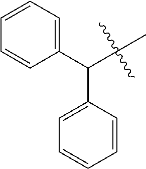 [SMILES: *C(c1ccccc1)c1ccccc1] |
| C31 | p-Chlorobenzhydryl | 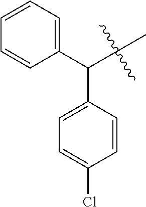 [SMILES: *C(c1ccccc1)c1ccc(Cl)cc1] |
| C32 | Piperazinyl (pip) | 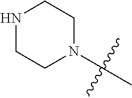 [SMILES: *N1CCNCC1] |
| C33 | Triphenylphos | 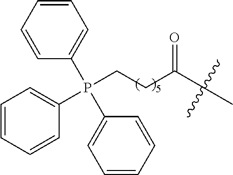 |
| C34 | Dimerized | 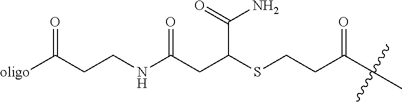 |

### C. Properties of the Conjugates

As noted above, the present disclosure is directed to conjugates of carrier peptides and oligonucleotide analogues (i.e., oligomers). The oligomers may comprise various modifications which impart desirable properties (e.g., increased antisense activity) to the oligomers. In certain embodiments, the oligomer comprises a backbone comprising a sequence of morpholino ring structures joined by intersubunit linkages, the intersubunit linkages joining a 3′-end of one morpholino ring structure to a 5′-end of an adjacent morpholino ring structure, wherein each morpholino ring structure is bound to a base-pairing moiety, such that the oligomer can bind in a sequence-specific manner to a target nucleic acid. The morpholino ring structures may have the following structure (i):

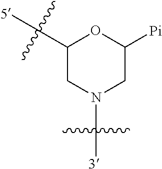

wherein Pi is, at each occurrence, independently a base-pairing moiety.

Each morpholino ring structure supports a base pairing moiety (Pi), to form a sequence of base pairing moieties which is typically designed to hybridize to a selected antisense target in a cell or in a subject being treated. The base pairing moiety may be a purine or pyrimidine found in native DNA or RNA (A, G, C, T, or U) or an analog, such as hypoxanthine (the base component of the nucleoside inosine) or 5-methyl cytosine. Analog bases that confer improved binding affinity to the oligomer can also be utilized. Exemplary analogs in this regard include C5-propynyl-modified pyrimidines, 9-(aminoethoxy)phenoxazine (G-clamp) and the like.

As noted above, the oligomer may be modified, in accordance with an aspect of the invention, to include one or more (B) linkages, e.g. up to about 1 per every 2-5 uncharged linkages, typically 3-5 per every 10 uncharged linkages. Certain embodiments also include one or more linkages of type (B). In some embodiments, optimal improvement in antisense activity is seen where up to about half of the backbone linkages are type (B). Some, but not maximum enhancement is typically seen with a small number e.g., 10-20% of (B) linkages.

In one embodiment, the linkage types (A) and (B) are interspersed along the backbone. In some embodiments, the oligomer does not have a strictly alternating pattern of (A) and (B) linkages along its entire length. In addition to the carrier peptide, the oligomers may optionally comprise a 5′ and/or 3′ modification as described above.

Also considered are oligomers having blocks of (A) linkages and blocks of (B) linkages; for example, a central block of (A) linkages may be flanked by blocks of (B) linkages, or vice versa. In one embodiment, the oligomer has approximately equal-length 5′,3; and center regions, and the percentage of (B) or (A) linkages in the center region is greater than about 50%, o greater than about 70%. Oligomers for use in antisense applications generally range in length from about 10 to about 40 subunits, more preferably about 15 to 25 subunits. For example, an oligomer of the invention having 19-20 subunits, a useful length for an antisense oligomer, may ideally have two to seven, e.g. four to six, or three to five, (B) linkages, and the remainder (A) linkages. An oligomer having 14-15 subunits may ideally have two to five, e.g. 3 or 4, (B) linkages and the remainder (A) linkages.

The morpholino subunits may also be linked by non-phosphorus-based intersubunit linkages, as described further below.

Other oligonucleotide analog linkages which are uncharged in their unmodified state but which could also bear a pendant amine substituent can also be used. For example, a 5′nitrogen atom on a morpholino ring could be employed in a sulfamide linkage (or a urea linkage, where phosphorus is replaced with carbon or sulfur, respectively).

In some embodiments for antisense applications, the oligomer may be 100% complementary to the nucleic acid target sequence, or it may include mismatches, e.g., to accommodate variants, as long as a heteroduplex formed between the oligomer and nucleic acid target sequence is sufficiently stable to withstand the action of cellular nucleases and other modes of degradation which may occur in vivo. Mismatches, if present, are less destabilizing toward the end regions of the hybrid duplex than in the middle. The number of mismatches allowed will depend on the length of the oligomer, the percentage of G:C base pairs in the duplex, and the position of the mismatch(es) in the duplex, according to well understood principles of duplex stability. Although such an antisense oligomer is not necessarily 100% complementary to the nucleic acid target sequence, it is effective to stably and specifically bind to the target sequence, such that a biological activity of the nucleic acid target, e.g., expression of encoded protein(s), is modulated.

The stability of the duplex formed between an oligomer and the target sequence is a function of the binding Tₘ and the susceptibility of the duplex to cellular enzymatic cleavage. The Tₘ of an antisense compound with respect to complementary-sequence RNA may be measured by conventional methods, such as those described by Hames et al., Nucleic Acid Hybridization, TRL Press, 1985, pp. 107-108 or as described in Miyada C.G. and Wallace R.B., 1987, Oligonucleotide hybridization techniques, Methods Enzymol. Vol. 154 pp. 94-107.

In some embodiments, each antisense oligomer has a binding Tₘ, with respect to a complementary-sequence RNA, of greater than body temperature or in other embodiments greater than 50 °C. In other embodiments Tₘ's are in the range 60-80 °C or greater. According to well known principles, the Tₘ of an oligomer compound, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligomer. For this reason, compounds that show high Tₘ (50 °C or greater) at a length of 20 bases or less are generally preferred over those requiring greater than 20 bases for high Tₘ values. For some applications, longer oligomers, for example longer than 20 bases may have certain advantages. For example, in certain embodiments longer oligomers may find particular utility for use in exon skipping or splice modulation.

The targeting sequence bases may be normal DNA bases or analogues thereof, e.g., uracil and inosine that are capable of Watson-Crick base pairing to target-sequence RNA bases.

The oligomers may also incorporate guanine bases in place of adenine when the target nucleotide is a uracil residue. This is useful when the target sequence varies across different viral species and the variation at any given nucleotide residue is either cytosine or uracil. By utilizing guanine in the targeting oligomer at the position of variability, the well-known ability of guanine to base pair with uracil (termed C/U:G base pairing) can be exploited. By incorporating guanine at these locations, a single oligomer can effectively target a wider range of RNA target variability.

The compounds (e.g., oligomers, intersubunit linkages, terminal groups) may exist in different isomeric forms, for example structural isomers (e.g., tautomers). With regard to stereoisomers, the compounds may have chiral centers and may occur as racemates, enantiomerically enriched mixtures, individual enantiomers, mixture or diastereomers or individual diastereomers. All such isomeric forms are included within the present invention, including mixtures thereof. The compounds may also possess axial chirality which may result in atropisomers. Furthermore, some of the crystalline forms of the compounds may exist as polymorphs, which are included in the present invention. In addition, some of the compounds may also form solvates with water or other organic solvents. Such solvates are similarly included within the scope of this invention.

The oligomers described herein may be used in methods of inhibiting production of a protein or replication of a virus. Accordingly, in one embodiment a nucleic acid encoding such a protein is exposed to an oligomer as disclosed herein. In further embodiments of the foregoing, the antisense oligomer comprises either a 5′ or 3′ modified terminal group or combinations thereof, as disclosed herein, and the base pairing moieties B form a sequence effective to hybridize to a portion of the nucleic acid at a location effective to inhibit production of the protein. In one embodiment, the location is an ATG start codon region of an mRNA, a splice site of a pre-mRNA, or a viral target sequence as described below.

In one embodiment, the oligomer has a Tₘ with respect to binding to the target sequence of greater than about 50 °C, and it is taken up by mammalian cells or bacterial cells. In another embodiment, the oligomer may be conjugated to a transport moiety, for example an arginine-rich peptide, as described herein to facilitate such uptake. In another embodiment, the terminal modifications described herein can function as a transport moiety to facilitate uptake by mammalian and/or bacterial cells.

The preparation and properties of morpholino oligomers is described in more detail below and in U.S. Pat. No. 5,185,444 and WO/2009/064471, each of which is hereby incorporated by reference in their entirety.

### D. Formulation and Administration of the Conjugates

The present disclosure also provides for formulation and delivery of the disclosed conjugate. Accordingly, in one embodiment the present disclosure is directed to a composition comprising a peptide-oligomer conjugate as disclosed herein and a pharmaceutically acceptable vehicle.

Effective delivery of the conjugate to the target nucleic acid is an important aspect of treatment. Routes of antisense oligomer delivery include, but are not limited to, various systemic routes, including oral and parenteral routes, e.g., intravenous, subcutaneous, intraperitoneal, and intramuscular, as well as inhalation, transdermal and topical delivery. The appropriate route may be determined by one of skill in the art, as appropriate to the condition of the subject under treatment. For example, an appropriate route for delivery of an antisense oligomer in the treatment of a viral infection of the skin is topical delivery, while delivery of a antisense oligomer for the treatment of a viral respiratory infection is by inhalation. The oligomer may also be delivered directly to the site of viral infection, or to the bloodstream.

The conjugate may be administered in any convenient vehicle which is physiologically and/or pharmaceutically acceptable. Such a composition may include any of a variety of standard pharmaceutically acceptable carriers employed by those of ordinary skill in the art. Examples include, but are not limited to, saline, phosphate buffered saline (PBS), water, aqueous ethanol, emulsions, such as oil/water emulsions or triglyceride emulsions, tablets and capsules. The choice of suitable physiologically acceptable carrier will vary dependent upon the chosen mode of administration.

The compounds (e.g., conjugates) of the present invention may generally be utilized as the free acid or free base. Alternatively, the compounds of this invention may be used in the form of acid or base addition salts. Acid addition salts of the free amino compounds of the present invention may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” of structure (I) is intended to encompass any and all acceptable salt forms.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of structure (I). Further, in the case of a carboxylic acid (—COOH), esters may be employed, such as methyl esters, ethyl esters, and the like.

In some instances, liposomes may be employed to facilitate uptake of the antisense oligonucleotide into cells. (See, e.g., Williams, S.A., Leukemia 10(12):1980-1989, 1996; Lappalainen et al., Antiviral Res. 23:119, 1994; Uhlmann et al., antisense oligonucleotides: a new therapeutic principle, Chemical Reviews, Volume 90, No. 4, pages 544-584, 1990; Gregoriadis, G., Chapter 14, Liposomes, Drug Carriers in Biology and Medicine, pp. 287-341, Academic Press, 1979). Hydrogels may also be used as vehicles for antisense oligomer administration, for example, as described in WO 93/01286. Alternatively, the oligonucleotides may be administered in microspheres or microparticles. (See, e.g., Wu, G.Y. and Wu, C.H., J. Biol. Chem. 262:4429-4432, 1987). Alternatively, the use of gas-filled microbubbles complexed with the antisense oligomers can enhance delivery to target tissues, as described in U.S. Pat. No. 6,245,747. Sustained release compositions may also be used. These may include semipermeable polymeric matrices in the form of shaped articles such as films or microcapsules.

In one embodiment, antisense inhibition is effective in treating infection of a host animal by a virus, by contacting a cell infected with the virus with an antisense agent effective to inhibit the replication of the specific virus. The antisense agent is administered to a mammalian subject, e.g., human or domestic animal, infected with a given virus, in a suitable pharmaceutical carrier. It is contemplated that the antisense oligonucleotide arrests the growth of the RNA virus in the host. The RNA virus may be decreased in number or eliminated with little or no detrimental effect on the normal growth or development of the host.

In one aspect of the method, the subject is a human subject, e.g., a patient diagnosed as having a localized or systemic viral infection. The condition of a patient may also dictate prophylactic administration of an antisense oligomer of the invention, e.g. in the case of a patient who (1) is immunocompromised; (2) is a burn victim; (3) has an indwelling catheter; or (4) is about to undergo or has recently undergone surgery. In one preferred embodiment, the oligomer is a phosphorodiamidate morpholino oligomer, contained in a pharmaceutically acceptable carrier, and is delivered orally. In another preferred embodiment, the oligomer is a phosphorodiamidate morpholino oligomer, contained in a pharmaceutically acceptable carrier, and is delivered intravenously (i.v.).

In another application of the method, the subject is a livestock animal, e.g., a chicken, turkey, pig, cow or goat, etc, and the treatment is either prophylactic or therapeutic. The invention also includes a livestock and poultry food composition containing a food grain supplemented with a subtherapeutic amount of an antiviral antisense compound of the type described above. Also contemplated is, in a method of feeding livestock and poultry with a food grain supplemented with subtherapeutic levels of an antiviral, an improvement in which the food grain is supplemented with a subtherapeutic amount of an antiviral oligonucleotide composition as described above.

In one embodiment, the conjugate is administered in an amount and manner effective to result in a peak blood concentration of at least 200-400 nM antisense oligomer. Typically, one or more doses of antisense oligomer are administered, generally at regular intervals, for a period of about one to two weeks. Preferred doses for oral administration are from about 1-1000 mg oligomer per 70 kg. In some cases, doses of greater than 1000 mg oligomer/patient may be necessary. For i.v. administration, preferred doses are from about 0.5 mg to 1000 mg oligomer per 70 kg. The conjugate may be administered at regular intervals for a short time period, e.g., daily for two weeks or less. However, in some cases the conjugate is administered intermittently over a longer period of time. Administration may be followed by, or concurrent with, administration of an antibiotic or other therapeutic treatment. The treatment regimen may be adjusted (dose, frequency, route, etc.) as indicated, based on the results of immunoassays, other biochemical tests and physiological examination of the subject under treatment.

An effective in vivo treatment regimen using the conjugates of the invention may vary according to the duration, dose, frequency and route of administration, as well as the condition of the subject under treatment (i.e., prophylactic administration versus administration in response to localized or systemic infection). Accordingly, such in vivo therapy will often require monitoring by tests appropriate to the particular type of viral infection under treatment, and corresponding adjustments in the dose or treatment regimen, in order to achieve an optimal therapeutic outcome. Treatment may be monitored, e.g., by general indicators of disease and/or infection, such as complete blood count (CBC), nucleic acid detection methods, immunodiagnostic tests, viral culture, or detection of heteroduplex.

The efficacy of an in vivo administered antiviral conjugate of the invention in inhibiting or eliminating the growth of one or more types of RNA virus may be determined from biological samples (tissue, blood, urine etc.) taken from a subject prior to, during and subsequent to administration of the antisense oligomer. Assays of such samples include (1) monitoring the presence or absence of heteroduplex formation with target and non-target sequences, using procedures known to those skilled in the art, e.g., an electrophoretic gel mobility assay; (2) monitoring the amount of viral protein production, as determined by standard techniques such as ELISA or Western blotting, or (3) measuring the effect on viral titer, e.g. by the method of Spearman-Karber. (See, for example, Pari, G. S. et al., Antimicrob. Agents and Chemotherapy 39(5):1157-1161, 1995; Anderson, K. P. et al., Antimicrob. Agents and Chemotherapy 40(9):2004-2011, 1996, Cottral, G.E. (ed) in: Manual of Standard Methods for Veterinary Microbiology, pp. 60-93, 1978).

### E. Preparation of the Conjugates

The morpholino subunits, the modified intersubunit linkages and oligomers comprising the same can be prepared as described in the examples and in U.S. Pat. Nos. 5,185,444 and 7,943,762 which are hereby incorporated by reference in their entirety. The morpholino subunits can be prepared according to the following general Reaction Scheme I.

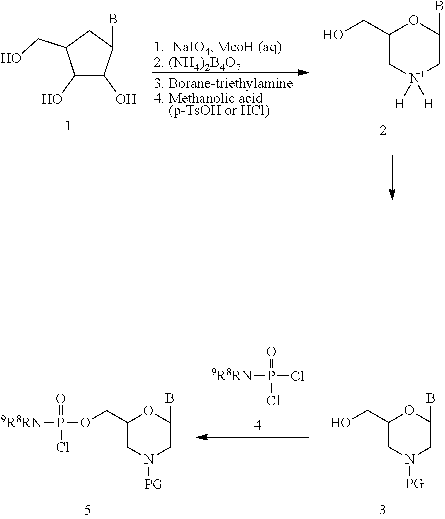

Referring to Reaction Scheme 1, wherein B represents a base pairing moiety and PG represents a protecting group, the morpholino subunits may be prepared from the corresponding ribinucleoside (1) as shown. The morpholino subunit (2) may be optionally protected by reaction with a suitable protecting group precursor, for example trityl chloride. The 3′ protecting group is generally removed during solid-state oligomer synthesis as described in more detail below. The base pairing poiety may be suitable protected for sold phase oligomer synthesis. Suitable protecting groups include benzoyl for adenine and cytosine, phenylacetyl for guanine, and pivaloyloxymethyl for hypoxanthine (I). The pivaloyloxymethyl group can be introduced onto the N1 position of the hypoxanthine heterocyclic base. Although an unprotected hypoxanthine subunit, may be employed, yields in activation reactions are far superior when the base is protected. Other suitable protecting groups include those disclosed in co-pending U.S. application Ser. No. 12/271,040, which is hereby incorporated by reference in its entirety.

Reaction of 3 with the activated phosphorous compound 4, results in morpholino subunints having the desired linkage moiety (5). Compounds of structure 4 can be prepared using any number of methods known to those of skill in the art. For example, such compounds may be prepared by reaction of the corresponding amine and phosphorous oxychloride. In this regard, the amine starting material can be prepared using any method known in the art, for example those methods described in the Examples and in U.S. Pat. No. 7,943,762. Although the above scheme depicts preparation of linkages of type (B) (e.g., X is —NR⁸R⁹), linkages of type (A) (e.g., X is dimethyl amine) can be prepared in an analogous manner.

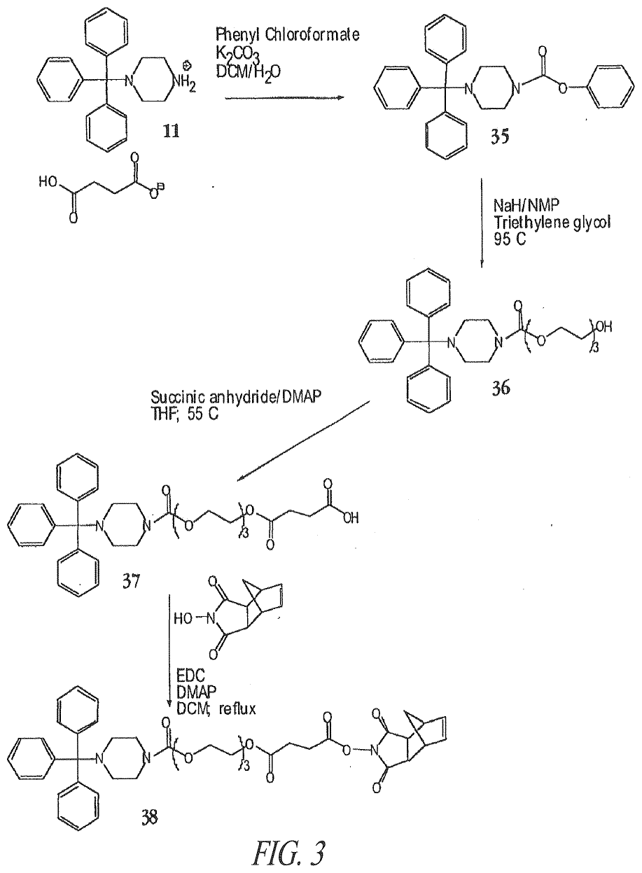

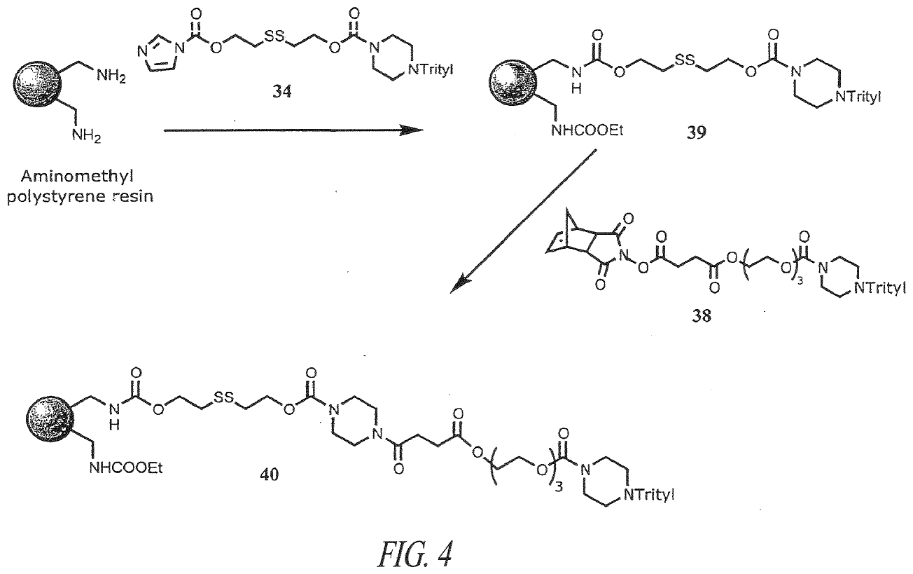

Compounds of structure 5 can be used in solid-phase automated oligomer synthesis for preparation of oligomers comprising the intersubunit linkages. Such methods are well known in the art. Briefly, a compound of structure 5 may be modified at the 5′ end to contain a linker to a solid support. For example, compound 5 may be linked to a solid support by a linker comprising L¹ and/or R¹⁹. An exemplary method is demonstrated in FIGS. 3 and 4. In this manner, the oligo may comprise a 5′-terminal modification after oligomer synthesis is complete and the oligomer is cleaved from the solid support. Once supported, the protecting group of 5 (e.g., trityl) is removed and the free amine is reacted with an activated phosphorous moiety of a second compound of structure 5. This sequence is repeated until the desired length oligo is obtained. The protecting group in the termina 5′ end may either be removed or left on if a 5′-modification is desired. The oligo can be removed from the solid support using any number of methods, or example treatment with a base to cleave the linkage to the solid support.

Peptide oligomer conjugates can be prepared by coupling the desired peptide (prepared according to standard peptide synthetic methods known in the art) with an oligomer comprising a free NH (for example the 3′ NH of morpholino oligomer) in the presence of an appropriate activating reagent (e.g., HATU). Conjugates may be purified using a number of techniques known in the art, for example SCX chromatography.

The preparation of modified morpholino subunits and peptide oligomer conjugates are described in more detail in the Examples. The peptide oligomer conjugates containing any number of modified linkages may be prepared using methods described herein, methods known in the art and/or described by reference herein. Also described in the examples are global modifications of PMO+ morpholino oligomers prepared as previously described (see e.g., PCT publication WO2008036127).

### F. Antisense Activity of the Oligomers

The present disclosure also provides a method of inhibiting production of a protein, the method comprising exposing a nucleic acid encoding the protein to a peptide-oligomer conjugate as disclosed herein. Accordingly, in one embodiment a nucleic acid encoding such a protein is exposed to a conjugate, as disclosed herein, where the base pairing moieties Pi form a sequence effective to hybridize to a portion of the nucleic acid at a location effective to inhibit production of the protein. The oligomer may target, for example, an ATG start codon region of an mRNA, a splice site of a pre-mRNA, or a viral target sequence as described below.

In another embodiment, the disclosure provides a method of enhancing antisense activity of a peptide oligomer conjugate comprising an oligonucleotide analogue having a sequence of morpholino subunits, joined by intersubunit linkages, supporting base-pairing moieties, the method comprises conjugating a carrier peptide as described herein to the oligonucleotide.

In some embodiments, enhancement of antisense activity may be evidenced by:
(i) a decrease in expression of an encoded protein, relative to that provided by a corresponding unmodified oligomer, when binding of the antisense oligomer to its target sequence is effective to block a translation start codon for the encoded protein, or
(ii) an increase in expression of an encoded protein, relative to that provided by a corresponding unmodified oligomer, when binding of the antisense oligomer to its target sequence is effective to block an aberrant splice site in a pre-mRNA which encodes said protein when correctly spliced. Assays suitable for measurement of these effects are described further below. In one embodiment, modification provides this activity in a cell-free translation assay, a splice correction translation assay in cell culture, or a splice correction gain of function animal model system as described herein. In one embodiment, activity is enhanced by a factor of at least two, at least five or at least ten.

Described below are various exemplary applications of the conjugates of the invention including antiviral applications, treatment of neuromuscular diseases, bacterial infections, inflammation and polycystic kidney disease. This description is not meant to limit the invention in any way but serves to exemplify the range of human and animal disease conditions that can be addressed using the conjugates described herein.

### G. Exemplary Therapeutic Uses of the Conjugates

The oligomers conjugated to the carrier peptide comprise good efficacy and low toxicity, thus resulting in a better therapeutic window than obtained with other oligomers or peptide-oligomer conjugates. The following description provides exemplary, but not limiting, example of therapeutic uses of the conjugates.

1. Targeting Stem-Loop Secondary Structure of ssRNA Viruses

One class of an exemplary antisense antiviral compound is a morpholino oligomer as described herein having a sequence of 12-40 subunits and a targeting sequence that is complementary to a region associated with stem-loop secondary structure within the 5′-terminal end 40 bases of the positive-sense RNA strand of the targeted virus. (See, e.g., PCT Pubn. No. WO/2006/033933 or U.S. Appn. Pubn. Nos. 20060269911 and 20050096291, which are incorporated herein by reference.)

The method comprises first identifying as a viral target sequence, a region within the 5′-terminal 40 bases of the positive strand of the infecting virus whose sequence is capable of forming internal stem-loop secondary structure. There is then constructed, by stepwise solid-phase synthesis, a morpholino oligomer having a targeting sequence of at least 12 subunits that is complementary to the virus-genome region capable of forming internal duplex structure, where the oligomer is able to form with the viral target sequence, a heteroduplex structure composed of the positive sense strand of the virus and the oligonucleotide compound, and characterized by a Tₘ of dissociation of at least 45 °C and disruption of such stem-loop structure. The oligomer is conjugated to a carrier peptide described herein.

The target sequence may be identified by analyzing the 5′-terminal sequences, e.g., the 5′-terminal 40 bases, by a computer program capable of performing secondary structure predictions based on a search for the minimal free energy state of the input RNA sequence.

In a related aspect, the conjugates can be used in methods of inhibiting in a mammalian host cell, replication of an infecting RNA virus having a single-stranded, positive-sense genome and selected from one of the Flaviviridae, Picornoviridae, Caliciviridae, Togaviridae, Arteriviridae, Coronaviridae, Astroviridae or Hepeviridae families. The method includes administering to the infected host cells, a virus-inhibitory amount of conjugate as described herein, having a targeting sequence of at least 12 subunits that is complementary to a region within the 5′-terminal 40 bases of the positive-strand viral genome that is capable of forming internal stem-loop secondary structure. The conjugate is effective, when administered to the host cells, to form a heteroduplex structure (i) composed of the positive sense strand of the virus and the oligonucleotide compound, and (ii) characterized by a Tₘ of dissociation of at least 45 °C and disruption of such stem-loop secondary structure. The conjugate may be administered to a mammalian subject infected with the virus, or at risk of infection with the virus.

Exemplary targeting sequences that target the terminal stem loop structures of the dengue and Japanese encephalitis viruses are listed below as SEQ ID NOs: 1 and 2, respectively.

Additional exemplary targeting sequences that target the terminal stem loop structures of ssRNA viruses can also be found in U.S. application Ser. No. 11/801,885 and PCT publication WO/2008/036127 which are incorporated herein by reference.

2. Targeting the First Open Reading Frame of ssRNA Viruses

A second class of exemplary conjugates is for use in inhibition of growth of viruses of the picornavirus, calicivirus, togavirus, coronavirus, and flavivirus families having a single-stranded, positive sense genome of less than 12 kb and a first open reading frame that encodes a polyprotein containing multiple functional proteins. In particular embodiments, the virus is an RNA virus from the coronavirus family or a West Nile, Yellow Fever or Dengue virus from the flavivirus family. The inhibiting conjugates comprise antisense oligomers described herein, having a targeting base sequence that is substantially complementary to a viral target sequence which spans the AUG start site of the first open reading frame of the viral genome. In one embodiment of the method, the conjugate is administered to a mammalian subject infected with the virus. See, e.g., PCT Pubn. No. WO/2005/007805 and US Appn. Pubn. No. 2003224353, which are incorporated herein by reference.

The preferred target sequence is a region that spans the AUG start site of the first open reading frame (ORF1) of the viral genome. The first ORF generally encodes a polyprotein containing non-structural proteins such as polymerases, helicases and proteases. By “spans the AUG start site” is meant that the target sequence includes at least three bases on one side of the AUG start site and at least two bases on the other (a total of at least 8 bases). Preferably, it includes at least four bases on each side of the start site (a total of at least 11 bases).

More generally, preferred target sites include targets that are conserved between a variety of viral isolates. Other favored sites include the IRES (internal ribosome entry site), transactivation protein binding sites, and sites of initiation of replication. Complex and large viral genomes, which may provide multiple redundant genes, may be efficiently targeted by targeting host cellular genes coding for viral entry and host response to viral presence.

A variety of viral-genome sequences are available from well known sources, such as the NCBI Genbank databases. The AUG start site of ORF1 may also be identified in the gene database or reference relied upon, or it may be found by scanning the sequence for an AUG codon in the region of the expected ORF1 start site.

The general genomic organization of each of the four virus families is given below, followed by exemplary target sequences obtained for selected members (genera, species or strains) within each family.

### 3. Targeting Influenza Virus

A third class of exemplary conjugates are used in inhibition of growth of viruses of the Orthomyxoviridae family and in the treatment of a viral infection. In one embodiment, the host cell is contacted with a conjugate as described herein, for example a conjugate comprising a base sequence effective to hybridize to a target region selected from the following: 1) the 5′ or 3′ terminal 25 bases of the negative sense viral RNA segments; 2) the terminal 25 bases of the 5′ or 3′ terminus of the positive sense cRNA; 3) 45 bases surrounding the AUG start codons of influenza viral mRNAs and; 4) 50 bases surrounding the splice donor or acceptor sites of influenza mRNAs subject to alternative splicing. (See, e.g., PCT Pubn. No. WO/2006/047683; U.S. Appn. Pubn. No. 20070004661; and PCT Appn. Num. 2010/056613 and U.S. application Ser. No. 12/945,081, which are incorporated herein by reference.) Exemplary conjugates in this regard include conjugates comprising oligomers comprising SEQ ID NO:3.

TABLE 4
Influenza targeting sequences that incorporate modified
intersubunit linkages or terminal groups

| | | |
|---|---|---|
| NG-10-0038 | PMOhex | CGG T^{h}TA GAA GAC ^{h}TCA TC^{h}T TT |
| NG-10-0039 | PMOhex | CGG T^{h}TA GAA GAC ^{h}TCA ^{h}TCT ^{h}TT |
| NG-10-0096 | PMOapn | CGG T^{a}TA GAA GAC ^{a}TCA ^{a}TTC TT |
| NG-10-0097 | PMOapn | CGG ^{a}T^{a}TA GAA GAC ^{a}TCA ^{a}TC^{a}T TT |
| NG-10-0099 | PMOpyr | CGG ^{p}T^{p}TA GAA GAC ^{p}TCA ^{p}TC^{p}T TT |
| NG-10-0107 | PMOthiol | CGG T^{SH}TA GAA GAC ^{SH}TCA TC^{SH}T TT |
| NG-10-0108 | PMOsucc | CGG T^{s}TA GAA GAC ^{s}TCA TC^{s}T TT |
| NG-10-0111 | PMOguan | CGG T^{g}TA GAA GAC ^{g}TCA TC^{g}T TT |
| NG-10-0141 | PMOpyr | CGG T^{p}TA GAA GAC ^{p}TCA TC^{p}T TT |
| NG-10-0142 | PMOpyr | CGG ^{p}TA GAA GAC ^{p}TCA ^{p}TC^{p}T TT |
| NG-10-0158 | PMOglutaric | CGG T^{glu}TA GAA GAC ^{glu}TCA TC^{glu}T TT |
| NG-10-0159 | PMOcyclo-glut | CGG T^{cpglu}TA GAA GAC ^{cpglu}TCA TC^{cpglu}T TT |
| NG-10-0160 | PMOcholic acid | CGG T^{ca}TA GAA GAC ^{ca}TCA TC^{ca}T TT |
| NG-10-0161 | PMOdeoxyCA | CGG T^{dca}TA GAA GAC ^{dca}TCA TC^{dca}T TT |
| NG-10-0180 | PMOapn | TT^{a}T CGA CA^{a}T CGG T^{a}TA GAA GAC ^{a}TCA T |
| NG-10-0174 | PMOm | CGG T^{m}TA GAA GAC ^{m}TCA TC^{m}T TT |
| NG-10-0222 | PMO MeT | CGG T^{Me}TA GAA GAC +TCA TC+T TT |
| NG-10-0223 | PMO FarnT | CGG T^{Farn}TA GAA GAC +TCA TC+T TT |
| NG-10-0538 | PMOapn-trityl | CGG T^{a}TA GAA GAC ^{a}TCA TC^{a}T TT |
| NG-10-0539 | PMOapn-trityl | CGG P^{p}TA GAA GAC ^{p}TCA TC^{p}T TT |
| NG-10-0015 | PMO | CGG TTA GAA GAC TCA TCT TT |
| NG-11-0170 | PMOplus | CGG +TTA GAA GAC +TCA TC+T TT |
| NG-11-0145 | PMOplus-benzhydryl | CGG T+TA GAA GAC +TCA TC+T TT** |
| NG-11-0148 | PMOisopropylPip | CGG TiprpipTA GAA GAC iprpipTCA TCiprpipT TT |
| NG-11-0173 | PMOpyr | CGG pTTA GAA GAC pTCA TCpT TT |
| NG-11-0291 | Trimethyl Gly | CGG T*+TA GAA GAC *+TCA TC*+T TT |

**3′-benzhydryl;
*+linkages are trimethyl glycine acylated at the PMOplus linkages; PMOm represents T bases with a methyl group on the 3-nitrogen position.

The conjugate s are particularly useful in the treatment of influenza virus infection in a mammal. The o conjugate may be administered to a mammalian subject infected with the influenza virus, or at risk of infection with the influenza virus.

### 4. Targeting Viruses of the Picornaviridae Family

A fourth class of exemplary conjugates are used in inhibition of growth of viruses of the Picornaviridae family and in the treatment of a viral infection. The conjugates are particularly useful in the treatment of Enterovirus and/or Rhinovirus infection in a mammal. In this embodiment, the conjugates comprise morpholino oligomers having a sequence of 12-40 subunits, including at least 12 subunits having a targeting sequence that is complementary to a region associated with viral RNA sequences within one of two 32 conserved nucleotide regions of the viral 5′ untranslated region. (See, e.g., PCT Pubn. Nos. WO/2007/030576 and WO/2007/030691 or copending and co-owned U.S. application Ser. Nos. 11/518,058 and 11/517,757, which are incorporated herein by reference.) An exemplary targeting sequence is listed below as SEQ NO: 6.

### 5. Targeting Viruses of the Flavivirus Family

A fifth class of exemplary conjugates are used in inhibition of replication of a flavivirus in animal cells. An exemplary conjugate of this class comprises a morpholino oligomer of between 8-40 nucleotide bases in length and having a sequence of at least 8 bases complementary to a region of the virus' positive strand RNA genome that includes at least a portion of the 5′-cyclization sequence (5′-CS) or 3′-CS sequences of the positive strand flaviviral RNA. A highly preferred target is the 3′-CS and an exemplary targeting sequence for dengue virus is listed below as SEQ ID NO: 7. (See, e.g., PCT Pubn. No. (WO/2005/030800) or copending and co-owned U.S. application Ser. No. 10/913,996, which are incorporated herein by reference.)

### 6. Targeting Viruses of the Nidovirus Family

A sixth class of exemplary conjugates are used in inhibition of replication of a nidovirus in virus-infected animal cells. An exemplary conjugate of this class comprises a morpholino oligomer containing between 8-25 nucleotide bases, and having a sequence capable of disrupting base pairing between the transcriptional regulatory sequences (TRS) in the 5′ leader region of the positive-strand viral genome and negative-strand 3′ subgenomic region (See, e.g., PCT Pubn. No. WO/2005/065268 or U.S. Appn. Pubn. No. 20070037763, which are incorporated herein by reference.)

### 7. Targeting of Filoviruses

In another embodiment, one or more conjugates as described herein can be used in a method of in inhibiting replication within a host cell of an Ebola virus or Marburg virus, by contacting the cell with a conjugate as described herein, for example a conjugate having a targeting base sequence that is complementary to a target sequence composed of at least 12 contiguous bases within an AUG start-site region of a positive-strand mRNA, as described further below.

The filovirus viral genome is approximately 19,000 bases of single-stranded RNA that is unsegmented and in the antisense orientation. The genome encodes 7 proteins from monocistronic mRNAs complementary to the vRNA.

Target sequences are positive-strand (sense) RNA sequences that span or are just downstream (within 25 bases) or upstream (within 100 bases) of the AUG start codon of selected Ebola virus proteins or the 3′ terminal 30 bases of the minus-strand viral RNA. Preferred protein targets are the viral polymerase subunits VP35 and VP24, although L, nucleoproteins NP and VP30, are also contemplated. Among these early proteins are favored, e.g., VP35 is favored over the later expressed L polymerase.

In another embodiment, one or more conjugates as described herein can be used in a method of in inhibiting replication within a host cell of an Ebola virus or Marburg virus, by contacting the cell with a conjugate as described herein having a targeting base sequence that is complementary to a target sequence composed of at least 12 contiguous bases within an AUG start-site region of a positive-strand mRNA of the Filovirus mRNA sequences. (See, e.g., PCT Pubn. No. WO/2006/050414 or U.S. Pat. Nos. 7,524,829 and 7,507,196, and continuation applications with U.S. application Ser. No. 12/402,455; 12/402,461; 12/402,464; and 12/853,180 which are incorporated herein by reference.)

### 8. Targeting of Arenaviruses

In another embodiment, a conjugate as described herein can be used in a method for inhibiting viral infection in mammalian cells by a species in the Arenaviridae family. In one aspect, the conjugates can be used in treating a mammalian subject infected with the virus. (See, e.g., PCT Pubn. No. WO/2007/103529 or U.S. Pat. No. 7,582,615, which are incorporated herein by reference.) Table 5 is an exemplary list of targeted viruses targeted by conjugates of the invention as organized by their Old World or New World Arenavirus classification.

TABLE 5
Targeted Arenaviruses

| Family | Genus | Virus |
|---|---|---|
| | | Old World Arenaviruses |
| Arenaviridae | Arenavirus | Lassa virus (LASV) |
| | | Lymphocytic choriomeningitis |
| | | virus (LCMV) |
| | | Mopeia virus (MOPV) |
| | | New World Arenaviruses |
| | | Guanarito virus (GTOV) |
| | | Junín virus (JUNV) |
| | | Machupo virus (MACV) |
| | | Pichinide virus (PICV) |
| | | Pirital virus (PIRV) |
| | | Sabiá virus (SABV) |
| | | Tacaribe virus (TCRV) |
| | | Whitewater Arroyo virus (WWAV) |

The genome of Arenaviruses consists of two single-stranded RNA segments designated S (small) and L (large). In virions, the molar ratio of S- to L-segment RNAs is roughly 2:1. The complete S-segment RNA sequence has been determined for several arenaviruses and ranges from 3,366 to 3,535 nucleotides. The complete L-segment RNA sequence has also been determined for several arenaviruses and ranges from 7,102 to 7,279 nucleotides. The 3′ terminal sequences of the S and L RNA segments are identical at 17 of the last 19 nucleotides. These terminal sequences are conserved among all known arenaviruses. The 5′-terminal 19 or 20 nucleotides at the beginning of each genomic RNA are imperfectly complementary with each corresponding 3′ end. Because of this complementarity, the 3′ and 5′ termini are thought to base-pair and form panhandle structures.

Replication of the infecting virion or viral RNA (vRNA) to form an antigenomic, viral-complementary RNA (vcRNA) strand occurs in the infected cell. Both the vRNA and vcRNA encode complementary mRNAs; accordingly, Arenaviruses are classified as ambisense RNA viruses, rather than negative- or positive-sense RNA viruses. The ambisense orientation of viral genes are on both the L- and S-segments. The NP and polymerase genes reside at the 3′ end of the S and L vRNA segments, respectively, and are encoded in the conventional negative sense (i.e., they are expressed through transcription of vRNA or genome-complementary mRNAs). The genes located at the 5′ end of the S and L vRNA segments, GPC and Z, respectively, are encoded in mRNA sense but there is no evidence that they are translated directly from genomic vRNA. These genes are expressed instead through transcription of genomic-sense mRNAs from antigenomes (i.e., the vcRNA), full-length complementary copies of genomic vRNAs that function as replicative intermediates.

An exemplary targeting sequence for the arenavirus family of viruses is listed below as SEQ ID NO: 8.

### 9. Targeting of Respiratory Syncytial Virus

Respiratory syncytial virus (RSV) is the single most important respiratory pathogen in young children. RSV-caused lower respiratory conditions, such as bronchiolitis and pneumonia, often require hospitalization in children less than one-year-old. Children with cardiopulmonary diseases and those born prematurely are especially prone to experience severe disorders from this infection. RSV infection is also an important illness in elderly and high-risk adults, and it is the second-most commonly identified cause of viral pneumonia in older persons (Falsey, Hennessey et al. 2005). The World Health Organization estimates that RSV is responsible for 64 million clinical infections and 160 thousand deaths annually worldwide. No vaccines are currently available for the prevention of RSV infection. Although many major advances in our understanding of RSV biology, epidemiology, pathophysiology, and host-immune-response have occurred over the past few decades, there continues to be considerable controversy regarding the optimum management of infants and children with RSV infection. Ribavirin is the only licensed antiviral drug for treating RSV infection, but its use is limited to high-risk or severely-ill infants. The utility of Ribavirin has been limited by its cost, variable efficacy, and tendency to generate resistant viruses (Marquardt 1995; Prince 2001). The current need for additional effective anti-RSV agents is well-acknowledged.

It is known that peptide conjugated PMO (PPMO) can be effective in inhibiting RSV both in tissue culture and in an in vivo animal model system (Lai, Stein et al. 2008). Two antisense PPMOs, designed to target the sequence that includes the 5′-terminal region and translation start-site region of RSV L mRNA, were tested for anti-RSV activity in cultures of two human airway cell lines. One of them, (RSV-AUG-2; SEQ ID NO 10), reduced viral titers by >2.0 log₁₀. Intranasal (i.n.) treatment of BALB/c mice with RSV-AUG-2 PPMO before the RSV inoculation produced a reduction in viral titer of 1.2 log₁₀ in lung tissue at day 5 postinfection (p.i.), and attenuated pulmonary inflammation at day 7 postinfection. These data showed that RSV-AUG-2 provided potent anti-RSV activity worthy of further investigation as a candidate for potential therapeutic application (Lai, Stein et al. 2008). Despite the success with RSV-AUG-2 PPMO as described above, it is desirable to use conjugates as disclosed herein to address toxicity associated with previous peptide conjugates. Therefore, in another embodiment of the present invention, one or more conjugates as described herein can be used in a method of inhibiting replication within a host cell of RSV, by contacting the cell with a conjugate as described herein, for example a conjugate having a targeting base sequence that is complementary to a target sequence composed of at least 12 contiguous bases within an AUG start-site region of anmRNA from RSV, as described further below.

The L gene of RSV codes for a critical component of the viral RNA dependent RNA polymerase complex. Antisense PPMO designed against the sequence spanning the AUG translation start-site codon of the RSV L gene mRNA in the form of RSV-AUG-2 PPMO is complementary to sequence from the ‘gene-start’ sequence (GS) present at the 5′ terminus of the L mRNA to 13 nt into the coding sequence. A preferred L gene targeting sequence is therefore complementary to any 12 contiguous bases from the 5′ end of the L gene mRNA extending 40 bases in the 3′ direction or 22 bases into the L gene coding sequence as shown below in Table 6 as SEQ ID NO: 9. Exemplary RSV L gene targeting sequences are listed below in Table 6 as SEQ ID NOs: 10-14. Any of the intersubunit modifications of the invention described herein can be incorporated in the oligomers to provide increased antisense activity, improved intracellular delivery and/or tissue specificity for improved therapeutic activity. Exemplary oligomers sequences containing intersubunit linkages of the invention are listed below in Table 6.

TABLE 6
RSV target and targeting sequences

| | | SEQ |
|---|---|---|
| Name | Sequence (5′ to 3′) | ID NO |
| L target | GGGACAAAATGGATCCCATTATTAATGGAAATTC | 9 |
| | TGCTAA | |
| RSV-AUG-2 | TAATGGGATCCATTTTGTCCC | 10 |
| RSV-AUG3 | AATAATGGGATCCATTTTGTCCC | 11 |
| RSV-AUG4 | CATTAATAATGGGATCCATTTTGTCCC | 12 |
| RSV-AUG5 | GAATTTCCATTAATAATGGGATCCATTTTG | 13 |
| RSV-AUG6 | CAGAATTTCCATTAATAATGGGATCCATT | 14 |
| RSV- | AATAA^{apn}TGGGA^{apn}TCCA^{apn}TT^{apn}TTG^{apn}TCCC | 11 |
| AUG3apn* | | |
| RSV- | AATAA^{guan}TGGGA^{guan}TCCA^{guanT}T^{guan}TTG^{guan} | 11 |
| AUG3guan | TCCC | |

### 10. Neuromuscular Diseases

In another embodiment, a therapeutic conjugate is provided for use in treating a disease condition associated with a neuromuscular disease in a mammalian subject. Antisense oligomers (e.g., SEQ ID NO: 16) have been shown to have activity in the MDX mouse model for Duchene Muscular Dystrophy (DMD). Exemplary oligomer sequences that incorporate the linkages used in some embodiments are listed below in Table 7. In some embodiments, the conjugates comprise an oligomer selected from:
(a) an antisense oligomer targeted against human myostatin, having a base sequence complementary to at least 12 contiguous bases in a target region of the human myostatin mRNA identified by SEQ ID NO: 18, for treating a muscle wasting condition, as described previously (See, e.g., U.S. patent application Ser. No. 12/493,140, which is incorporated herein by reference; and PCT publication WO2006/086667). Exemplary murine targeting sequences are listed as SEQ ID NOs: 19-20; and
(b) an antisense oligomer capable of producing exon skipping in the DMD protein (dystrophin), such as a PMO having a sequence selected from SEQ ID NOs: 22 to 35, to restore partial activity of the dystrophin protein, for treating DMD, as described previously (See, e.g., PCT Pubn. Nos. WO/2010/048586 and WO/2006/000057 or U.S. Ser. No. 09/061,960 all of which are incorporated herein by reference).

Several other neuromuscular diseases can be treated using the modified linkages and terminal groups of the present invention. Exemplary compounds for treating spinal muscle atrophy (SMA) and myotonic dystrophy (DM) are discussed below.

SMA is an autosomal recessive disease caused by chronic loss of alpha-motor neurons in the spinal cord and can affect both children and adults. Reduced expression of survival motor neuron (SMN) is responsible for the disease (Hua, Sahashi et al. 2010). Mutations that cause SMA are located in the SMN1 gene but a paralogous gene, SMN2, can allow viability by compensating for loss of SMN1 if expressed from an alternative splice form lacking exon 7 (delta7 SMN2). Antisense compounds targeted to inton 6, exon 7 and intron 7 have all been shown to induce exon 7 inclusion to varying degrees. Antisense compounds targeted to intron 7 are preferred (see e.g., PCT Publication Nos. WO/2010/148249, WO/2010/120820, WO/2007/002390 and U.S. Pat. No. 7,838,657). Exemplary antisense sequences that target the SMN2 pre-mRNA and induce improved exon 7 inclusion are listed below as SEQ ID NOs: 36-38. It is contemplated that selected modifications of these oligomer sequences using the modified linkages and terminal groups described herein would have improved properties compared to those known in the art. Furthermore, it is contemplated that any oligomer targeted to intron 7 of the SMN2 gene and incorporating the features of the present invention has the potential to induce exon 7 inclusion and provide a therapeutic benefit to SMA patients. Myotonic Dystrophy type 1 (DM1) and type 2 (DM2) are dominantly inherited disorders caused by expression of a toxic RNA leading to neuromuscular degeneration. DM1 and DM2 are associated with long polyCUG and polyCCUG repeats in the 3′-UTR and intron 1 regions of the transcript dystrophia myotonica protein kinase (DMPK) and zinc finger protein 9 (ZNF9), respectively (see e.g., WO2008/036406). While normal individuals have as many as 30 CTG repeats, DM1 patients carry a larger number of repeats ranging from 50 to thousands. The severity of the disease and the age of onset correlates with the number of repeats. Patients with adult onsets show milder symptoms and have less than 100 repeats, juvenile onset DM1 patients carry as many as 500 repeats and congenital cases usually have around a thousand CTG repeats. The expanded transcripts containing CUG repeats form a secondary structure, accumulate in the nucleus in the form ofnuclear foci and sequester RNA-binding proteins (RNA-BP). Several RNA-BP have been implicated in the disease, including muscleblind-like (MBNL) proteins and CUG-binding protein (CUGBP). MBNL proteins are homologous to Drosophila muscleblind (Mbl) proteins necessary for photoreceptor and muscle differentiation. MBNL and CUGBP have been identified as antagonistic splicing regulators of transcripts affected in DM1 such as cardiac troponin T (cTNT), insulin receptor (IR) and muscle-specific chloride channel (ClC-1).

It is known in the art that antisense oligonucleotides targeted to the expanded repeats of the DMPK gene can displace RNA-BP sequestration and reverse myotonia symptoms in an animal model of DM1 (WO2008/036406). It is contemplated that oligomers incorporating features of the present invention would provide improved activity and therapeutic potential for DM1 and DM2 patients. Exemplary sequences targeted to the polyCUG and polyCCUG repeats described above are listed below as SEQ ID NOs: 39-55 and further described in U.S. application Ser. No. 13/101,942 which is incorporated herein in its entirety.

Additional embodiments of the present invention for treating neural muscular disorders are anticipated and include oligomers designed to treat other DNA repeat instability genetic disorders. These diseases include Huntington's disease, spino-cerebellar ataxia, X-linked spinal and bulbar muscular atrophy and spinocerebellar ataxia type 10 (SCA10) as described in WO2008/018795.

TABLE 7
M23D sequences (SEQ ID NO: 15) that incorporate modified intersubunit
linkages and/or 3′ and/or 5′ terminal groups

| | PMO-X | | | |
|---|---|---|---|---|
| NG | Modification | 5′ | Sequence | 3′ |
| NG-10-0383 | PMO | EG3 | GCC CAA ACC TCG GCT TAC CTG AAA T | triphenylacetyl |
| NG-10-0325 | triphenylphos | OH | GGC CAA ACC FCG GCF TAC CFG AAA T | triphenylphos |
| NG-10-0272 | PMO-farnesyl | OH | GGC CAA ACC TCG GCT TAC CTG AAA T | farnesyl |
| NG-10-0102 | PMO | OH | GGC CAA ACC TCG GCT TAC CTG AAA T | trityl |
| MG-10-0330 | trimethoxy- | EG3 | GGC CAA ACC TCG GCT TAC CTG AAA T | trimethoxy- |
| | benzoyl | | | benzoyl |
| NG-10-0056 | PMOplus | EG3 | GGC C⁺A⁺A ⁺ACC TCG GCT TAC CTG AAA | H |
| | 5′-pol | | T | |
| NG-07-0064 | PMO-3′-trityl | H-Pip | GGC CAA ACC TCG GCT TAC CTG AAA T | trityl |
| NG-10-0382 | PMO | EG3 | GGC CAA ACC TCG GCT TAC CTG AAA T | triphenyl- |
| | | | | propionyl |
| NG-10-0278 | PMOpyr | EG3 | GGC CAA ACC pTCG GCpT pTAC CpTG | H |
| | | | AAA pT | |
| NG-10-0210 | PMOapn | EG3 | GGC C^{a}A^{a}A ^{a}ACC TCG GCT TAC CTG AAA | H |
| | | | T | |
| NG-10-0098 | PMOpyr | EG3 | GGC CAA ACC ^{p}TCG GC^{p}T TAC C^{p}TG AAA | H |
| | | | T | |
| NG-10-0070 | PMOapn | EG3 | GGC CAA ACC ^{a}TCG GC^{a}T TAC C^{a}TG AAA | H |
| | | | ^{a}T | |
| NG-10-0095 | PMOapn | EG3 | GGC CAA ACC ^{a}TCG GC^{a}T ^{a}TAC C^{a}T G | H |
| | | | AAA ^{a}T | |
| NG-10-0317 | PMO | EG3 | GGC CAA ACC TCG GCT TAC CTG AAA T | farnesyl |
| NG-10-0477 | PMO triMe | EG3 | GGC CAA ACC FCG GCF TAC CFG AAA F | trimethyl |
| | Gly | | | Glycine |
| NG-10-0133 | PMOapn | OH | GGC C^{a}AA ^{a}ACC ^{a}TCG GC^{a}T ^{a}TAC C^{a}TG | H |
| | | | AAA ^{a}T | |
| NG-10-0387 | PMO | EG3 | GGC CAA ACC TCG GCT TAC CTG AAA T | 2-OH, |
| | | | | diphenylacet |
| NG-10-0104 | PMOguan | EG3 | GGC CAA ACC ^{g}TCG GC^{g}T TAC C^{g}T G | ^{g} |
| | | | AAA T | |
| NG-10-0420 | PMOplus | EG3 | GGC CAA ACC ^{m+}TCG GC^{m+}T TAC C^{m+}TG | Trityl |
| | methyl | | AAA ^{m+}T | |
| NG-10-0065 | PMOtri | EG3 | GGC CAA ACC ^{t}TCG GC^{t}T TAC C^{t}T G AAA | H |
| | | | T | |
| NG-10-0607 | PMO-X | EG3 | GGC CAA ACC TCG GCT TAC CTG AAA T | 6-fluorene- |
| | | | | carboxyl |
| NG-10-0060 | PMOcp | EG3 | GGC CAA ACC ^{cp}TCG GC^{cp}T TAC C^{cp}T G | H |
| | | | AAA T | |
| NG-10-0162 | PMO-COCH₂SH | EG3 | GGC CAA ACC TCG GCT TAC CTG AAA T | COCH₂SH |
| NG-10-0328 | diphenylacetyl | EG3 | GGC CAA ACC TCG GCT TAC CTG AAA T | diphenylacetyl |
| NG-10-0134 | PMOapnPMOtri | OH | GGC C^{a}AA ^{a}ACC ^{t}TCG GC^{t}T ^{t}TAC C^{t}TG | H |
| | | | AAA ^{t}T | |
| NG-10-0386 | PMO | DPA | GGC CAA ACC TCG GCT TAC CTG AAA T | 5′-diphenylac, |
| | | | | 3′-trity |
| NG-07-0064 | PMO-3′-trityl | H-Pip | GGC CAA ACC TCG GCT TAC CTG AAA T | trityl |
| NG-10-0059 | PMOcp | EG3 | GGC CAA ACC ^{cp}TCG GC^{cp}T ^{cp}TAC C^{cp}T G | H |
| | | | AAA ^{cp}T | |
| NG-10-0135 | PMOtri | OH | GGC CAA ACC ^{t}TCG GC^{t}T ^{t}TAC C^{t}TG | H |
| | | | AAA ^{t}T | |
| NG-10-0168 | PMOapn | OH | GGC CAA ACC ^{a}TCG GC^{a}T ^{a}TAC C^{a}TG | H |
| | PMOcys | | AAA ^{SHe}T | |
| NG-10-0113 | PMOapnPMOtri | OH | GGC CAA ACC ^{a}TCG GC^{t}T ^{t}TAC C^{a}TG | H |
| | | | AAA ^{a}T | |
| NG-10-0385 | PMO | EG3 | GGC CAA ACC TCG GCT TAC CTG AAA T | diphenyl- |
| | | | | phosphoryl |
| NG-10-0279 | PMO | OH | GGC CAA ACC TCG GCT TAC CTG AAA T | geranyl |
| NG-10-0055 | PMOplus disp | EG3 | GGC C⁺AA ⁺ACC ⁺TCG GC⁺T TAC C⁺TG | H |
| | | | AAA T | |
| NG-10-0105 | PMOsucc | EG3 | GGC CAA ACC ^{s}TCG GC^{s}T TAC C^{s}T G | ^{s} |
| | | | AAA T | |
| NG-10-0805 | PMO-X | EG3 | GGC CAA ACC ^{Etpip}TCG GC^{Etpip}T TAC | H |
| | | | C^{Etpip}TG AAA ^{Etpip}T | |
| NG-10-0811 | PMO-X | EG3 | GGC CAA CCC ^{pyrQMe}TCG GC^{pyrQMe}T TAC | H |
| | | | C^{pyrQMe}TG AAA ^{pyrQMe}T | |
| NG-10-0057 | PMOplus | EG3 | GGC CAA ACC TCG GCT TAC C⁺TG | H |
| | 3′-pol | | ⁺A⁺A⁺A T | |
| NG-10-0625 | PMO-X | EG3 | GGC CAA ACC TCG GCT TAC CTG AAA T | 5-carboxy- |
| | | | | fluorescein |
| NG-10-0804 | dimer | EG3 | GGC CAA ACC TCG GCT TAC CTG AAA T | dimerized |
| NG-10-0066 | PMOtri | EG3 | GGC CAA ACC ⁺TCG GC⁺T TAC C⁺T G | H |
| | | | AAA ⁺T | |
| NG-10-0280 | PMO disulfide | EG3 | GGC CAA ACC TCG GCT TAC CTG AAA T | COCH₂CH₂SSPy |
| NG-10-0210 | PMOapn | EG3 | GGC CaAaA aACC aTCG GCaT aTaAC | H |
| | | | CaTG aAaAaA aT | |
| NG-10-0156 | 3′-MeOtrityl | EG3 | GGC CAA ACC TCG GCT TAC CTG AAA T | MeO-Tr |
| NG-10-0062 | PMOhex | EG3 | GGC CAA ACC ^{h}TCG GC^{h}T TAC C^{h}T G | H |
| | | | AAA ^{h}T | |
| NG-11-0043 | PMO-X | EG3 | GGC CAA ACC TCG GCT TAC CTG AAA T | guanidinyl |
| NG-10-0206 | PMOplus | EG3 | GGC C+A+A +ACC +TCG GC+T +T+AC | H |
| | | | C+TG +A+A+A +T | |
| NG-10-0383 | PMO | EG3 | GGC CAA ACC TCG GCT TAC CTG AAA T | triphenylacetyl |
| NG-10-0325 | triphenylphos | OH | GGC CAA ACC FCG GCF TAC CFG AAA T | triphenylphos |
| NG-10-0272 | PMO-farnesyl | OH | GGC CAA ACC TCG GCT TAC CTG AAA T | farnesyl |

*Dimerized indicates the oligomer is dimerized by a linkage linking the 3′ ends of the two monomers. For example, the linkage may be —COCH₂CH₂—S—CH(CONH₂)CH₂—CO—NHCH₂CH₂CO— or any other suitable linkage. EG3 refers to a triethylene glycol tail (see e.g., conjugates in examples 30 and 31).

### 11. Antibacterial Applications

The invention includes, in another embodiment, a conjugate comprising an antibacterial antisense oilgomer for use in treating a bacterial infection in a mammalian host. In some embodiments, the oligomer comprises between 10-20 bases and a targeting sequence of at least 10 contiguous bases complementary to a target region of the infecting bacteria's mRNA for acyl carrier protein (acpP), gyrase A subunit (gyrA), ftsZ, ribosomal protein S10 (rpsJ), leuD, mgtC, pirG, pcaA, and cmal genes, where the target region contains the translational start codon of the bacterial mRNA, or a sequence that is within 20 bases, in an upstream (i.e., 5′) or downstream (i.e., 3′) direction, of the translational start codon, and where the oligomer binds to the mRNA to form a heteroduplex thereby to inhibit replication of the bacteria.

### 12. Modulating Nuclear Hormone Receptors

In another embodiment the present invention relates to compositions and methods for modulating expression of nuclear hormone receptors (NHR) from the nuclear hormone receptor superfamily (NHRSF), mainly by controlling or altering the splicing of pre-mRNA that codes for the receptors. Examples of particular NHRs include glucocorticoid receptor (GR), progesterone receptor (PR) and androgen receptor (AR). In certain embodiments, the conjugates described herein lead to increased expression of ligand-independent or other selected forms of the receptors, and decreased expression of their inactive forms.

Embodiments of the present invention include conjugates comprising oligomers, for example oligomers that are complementary to selected exonic or intronic sequences of an NHR, including the “ligand-binding exons” and/or adjacent introns of a NHRSF pre-mRNA, among other NHR-domains described herein. The term “ligand-binding exons” refers to exon(s) that are present in the wild-type mRNA but are removed from the primary transcript (the “pre-mRNA”) to make a ligand-independent form of the mRNA. In certain embodiments, complementarity can be based on sequences in the sequence of pre-mRNA that spans a splice site, which includes, but is not limited to, complementarity based on sequences that span an exon-intron junction. In other embodiments, complementarity can be based solely on the sequence of the intron. In other embodiments, complementarity can be based solely on the sequence of the exon. (See, e.g., U.S. application Ser. No. 13/046,356, which is incorporated herein by reference.)

NHR modulators may be useful in treating NHR-associated diseases, including diseases associated with the expression products of genes whose transcription is stimulated or repressed by NHRs. For instance, modulators of NHRs that inhibit AP-1 and/or NF-κB can be useful in the treatment of inflammatory and immune diseases and disorders such as osteoarthritis, rheumatoid arthritis, multiple sclerosis, asthma, inflammatory bowel disease, transplant rejection, and graft vs. host disease, among others described herein and known in the art. Compounds that antagonize transactivation can be useful in treating metabolic diseases associated with increased levels of glucocorticoid, such as diabetes, osteoporosis and glaucoma, among others. Also, compounds that agonize transactivation can be useful in treating metabolic diseases associated with a deficiency in glucocorticoid, such as Addison's disease and others.

Embodiments of the present invention include methods of modulating nuclear NHR activity or expression in a cell, comprising contacting the cell with a conjugate comprising the carrier protein and an antisense oligomer composed of morpholino subunits linked by phosphorus-containing intersubunit linkages joining a morpholino nitrogen of one subunit to a 5′ exocyclic carbon of an adjacent subunit, wherein the oligonucleotide contains between 10-40 bases and a targeting sequence of at least 10 contiguous bases complementary to a target sequence, wherein the target sequence is a pre-mRNA transcript of the NHR, thereby modulating activity or expression of the NHR. In certain embodiments, the oligomer alters splicing of the pre-mRNA transcript and increases expression of a variant of the NHR. In some embodiments, the oligomer induces full or partial exon-skipping of one or more exons of the pre-mRNA transcript. In certain embodiments, the one or more exons encode at least a portion of a ligand-binding domain of the NHR, and the variant is a ligand independent form of the NHR. In certain embodiments, the one or more exons encode at least a portion of a transactivation domain of the NHR, and the variant has reduced transcriptional activation activity. In certain embodiments, the one or more exons encode at least a portion of a DNA-binding domain of the NHR. In certain embodiments, the one or more exons encode at least a portion of an N-terminal activation domain of the NHR. In certain embodiments, the one or more exons encode at least a portion of a carboxy-terminal domain of the NHR. In specific embodiments, the variant binds to NF-KB, AP-1, or both, and reduces transcription of one or more of their pro-inflammatory target genes.

In certain embodiments, the oligomer agonizes a transactivational transcriptional activity of the NHR. In other embodiments, the oligomer antagonizes a transactivational transcriptional activity of the NHR. In certain embodiments, the oligomer agonizes a transrepression activity of the NHR. In other embodiments, the oligomer antagonizes a transrepression activity of the NHR. In specific embodiments, the oligomer antagonizes a transactivational transcriptional activity of the NHR and agonizes a transrepression activity of the NHR. (See, e.g., U.S. application No. 61/313,652, which is incorporated herein by reference.)

### EXAMPLES

Unless otherwise noted, all chemicals were obtained from Sigma-Aldrich-Fluka. Benzoyl adenosine, benzoyl cytidine, and phenylacetyl guanosine were obtained from Carbosynth Limited, UK.

Synthesis of PMO, PMO+, PPMO and PMO containing further linkage modifications as described herein was done using methods known in the art and described in pending U.S. application Ser. Nos. 12/271,036 and 12/271,040 and PCT publication number WO/2009/064471, which are hereby incorporated by reference in their entirety.

PMO with a 3′ trityl modification are synthesized essentially as described in PCT publication number WO/2009/064471 with the exception that the detritylation step is omitted.

### Example 1

### Tert-Butyl 4-(2,2,2-Trifluoroacetamido)Piperidine-1-Carboxylate

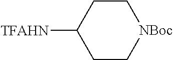

To a suspension of tert-butyl 4-aminopiperidine-1-carboxylate (48.7 g, 0.243 mol) and DIPEA (130 mL, 0.749 mol) in DCM (250 mL) was added ethyl trifluoroacetate (35.6 mL, 0.300 mol) dropwise while stirring. After 20 hours, the solution was washed with citric acid solution (200 mL×3, 10% w v aq) and sodium bicarbonate solution (200 mL×3, conc aq), dried (MgSO₄), and filtered through silica (24 g). The silica was washed with DCM and the combined eluant was partially concentrated (100 mL), and used directly in the next step. APCI/MS calcd. for C₁₂H₁₉F₃N₂O₃ 296.1, found m/z 294.9 (M−1).

### Example 2

### 2,2,2-Trifluoro-N-(Piperidin-4-yl)Acetamide Hydrochloride

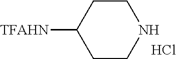

To a stirred DCM solution of the title compound of Example 1 (100 mL) was added dropwise a solution of hydrogen chloride (250 mL, 1.0 mol) in 1,4-dioxane (4 M). Stirring was continued for 6 hours, then the suspension was filtered, and the solid washed with diethyl ether (500 mL) to afford the title compound (54.2 g, 96% yield) as a white solid. APCI/MS calcd. for C₇H₁₁F₃N₂O 196.1, found m/z 196.9 (M+1).

### Example 3

### (4-(2,2,2-Trifluoroacetamido)Piperidin-1-yl)Phosphonic Dichloride

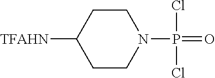

To a cooled (ice/water bath) suspension of the title compound of Example 2 (54.2 g, 0.233 mol) in DCM (250 mL) was added dropwise phosphorus oxychloride (23.9 mL, 0.256 mol) and DIPEA (121.7 mL, 0.699 mol) and stirred. After 15 minutes, the bath was removed and with continued stirring the mixture allowed to warm to ambient temperature. After 1 hour, the mixture was partially concentrated (100 mL), the suspension filtered, and the solid washed with diethyl ether to afford the title compound (43.8 g, 60% yield) as a white solid. The eluant was partially concentrated (100 mL), the resulting suspension filtered, and the solid washed with diethyl ether to afford additional title compound (6.5 g, 9% yield). ESI/MS calcd. for 1-(4-nitrophenyl)piperazine derivative C₁₇H₂₂ClF₃N₅O₄P 483.1, found m/z 482.1 (M−1).

### Example 4

### ((2S,6S)-6-((R)-5-Methyl-2,6-Dioxo-1,2,3,6-Tetrahydropyridin-3-yl)-4-Tritylmorpholin-2-yl)Methyl (4-(2,2,2-Trifluoroacetamido)Piperidin-1-yl)Phosphonochloridate

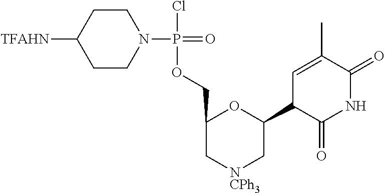

To a stirred, cooled (ice/water bath) solution of the title compound of Example 3 (29.2 g, 93.3 mmol) in DCM (100 mL) was added dropwise over 10 minutes a DCM solution (100 mL) of Mo(Tr)T #(22.6 g, 46.7 mmol), 2,6-Lutidine (21.7 mL, 187 mmol), and 4-(dimethylamino)pyridine (1.14 g, 9.33 mmol). The bath was allowed to warm to ambient temperature. After 15 hours, the solution was washed with a citric acid solution (200 mL×3, 10% w v aq), dried (MgSO₄), concentrated, and the crude oil was loaded directly onto column. Chromatography [SiO₂ column (120 g), hexanes/EtOAc eluant (gradient 1:1 to 0:1), repeated ×3] fractions were concentrated to provide the title compound (27.2 g, 77% yield) as a white solid. ESI/MS calcd. for the 1-(4-nitrophenyl)piperazine derivative C₄₆H₅₀F₃N₈O₈P 930.3, found m/z 929.5 (M−1).

### Example 5

### ((2S,6R)-6-(6-Benzamido-9H-Purin-9-yl)-4-Tritylmorpholin-2-yl)Methyl (4-(2,2,2-Trifluoroacetamido)Piperidin-1-yl)Phosphonochloridate

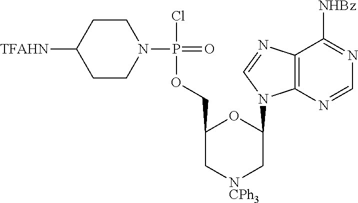

The title compound was synthesized in a manner analogous to that described in Example 4 to afford the title compound (15.4 g, 66% yield) as a white solid. ESI/MS calcd. for 1-(4-nitrophenyl)piperazine derivative C₈₃H₅₃F₃N₁₁O₇P 1043.4, found m/z 1042.5 (M−1).

### Example 6

### (R)-Methyl(1-Phenylethyl)Phosphoramidic Dichloride

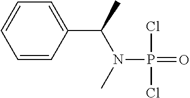

To a cooled (ice/water bath) solution of phosphorus oxychloride (2.83 mL, 30.3 mmol) in DCM (30 mL) was added sequentially, dropwise, and with stirring 2,6-lutidine (7.06 mL, 60.6 mmol) and a DCM solution of (R)-(+)—N,a-dimethylbenzylamine (3.73 g, 27.6 mmol). After 5 minutes, the bath was removed and reaction mixture allowed to warm to ambient temperature. After 1 hour, the reaction solution was washed with a citric acid solution (50 mL×3, 10% w v aq), dried (MgSO₄), filtered through SiO₂ and concentrated to provide the title compound (3.80 g) as a white foam. ESI/MS calcd. for 1-(4-nitrophenyl)piperazine derivative C₁₉H₂₅N₄O₄P 404.2, found m/z 403.1 (M−1).

### Example 7

### (S)-Methyl(1-Phenylethyl)Phosphoramidic Dichloride

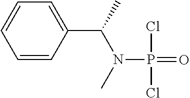

The title compound was synthesized in a manner analogous to that described in Example 6 to afford the title compound (3.95 g) as a white foam. ESI/MS calcd. for 1-(4-nitrophenyl)piperazine derivative C₁₉H₂₅N₄O₄P 404.2, found m/z 403.1 (M−1).

### Example 8

### ((2S,6R)-6-(5-Methyl-2,4-Dioxo-3,4-Dihydropyrimidin-1(2H)-yl)-4-Tritylmorpholin-2-yl)Methyl Methyl((R)-1-Phenylethyl)Phosphoramidochloridate

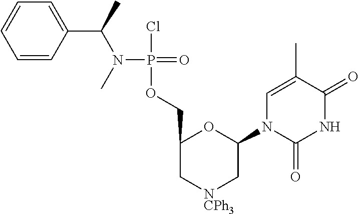

The title compound was synthesized in a manner analogous to that described in Example 4 to afford the title chlorophosphoroamidate (4.46 g, 28% yield) as a white solid. ESI/MS calcd. for C₃₈H₄₀ClN₄O₅P 698.2, found m/z 697.3 (M−1).

### Example 9

### ((2S,6R)-6-(5-Methyl-2,4-Dioxo-3,4-Dihydropyrimidin-1(2H)-yl)-4-Tritylmorpholin-2-yl)Methyl Methyl((S)-1-Phenylethyl)Phosphoramidochloridate

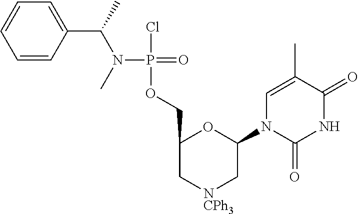

The title compound was synthesized in a manner analogous to that described in Example 4 to afford the title chlorophosphoroamidate (4.65 g, 23% yield) as a white solid. ESI/MS calcd. for C₃₈H₄₀ClN₄O₅P 698.2, found m/z 697.3 (M−1).

### Example 10

### (4-(Pyrrolidin-1-yl)Piperidin-1-yl)Phosphonic Dichloride Hydrochloride

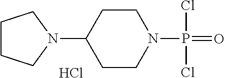 [SMILES: O=P(Cl)(Cl)N1CCC(N2CCCC2)CC1 ; Cl]

To a cooled (ice/water bath) solution of phosphorus oxychloride (5.70 mL, 55.6 mmol) in DCM (30 mL) was added 2,6-lutidine (19.4 mL, 167 mmol) and a DCM solution (30 mL) of 4-(1-pyrrolidinyl)-piperidine (8.58 g, 55.6 mmol) and stirred for 1 hour. The suspension was filtered and solid washed with excess diethyl ether to afford the title pyrrolidine (17.7 g, 91% yield) as a white solid. ESI/MS calcd. for 1-(4-nitrophenyl)piperazine derivative C₁₉H₃₀N₅O₄P 423.2, found m/z 422.2 (M−1).

### Example 11

### ((2S,6R)-6-(5-Methyl-2,4-Dioxo-3,4-Dihydropyrimidin-1(2H)-yl)-4-Tritylmorpholin-2-yl)Methyl (4-(Pyrrolidin-1-yl)Piperidin-1-yl)Phosphonochloridate Hydrochloride

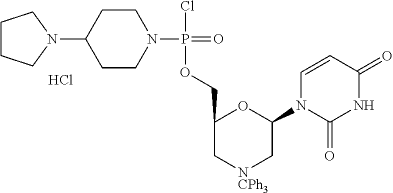

To a stirred, cooled (ice/water bath) solution of the dichlorophosphoramidate 8 (17.7 g, 50.6 mmol) in DCM (100 mL) was added a DCM solution (100 mL) of Mo(Tr)T #(24.5 g, 50.6 mmol), 2,6-Lutidine (17.7 mL, 152 mmol), and 1-methylimidazole (0.401 mL, 5.06 mmol) dropwise over 10 minutes. The bath was allowed to warm to ambient temperature as suspension was stirred. After 6 hours, the suspension was poured onto diethyl ether (1 L), stirred 15 minutes, filtered and solid washed with additional ether to afford a white solid (45.4 g). The crude product was purified by chromatography [SiO₂ column (120 gram), DCM/MeOH eluant (gradient 1:0 to 6:4)], and the combined fractions were poured onto diethyl ether (2.5 L), stirred 15 min, filtered, and the resulting solid washed with additional ether to afford the title compound (23.1 g, 60% yield) as a white solid. ESI/MS calcd. for 1-(4-nitrophenyl)piperazine derivative C₄₈H₅₇N₈O₇P 888.4, found m/z 887.6 (M−1).

### Example 12

### 3-(Tert-Butyldisulfanyl)-2-(Isobutoxycarbonylamino)Propanoic Acid

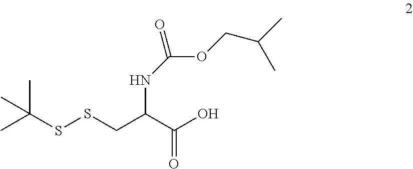 [SMILES: CC(C)COC(=O)NC(CSSC(C)(C)C)C(=O)O]

To S-tert-butylmercapto-L-cysteine (10 g, 47.8 mmol) in CH₃CN (40 mL) was added K₂CO₃ (16.5 g, 119.5 mmol) in H₂O (20 mL). After stirring for 15 minutes, iso-butyl chloroformate (9.4 mL, 72 mmol) was injected slowly. The reaction was allowed to run for 3 hours. The white solid was filtered through Celite; the filtrate was concentrated to remove CH₃CN. The residue was dissolved in ethyl acetate (200 mL), washed with 1N HCl (40 ml×3), brine (40×1), dried over Na₂SO₄. Desired product (2) was obtained after chromatography (5% MeOH/DCM).

### Example 13

### Tert-Butyl 4-(3-(Tert-Butyldisulfanyl)-2-(Isobutoxycarbonylamino)Propanamido)Piperidine-1-Carboxylate

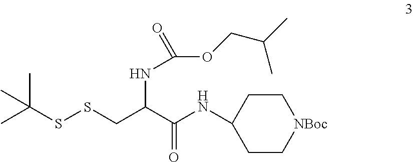 [SMILES: CC(C)COC(=O)NC(CSSC(C)(C)C)C(=O)NC1CCN(C(=O)OC(C)(C)C)CC1]

To the acid (compound 2 from Example 12, 6.98 g, 22.6 mmol) in DMF (50 ml was added HATU (8.58 g, 22.6 mmol). After 30 min, Hunig base (4.71 ml, 27.1 mmol) and 1-Boc-4-amino piperidine (5.43 g, 27.1 mmol) were added to the mixture. The reaction was continued stirring at RT for another 3h. DMF was removed at high vacuum, the crude residue was dissolved in EtAc (300 ml), washed with H₂O (50 ml×3). The final product (3) was obtained after ISCO purification (5% MeOH/DCM).

### Example 14

### Isobutyl 3-(Tert-Butyldisulfanyl)-1-Oxo-1-(Piperidin-4-ylamino)Propan-2-ylcarbamate

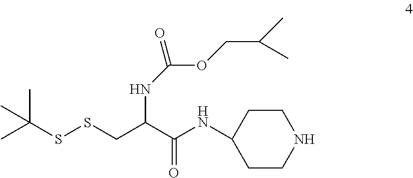 [SMILES: CC(C)COC(=O)NC(CSSC(C)(C)C)C(=O)NC1CCNCC1]

To compound 3 prepared in Example 13 (7.085 g, 18.12 mmol) was added 30 ml of 4M HCl/Dioxane. The reaction was completed after 2 h at RT. The HCl salt (4) was used for the next step without further purification.

### Example 15

### Isobutyl 3-(Tert-Butyldisulfanyl)-1-(1-(Dichlorophosphoryl)Piperidin-4-ylamino)-1-Oxopropan-2-ylcarbamate

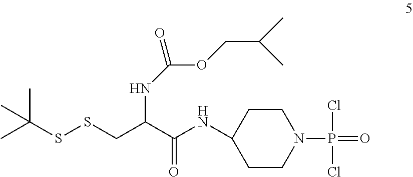 [SMILES: CC(C)COC(=O)NC(CSSC(C)(C)C)C(=O)NC1CCN(P(=O)(Cl)Cl)CC1]

To compound 4 prepared in Example 15 (7.746 g, 18.12 mmol) in DCM (200 ml) at −78 °C was slowly injected POCl₃ (1.69 ml, 18.12 mmol) under Ar, followed by the addition of Et₃N (7.58 ml, 54.36 mmol). The reaction was stirred at RT for 5h, concentrated to remove excess base and solvent. The product (5) was given as white solid after ISCO purification (50% EtAc/Hexane).

### Example 16

### Isobutyl 3-(Tert-Butyldisulfanyl)-1-(1-(Chloro(((2S,6R)-6-(5-Methyl-2,4-Dioxo-3,4-Dihydropyrimidin-1(2H)-yl)-4-Tritylmorpholin-2-yl)Methoxy)Phosphoryl)Piperidin-4-ylamino)-1-Oxopropan-2-ylcarbamate

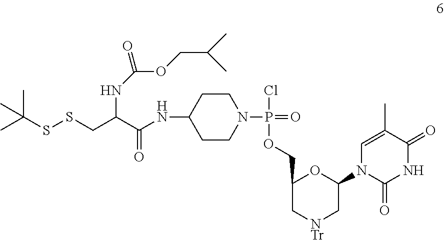

To 1-((2R,6S)-6-(hydroxymethyl)-4-tritylmorpholin-2-yl)-5-methylpyrimidine-2,4(1H,3H)-dione (moT(Tr)) (5.576 g, 10.98 mmol) in DCM (100 ml) at 0 °C, was added lutidine (1.92 ml, 16.47 mmol) and DMAP (669 mg, 5.5 mmol), followed by the addition of 4 (6.13 g, 12.08 mmol). The reaction was left stirring at RT for 18 h. The desired product (6) was obtained after ISCO purification (50% EtAc/Hexane).

### Example 17

### ((2S,6R)-6-(5-Methyl-2,4-Dioxo-3,4-Dihydropyrimidin-1(2H)-yl)-4-Tritylmorpholin-2-yl)Methyl Hexyl(Methyl)Phosphoramidochloridate

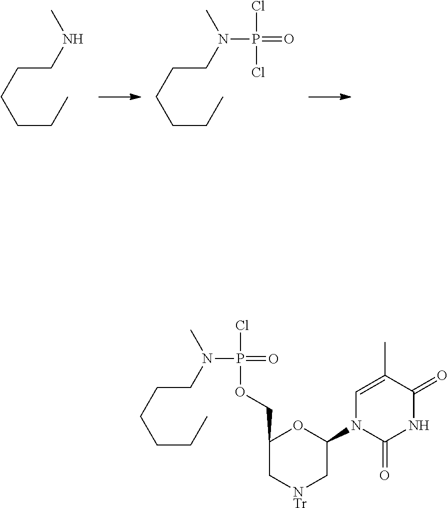

A DCM (80 ml) solution of N-hydroxylmethylamine (4.85 ml, 32 mmol) was cooled down to −78 °C under N2. A solution of phosphoryl chloride (2.98 ml, 32 mmol) in DCM (10 ml), followed by a solution of Et₃N (4.46 ml, 32 mmol) in DCM (10 ml), was added slowly. The stirring was continued while the reaction was allowed to warm to RT overnight. The desired product (1) was given as clear oil after ISCO purification (20% EtAc/Hexane).

To moT(Tr) (5.10 g, 10.54 mmol) in DCM (100 ml) at 0 °C, was added lutidine (3.68 ml, 31.6 mmol) and DMAP (642 mg, 5.27 mmol), followed by the addition of 1 (4.89 g, 21.08 mmol). The reaction was left stirring at RT for 18 h. The desired product (2) was obtained after ISCO purification (50% EtOAc/Hexane).

### Example 18

### ((2S,6R)-6-(5-Methyl-2,4-Dioxo-3,4-Dihydropyrimidin-1(2H)-yl)-4-Tritylmorpholin-2-yl)Methyl Dodecyl(Methyl)Phosphoramidochloridate

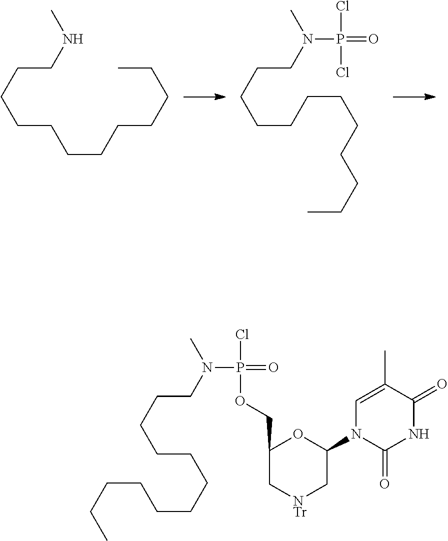

The title compound was prepared according to the general procedures described in Examples 6 and 8.

### Example 19

### ((2S,6R)-6-(5-Methyl-2,4-Dioxo-3,4-Dihydropyrimidin-1(2H)-yl)-4-Tritylmorpholin-2-yl)Methyl Morpholinophosphonochloridate

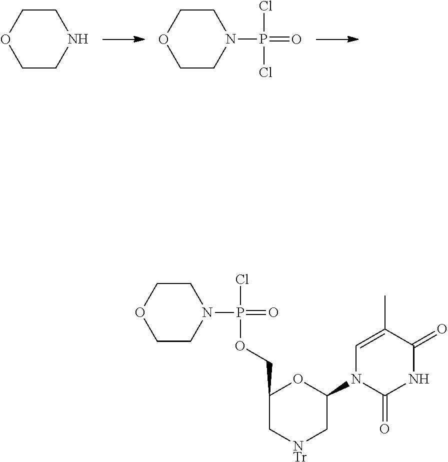

The title compound was prepared according to the general procedures described in Examples 6 and 8.

### Example 20

### ((2S,6R)-6-(5-Methyl-2,4-Dioxo-3,4-Dihydropyrimidin-1(2H)-yl)-4-Tritylmorpholin-2-yl)Methyl (S)-2-(Methoxymethyl)Pyrrolidin-1-ylphosphonochloridate

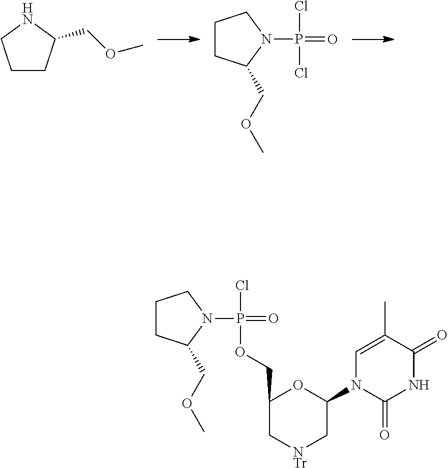

The title compound was prepared according to the general procedures described in Examples 6 and 8.

### Example 21

### ((2S,6R)-6-(5-Methyl-2,4-Dioxo-3,4-Dihydropyrimidin-1(2H)-yl)-4-Tritylmorpholin-2-yl)Methyl 4-(3,4,5-Trimethoxybenzamido)Piperidin-1-ylphosphonochloridate

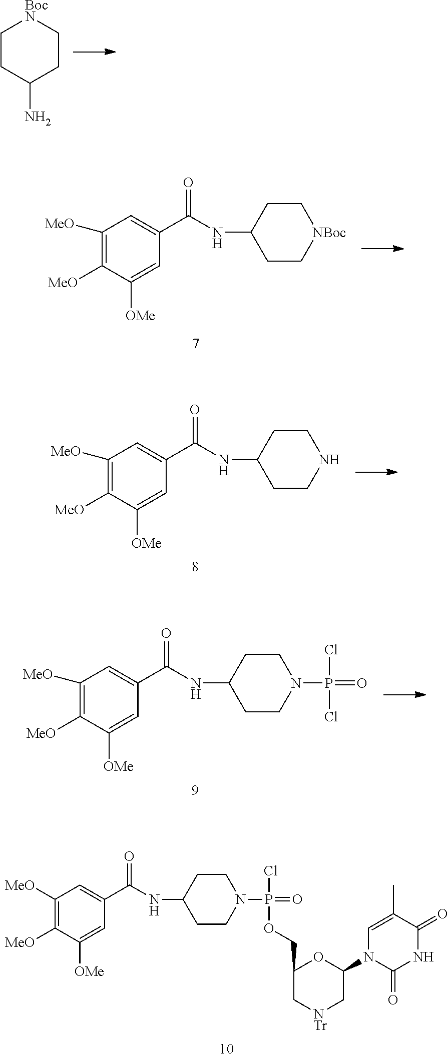

To 1-Boc-4-piperidine (1 g, 5 mmol) in DCM (20 ml) was added Hunig base (1.74 ml, 10 mmol), followed by the addition of 3,4,5-trimethoxybenzoyl chloride (1.38 g, 6 mmol). The reaction was run at RT for 3h, concentrated to remove solvent and excess base. The residue was dissolved in EtAc (100 ml), washed with 0.05N HCl (3×15 ml), sat. NaHCO₃ (2×15 ml), dried over Na₂SO₄. Product (1) was obtained after ISCO purification (5% MeOH/DCM).

To 7 was added 15 ml of 4N HCl/Dioxane, reaction was terminated after 4h. 8 was obtained as white solid.

A DCM (20 ml) solution of 8 (1.23 g, 4.18 mmol) was cooled down to −78 °C under N₂. A solution of phosphoryl chloride (0.39 ml, 4.18 mmol) in DCM (2 ml), followed by a solution of Et₃N (0.583 ml, 4.18 mmol) in DCM (2 ml), was added slowly. The stirring was continued while the reaction was allowed to warm to RT overnight. The desired product (9) was obtained after ISCO purification (50% EtAc/Hexane).

To moT(Tr) (1.933 g, 4.0 mmol) in DCM (20 ml) at 0 °C, was added lutidine (0.93 ml, 8 mmol) and DMAP (49 mg, 0.4 mmol), followed by the addition of 9 (1.647 g, 4 mmol). The reaction was left stirring at RT for 18 h. The desired product (10) was obtained after ISCO purification (50% EtAc/Hexane).

### Example 22

### Synthesis of Cyclophosphoramide Containing Subunit (^{CP}T)

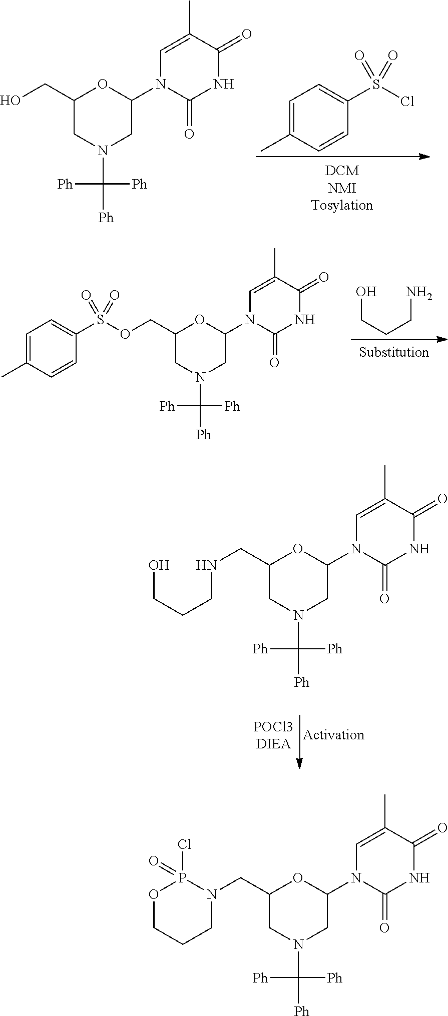

The moT subunit (25 g) was suspended in DCM (175 ml) and NMI (N-methylimidazole, 5.94 g, 1.4 eq.) was added to obtain a clear solution. Tosyl chloride was added to the reaction mixture, and the reaction progress was monitored by TLC until done (about 2 hours). An aqueous workup was performed by washing with 0.5 M citric acid buffer (pH=5), followed by brine. The organic layer was separated and dried over Na2SO4. Solvent was removed with a rotoevaporator to obtain the crude product which was used in the next step without further purification.

The moT Tosylate prepared above was mixed with propanolamine (1 g/10 ml). The reaction mixture was then placed in an oven at 45 °C overnight followed by dilution with DCM (10 ml). An aqueous workup was performed by washing with 0.5 M citric acid buffer (pH=5), followed by brine. The organic layer was separated and dried over Na₂SO₄. Solvent was removed with a rotavaporator to obtain the crude product. The curde product was analyzed by NMR and HPLC and determined to be ready for the next step without further purification.

The crude product was dissolved in DCM (2.5 ml DCM/g, 1 eq.) and mixed with DIEA (3 eq.). This solution was cooled with dry ice-acetone and POCl₃ was added dropwise (1.5 eq.). The resultant mixture was stirred at room temperature overnight. An aqueous workup was performed by washing with 0.5 M citric acid buffer (pH=5), followed by brine. The organic layer was separated and dried over Na₂SO₄. Solvent was removed with a rotavaporator to obtain the crude product as a yellowish solid. The crude product was purified by silica gel chromatography (crude product/silica=1 to 5 ratio, gradient DCM to 50% EA/DCM), and fractions were pooled according to TLC analysis. Solvent was removed to obtain the desired product as a mixture of diastereomers. The purified product was analyzed by HPLC (NPP quench) and NMR (H-1 and P-31).

The diastereomeric mixture was separated according to the following procedure. The mixture (2.6 g) was dissolved in DCM. This sample was loaded on a RediSepRf column (80 g normal phase made by Teledyne Isco) and eluted with 10% EA/DCM to 50% EA/DCM over 20 minutes. Fractions were collected and analyzed by TLC. Fractions were pooled according to TLC analysis, and solvent was removed with a rotavaporator at room temperature. The diastereomeric ratio of there pooled fractions was determined by P-31 NMR and NPP-TFA analysis. If needed, the above procedure was repeated until the diastereomeric ratio reached 97%.

### Example 23

### Global Cholic Acid Modification of PMOₚₗᵤₛ

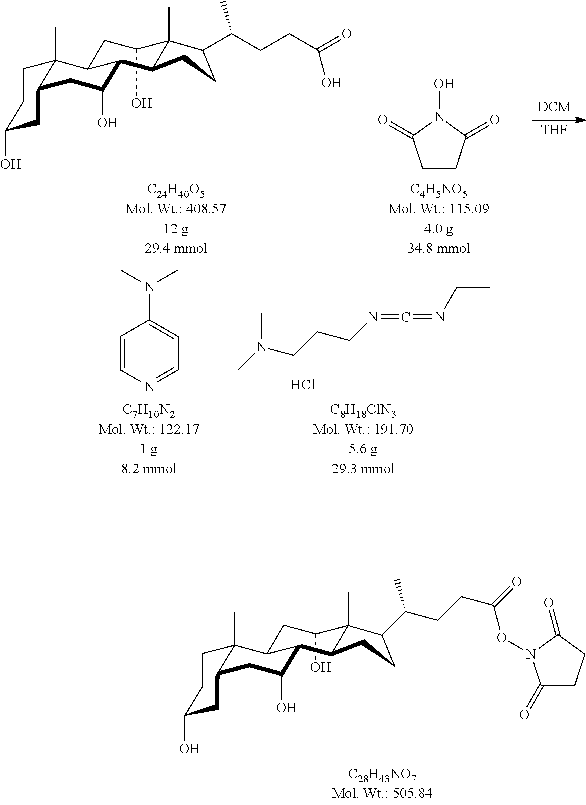

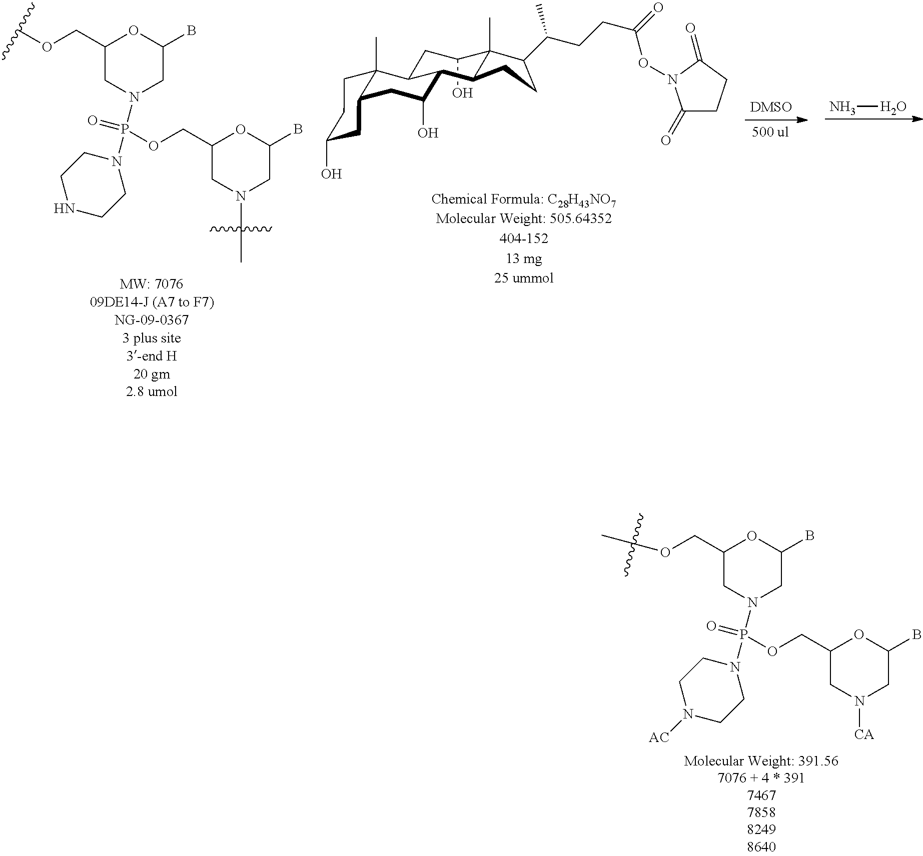

The succinimide activated cholic acid derivative was prepared according to the following procedure. Cholic acid (12 g, 29.4 mmol), N-hydroxysuccinimide (4.0 g, 34.8 mmol), EDCI (5.6 g, 29.3 mmol), and DMAP (1 g, 8.2 mmol) were charged to a round bottom flask. DCM (400 ml) and THE (40 ml) were added to dissolve. The reaction mixture was stirred at room temperature overnight. Water (400 ml) was then added to the reaction mixture, the organic layer separated and washed with water (2×400 ml), followed by sat. NaHCO₃ (300 ml) and brine (300 ml). The organic layer was then dried over Na₂SO₄. Solvent was removed with rotavaporator to obtain a white solid. The crude product was dissolved in chloroform (100 ml) and precipitated into heptane (1000 ml). The solid was collected by filtration, analyzed by HPLC and NMR and used without further purification.

An appropriate amount of PMOplus (20 mg, 2.8 μmol) was weighed into a vial (4 ml) and dissolved in DMSO (500 ul). The activated cholate ester (13 mg, 25 μmol) was added to the reaction mixture according to the ratio of two equivalent of active ester per modification site followed by stirring at room temperature overnight. Reaction progress was determined by MALDI and HPLC (C-18 or SAX).

After the reaction was complete (as determined by disappearance of starting PMOplus), 1 ml of concentrated ammonia was added to the reaction mixture once the reaction is complete. The reaction vial was then placed in an oven (45 °C) overnight (18 hours) followed by cooling to room temperature and dilution with 1% ammonia in water (10 ml). This sample was loaded on to an SPE column (2 cm), and the vial rinsed with 1% ammonia solution (2×2 ml). The SPE column was washed with 1% ammonia in water (3×6 ml), and the product eluted with 45% acetonitrile in 1% ammonia in water (6 ml). Fractions containing oligomer were identified by UV optical density measurement. Product was isolated by lyophilization. Purity and identity were determined by MALDI and HPLC (C-18 and/or SAX).

This same procedure is applicable to deoxycholic acid activation and conjugation to a PMO*.

### Example 24

### Global Guanidinylation of PMOplus

An appropriate amount of PMOplus (25 mg, 2.8 μmol) was weighed into a vial (6 ml). 1H-Pyrozole-1-carboxamidine chloride (15 mg, 102 μmol) and potassium carbonate (20 mg, 0.15 mmol) were added to the vial. Water was added (500 ul), and the reaction mixture was stirred at room temperature overnight (about 18 hours). Reaction completion was determined by MALDI.

Once complete, the reaction was diluted with 1% ammonia in water (10 ml) and loaded on to an SPE column (2 cm). The vial was rinsed with 1% ammonia solution (2×2 ml), and the SPE column was washed with 1% ammonia in water (3×6 ml). Product was eluted with 45% acetonitrile in 1% ammonia in water (6 ml). Fractions containing oligomer were identified by UV optical density measurement. Product was isolated by lyophilization. Purity and identity were determined by MALDI and HPLC (C-18 and/or SAX).

### Example 25

### Global Thioacetyl Modification of PMOplus (M23D)

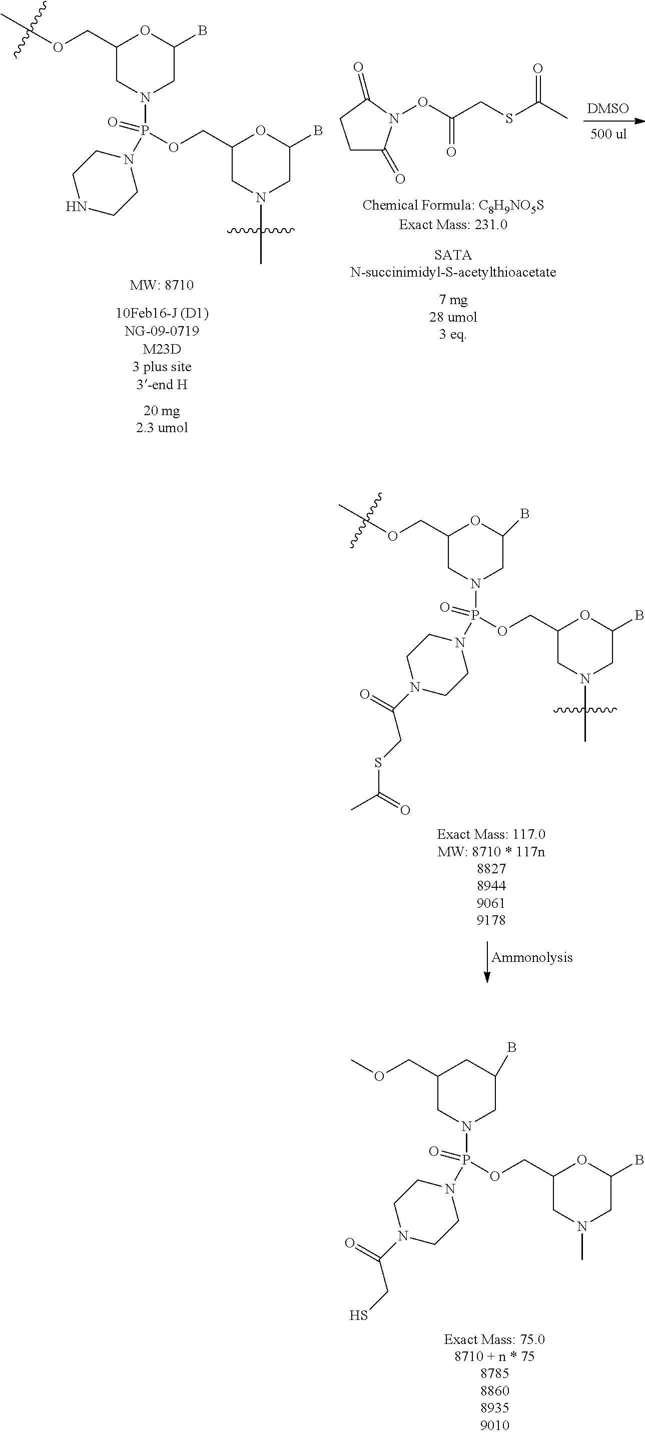

An appropriate amount of PMOplus (20 mg, 2.3 μmol) was weighed in to a vial (4 ml) and dissolved in DMSO (500 ul). N-succinimidyl-S-acetylthioacetate (SATA) (7 mg, 28 μmol) was added to the reaction mixture, and it was allowed to stir at room temperature overnight. Reaction progress was monitored by MALDI and HPLC.

Once complete, 1% ammonia in water was added to the reaction mixture, and it was stirred at room temperature for 2 hours. This solution was loaded on to an SPE column (2 cm), The vial was rinsed with 1% ammonia solution (2×2 ml), and the SPE column was washed with 1% ammonia in water (3×6 ml). Product was eluted with 45% acetonitrile in 1% ammonia in water (6 ml). Fractions containing oligomer were identified by UV optical density measurement. Product was isolated by lyophilization. Purity and identity were determined by MALDI and HPLC (C-18 and/or SAX).

### Example 26

### Global Succinic Acid Modification of PMOplus

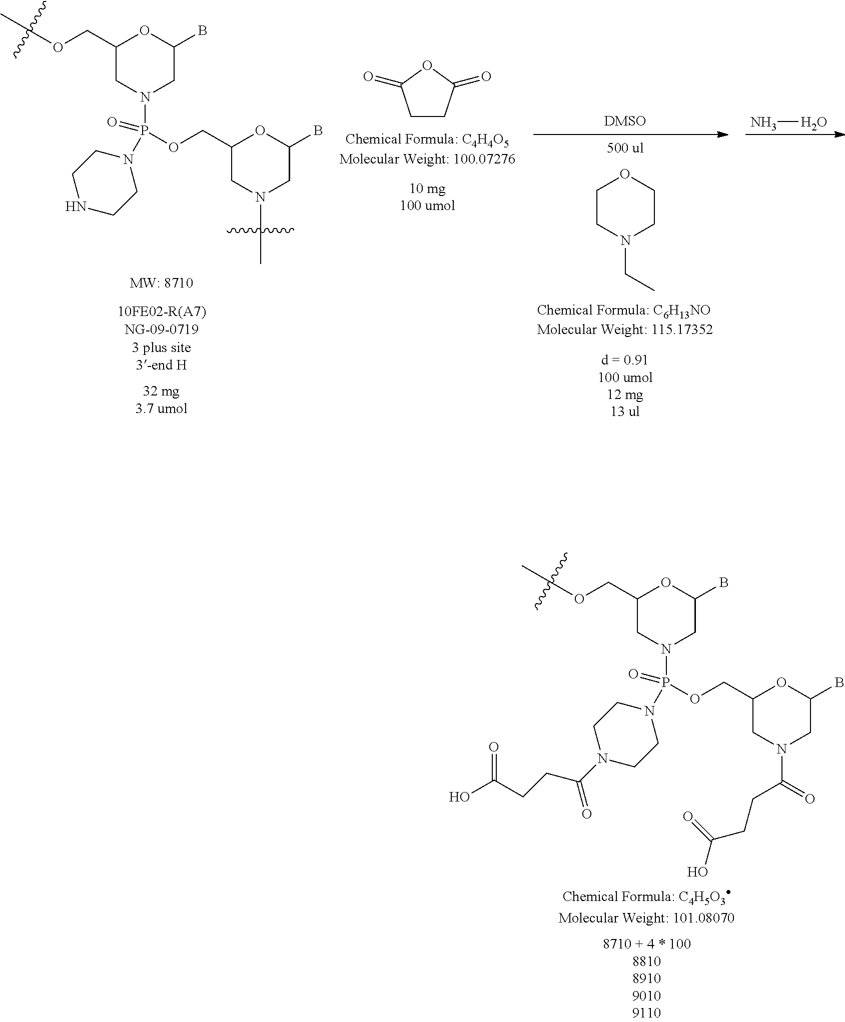

An appropriate amount of PMOplus (32 mg, 3.7 μmol) was weighed in to a vial (4 ml) and dissolved in DMSO (500 ul). N-ethyl morpholino (12 mg, 100 μmol) and succinic anhydride (10 mg, 100 μmol) were added to the reaction mixture, and it was allowed to stir at room temperature overnight. Reaction progress was monitored by MALDI and HPLC.

Once complete, 1% ammonia in water was added to the reaction mixture, and it was stirred at room temperature for 2 hours. This solution was loaded on to an SPE column (2 cm), The vial was rinsed with 1% ammonia solution (2×2 ml), and the SPE column was washed with 1% ammonia in water (3×6 ml). Product was eluted with 45% acetonitrile in 1% ammonia in water (6 ml). Fractions containing oligomer were identified by UV optical density measurement. Product was isolated by lyophilization. Purity and identity were determined by MALDI and HPLC (C-18 and/or SAX).

The above procedure is applicable to glutartic acid (glutaric anhydride) and tetramethyleneglutaric acid (tetramethyleneglutaric anhydride) modification of PMOplus as well.

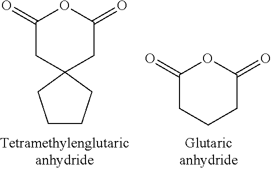 [SMILES: O=C1CC2(CCCC2)CC(=O)O1 ; O=C1CCCC(=O)O1]

### Example 27

### Preparation of an Oligonucleotide Analogue Comprising a Modified Terminal Group

To a solution of a 25-mer PMO containing a free 3′-end (27.7 mg, 3.226 μmol) in DMSO (300 μL) was added farnesyl bromide (1.75 1, 6.452 μmol) and diisopropylethylamine (2.24 μL, 12.9 μmol). The reaction mixture was stirred at room temperature for 5 hours. The crude reaction mixture was diluted with 10 mL of 1% aqueous NH₄OH, and then loaded onto a 2 mL Amberchrome CG300M column. The column was then rinsed with 3 column volumes of water, and the product was eluted with 6 mL of 1:1 acetonitrile and water (v/v). The solution was then lyophilized to obtain the title compound as a white solid.

### Example 28

### Preparation of Morpholino Oligomers

Preparation of trityl piperazine phenyl carbamate 35 (see FIG. 3): To a cooled suspension of compound 11 in dichloromethane (6 mL/g 11) was added a solution of potassium carbonate (3.2 eq) in water (4 mL/g potassium carbonate). To this two-phase mixture was slowly added a solution of phenyl chloroformate (1.03 eq) in dichloromethane (2 g/g phenyl chloroformate). The reaction mixture was warmed to 20 °C. Upon reaction completion (1-2 hr), the layers were separated. The organic layer was washed with water, and dried over anhydrous potassium carbonate. The product 35 was isolated by crystallization from acetonitrile. Yield=80%

Preparation of carbamate alcohol 36: Sodium hydride (1.2 eq) was suspended in 1-methyl-2-pyrrolidinone (32 mL/g sodium hydride). To this suspension were added triethylene glycol (10.0 eq) and compound 35 (1.0 eq). The resulting slurry was heated to 95 °C. Upon reaction completion (1-2 hr), the mixture was cooled to 20 °C. To this mixture was added 30% dichloromethane/methyl tert-butyl ether (v:v) and water. The product-containing organic layer was washed successively with aqueous NaOH, aqueous succinic acid, and saturated aqueous sodium chloride. The product 36 was isolated by crystallization from dichloromethane/methyl tert-butyl ether/heptane. Yield=90%.

Preparation of Tail acid 37: To a solution of compound 36 in tetrahydrofuran (7 mL/g 36) was added succinic anhydride (2.0 eq) and DMAP (0.5 eq). The mixture was heated to 50 °C. Upon reaction completion (5 hr), the mixture was cooled to 20 °C and adjusted to pH 8.5 with aqueous NaHCO₃. Methyl tert-butyl ether was added, and the product was extracted into the aqueous layer. Dichloromethane was added, and the mixture was adjusted to pH 3 with aqueous citric acid. The product-containing organic layer was washed with a mixture of pH=3 citrate buffer and saturated aqueous sodium chloride. This dichloromethane solution of 37 was used without isolation in the preparation of compound 38.

Preparation of 38: To the solution of compound 37 was added N-hydroxy-5-norbornene-2,3-dicarboxylic acid imide (HONB) (1.02 eq), 4-dimethylaminopyridine (DMAP) (0.34 eq), and then 1-(3-dimethylaminopropyl)-N′-ethylcarbodiimide hydrochloride (EDC) (1.1 eq). The mixture was heated to 55 °C. Upon reaction completion (4-5 hr), the mixture was cooled to 20 °C and washed successively with 1:1 0.2 M citric acid/brine and brine. The dichloromethane solution underwent solvent exchange to acetone and then to N,N-dimethylformamide, and the product was isolated by precipitation from acetone/N,N-dimethylformamide into saturated aqueous sodium chloride. The crude product was reslurried several times in water to remove residual N,N-dimethylformamide and salts. Yield=70% of 38 from compound 36. Introduction of the activated “Tail” onto the disulfide anchor-resin was performed in NMP by the procedure used for incorporation of the subunits during solid phase synthesis.

Preparation of the Solid Support for Synthesis of Morpholino Oligomers: This procedure was performed in a silanized, jacketed peptide vessel (custom made by ChemGlass, NJ, USA) with a coarse porosity (40-60 μm) glass frit, overhead stirrer, and 3-way Teflon stopcock to allow N2 to bubble up through the frit or a vacuum extraction. Temperature control was achieved in the reaction vessel by a circulating water bath.

The resin treatment/wash steps in the following procedure consist of two basic operations: resin fluidization and solvent/solution extraction. For resin fluidization, the stopcock was positioned to allow N2 flow up through the frit and the specified resin treatment/wash was added to the reactor and allowed to permeate and completely wet the resin. Mixing was then started and the resin slurry mixed for the specified time. For solvent/solution extraction, mixing and N2 flow were stopped and the vacuum pump was started and then the stopcock was positioned to allow evacuation of resin treatment/wash to waste. All resin treatment/wash volumes were 15 mL/g of resin unless noted otherwise.

To aminomethylpolystyrene resin (100-200 mesh; ~1.0 mmol/g N2 substitution; 75 g, 1 eq, Polymer Labs, UK, part #1464-X799) in a silanized, jacketed peptide vessel was added 1-methyl-2-pyrrolidinone (NMP; 20 ml/g resin) and the resin was allowed to swell with mixing for 1-2 hr. Following evacuation of the swell solvent, the resin was washed with dichloromethane (2×1-2 min), 5% diisopropylethylamine in 25% isopropanol/dichloromethane (2×3-4 min) and dichloromethane (2×1-2 min). After evacuation of the final wash, the resin was fluidized with a solution of disulfide anchor 34 in 1-methyl-2-pyrrolidinone (0.17 M; 15 mL/g resin, ~2.5 eq) and the resin/reagent mixture was heated at 45 °C for 60 hr. On reaction completion, heating was discontinued and the anchor solution was evacuated and the resin washed with 1-methyl-2-pyrrolidinone (4×3-4 min) and dichloromethane (6×1-2 min). The resin was treated with a solution of 10% (v/v) diethyl dicarbonate in dichloromethane (16 mL/g; 2×5-6 min) and then washed with dichloromethane (6×1-2 min). The resin 39 (see FIG. 4) was dried under a N2 stream for 1-3 hr and then under vacuum to constant weight (±2%). Yield: 110-150% of the original resin weight.

Determination of the Loading of Aminomethylpolystyrene-disulfide resin: The loading of the resin (number of potentially available reactive sites) is determined by a spectrometric assay for the number of triphenylmethyl (trityl) groups per gram of resin.

A known weight of dried resin (25±3 mg) is transferred to a silanized 25 ml volumetric flask and −5 mL of 2% (v/v) trifluoroacetic acid in dichloromethane is added. The contents are mixed by gentle swirling and then allowed to stand for 30 min. The volume is brought up to 25 mL with additional 2% (v/v) trifluoroacetic acid in dichloromethane and the contents thoroughly mixed. Using a positive displacement pipette, an aliquot of the trityl-containing solution (500 μL) is transferred to a 10 mL volumetric flask and the volume brought up to 10 mL with methanesulfonic acid.

The trityl cation content in the final solution is measured by UV absorbance at 431.7 nm and the resin loading calculated in trityl groups per gram resin (μmol/g) using the appropriate volumes, dilutions, extinction coefficient (ε: 41 μmol-1 cm-1) and resin weight. The assay is performed in triplicate and an average loading calculated.

The resin loading procedure in this example will provide resin with a loading of approximately 500 μmol/g. A loading of 300-400 in mol/g was obtained if the disulfide anchor incorporation step is performed for 24 hr at room temperature.

Tail loading: Using the same setup and volumes as for the preparation of aminomethylpolystyrene-disulfide resin, the Tail can be introduced into the molecule. For the coupling step, a solution of 38 (0.2 M) in NMP containing 4-ethylmorpholine (NEM, 0.4 M) was used instead of the disulfide anchor solution. After 2 hr at 45 °C, the resin 39 was washed twice with 5% diisopropylethylamine in 25% isopropanol/dichloromethane and once with DCM. To the resin was added a solution of benzoic anhydride (0.4 M) and NEM (0.4 M). After 25 min, the reactor jacket was cooled to room temperature, and the resin washed twice with 5% diisopropylethylamine in 25% isopropanol/dichloromethane and eight times with DCM. The resin 40 was filtered and dried under high vacuum. The loading for resin 40 is defined to be the loading of the original aminomethylpolystyrene-disulfide resin 39 used in the Tail loading.

Solid Phase Synthesis: Morpholino Oligomers were prepared on a Gilson AMS-422 Automated Peptide Synthesizer in 2 mL Gilson polypropylene reaction columns (Part #3980270). An aluminum block with channels for water flow was placed around the columns as they sat on the synthesizer. The AMS-422 will alternatively add reagent/wash solutions, hold for a specified time, and evacuate the columns using vacuum.

For oligomers in the range up to about 25 subunits in length, aminomethylpolystyrene-disulfide resin with loading near 500 μmol/g of resin is preferred. For larger oligomers, aminomethylpolystyrene-disulfide resin with loading of 300-400 μmol/g of resin is preferred. If a molecule with 5′-Tail is desired, resin that has been loaded with Tail is chosen with the same loading guidelines.

The following reagent solutions were prepared:

Detritylation Solution: 10% Cyanoacetic Acid (w/v) in 4:1 dichloromethane/acetonitrile; Neutralization Solution: 5% Diisopropylethylamine in 3:1 dichloromethane/isopropanol; Coupling Solution: 0.18 M (or 0.24 M for oligomers having grown longer than 20 subunits) activated Morpholino Subunit of the desired base and linkage type and 0.4 M N ethylmorpholine, in 1,3-dimethylimidazolidinone. Dichloromethane (DCM) was used as a transitional wash separating the different reagent solution washes.

On the synthesizer, with the block set to 42 °C, to each column containing 30 mg of aminomethylpolystyrene-disulfide resin (or Tail resin) was added 2 mL of 1-methyl-2-pyrrolidinone and allowed to sit at room temperature for 30 min. After washing with 2 times 2 mL of dichloromethane, the following synthesis cycle was employed:

| Step | Volume | Delivery | Hold time |
|---|---|---|---|
| Detritylation | 1.5 mL | Manifold | 15 seconds |
| Detritylation | 1.5 mL | Manifold | 15 seconds |
| Detritylation | 1.5 mL | Manifold | 15 seconds |
| Detritylation | 1.5 mL | Manifold | 15 seconds |
| Detritylation | 1.5 mL | Manifold | 15 seconds |
| Detritylation | 1.5 mL | Manifold | 15 seconds |
| Detritylation | 1.5 mL | Manifold | 15 seconds |
| DCM | 1.5 mL | Manifold | 30 seconds |
| Neutralization | 1.5 mL | Manifold | 30 seconds |
| Neutralization | 1.5 mL | Manifold | 30 seconds |
| Neutralization | 1.5 mL | Manifold | 30 seconds |
| Neutralization | 1.5 mL | Manifold | 30 seconds |
| Neutralization | 1.5 mL | Manifold | 30 seconds |
| Neutralization | 1.5 mL | Manifold | 30 seconds |
| DCM | 1.5 mL | Manifold | 30 seconds |
| Coupling | 350 uL-500 uL | Syringe | 40 minutes |
| DCM | 1.5 mL | Manifold | 30 seconds |
| Neutralization | 1.5 mL | Manifold | 30 seconds |
| Neutralization | 1.5 mL | Manifold | 30 seconds |
| DCM | 1.5 mL | Manifold | 30 seconds |
| DCM | 1.5 mL | Manifold | 30 seconds |
| DCM | 1.5 mL | Manifold | 30 seconds |

The sequences of the individual oligomers were programmed into the synthesizer so that each column receives the proper coupling solution (A,C,G,T,I) in the proper sequence. When the oligomer in a column had completed incorporation of its final subunit, the column was removed from the block and a final cycle performed manually with a coupling solution comprised of 4-methoxytriphenylmethyl chloride (0.32 M in DMI) containing 0.89 M 4-ethylmorpholine.

Cleavage from the resin and removal of bases and backbone protecting groups: After methoxytritylation, the resin was washed 8 times with 2 mL 1-methyl-2-pyrrolidinone. One mL of a cleavage solution consisting of 0.1 M 1,4-dithiothreitol (DTT) and 0.73 M triethylamine in 1-methyl-2-pyrrolidinone was added, the column capped, and allowed to sit at room temperature for 30 min. After that time, the solution was drained into a 12 mL Wheaton vial. The greatly shrunken resin was washed twice with 300 μL of cleavage solution. To the solution was added 4.0 mL conc aqueous ammonia (stored at −20 °C), the vial capped tightly (with Teflon lined screw cap), and the mixture swirled to mix the solution. The vial was placed in a 45 °C oven for 16-24 hr to effect cleavage of base and backbone protecting groups.

Initial Oligomer Isolation: The vialed ammonolysis solution was removed from the oven and allowed to cool to room temperature. The solution was diluted with 20 mL of 0.28% aqueous ammonia and passed through a 2.5×10 cm column containing Macroprep HQ resin (BioRad). A salt gradient (A: 0.28% ammonia with B: 1 M sodium chloride in 0.28% ammonia; 0-100% B in 60 min) was used to elute the methoxytrityl containing peak. The combined fractions were pooled and further processed depending on the desired product.

Demethoxytritylation of Morpholino Oligomers: The pooled fractions from the Macroprep purification were treated with 1 M H3PO4 to lower the pH to 2.5. After initial mixing, the samples sat at room temperature for 4 min, at which time they are neutralized to pH 10-11 with 2.8% ammonia/water. The products were purified by solid phase extraction (SPE).

Amberchrome CG-300M (Rohm and Haas; Philadelphia, PA) (3 mL) is packed into 20 mL fritted columns (BioRad Econo-Pac Chromatography Columns (732-1011)) and the resin rinsed with 3 mL of the following: 0.28% NH₄OH/80% acetonitrile; 0.5M NaOH/20% ethanol; water; 50 mM H3PO4/80% acetonitrile; water; 0.5 NaOH/20% ethanol; water; 0.28% NH₄OH.

The solution from the demethoxytritylation was loaded onto the column and the resin rinsed three times with 3-6 mL 0.28% aqueous ammonia. A Wheaton vial (12 mL) was placed under the column and the product eluted by two washes with 2 mL of 45% acetonitrile in 0.28% aqueous ammonia. The solutions were frozen in dry ice and the vials placed in a freeze dryer to produce a fluffy white powder. The samples were dissolved in water, filtered through a 0.22 micron filter (Pall Life Sciences, Acrodisc 25 mm syringe filter, with a 0.2 micron HT Tuffryn membrane) using a syringe and the Optical Density (OD) was measured on a UV spectrophotometer to determine the OD units of oligomer present, as well as dispense sample for analysis. The solutions were then placed back in Wheaton vials for lyophilization.

Analysis of Morpholino Oligomers: MALDI-TOF mass spectrometry was used to determine the composition of fractions in purifications as well as provide evidence for identity (molecular weight) of the oligomers. Samples were run following dilution with solution of 3,5-dimethoxy-4-hydroxycinnamic acid (sinapinic acid), 3,4,5-trihydoxyacetophenone (THAP) or alpha-cyano-4-hydoxycinnamic acid (HCCA) as matrices.

Cation exchange (SCX) HPLC was performed using a Dionex ProPac SCX-10, 4×250 mm column (Dionex Corporation; Sunnyvale, CA) using 25 mM pH=5 sodium acetate 25% acetonitrile (Buffer A) and 25 mM pH=5 sodium acetate 25% acetonitrile 1.5 M potassium chloride (buffer B) (Gradient 10-100% B in 15 min) or 25 mM KH2PO4 25% acetonitrile at pH=3.5 (buffer A) and 25 mM KH2PO4 25% acetonitrile at pH=3.5 with 1.5 M potassium chloride (buffer B) (Gradient 0-35% B in 15 min). The former system was used for positively charged oligomers that do not have a peptide attached, while the latter was used for peptide conjugates.

Purification of Morpholino Oligomers by Cation Exchange Chromatography: The sample is dissolved in 20 mM sodium acetate, pH=4.5 (buffer A) and applied to a column of Source 30 cation exchange resin (GE Healthcare) and eluted with a gradient of 0.5 M sodium chloride in 20 mM sodium acetate and 40% acetonitrile, pH=4.5 (buffer B). The pooled fractions containing product are neutralized with conc aqueous ammonia and applied to an Amberchrome SPE column. The product is eluted, frozen, and lyophilized as above.

### Example 29

### Preparation of an Exemplary Conjugate

The peptide sequence AcR₆G was prepared according to standard peptide syhtentic methods known in the art. To a solution of the PMO (NG-05-0225, 3′-H: M23D: 5′-EG3, a sequence for binding to exon 23 of the mdx mouse, 350 mg, 1 eq), AcR₆G (142 mg, 2 eq), HATU (31 mg, 2 eq) in DMSO (3 mL) was added diisopropylethylamine (36 μL, 5 eq) at room temperature. After 1 hour, the reaction was worked up and the desired peptide-oligomer conjugate was purified by SCX chromatography (eluting with a gradient: A: 20 mM NaH2PO4 in 25% acetonitrile/H₂O, pH 7.0; B: 1.5 M guanidine HCl and 20 mM NaH2PO4 in 25% acetonitrile/H₂O, pH 7.0). The combined fractions were subjected to solid phase extraction (1M NaCl, followed by water elution). The conjugate was obtained as a white powder (257 mg, 65.5% yield) after lyophilization.

### Example 30

### Treatment of MDX Mice with Exemplary Conjugates of the Invention

The MDX mouse is an accepted and well-characterized animal model for Duchene muscular dystrophy (DMD) containing a mutation in exon 23 of the dystrophin gene. The M23D antisense sequence (SEQ ID NO:15) is known to induce exon 23 skipping and restoration of functional dystrophin expression. MDX mice were dosed once (50 mg/kg) by tail vein injection with one of the following conjugates:
1. 5′-EG3-M23D-BX(RXRRBR)₂ (AVI5225);
2. 5′-EG3-M23D-G(R)₅ (NG-11-0045);
3. 5′-EG3-M23D-G(R)₆ (NG-11-0009);
4. 5′-EG3-M23D-G(R)₇ (NG-11-0010); or
5. 5′-EG3-M23D-G(R)₈ (NG-11-0216)
   wherein M23D is a morpholino oligonucleotide having the sequence GGCCAAACCTCGGCTTACCTGAAAT (SEQ ID NO: 15) and “EG3” refers to the following structure:

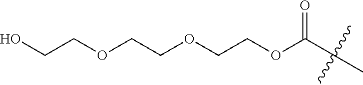 [SMILES: *C(=O)OCCOCCOCCO]

linked to the 5′ end of the oligomer via a piperazine linker (i.e., structure XXIX).

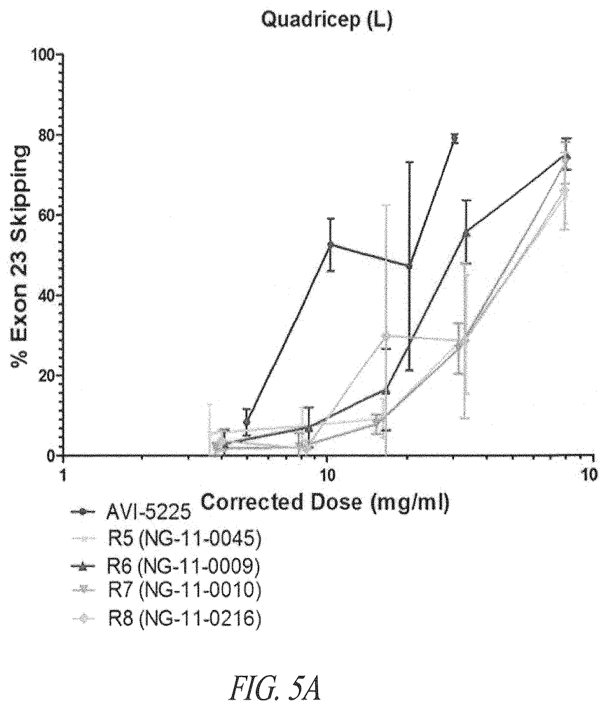

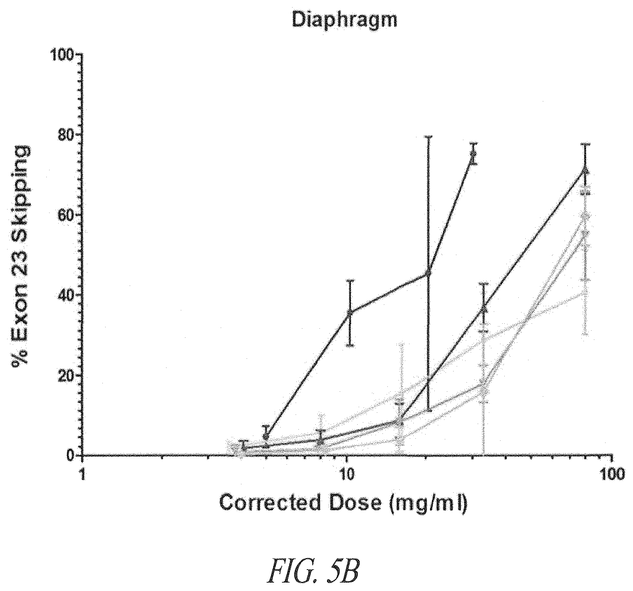

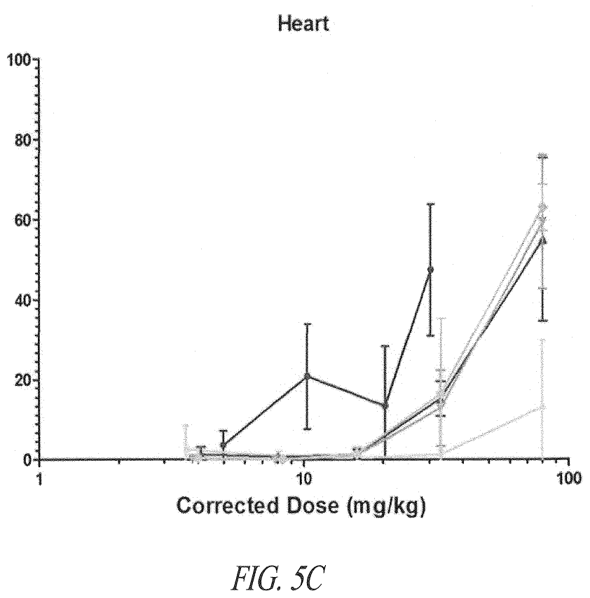

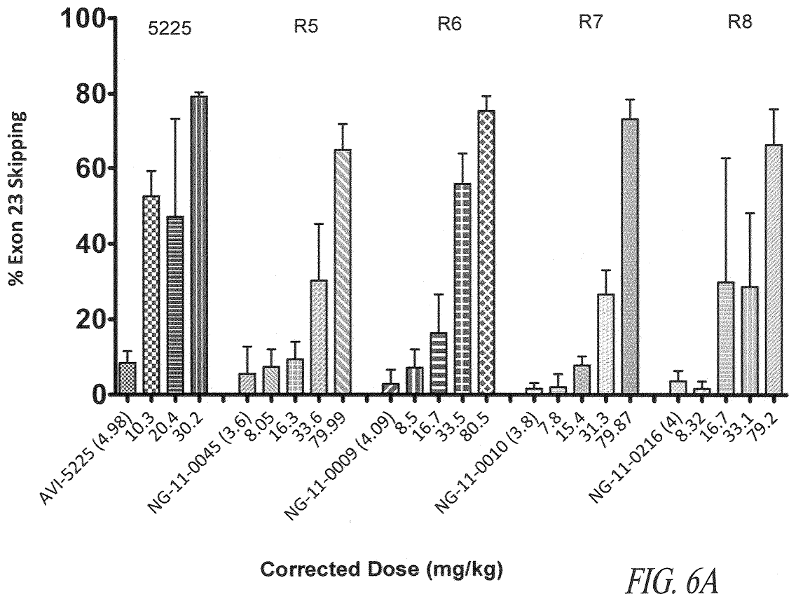

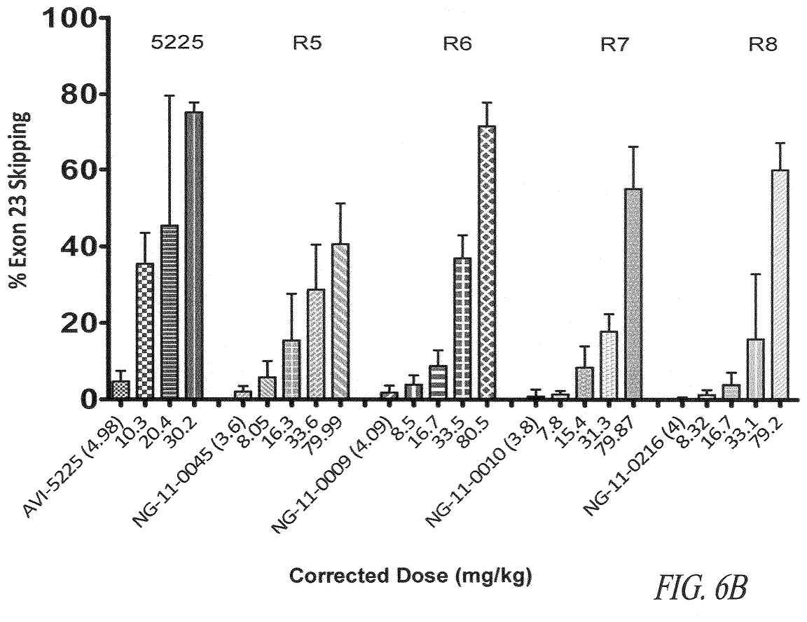

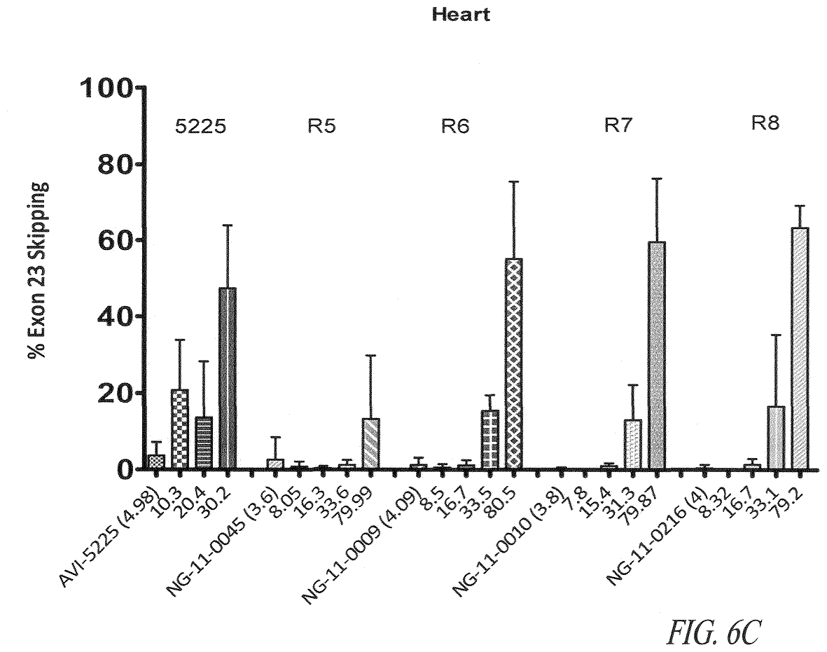

One week post-injection, the MDX mice were sacrificed and RNA was extracted from various muscle tissues. End-point PCR was used to determine the relative abundance of dystrophin mRNA containing exon 23 and mRNA lacking exon 23 due to antisense-induced exon skipping. Percent exon 23 skipping is a measure of antisense activity in vivo. FIGS. 5 and 6 show shows the results from the quadriceps (QC, FIGS. 5A and 6A), diaphragm (DT, FIGS. 5B and 6B) and heart (HT, FIGS. 5C and 6C), respectively one week post-treatment. The dose response between AVI-5225 and the other conjugates was similar. Amongst the arginine series, the R₆G peptide has the highest efficacy in quadriceps and diaphragm and was similar to the other arginine series peptides in heart.

### Example 31

### BUN Levels and Survival Rates of Mice Treated with Exemplary Conjugates

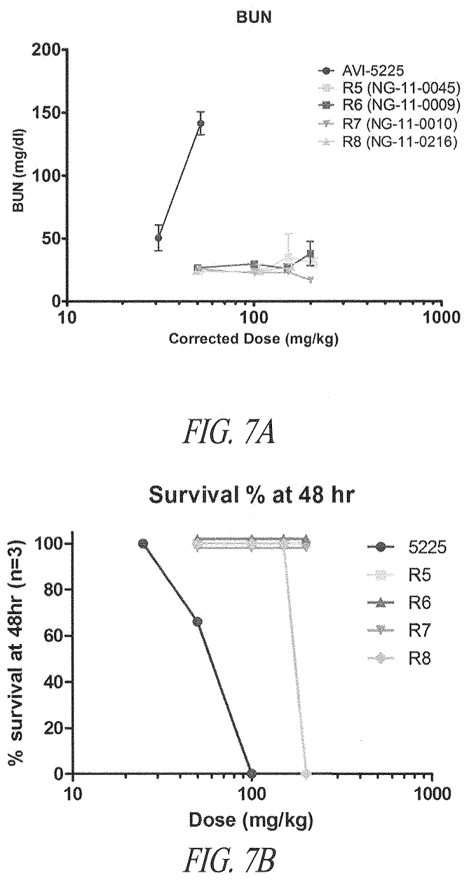

Mice were treated with the conjugates described in Example 30, and KIM-1 levels, BUN levels and survival rate were determined according to the general procedures described in Example 32 below and known in the art. Surprisingly, FIG. 7A shows that all glycine linked conjugates had significantly lower BUN levels than the XB linked conjugate (AVI-5225). In addition, mice treated with glycine linked conjugates survived longer at higher doses than the XB linked conjugate (FIG. 7B), with the R₈G conjugate being the least tolerated of the arginine polymers. All mice treated with the R₆G conjugate (NG-11-0009) survived at doses up to 400 mg/kg (data not shown).

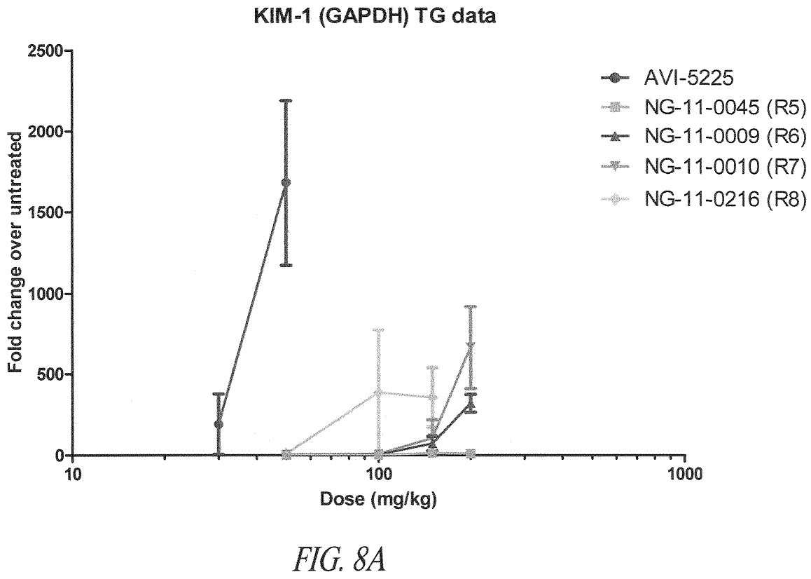

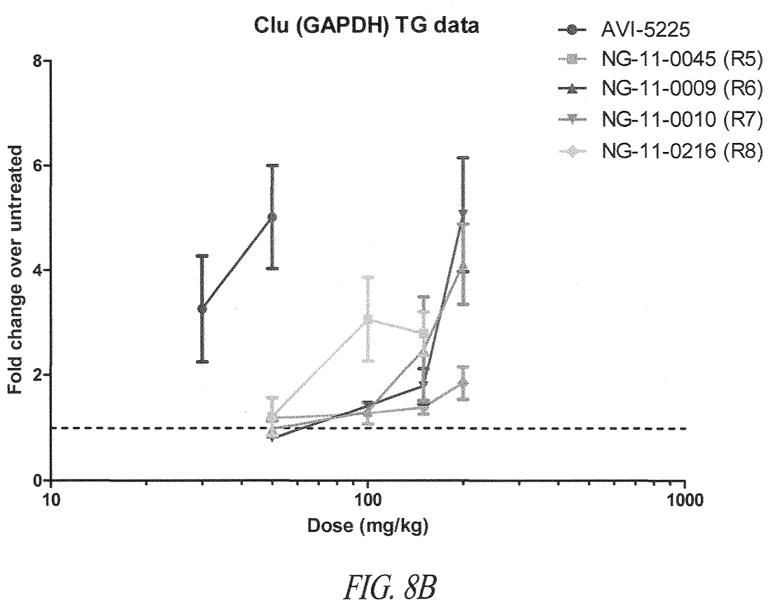

The KIM-1 (FIG. 8A) and Clusterin (FIG. 8B) levels of mice treated with the glycine linked conjugates was significantly lower than mice treated with AVI-5225. This data indicates that the conjugates of the present invention have lower toxicity than prior conjugates, and as shown above in Example 30, the efficacy of the conjugates is not decreased. Accordingly, the present conjugates have a better therapeutic window than other known conjugates and are potentially better drug candidates.

### Example 32

### Toxicology of Exemplary Conjugates

Four exemplary conjugates of the invention were tested for their toxicology in mice. The conjugates were as follows:
1. 5′-EG3-M23D-BX(RXRRBR)₂ (AVI5225);
2. 5′-EG3-M23D-G(RXRRBR)₂ (NG-11-0654);
3. 5′-EG3-M23D-BX(R)₆ (NG-11-0634); and
4. 5′-EG3-M23D-G(R)₆ (NG-11-0009)
   wherein M23D is a morpholino oligonucleotide having the sequence GGCCAAACCTCGGCTTACCTGAAAT and “EG3” refers to the following structure:

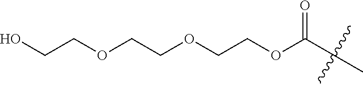 [SMILES: *C(=O)OCCOCCOCCO]

linked to the 5′ end of the oligomer via a piperazine linker (i.e., structure XXIX).

Eight week old male mice (C87/BL6; Jackson Laboratories, 18-22 grams) were treated with the above conjugates formulated in saline. The mice were acclimated for a minimum of five days prior to the commencement of the experimental procedures.

The animals were housed up to 3 per cage in clear polycarbonate microisolator cages with certified irradiated contact bedding. The cages conformed to standards set forth in the Animal Welfare Act (with all amendments) and the Guide for the Care and Use of Laboratory Animals, National Academy Press, Washington, D.C., 2010.

Animals were randomized into treatment groups based on cage weights specified in the table below. Group allocation was documented in the study records.

TABLE 8
Toxicology Study Design

| Group | | Dose per injection | | Route of |
|---|---|---|---|---|
| n = 3 | Oligo | (mg/kg) | Regimen | Admin. |
| 1 | NG-11-0654 | 50 | Single | Tail Vein, i.v. |
| 2 | NG-11-0654 | 100 | injection | 200 μl |
| 3 | NG-11-0654 | 150 | | |
| 4 | NG-11-0654 | 200 | | |
| 5 | NG-11-0634 | 50 | | |
| 6 | NG-11-0634 | 100 | | |
| 7 | NG-11-0634 | 150 | | |
| 8 | NG-11-0634 | 200 | | |
| 9 | NG-11-0009 | 50 | | |
| 10 | NG-11-0009 | 100 | | |
| 11 | NG-11-0009 | 150 | | |
| 12 | NG-11-0009 | 200 | | |
| 13 | AVI-5225 | 25 | | |
| 14 | AVI-5225 | 50 | | |
| 15 | AVI-5225 | 100 | | |
| 16 | Vehicle | 0 | | |

The day of dosing on the study was designated as Study Day 1. Conjugate was administered via tail vein as a slow push bolus (~5 seconds). All animals were dosed over two days. Groups 1-8 were dosed on the first day and Groups 9-16 were dosed on the second day. Treatment Groups (TG) 13-16 were dosed per the table above. Results from these TGs did not affect progression to other TG. The first 2 TG of each conjugate were dosed per the table above. If all animals in 100 mg/kg group died then the remaining TGs of that test article would not be dosed and the study would end. If at least one animal survived 2 hours post-dose in the 100 mg/kg group, then the 150 mg/kg group was dosed. If all animals in the 150 mg/kg group died then the remaining TGs of that test article would not be dosed and the study would end. If at least one animal survived 2 hours post-dose in the 150 mg/kg group, then the 200 mg/kg group was dosed.

Animals were observed for moribundity and mortality once daily. Any animal showing signs of distress, particularly if death appeared imminent was humanely euthanized according to Numira Biosciences Standard Operating Procedures. Body weights were recorded on the day after arrival, the day of dosing, and the day of necropsy. Detailed clinical observations were conducted and recorded at 0 minutes, 15 minutes, and 2 hours post-dose to assess tolerability of injections.

Blood samples (maximum volume, approximately 1 mL) were obtained from all animals via cardiac puncture 3 days post-dose prior to necropsy. Blood samples were collected into red top microtainer tubes and held at room temperature for at least 30 minutes but no longer than 60 minutes prior to centrifugation. Samples were centrifuged at approximately 1500-2500 rpm for 15-20 minutes to obtain serum.

Animals unlikely to survive until the next scheduled observation were weighed and euthanized. Animals found dead were weighed and the time of death was estimated as closely as possible. Blood and tissue samples were not collected.

Day 3 (2 days post-dose), all animals were humanely euthanized with carbon dioxide. Euthanasia was performed in accordance with accepted American Veterinary Medical Association (AVMA) guidelines on Euthanasia, June 2007.

The partial gross necropsy included examination and documentation of findings. All external surfaces and orifices were evaluated. All abnormalities observed during the collection of the tissues were described completely and recorded. No additional tissues were taken.

The right and left kidneys were collected. Tissues were collected within 15 minutes or less of euthanasia. All instruments and tools used were changed between treatment groups. All tissues were flash frozen and stored at <−70 °C as soon as possible after collection.

Kidney injury marker data was obtained as follows. RNA from mouse kidney tissue was purified using Quick Gene Mini80 Tissue Kit SII (Fuji Film). Briefly, approximately 40 mg of tissue was added to 0.5 ml lysis buffer (5 μl 2-mercaptoethanol in 0.5 ml lysis buffer) in a MagnaLyser Green Bead vial (Roche) and homogenized using MagNA Lyser (Roche) with 2 sets of 3×3800 RPM and 3 sets of 1×6500 RPM. Samples were cooled on ice 3-4 minutes between each low speed set and between each higher speed run. Homogenates were centrifuged 5 minute at 400×g at room temperature. The homogenate was immediately processed for RNA purification according to the Quick Gene Mini80 protocol. Samples underwent an on-column DNA digestion with DNase I (Qiagen) for 5 minutes. Total RNA was quantitated with a NanoDrop 2000 spectrophotometer (Thermo Scientific).

qRT-PCR was Performed Using Applied Biosystems Reagents (One-Step RT-PCR) and Pre-Designed Primer/Probe Sets (ACTB, GAPDH, KIM-1, Clusterin—FAM Reporter)

| Reagent | Company | Cat. No. |
|---|---|---|
| One-step PCR kit | Applied Biosystems | 4309169 |
| GAPDH mouse primer/ | Applied Biosystems | 4352932E |
| probe set | | |
| KIM-1 mouse primer/ | Applied Biosystems | Mm00506686_m1 |
| probe set | | |

Each reaction contained the following (30 ul total):
15 ul 2×qRT-PCR Buffer from ABI One-Step Kit
1.5 ul Primer/Probe mix
8.75 ul Nuclease-free water
0.75 ul 40× multiscript+RNase inhibitor
4 ul RNA template (100 ng/ul)

The qRT One-Step Program was run as follows:
1. 48 C for 30 minutes
2. 95 C for 10 minutes
3. 95 C for 15 seconds
4. 60 C for 1 minute
5. Repeat Steps 3-4 39 times for a total of 40 cycles

Samples were run in triplicate wells and averaged for further analysis. Analysis was performed using ΔΔCt method. Briefly, Experimental ΔCt [Ct(Target)−Ct(Reference)] subtracted by Control ΔCt [Ct(Target)−Ct(Reference)]=ΔΔCt. Fold change range calculated: 2\hat{ }−(ΔΔCt+SD) to 2\hat{ }−(ΔΔCt−SD). Control=vehicle treated animal group (pooled), Target=KIM−1; Reference=GAPDH; SD=Sqrt[(SDtarget\hat{ }2)+(SDref\hat{ }2)].

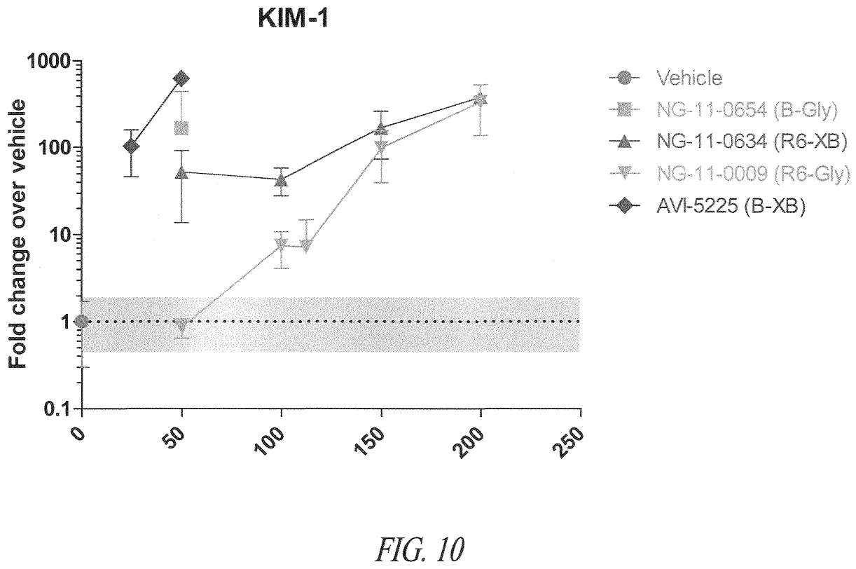

Results of KIM data are shown in FIG. 10. Conjugates comprising carrier peptides with terminal glycines had lower KIM concentrations with the R₆G peptide having the lowest. Both the terminal G and the presence of unnatural amino acids (aminohexanoic acid) appear to play a role in the toxicity of the conjugates.

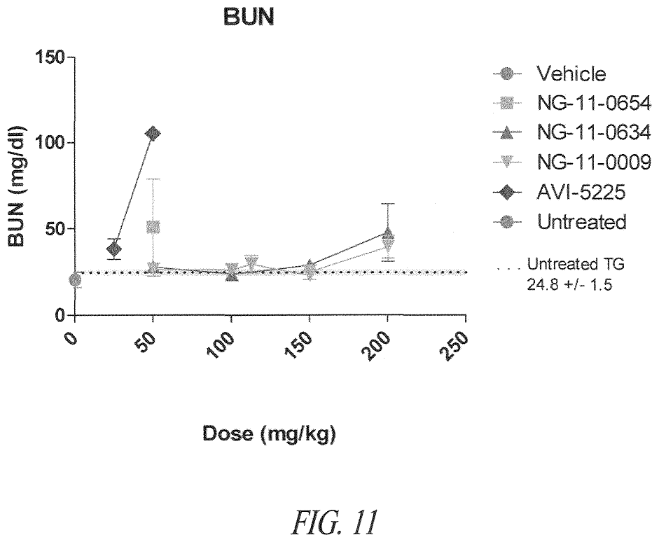

Frozen serum samples were sent on dry ice to IDEXX Laboratories (West Sacramento, CA) for processing. Serum dilution was performed per IDEXX Standard Operating Procedures (SOPs) when necessary. Blood chemistry results were were analyzed. Blood urea nitrogen levels are shown in FIG. 11. Again, the G-linked conjugate had lower BUN levels and the both the terminal G and overall peptide sequence appear to play a role in the toxicological profile of the conjugates.

Kidney tissues (approx. 150 mg) were weighed accurately in a 2 mL screw cap vial partially filled with ceramic beads. Five volume parts Tissue PE LB buffer (G Biosciences) containing 10 U/mL Proteinase K (Sigma) were added to 1 part tissue. Samples were homogenized with a Roche MagnaLyser (4×40 sec @7,000 rpm, with cooling between runs) and incubated for 30 min at 40 °C. When required, tissue homogenates were diluted with BSAsal (3 mg/mL BSA+20 mM NaCl) to bring high sample concentrations into the calibration range.

Calibration samples were prepared by spiking a solution of 3 mg/mL of BSA in 20 mM NaCl with known amounts of an appropriate analytical reference standard. Duplicate sets of eight samples each were prepared. The ULOQ was 40 μg/mL and LLOQ was 0.065536 μg/mL. An internal standard (NG-07-0775) was added to all samples except some blank samples designated as double blanks (no drug, no internal standard). Samples were extracted by vortexing 100 μL aliquots with 3 volumes of methanol.

After centrifugation (15 min, 14,000 rpm) supernatants were transferred to new tubes and dried in a Speedvac. Dried samples were reconstituted with an appropriate amount of FDNA (5′ d FAM-ATTTCAGGTAAGCCGAGGTTTGGCC 3′) in [10 mM Tris pH 8.0±1 mM EDTA+100 mM NaCl]-acetonitrile (75-25).

Samples were analyzed on the Dionex UltiMate 3000 HPLC using anion-exchange chromatography (Dionex DNAPac 4×250 mm column). Injection volume was 5 μL. Mobile phase was composed of 20% acetonitrile and 80% water containing 25 mM Tris pH 8.0 and a gradient of increasing NaCl concentration. Flow rate was 1 mL/min, and run time was 10 min per sample. The fluorescence detector was set to EX 494 nm and EM 520 nm. Peak identification was based on retention time. Peak height ratios (analyte:internal standard) were used for quantitation. Calibration curves were calculated based on the averaged response factors of duplicate calibration samples (one set run at the beginning of the batch, the other at the end of the batch. Linear curve fit with 1/x weighting factor was used. Blank samples (calibration sample with no reference compound added) and double blank samples (on internal standard added) were used to ensure assay specificity and absence of carryover.

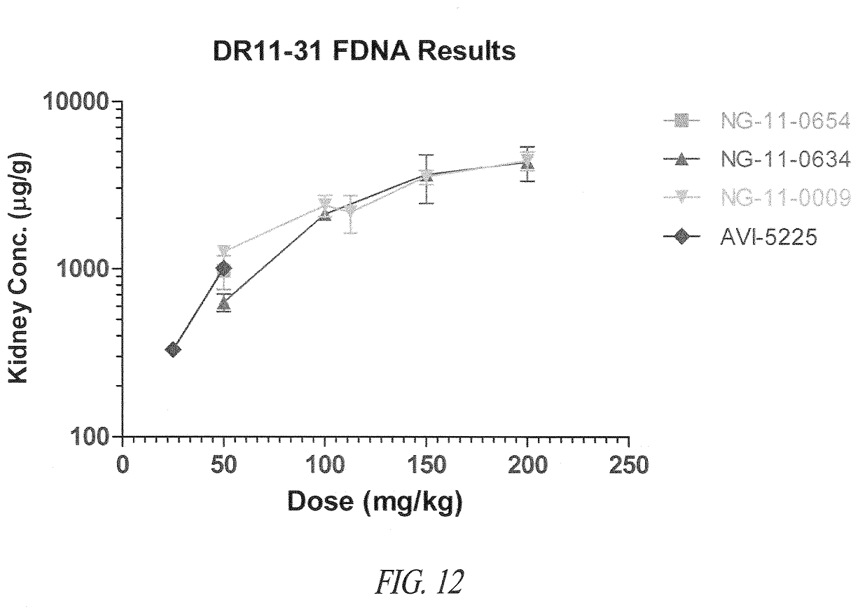

FIG. 12 shows that kidney concentrations were similar amongst the tested conjugates.

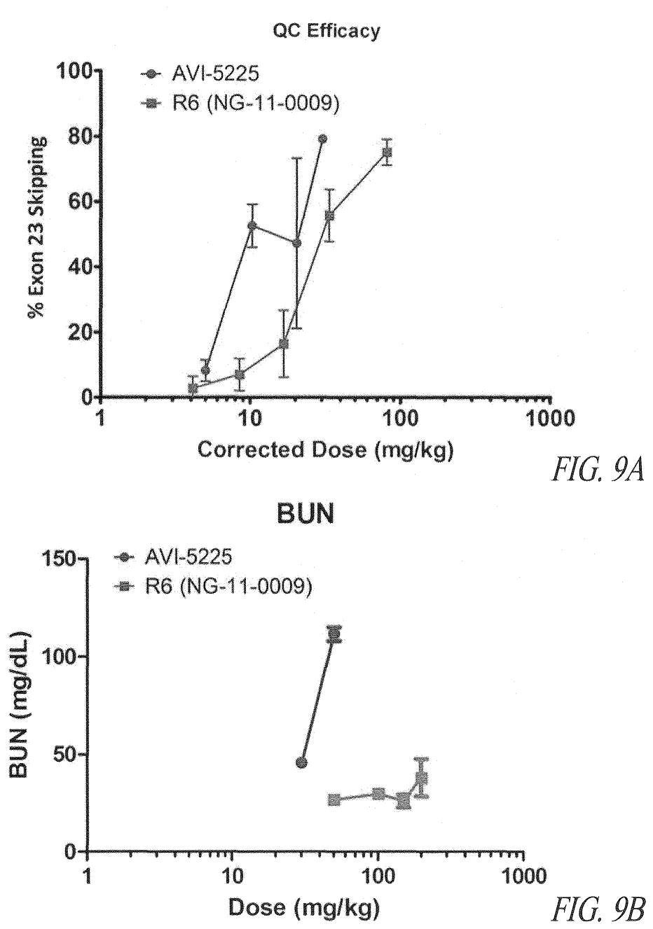

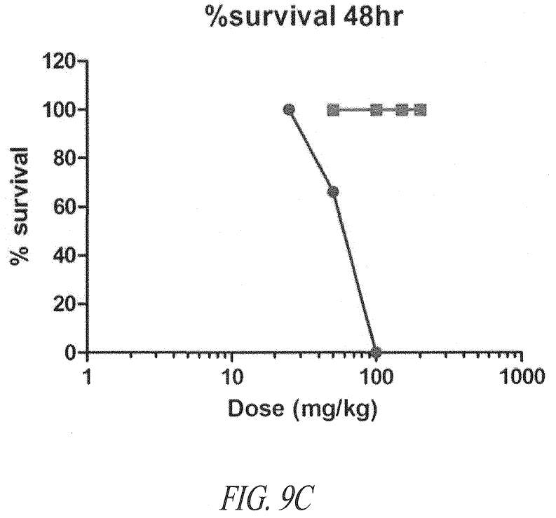

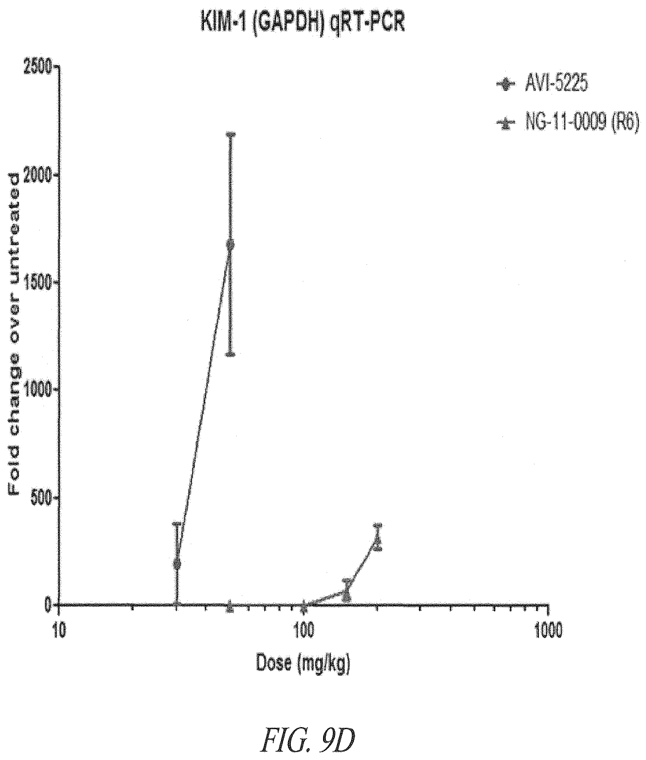

The above data shows that conjugates of the invention have similar efficacy and improved toxicity compared to other conjugates. FIGS. 9A-D summarizes these results with respect to an R₆G conjugate (NG-11-0009).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A conjugate comprising:
(a) a carrier peptide comprising amino acid subunits; and
(b) a nucleic acid analogue comprising a substantially uncharged backbone and a targeting base sequence for sequence-specific binding to a target nucleic acid;
wherein:
two or more of the amino acid subunits are positively charged amino acids, the carrier peptide comprises a glycine (G) or proline (P) amino acid subunit at a carboxy terminus of the carrier peptide, no more than seven contiguous amino acid subunits are arginine, and the carrier peptide is covalently attached to the nucleic acid analogue.

2. The conjugate of claim 1, wherein the carrier peptide comprises a glycine amino acid at the carboxy terminus.

3. The conjugate of claim 1, wherein the carrier peptide comprises a proline amino acid at the carboxy terminus.

4. The conjugate of claim 1, wherein the carrier peptide comprises from 4 to 40 amino acid subunits.

5. The conjugate of claim 1, wherein the carrier peptide comprises from 6 to 20 amino acid subunits.

6. The conjugate of claim 1, wherein the positively charged amino acids are histidine (H), lysine (K), arginine (R) or combinations thereof.

7. The conjugate of claim 1, wherein at least one of the positively charged amino acids is arginine.

8. The conjugate of claim 1, wherein each of the amino acid subunits, except the carboxy terminal glycine or proline, are positively charged amino acids.

9. The conjugate of claim 1, wherein each of the positively charged amino acids is arginine.

10. The conjugate of claim 1, wherein at least one of the positively charged amino acids is an arginine analog, the arginine analog being a cationic α-amino acid comprising a side chain of the structure R^{a}N═C(NH₂)R^{b}, where R^{a} is H or R^{c}; R^{b} is R^{c}, NH₂, NHR, or N(R^{c})₂, where R^{c} is lower alkyl or lower alkenyl and optionally comprises oxygen or nitrogen or R^{a} and R^{b} may together form a ring; and wherein the side chain is linked to the amino acid via R^{a} or R^{b}.

11. The conjugate of claim 1, wherein at least 20% of the amino acid subunits are positively charged amino acids.

12. The conjugate of claim 1, wherein at least 50% of the amino acid subunits are positively charged amino acids.

13. The conjugate of claim 1, wherein at least 80% of the amino acid subunits are positively charged amino acids.

14. The conjugate of claim 1, wherein all of the amino acid subunits, except the carboxy terminal glycine or proline are positively charged amino acids.

15. The conjugate of claim 1, wherein the carrier peptide comprises the sequence (R^{d})ₘ, wherein R^{d} is independently, at each occurrence, a positively charged amino acid and m is an integer ranging from 2 to 12.

16. The conjugate of claim 15, wherein R^{d} is independently, at each occurrence, arginine, hystidine or lysine.

17. The conjugate of claim 15, wherein each R^{d} is arginine.

18. The conjugate of claim 1, wherein each amino acid subunit, except the carboxy terminal glycine or proline, is arginine.

19. The conjugate of claim 1, wherein the carrier peptide comprises at least one hydrophobic amino acid, the hydrophobic amino acid comprising a substituted or unsubstituted alkyl, alkenyl, alkynyl, aryl or aralkyl side chain wherein the alkyl, alkenyl and alkynyl side chain includes at most one heteroatom for every six carbon atoms.

20. The conjugate of claim 19, wherein the carrier peptide comprises two or more hydrophobic amino acids.

21-65. (canceled)
